(19) United States
(12) Patent Application Publication
(10) Pub. No.: US 2024/0327351 A1
(43) Pub. Date: Oct. 3, 2024

(54) AMIDE AND ETHER SUBSTITUTED N-(1H-INDOL-7-YL)BENZENESULFONAMIDES AND USES THEREOF

(71) Applicant: TRIANA BIOMEDICINES, INC., Boston, MA (US)

(72) Inventors: William J. Greenlee, Teaneck, NJ (US); Nicholas Calandra, Weston, MA (US); Soumya Ray, Quincy, MA (US); Arthur F. Kluge, Gainesville, FL (US)

(21) Appl. No.: 18/276,433

(22) PCT Filed: Feb. 9, 2022
(86) PCT No.: PCT/US2022/015786

§ 371 Date: Aug. 8, 2023

Related U.S. Application Data
(60) Provisional application No. 63/147,430, filed on Feb. 9, 2021.

(51) Int. Cl. C07D 209/30; A61K 31/404; A61K 31/4155; A61K 31/4178; A61K 31/4184; A61K 31/4192; A61K 31/4196; A61K 31/423; A61K 31/4245; A61K 31/437; A61K 31/438; A61K 31/4439; A61K 31/454; A61K 31/4545; A61K 31/4709; A61K 31/496; A61K 31/497; A61K 31/506; A61K 31/5377; A61K 31/5386; A61K 31/55; C07D 401/12; C07D 401/14; C07D 403/12; C07D 403/14; C07D 405/12; C07D 409/12; C07D 413/12; C07D 471/04; C07D 491/107; C07D 498/10

(52) U.S. Cl. CPC C07D 209/30; A61K 31/404; A61K 31/4155; A61K 31/4178; A61K 31/4184; A61K 31/4192; A61K 31/4196; A61K 31/423; A61K 31/4245; A61K 31/437; A61K 31/438; A61K 31/4439; A61K 31/454; A61K 31/4545; A61K 31/4709; A61K 31/496; A61K 31/497; A61K 31/506; A61K 31/5377; A61K 31/5386; A61K 31/55; C07D 401/12; C07D 401/14; C07D 403/12; C07D 403/14; C07D 405/12; C07D 409/12; C07D 413/12; C07D 471/04; C07D 491/107; C07D 498/10

(57) ABSTRACT

The present application discloses novel compounds, pharmaceutical compositions containing these compounds and methods of inducing degradation of a protein, comprising contacting the protein with an effective amount of a compound of the disclosure. Methods of treating disease and disorders that results from abnormal activity of a target protein in a subject, are also disclosed.

### RELATED APPLICATIONS

This application claims the benefit of priority to U.S. Provisional Application No. 63/147,430, filed Feb. 9, 2021, the entire contents of which are incorporated herein by reference.

### SUMMARY OF THE INVENTION

Various embodiments provide novel compounds, pharmaceutical compositions comprising such compounds, and methods of inducing degradation of a protein.

Some embodiments of the present disclosure describe a compound Formula I

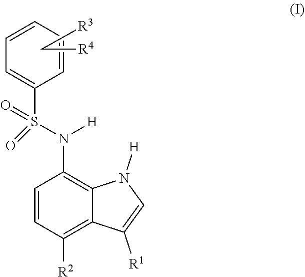

or a pharmaceutically acceptable salt thereof, wherein:
R¹ is selected from the group consisting of chloro, bromo, fluoro, and iodo;
R² is selected from the group consisting of H, chloro, fluoro, and methyl;
R³ is selected from the group consisting of H, chloro, fluoro, cyano, and methyl;
R⁴ is selected from the group consisting of
   (a)

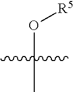

wherein:
   R⁵ is selected from the group consisting of optionally substituted C₃ to C₇ cycloalkyl, optionally substituted 4 to 12-membered heterocyclyl, optionally substituted C₆-C₁₀ aryl, and substituted C₁-C₈ alkyl;
   wherein the C₁-C₈ alkyl is substituted with at least one substituent selected from the group consisting of halo, C₁-C₆ alkoxy, (optionally substituted C₃-C₇ cycloalkyl amino)carbonyl, (C₁-C₆ alkoxycarbonyl)(C₁-C₆ alkyl)amino, (C₁-C₆ alkyl)(C₁-C₆ alkyl)N—, C₁-C₆ alkylsulfonyl, optionally substituted 4 to 8-membered heterocyclyl, optionally substituted benzo[d][1,3]dioxolyl, optionally substituted benzooxazolonyl, optionally substituted tetrahydroquinolinyl, optionally substituted tetrahydroisoquinolinyl, optionally substituted C₆-C₁₀ aryl, optionally substituted isoindolinyl, and optionally substituted 5 to 10-membered heteroaryl;
(b)

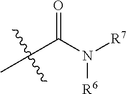

wherein
   R⁶ is selected from the group consisting of H and CH₃;
   R⁷ is selected from the group consisting of optionally substituted C₃-C₇ cycloalkyl, optionally substituted 2,3-dihydro-1H-indenyl, optionally substituted 3 to 7-membered heterocyclyl, and substituted C₁-C₈ alkyl;
   wherein the C₁-C₈ alkyl is substituted with at least one substituent selected from the group consisting of optionally substituted C₁-C₆ alkoxy, optionally substituted 4 to 8-membered heterocyclyoxy, —C(O)NR⁸R⁹, C₁-C₆ alkylsulfonyl, optionally substituted C₃-C₇ cycloalkyl, optionally substituted bicyclooctatrienyl, optionally substituted 4 to 8-membered heterocyclyl, optionally substituted C₆-C₁₀ aryl, optionally substituted dihydrobenzodioxinyl, and optionally substituted 5 to 10-membered heteroaryl;
   wherein R⁸ and R⁹ together with the nitrogen atom to which they are attached form an optionally substituted 5-6 membered heterocyclyl; or alternatively,
   R⁶ and R⁷ together with the nitrogen atom to which they are attached form a group selected from optionally substituted 4 to 12-membered optionally substituted heterocyclyl, and optionally substituted 4,5,6,7-tetrahydro-1H-imidazo[4,5-c]pyridinyl; with the proviso that the compound is not

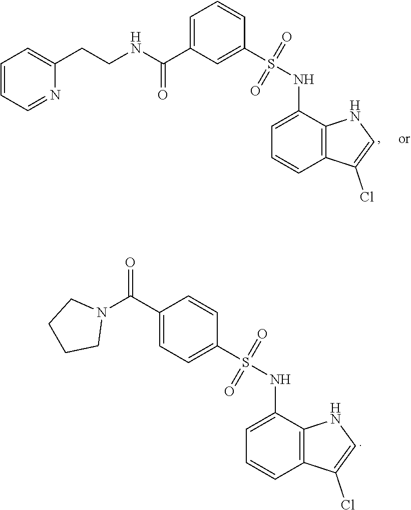 [SMILES: O=C(NCCc1ccccn1)c1cccc(S(=O)(=O)Nc2cccc3c(Cl)c[nH]c23)c1 ; O=C(c1ccc(S(=O)(=O)Nc2cccc3c(Cl)c[nH]c23)cc1)N1CCCC1]

Some embodiments of the present disclosure are directed to a compound of Formula II

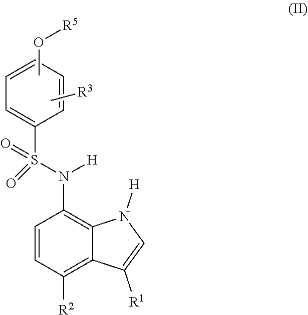

or a pharmaceutically acceptable salt thereof, wherein
R¹ is selected from the group consisting of chloro, bromo, fluoro, and iodo;
R² is selected from the group consisting of H, chloro, fluoro, and methyl;
R³ is selected from the group consisting of H, chloro, fluoro, cyano, and methyl;
R⁵ is selected from the group consisting of optionally substituted C₃ to C₇ cycloalkyl, optionally substituted 4 to 12-membered heterocyclyl, optionally substituted C₆-C₁₀ aryl, and substituted C₁-C₈ alkyl;
wherein, the C₁-C₈ alkyl is substituted with at least one substituent selected from the group consisting of halo, C₁-C₆ alkoxy, (optionally substituted C₃-C₇ cycloalkyl amino)carbonyl, (C₁-C₆ alkoxycarbonyl)(C₁-C₆ alkyl)amino, —N(C₁-C₆ alkyl)(C₁-C₆ alkyl), C₁-C₆ alkylsulfonyl, optionally substituted 4 to 8-membered heterocyclyl, optionally substituted benzo[d][1,3]dioxolyl, optionally substituted benzooxazolonyl, optionally substituted tetrahydroquinolinyl, optionally substituted tetrahydroisoquinolinyl, optionally substituted C₆-C₁₀ aryl, optionally substituted isoindolinyl, and optionally substituted 5 to 10-membered heteroaryl.

Some embodiments of the present disclosure are directed to a compound of Formula III

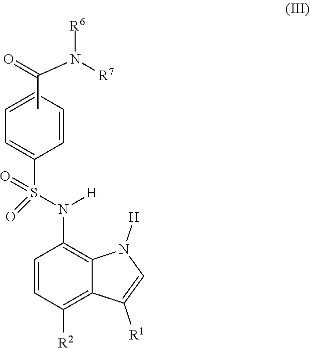

or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl;
R⁶ is selected from the group consisting of H and CH₃;
R⁷ is selected from the group consisting of optionally substituted C₃-C₇ cycloalkyl, optionally substituted 2,3-dihydro-1H-indenyl, optionally substituted 3 to 7-membered heterocyclyl, and substituted C₁-C₈ alkyl;
wherein the C₁-C₅ alkyl is substituted with at least one substituent selected from the group consisting of optionally substituted C₁-C₆ alkoxy, optionally substituted 4 to 8-membered heterocyclyoxy, —C(O)NR⁸R⁹, C₁-C₆ alkylsulfonyl, optionally substituted C₃-C₇ cycloalkyl, optionally substituted bicyclooctatrienyl, optionally substituted 4 to 8-membered heterocyclyl, optionally substituted C₆-C₁₀ aryl, optionally substituted dihydrobenzodioxinyl, and optionally substituted 5 to 10-membered heteroaryl;
wherein R⁸ and R⁹ together with the nitrogen atom to which they are attached form an optionally substituted 5-6 membered heterocyclyl; or alternatively,
R⁶ and R⁷ together with the nitrogen atom to which they are attached form a group selected from optionally substituted 4 to 12-membered optionally substituted heterocyclyl, and optionally substituted 4,5,6,7-tetrahydro-1H-imidazo[4,5-c]pyridinyl; with the proviso that the compound is not

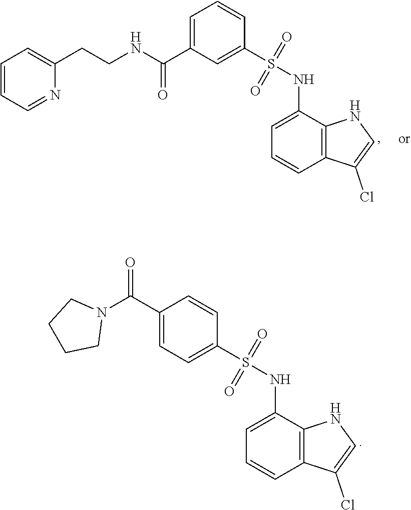 [SMILES: O=C(NCCc1ccccn1)c1cccc(S(=O)(=O)Nc2cccc3c(Cl)c[nH]c23)c1 ; O=C(c1ccc(S(=O)(=O)Nc2cccc3c(Cl)c[nH]c23)cc1)N1CCCC1]

Some embodiments of the present disclosure are directed to a compound selected from the group consisting of

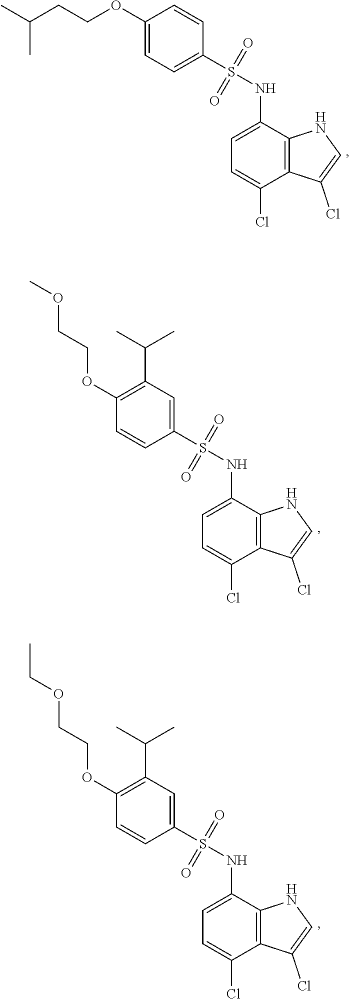 [SMILES: CC(C)CCOc1ccc(S(=O)(=O)Nc2ccc(Cl)c3c(Cl)c[nH]c23)cc1 ; COCCOc1ccc(S(=O)(=O)Nc2ccc(Cl)c3c(Cl)c[nH]c23)cc1C(C)C ; CCOCCOc1ccc(S(=O)(=O)Nc2ccc(Cl)c3c(Cl)c[nH]c23)cc1C(C)C]

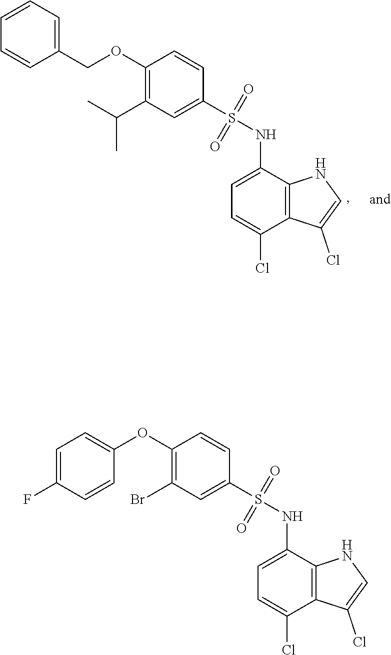 [SMILES: CC(C)c1cc(S(=O)(=O)Nc2ccc(Cl)c3c(Cl)c[nH]c23)ccc1OCc1ccccc1 ; O=S(=O)(Nc1ccc(Cl)c2c(Cl)c[nH]c12)c1ccc(Oc2ccc(F)cc2)c(Br)c1]

or a pharmaceutically acceptable salt thereof.

Some embodiments of the present disclosure are directed to a compound selected from the group consisting of

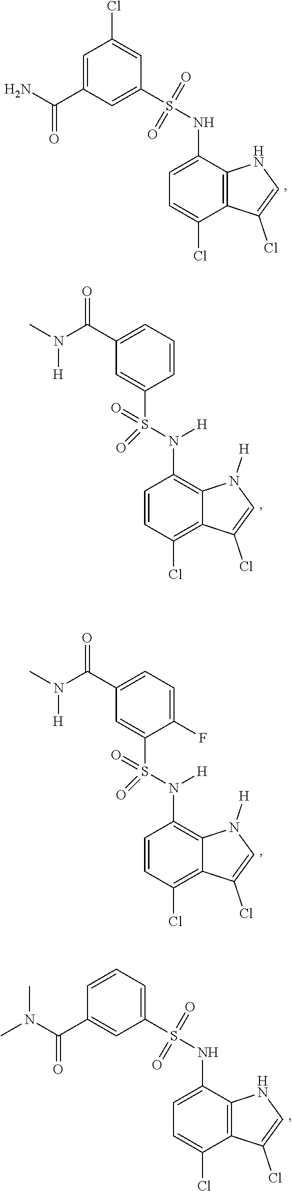 [SMILES: NC(=O)c1cc(Cl)cc(S(=O)(=O)Nc2ccc(Cl)c3c(Cl)c[nH]c23)c1 ; CNC(=O)c1cccc(S(=O)(=O)Nc2ccc(Cl)c3c(Cl)c[nH]c23)c1 ; CNC(=O)c1ccc(F)c(S(=O)(=O)Nc2ccc(Cl)c3c(Cl)c[nH]c23)c1 ; CN(C)C(=O)c1cccc(S(=O)(=O)Nc2ccc(Cl)c3c(Cl)c[nH]c23)c1]

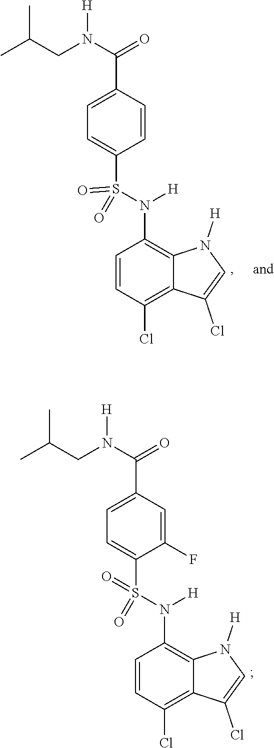 [SMILES: CC(C)CNC(=O)c1ccc(S(=O)(=O)Nc2ccc(Cl)c3c(Cl)c[nH]c23)cc1 ; CC(C)CNC(=O)c1ccc(S(=O)(=O)Nc2ccc(Cl)c3c(Cl)c[nH]c23)c(F)c1]

or a pharmaceutically acceptable salt thereof.

Some embodiments of the present disclosure are directed to a method of inducing degradation of a protein, comprising contacting the protein with an effective amount of a compound or composition according to embodiments described herein.

Some embodiments of the present disclosure are directed to a method of inducing degradation of a protein, in a patient in need thereof, comprising administering to the patient, a therapeutically effective amount of a compound according to embodiments herein.

Some embodiments of the present disclosure are directed to a method of treating a disease or disorder that results from abnormal activity of a target protein in a subject, comprising administering to the subject, a therapeutically effective amount of a compound according to embodiments herein, wherein the compound induces degradation of the protein thereby treating the disease or disorder.

Some embodiments of the present disclosure are directed to a method of treating a disease or disorder that results from abnormal expression of a gene that encodes a target protein in a subject, comprising administering to the subject, a therapeutically effective amount of a compound according to embodiments herein, wherein the compound induces degradation of the protein thereby treating the disease or disorder.

### DETAILED DESCRIPTION

Molecular glue degraders are small molecules that bring about targeted protein degradation by promoting the association of a target protein with a ubiquitin E3 ligase. This results in ubiquitination of the target protein, followed by its degradation by the proteasome. During protein degradation, the molecular glue dissociates, freeing it up to form a new target protein-E3 ligase complex. Examples of molecular glues include the IMiDs (immune modulatory drugs; e.g., thalidomide), which promote a novel interaction between a substrate (e.g., IKZF1/3) and cereblon, a substrate receptor (also known as DCAF) for Cullin-RING ubiquitin ligase 4 (CRL4). More recently, the small molecule indisulam was reported to be a molecular glue that enhances the binding of DCAF15, another CRL4 substrate receptor, to a novel substrate, the pre-mRNA splicing factor RBM39, promoting its degradation.

Molecular glues have several advantages over other therapeutic modalities for treating a variety of diseases and disorders. By using the function of specific effector proteins, in this case a ligase in a cell to eliminate a protein of interest or target, molecular glues can provide highly specific drug therapy options with fewer side effects, such as off target effects and/or toxicity. Furthermore, more than three quarters of human proteins have remained beyond the reach of therapeutic development, despite enormous efforts to advance traditional pharmacology approaches. Compounds that bring about targeted protein degradation offer one approach that can overcome this limitation. Thus, there is a need for new compounds that function as molecular glues and induce protein degradation for the treatment of diseases and disorders.

### Definitions

Unless defined otherwise, all technical and scientific terms used herein have the same meanings as commonly understood by one of ordinary skill in the art.

The articles “a” and “an” as used herein mean “one or more” or “at least one.” unless otherwise indicated. That is, reference to any element of the present invention by the indefinite article “a” or “an” does not exclude the possibility that more than one of the element is present.

As used herein, the term “about” means plus or minus 10% of the numerical value of the number with which it is being used. Therefore, about 50% means in the range of 45%-55%.

“Administering.” or “administration” and the like, when used in conjunction with the compounds of the disclosure refers to providing the compounds or pharmaceutical compositions according to any of the embodiments described herein, to a subject in need of treatment. Preferably the subject is a mammal, more preferably a human. In one aspect, the present invention comprises administering the compound or pharmaceutical composition of the invention alone or in conjunction with another therapeutic agent. When a compound or pharmaceutical composition of the invention is administered in conjunction with another therapeutic agent, the compound or pharmaceutical composition of the invention and the other therapeutic agent can be administered at the same time or different times, and by the same routes of administration or by different routes of administration.

As used herein, the term “alkyl” is intended to include both branched and straight-chain saturated aliphatic hydrocarbon groups having the specified number of carbon atoms. For example, “C₁-C₈ alkyl” or “C₁₋₈ alkyl” denotes alkyl having 1 to 8 carbon atoms. Where specified, an alkyl group can be substituted with at least one hydrogen being replaced by another chemical group. In some embodiments the one of more hydrogen atoms is replaced by a chemical group selected from any group as disclosed herein. Examples of alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl). Examples of substituted alkyl includes, but is not limited to, —CH₂N(CH₃)₂, —CH₂CH₂N(CH₃)₂, and —CH₂CH₂CH₂N(CH₃)₂. The term “animal” as used herein includes, but is not limited to, humans and non-human vertebrates such as wild, domestic and farm animals.

The term “alkoxy” or “alkyloxy” refers to an —O-alkyl group. “C₁-C₆ alkoxy” or “C₁₋₆ alkoxy” (or alkyloxy), is intended to include C₁, C₂, C₃, C₄, C₅, and C₆, alkoxy groups. In one aspect, an alkoxy group can be substituted with at least one hydrogen being replaced by another chemical group. In some embodiments the one of more hydrogen atoms is replaced by a chemical group selected from any group as disclosed herein. Examples of alkoxy groups include, but are not limited to, methoxy, ethoxy, propoxy (e.g., n-propoxy and isopropoxy), and t-butoxy.

The term “alkoxyalkyl” denotes an alkoxy group bound to an alkyl radical. For example —CH₂OCH₃ is the alkoxyalkyl group methoxymethyl. In one aspect, an alkoxyalkyl group can be substituted with at least one hydrogen being replaced by another chemical group. In some embodiments the one of more hydrogen atoms is replaced by a chemical group selected from any group as disclosed herein.

“Aryl” groups refer to monocyclic or polycyclic aromatic hydrocarbons, including, for example, phenyl, and naphthyl. “C₆-C₁₀ aryl” or “C₆₋₁₀ aryl” refers to phenyl and naphthyl. In one aspect, “aryl”, “C₆-C₁₀ aryl,” “C₆₋₁₀ aryl,” or “aromatic residue” may be substituted with 1 to 5 groups selected from —OH, —OCH₃, —Cl, —F, —Br, —I, —CN, —NO₂, —NH₂, —NH(CH₃), —N(CH₃)₂, —CF₃, —OCF₃, —C(O)CH₃, —SCH₃, —S(O)CH₃, —S(O)₂CH₃, —CH₃, —CH₂CH₃, —CO₂H, and —CO₂CH₃ or by any chemical group as defined herein.

The term “arylalkyl” refers to an aryl group bound to an alkyl radical. For example —CH₂CH₂(C₆H₄) is the arylalkyl group phenylethyl. Benzyl is another example of an arylalkyl group. In one aspect, the benzyl group can be substituted with at least one hydrogen being replaced by another chemical group. In some embodiments the one of more hydrogen atoms is replaced by a chemical group selected from any group as disclosed herein.

The term “benzyl”, as used herein, refers to a methyl group on which one of the hydrogen atoms is replaced by a phenyl group. In one aspect, said phenyl group may be substituted by one to five, preferably one to three, substituents independently selected from methyl, trifluoromethyl (—CF₃), hydroxyl (—OH), methoxy (—OCH₃), halogen, cyano (—CN), nitro (—NO₂), —CO₂Me, —CO₂Et, and —CO₂H, or any group as disclosed herein. Representative examples of benzyl group include, but are not limited to, PhCH₂—, 4-MeO—C₆H₄CH₂—, 2,4,6-tri-methyl-C₆H₂CH₂—, and 3,4-di-C₁-C₆H₃CH₂—.

“Benzyloxy” refers to the group-OCH₂(C₆H₄). In one aspect, a benzyloxy group may be substituted with at least one hydrogen being replaced by another chemical group, for example, any group disclosed herein.

The term “benzyloxy(alkyl)”, as used herein, refers to a benzyloxy group bound to an alkyl radical. For example —CH₂OCH₂(C₆H₄) is a benzyloxymethyl group. In one aspect, a benzyloxymethyl group can be unsubstituted or substituted with at least one hydrogen being replaced by another chemical group, for example, any group disclosed herein.

The term “cycloalkyl” or “cycloalkyl ring” is defined as a saturated or partially unsaturated carbocyclic ring in a single or fused carbocyclic ring system having from three to twelve ring members. In a preferred embodiment, a cycloalkyl is a ring system having three to seven ring members. In one aspect, one or more hydrogen atoms may be replaced by a substituent group selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl, and formyl or any other group as disclosed herein. Examples of a cycloalkyl group include, without limitation, cyclopropyl, cyclobutyl, cyclohexyl, and cycloheptyl.

The term “(cycloalkyl)alkoxyl” refers to an oxygen radical bound to a cycloalkyl group. For example, —O—(C₆H₁₁) is a cyclohexylalkoxy group. In one aspect, a (cycloalkyl)alkoxy group may be substituted with at least one hydrogen being replaced by another chemical group, for example, any group disclosed herein.

The term “(cycloalkyl)alkoxyalkyl refers to a cycloalkoxy group bound to an alkyl radical. In one aspect, a (cycloalkyl)alkoxyalkyl group may be unsubstituted or substituted with at least one hydrogen being replaced by another chemical group, for example, any group disclosed herein.

A “dialkylaminocarbonyl” or “caboxamido” denotes a carbonyl radical adjacent to an dialkylamino group. In one aspect, a dialkylaminocarbonyl group may be substituted with at least one hydrogen being replaced by another chemical group, for example, any group disclosed herein.

The terms “formula” and “structure” are used interchangeably herein.

The term “halo” or “halogen” refers to fluoro, chloro, bromo, and iodo.

As used herein, the term “heteroaryl” is intended to mean stable monocyclic and polycyclic aromatic hydrocarbons that include at least one heteroatom ring member, such as sulfur, oxygen, or nitrogen.

Heteroaryl groups include, without limitation, pyridinyl, pyrimidinyl, pyrazinyl, pyridazinyl, triazinyl, furanyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-oxadiazolyl, 1,2,4-thiadiazolyl, isothiazolyl, purinyl, carbazolyl, benzimidazolyl, and indolinyl. In one aspect, heteroaryl groups may be substituted with 1 to 5 groups selected from —OH, —OCH₃, —Cl, —F, —Br, —I, —CN, —NO₂, —NH₂, —NH(CH₃), —N(CH₃)₂, —CF₃, —OCF₃, —C(O)CH₃, —SCH₃, —S(O)CH₃, —S(O)₂CH₃, —CH₃, —CH₂CH₃, —CO₂H, and —CO₂CH₃, as well as any other group disclosed herein. The nitrogen atom is substituted or unsubstituted (i.e., N or NR wherein R is H or another substituent, if defined). The nitrogen and sulfur heteroatoms may optionally be oxidized (i.e., N→0 and S(O)ₚ, wherein p is 0, 1 or 2).

As used herein, the term “heteroarylalkyl” denote a heteroaryl group adjacent to an alkyl radical.

The term “heterocyclyl.” “heterocyclic” or “heterocyclyl ring” is defined as a saturated or partially unsaturated ring containing one to four hetero atoms or hetero groups selected from O. N, NH, —N(R^{Z})—, —S(O)— and —S(O)₂—, wherein R^{Z} is selected from alkyl, alkenyl, alkynyl, aryl, heteroaryl, cycloalkyl, in a single, fused, spiro or bridged heterocyclic ring system having from three to twelve ring members. In one aspect, the hetero groups in the “heterocyclyl,” “heterocyclic” or “heterocyclyl ring” are selected from O, N, NH, —S(O)— and —S(O)₂—. In one aspect, a “heterocyclyl,” “heterocyclic” or “heterocyclyl ring” may be substituted with any group as defined herein. In a preferred embodiment, a heterocyclyl is a ring system having three to seven ring members. Examples of a heterocyclyl group include, without limitation, azapan-2-on-yl, azetidinyl, diazacyclohexyl, morpholinyl, oxanyl, piperidinyl, piperidin-2-on-yl, piperazinyl, piperazin-2-one, benzodioxolanyl, benzodioxane, and pyrrolidinyl.

The term “heterocyclyloxy refers to a heterocyclyl bound to an oxygen radical.

The phrase “molecular glue” as used herein is used to describe a compound that induces an interaction between a substrate receptor of an E3 ligase and a target protein, leading to degradation of the protein.

The phrase “pharmaceutically acceptable” refers to those compounds, materials, pharmaceutical compositions, and/or dosage forms that are, within the scope of sound medical judgment, generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal government or a state government or listed in the U.S. Pharmacopoeia or other generally recognized pharmacopoeia for use in animals, and more particularly in humans.

As used herein, the term “pharmaceutically acceptable carrier” is a substrate used to deliver the compounds of the invention. Carriers as used herein include excipients and diluents and may depend upon the mode of administration of the compounds as described herein. Carriers for example may be edible carriers, liquid carries, carriers that will protect the compounds against rapid elimination from the body, as well as liposomal suspensions. Carriers are well known in the art and include, for example, phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

As used herein, “pharmaceutically acceptable salts” refer to non-toxic acid or base salts of the disclosed compounds. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, P A, 1990, the disclosure of which is hereby incorporated by reference.

The terms “subject,” “individual” or “patient” are used interchangeably and as used herein are intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients. The methods are particularly suitable for treating human patients having a condition, disease or disorder described herein.

As used herein, the term “therapeutic” means an agent utilized to treat, combat, ameliorate, prevent or improve an unwanted condition or disease of a patient.

A “therapeutically effective amount” of a compound, pharmaceutically acceptable salt thereof or pharmaceutical composition according to any embodiment described herein, is an amount sufficient to produce a selected effect on at least one symptom or parameter of a specific disease or disorder. The therapeutic effect may be objective (i.e., measurable by some test or marker) or subjective (i.e., subject gives an indication of or feels an effect or physician observes a change). The effect contemplated herein, includes both medical therapeutic and/or prophylactic treatment, as appropriate. The specific dose of a compound administered according to this disclosure to obtain therapeutic and/or prophylactic effects is determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment. The therapeutically effective amount administered will be determined by the physician in the light of the foregoing relevant circumstances and the exercise of sound medical judgment. A therapeutically effective amount of a compound, according to any embodiment described herein, is typically an amount such that when it is administered in a physiologically tolerable excipient composition, it is sufficient to achieve an effective systemic concentration or local concentration in the tissue.

The terms “treat,” “treated,” or “treating” as used herein, refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment. In one aspect, the treatment methods described herein are therapeutic treatments.

### Compounds

Some embodiments of the present disclosure describe a compound Formula

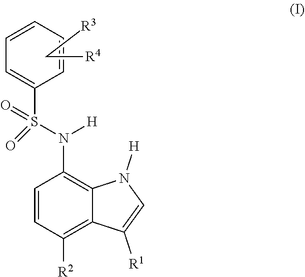

or a pharmaceutically acceptable salt thereof, wherein:
R¹ is selected from the group consisting of chloro, bromo, fluoro, and iodo;
R² is selected from the group consisting of H, chloro, fluoro, and methyl;
R³ is selected from the group consisting of H, chloro, fluoro, cyano, and methyl;
R⁴ is selected from the group consisting of
(a)

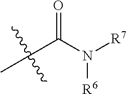

wherein:
   R⁵ is selected from the group consisting of optionally substituted C₃ to C₇ cycloalkyl, optionally substituted 4 to 12-membered heterocyclyl, optionally substituted C₆-C₁₀ aryl, and substituted C₁-C₈ alkyl;
   wherein the C₁-C₈ alkyl is substituted with at least one substituent selected from the group consisting of halo, C₁-C₆ alkoxy, (optionally substituted C₃-C₇ cycloalkyl amino)carbonyl, (C₁-C₆ alkoxycarbonyl)(C₁-C₆ alkyl)amino, (C₁-C₆ alkyl)(C₁-C₆ alkyl)N—, C₁-C₆ alkylsulfonyl, optionally substituted 4 to 8-membered heterocyclyl, optionally substituted benzo[d][1,3]dioxolyl, optionally substituted benzooxazolonyl, optionally substituted tetrahydroquinolinyl, optionally substituted tetrahydroisoquinolinyl, optionally substituted C₆-C₁₀ aryl, optionally substituted isoindolinyl, and optionally substituted 5 to 10-membered heteroaryl;
(b)

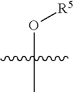

wherein
   R⁶ is selected from the group consisting of H and CH₃;
   R⁷ is selected from the group consisting of optionally substituted C₃-C₇ cycloalkyl, optionally substituted 2,3-dihydro-1H-indenyl, optionally substituted 3 to 7-membered heterocyclyl, and substituted C₁-C₈ alkyl;
   wherein the C₁-C₅ alkyl is substituted with at least one substituent selected from the group consisting of optionally substituted C₁-C₆ alkoxy, optionally substituted 4 to 8-membered heterocyclyoxy, —C(O)NR⁸R⁹, C₁-C₆ alkylsulfonyl, optionally substituted C₃-C₇ cycloalkyl, optionally substituted bicyclooctatrienyl, optionally substituted 4 to 8-membered heterocyclyl, optionally substituted C₆-C₁₀ aryl, optionally substituted dihydrobenzodioxinyl, and optionally substituted 5 to 10-membered heteroaryl;
   wherein R⁸ and R⁹ together with the nitrogen atom to which they are attached form an optionally substituted 5-6 membered heterocyclyl; or alternatively,
   R⁶ and R⁷ together with the nitrogen atom to which they are attached form a group selected from optionally substituted 4 to 12-membered optionally substituted heterocyclyl, and optionally substituted 4,5,6,7-tetrahydro-1H-imidazo[4,5-c]pyridinyl; with the proviso that the compound is not

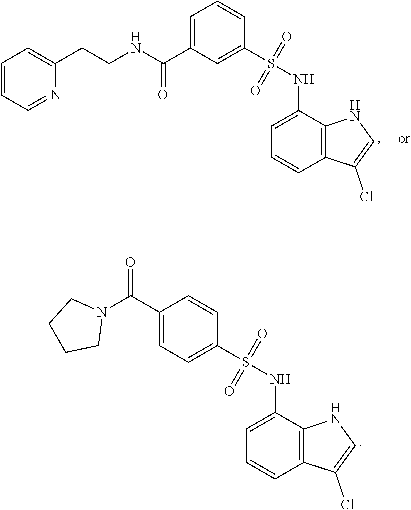 [SMILES: O=C(NCCc1ccccn1)c1cccc(S(=O)(=O)Nc2cccc3c(Cl)c[nH]c23)c1 ; O=C(c1ccc(S(=O)(=O)Nc2cccc3c(Cl)c[nH]c23)cc1)N1CCCC1]

### Compounds of Formula II

In some embodiments the compound of Formula I is a compound of Formula II

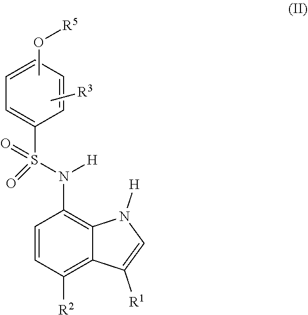

or a pharmaceutically acceptable salt thereof.

Substituent R¹ of Formula II is selected from the group consisting of chloro, bromo, fluoro, and iodo. In some embodiments, R¹ is selected from the group consisting of chloro, bromo, and iodo. In some embodiments, R¹ is chloro.

Substituent R² of Formula II is selected from the group consisting of H, chloro, fluoro and methyl. In some embodiments R² of Formula II is selected from the group consisting of hydrogen and chloro.

Substituent R³ of Formula II is selected from the group consisting of H, chloro, fluoro, cyano and methyl. In some embodiments R³ is selected from the group consisting H and F. In some embodiments, R³ is H. In some embodiments, R³ is F.

Substituent R⁵ of Formula II is selected from the group consisting of optionally substituted C₃ to C₇ cycloalkyl, optionally substituted 4 to 12-membered heterocyclyl, optionally substituted C₆-C₁₀ aryl, and substituted C₁-C₈ alkyl. The C₁-C₈ alkyl of Formula II is substituted with at least one substituent selected from the group consisting of halo, C₁-C₆ alkoxy, (optionally substituted (C₃-C₇ cycloalkylamino)carbonyl, (C₁-C₆ alkoxycarbonyl)(C₁-C₆ alkyl)amino, —N(C₁-C₆ alkyl)(C₁-C₆ alkyl), C₁-C₆ alkylsulfonyl, optionally substituted 4 to 8-membered heterocyclyl, optionally substituted benzo[d][1,3]dioxolyl, optionally substituted benzooxazolonyl, optionally substituted tetrahydroquinolinyl, optionally substituted tetrahydroisoquinolinyl, optionally substituted C₆-C₁₀ aryl, optionally substituted isoindolinyl, and optionally substituted 5 to 10-membered heteroaryl.

In some embodiments, R⁵ of Formula II is selected from the group consisting of:
monocyclic or bicyclic optionally substituted 6-membered cycloalkyl; optionally substituted 6 to 11-membered heterocyclyl; optionally substituted phenyl;
C₁ alkyl substituted with at least one substituent selected from the group consisting of halo, (optionally substituted cyclopropylamino)carbonyl, 4 to 8-membered optionally substituted heterocyclyl, optionally substituted phenyl, optionally substituted isoindolinyl, and optionally substituted 5 to 9-membered heteroaryl;
C₂ alkyl substituted with at least one substituent selected from the group consisting of C₁-C₆ alkoxy, C₁-C₆ alkylsulfonyl, optionally substituted 5 to 6-membered heterocyclyl, optionally substituted phenyl, and optionally substituted 9-membered heteroaryl;
C₃ alkyl substituted with at least one substituent selected from the group consisting of optionally substituted 5 to 7-membered heterocyclyl, optionally substituted benzo[d][1,3]dioxolyl, optionally substituted benzooxazolonyl, optionally substituted tetrahydroquinolinyl, and optionally substituted 5 to 9-membered heteroaryl;
C₄ alkyl substituted with at least one substituent selected from the group consisting of optionally substituted 6-membered heterocyclyl, optionally substituted tetrahydroquinolinyl, optionally substituted 5 to 6-membered heteroaryl;
C₅ alkyl substituted with at least one substituent selected from the group consisting of (C₁-C₄ alkoxycarbonyl)(methyl) amino, optionally substituted 6-membered heterocyclyl;
C₆ alkyl substituted with at least one substituent selected from the group consisting of (C₁-C₄ alkoxycarbonyl)(methyl)amino, optionally substituted tetrahydroisoquinolinyl; and
C₈ alkyl substituted with —N(C₁-C₆ alkyl)(C₁-C₆ alkyl).

In some embodiments, R⁵ of Formula II is selected from the group consisting of:

optionally substituted cyclohexyl, bicyclo[3.1.0]hexanyl, optionally substituted piperidinyl, optionally substituted 2-azaspiro[3.3]heptyl, optionally substituted 2-azaspiro[3.3]heptyl, optionally substituted 1,4-dioxaspiro[4.5]decyl, optionally substituted -oxaspiro[4.5]decan-1-on-yl, optionally substituted 3-azaspiro[5.5]undecyl, optionally substituted phenyl;
C₁ alkyl substituted with at least one substituent selected from the group consisting of halo, (optionally substituted cyclopropylamino)carbonyl, optionally substituted oxetanyl, optionally substituted piperidinyl, optionally substituted azabicyclo[3.1.0]hexyl, optionally substituted octahydrocyclopenta[c]pyrrolyl, optionally substituted phenyl, optionally substituted isoindolinyl, optionally substituted triazolyl, and optionally substituted benzotriazolyl,
C₂ alkyl substituted with at least one substituent selected from the group consisting of methoxy, ethoxy, ethylsulfonyl, optionally substituted pyrrolidinyl, optionally substituted piperidinyl, optionally substituted phenyl, optionally substituted indolyl, and optionally substituted isoindolinyl;
C₃ alkyl substituted with at least one substituent selected from the group consisting of optionally substituted pyrrolidinyl, optionally substituted piperidinyl, optionally substituted piperazinyl, optionally substituted, 2-oxa-6-azaspiro[3.3]heptyl, optionally substituted benzo[d][1,3]dioxolyl, optionally substituted benzooxazolonyl, optionally substituted tetrahydroquinolinyl, optionally substituted pyrazolyl, optionally substituted triazolyl, optionally substituted pyridinyl, and optionally substituted benzoimidazolyl;
C₄ alkyl substituted with at least one substituent selected from the group consisting of optionally substituted piperidinyl, optionally substituted piperazinyl, optionally substituted thiomorpholine 1,1-dioxidyl, optionally substituted tetrahydroquinolinyl, optionally substituted pyrazolyl, and optionally substituted pyridinyl;
C₅ alkyl substituted with at least one substituent selected from the group consisting of (C₁-C₄ alkoxycarbonyl)(methyl)amino, and optionally substituted piperidinyl;
C₆ alkyl substituted with at least one substituent selected from the group consisting of (C₁-C₆ alkoxycarbonyl)(CH₃)amino, optionally substituted tetrahydroisoquinolinyl; and
C₈ alkyl substituted with —N(CH₃)₂.

In some embodiments, R⁵ of Formula II is selected from the group consisting of phenyl, —CHF₂, benzoyl, —CH₂CH₂OCH₃, —CH₂CH₂OCH₂CH₃, —CH₂CH₂S(O)₂CH₂CH₃,

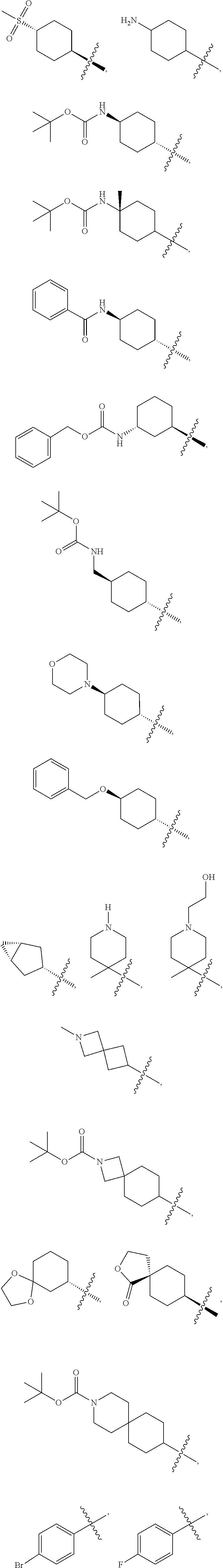

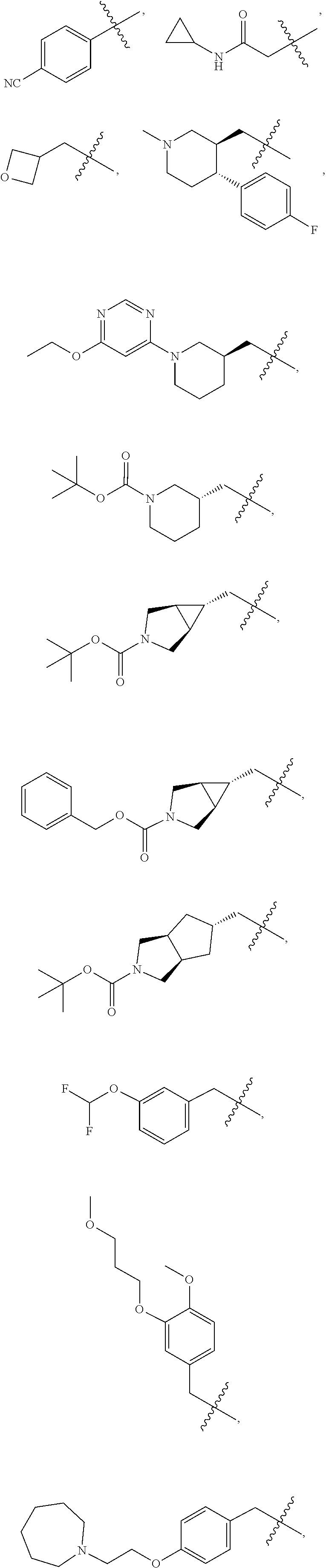

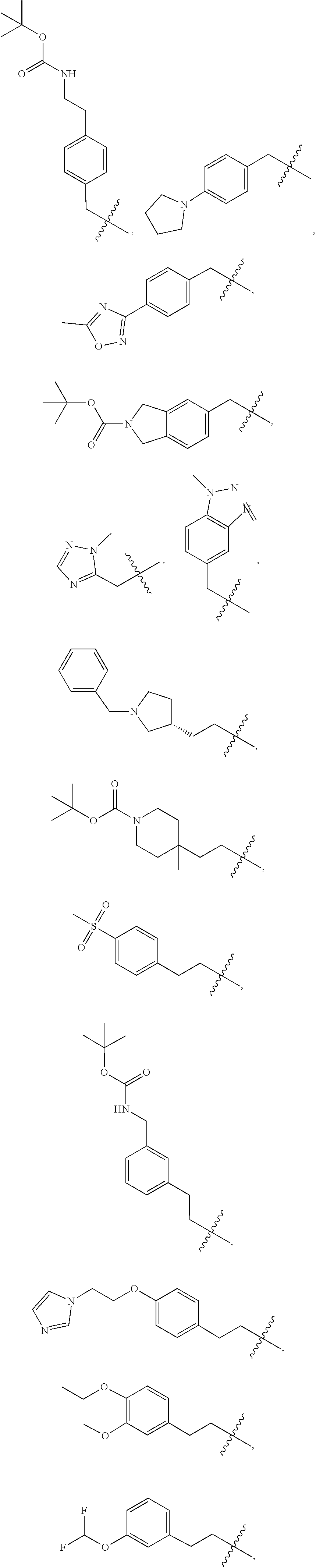

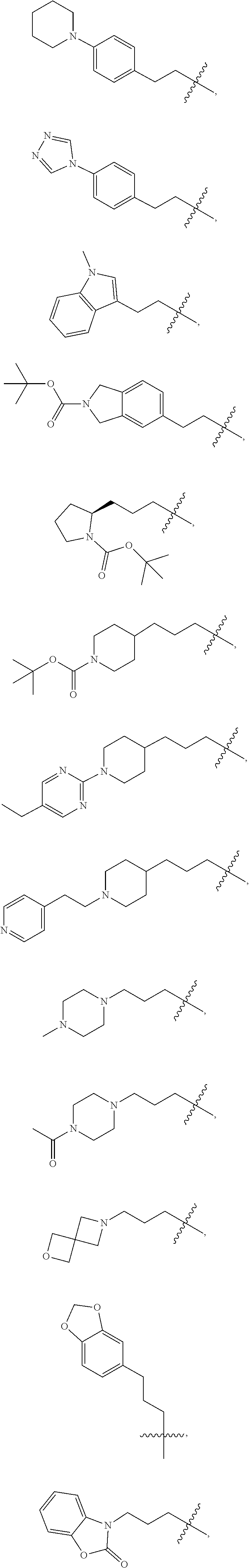

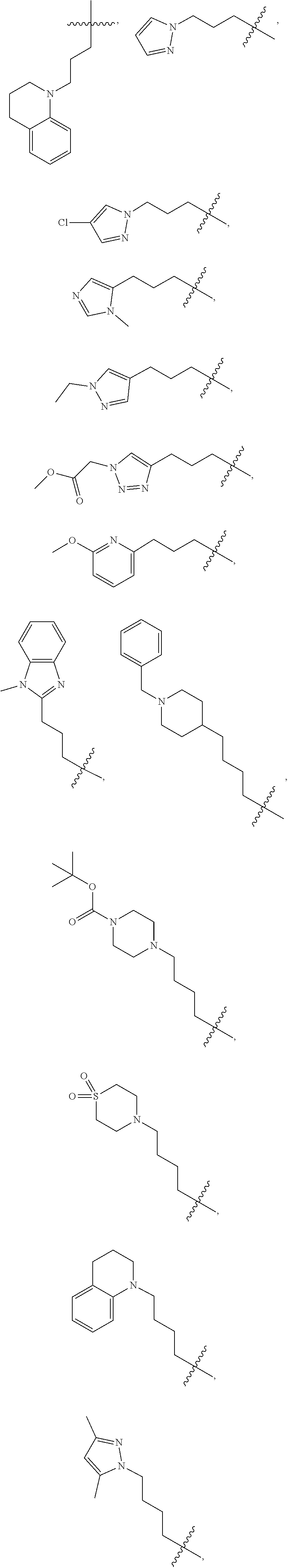

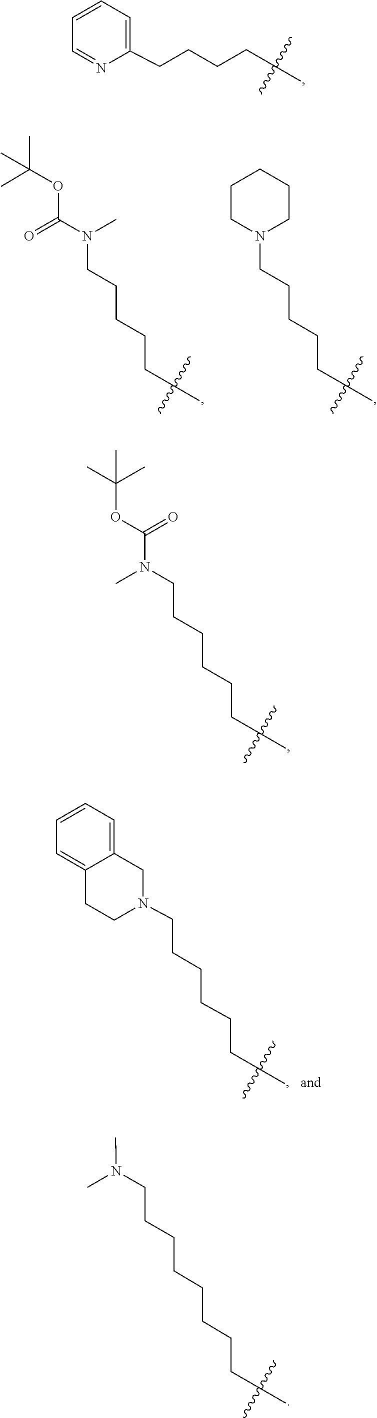

In other embodiments. R⁵ of Formula II is selected from the group consisting of phenyl, —CHF₂, benzoyl, —CH₂CH₂OCH₃, —CH₂CH₂OCH₂CH₃, —CH₂CH₂S(O)₂CH₂CH₃,

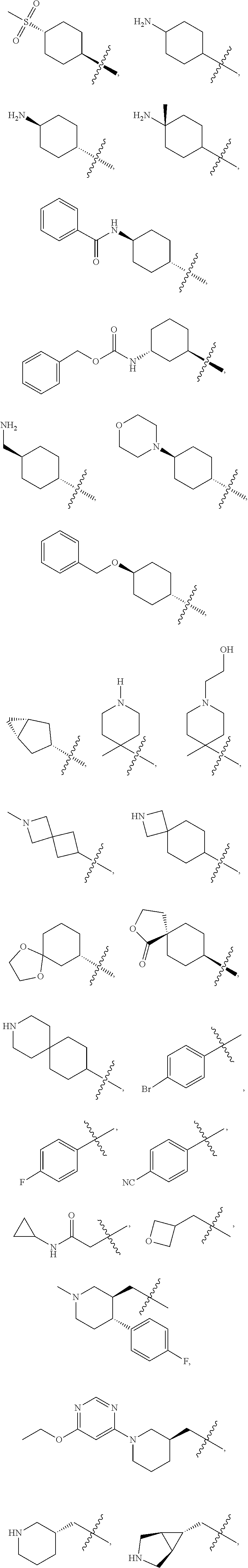

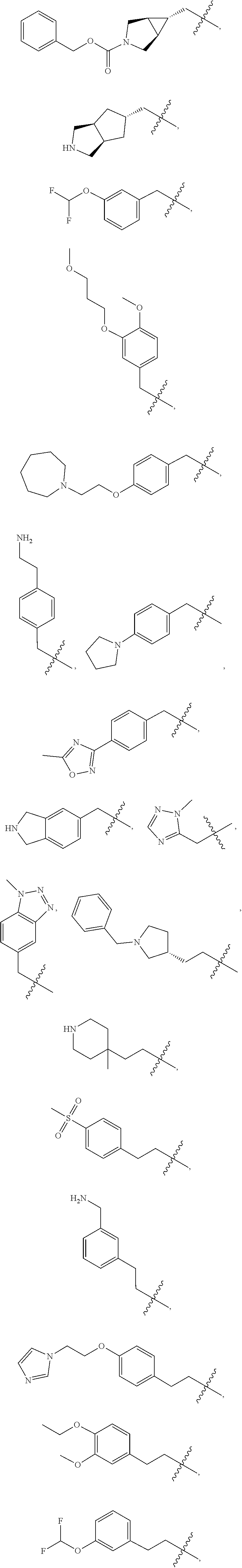

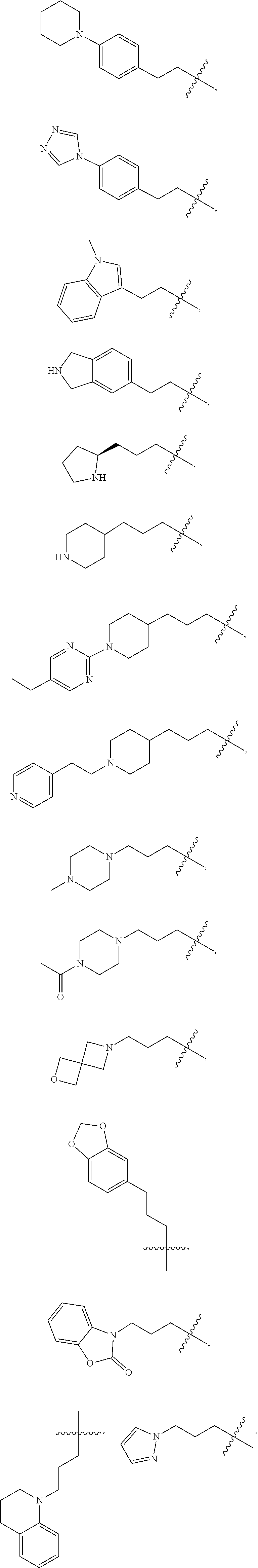

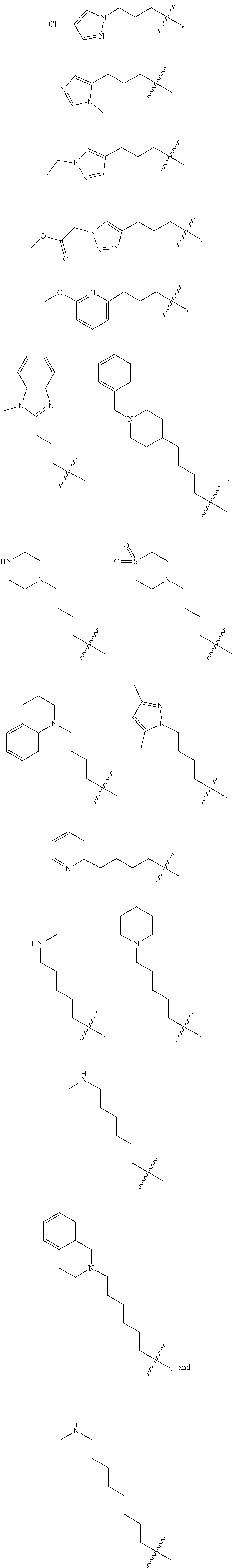

Some embodiments describe a compound of Formula II wherein
R¹ is chloro;
R² is selected from the group consisting of H and chloro;
R³ is selected from the group consisting H and P; and
R⁵ is selected from the group consisting of phenyl, —CHF₂, benzoyl, —CH₂CH₂OCH₃, —CH₂CH₂OCH₂CH₃, —CH₂CH₂S(O)₂CH₂CH₃.

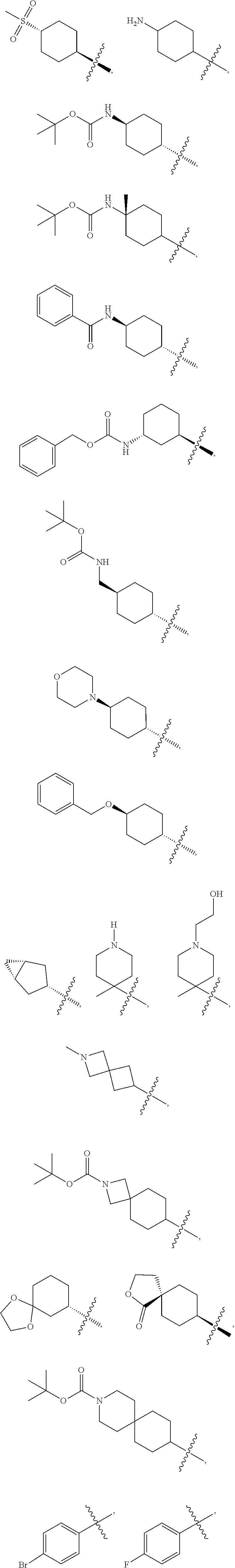

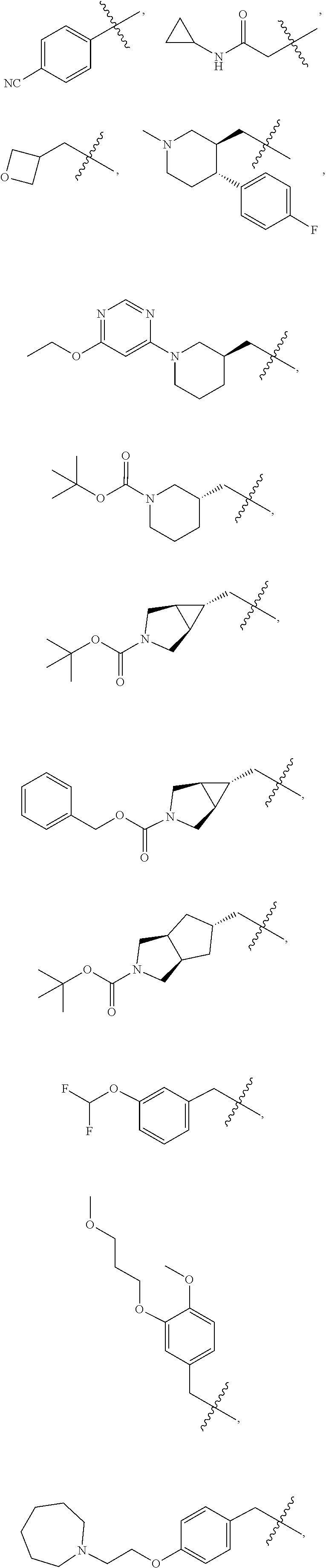

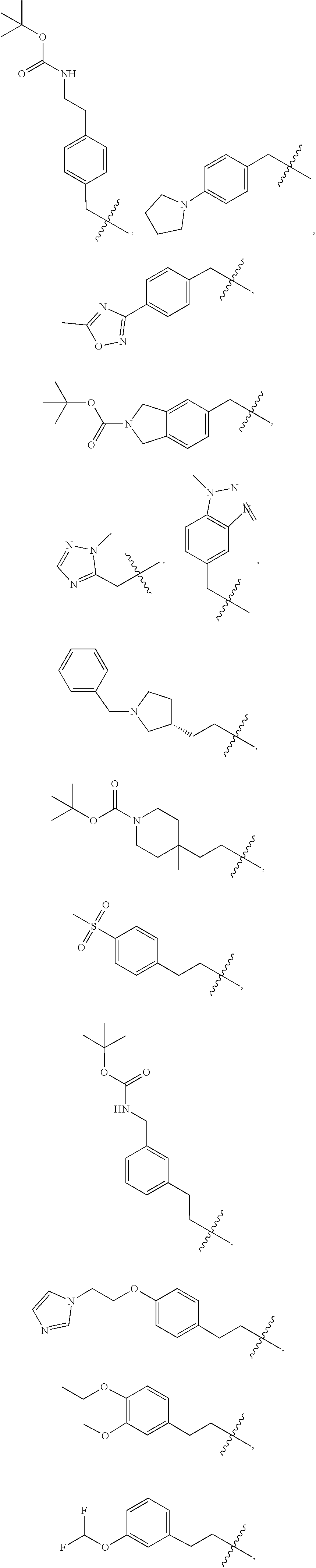

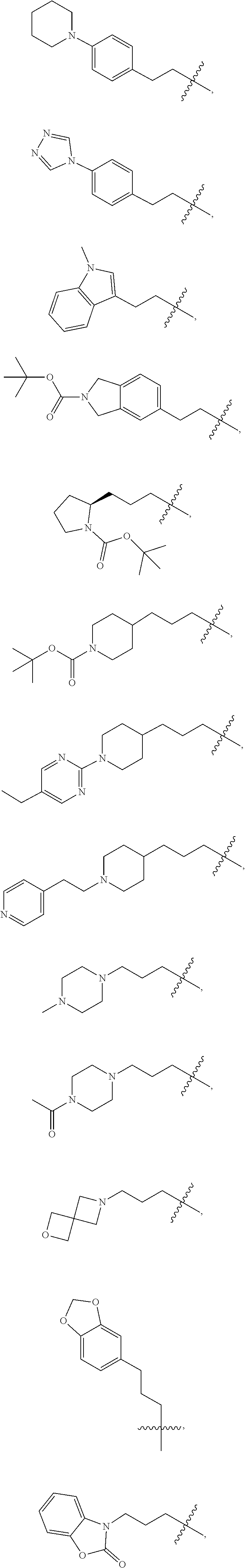

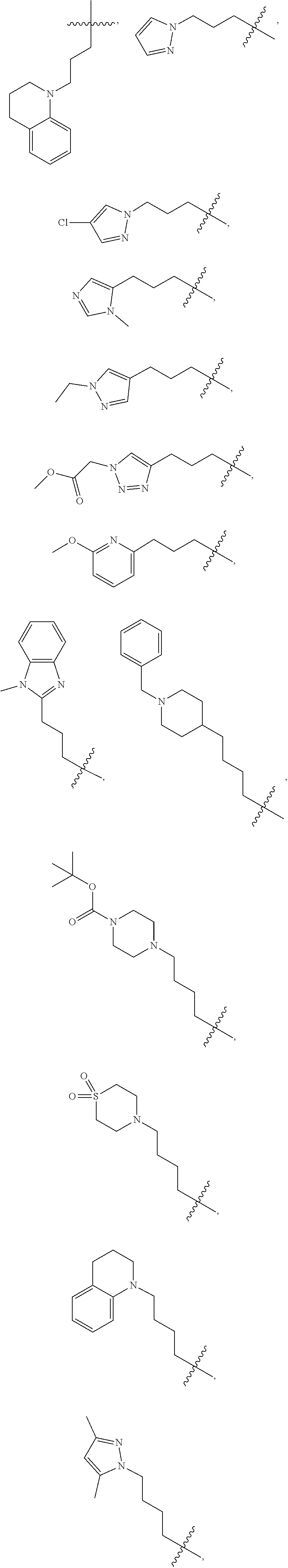

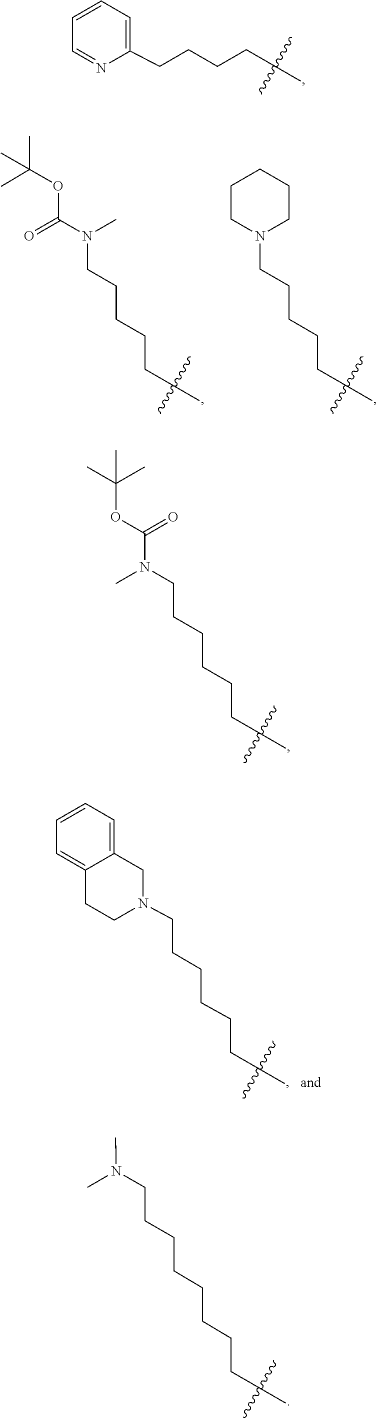

In another embodiment. R⁵ is selected from the group consisting of phenyl, —CHF₂, benzoyl, —CH₂CH₂OCH₃, —CH₂CH₂OCH₂CH₃, —CH₂CH₂S(O)₂CH₂CH₃,

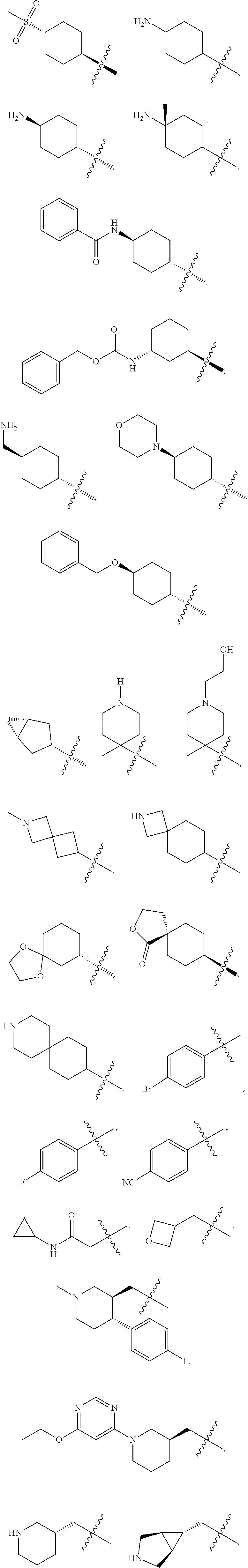

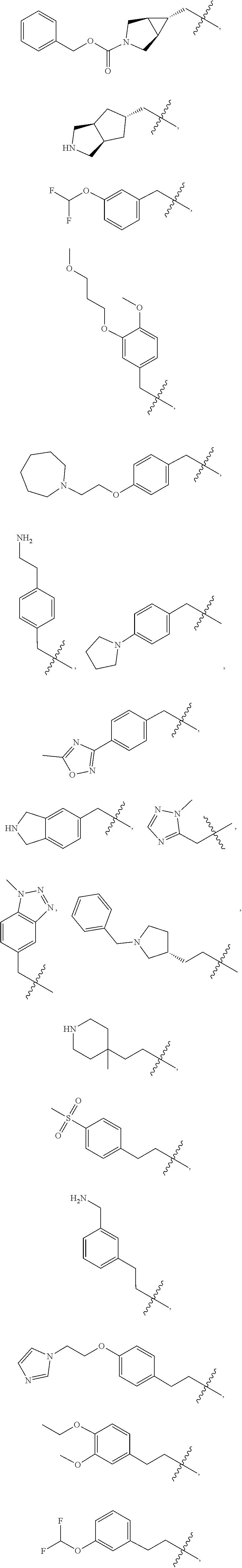

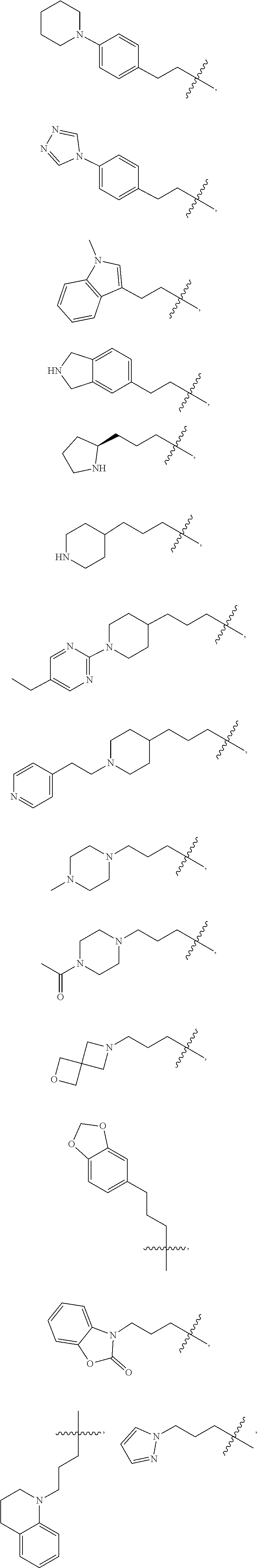

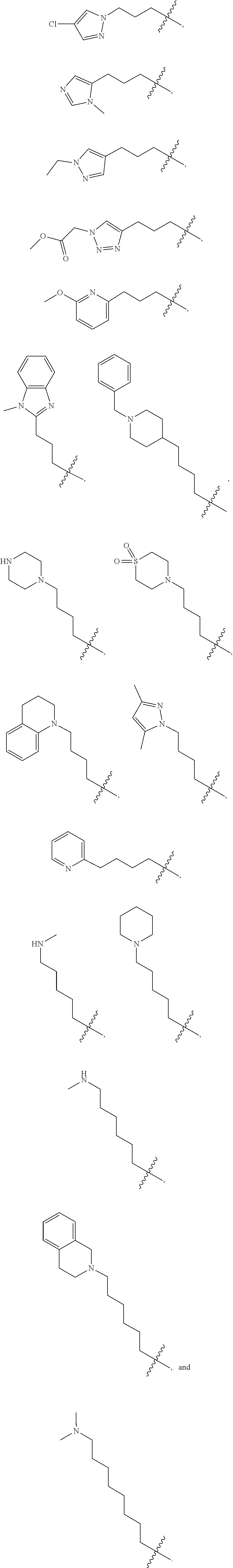

In some embodiments, the compound of Formula II is a compound of Formula IIa or Formula IIb:

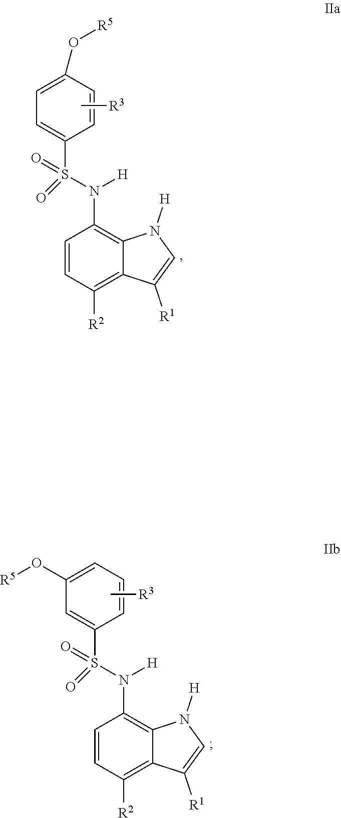

or a pharmaceutically acceptable salt thereof, wherein R¹, R² R³, and R⁵ are as described for any embodiment of the compounds of Formula II.

In some embodiments a compound of Formula II is a compound selected from any one of the following:

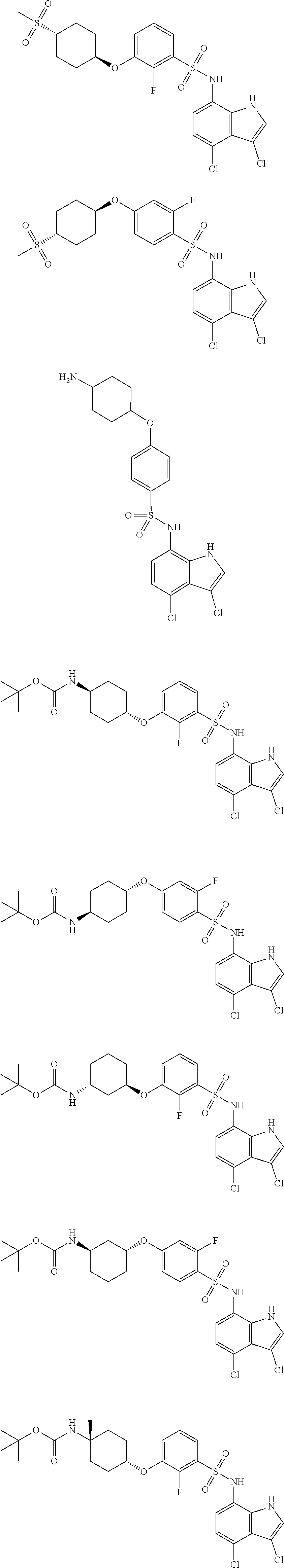

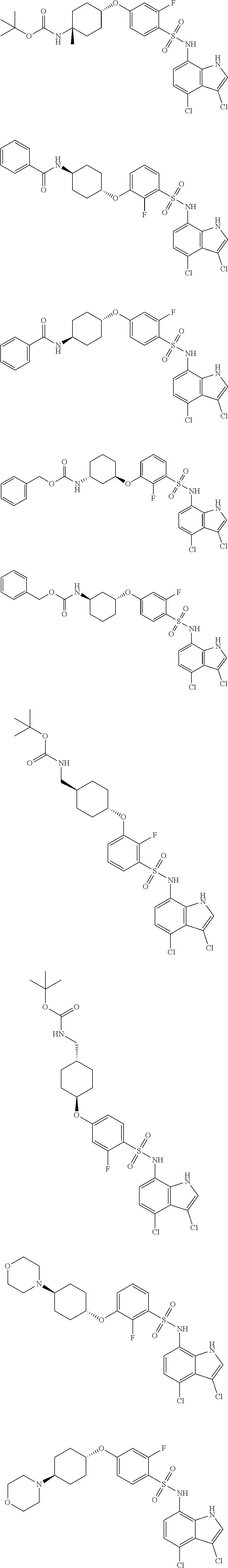

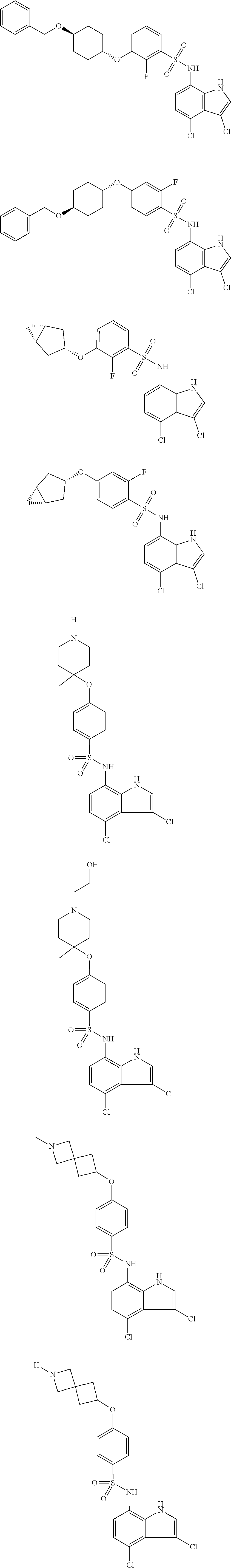

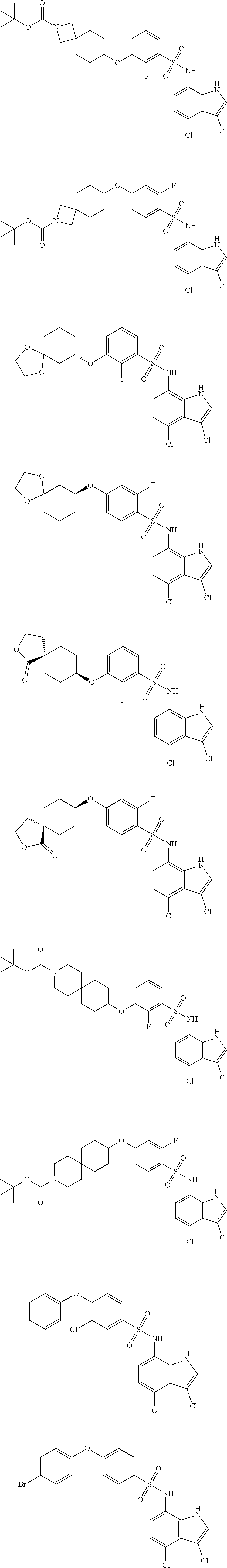

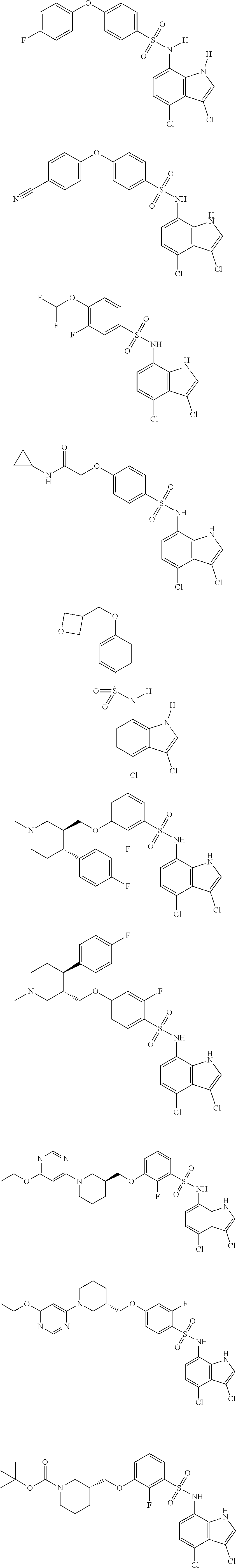

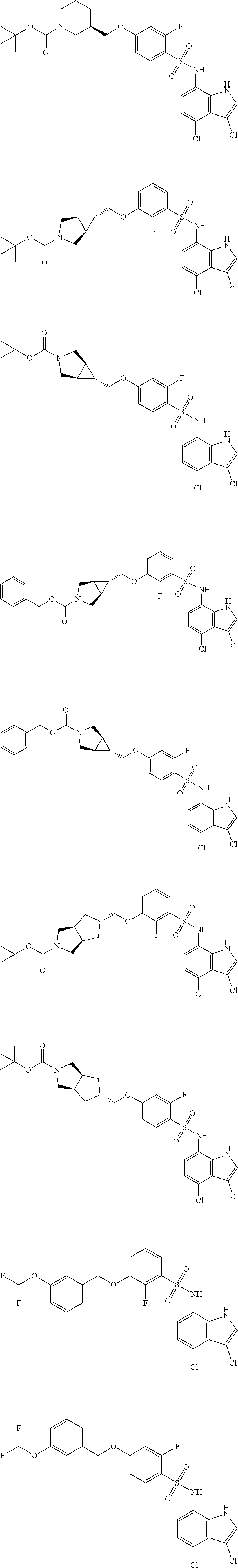

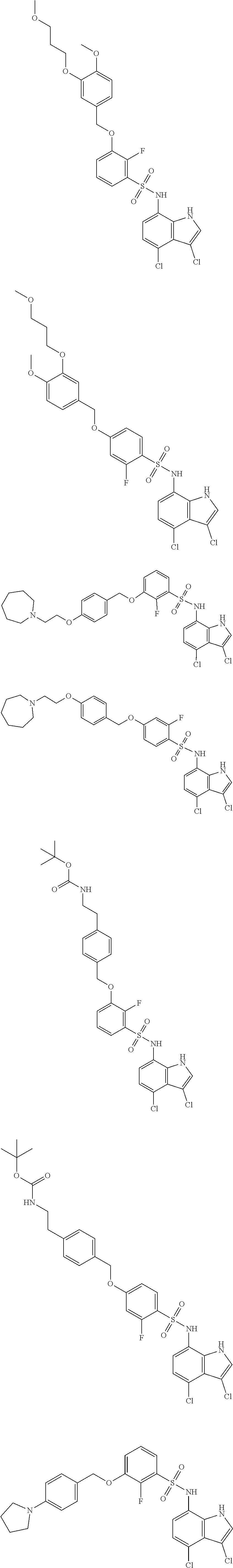

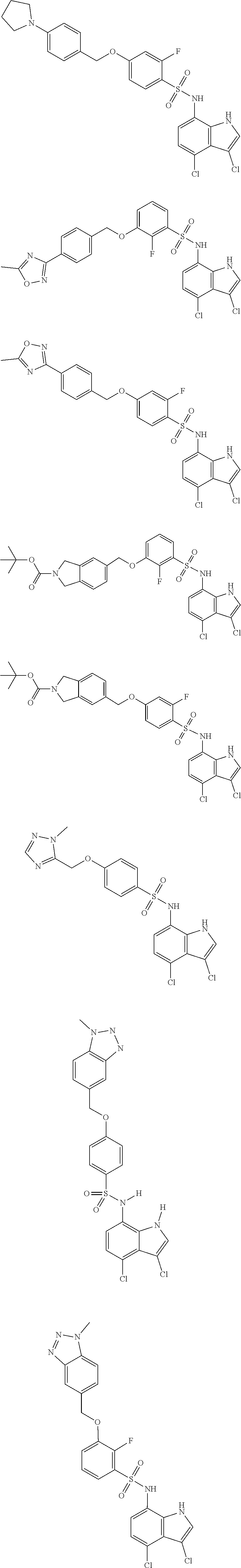

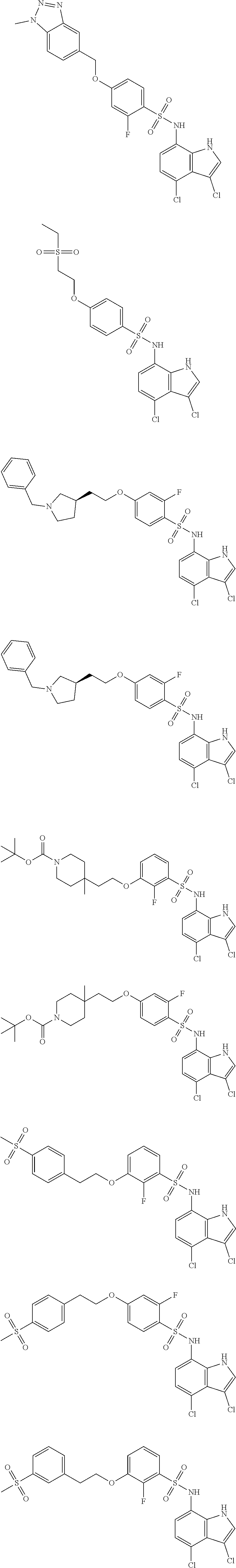

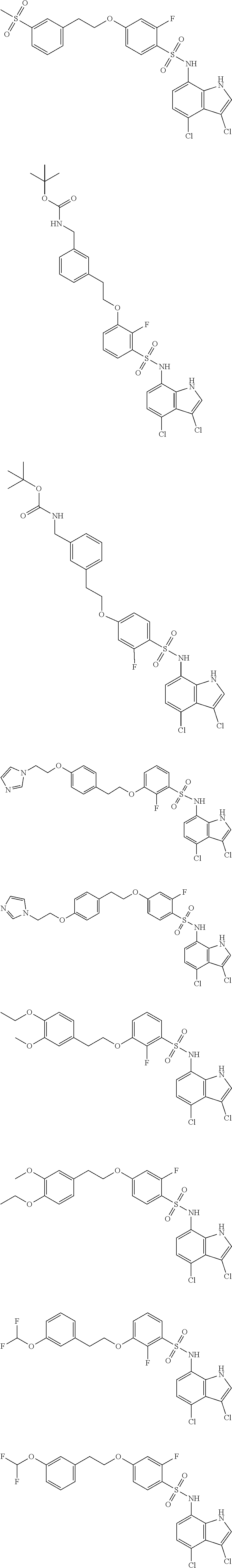

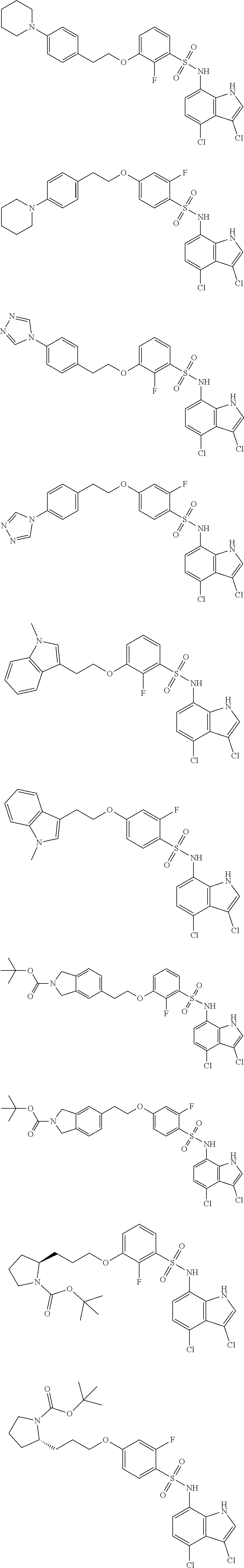

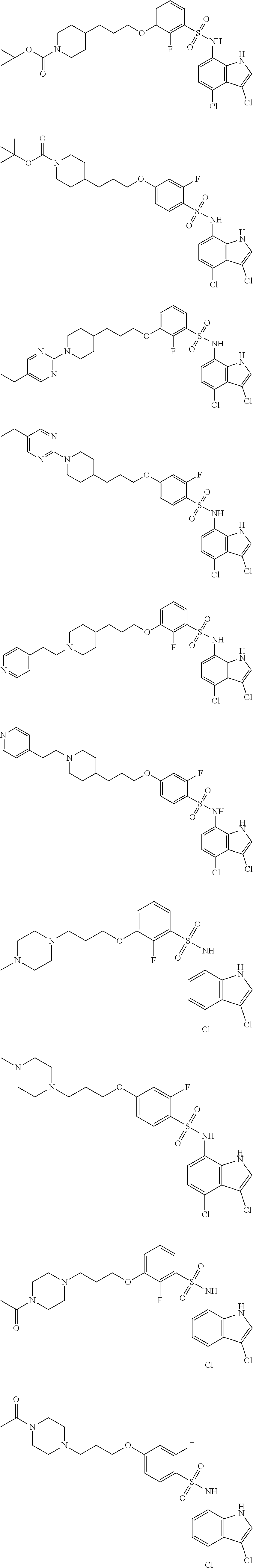

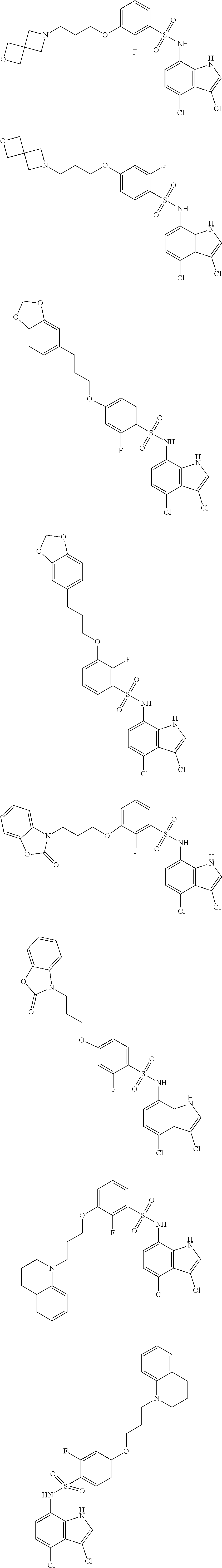

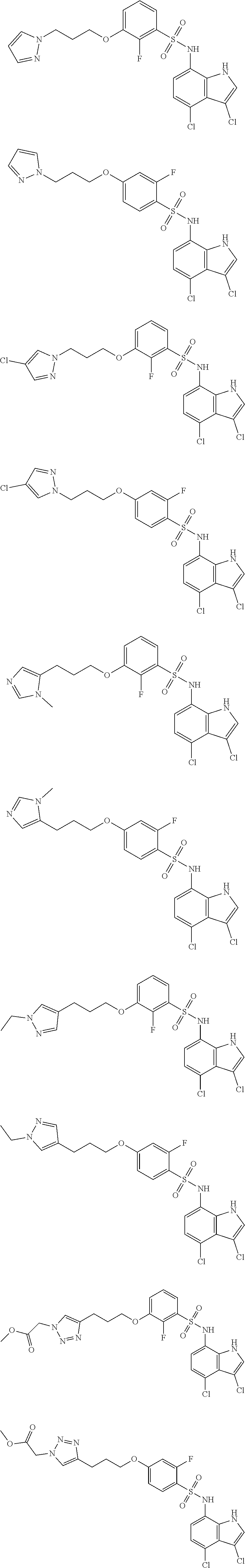

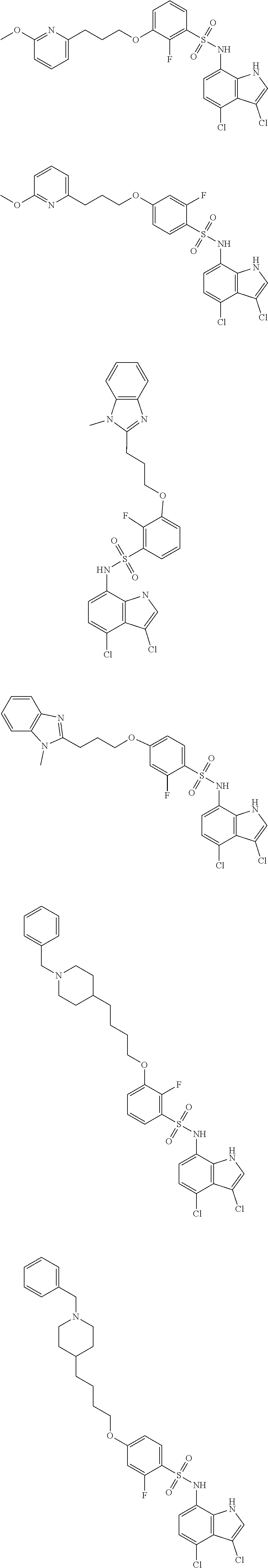

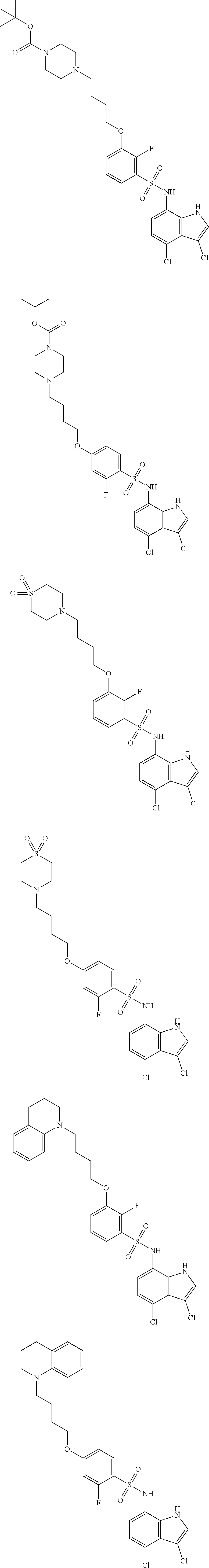 [SMILES: CC(C)(C)OC(=O)N1CCN(CCCCOc2cccc(S(=O)(=O)Nc3ccc(Cl)c4c(Cl)c[nH]c34)c2F)CC1 ; CC(C)(C)OC(=O)N1CCN(CCCCOc2ccc(S(=O)(=O)Nc3ccc(Cl)c4c(Cl)c[nH]c34)c(F)c2)CC1 ; O=S1(=O)CCN(CCCCOc2cccc(S(=O)(=O)Nc3ccc(Cl)c4c(Cl)c[nH]c34)c2F)CC1 ; O=S1(=O)CCN(CCCCOc2ccc(S(=O)(=O)Nc3ccc(Cl)c4c(Cl)c[nH]c34)c(F)c2)CC1 ; O=S(=O)(Nc1ccc(Cl)c2c(Cl)c[nH]c12)c1cccc(OCCCCN2CCCc3ccccc32)c1F ; O=S(=O)(Nc1ccc(Cl)c2c(Cl)c[nH]c12)c1ccc(OCCCCN2CCCc3ccccc32)cc1F]

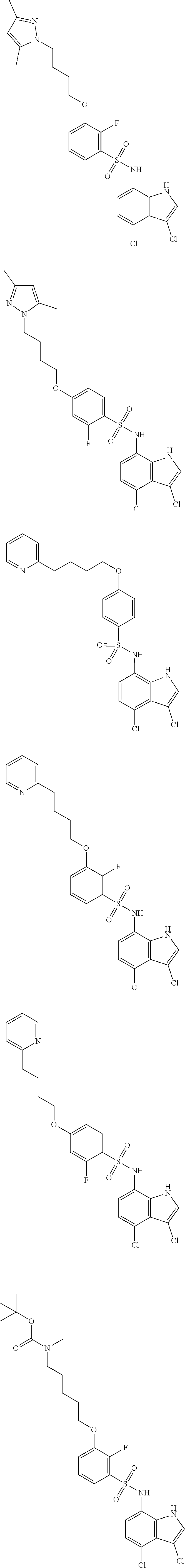 [SMILES: Cc1cc(C)n(CCCCOc2cccc(S(=O)(=O)Nc3ccc(Cl)c4c(Cl)c[nH]c34)c2F)n1 ; Cc1cc(C)n(CCCCOc2ccc(S(=O)(=O)Nc3ccc(Cl)c4c(Cl)c[nH]c34)c(F)c2)n1 ; O=S(=O)(Nc1ccc(Cl)c2c(Cl)c[nH]c12)c1ccc(OCCCCc2ccccn2)cc1 ; O=S(=O)(Nc1ccc(Cl)c2c(Cl)c[nH]c12)c1cccc(OCCCCc2ccccn2)c1F ; O=S(=O)(Nc1ccc(Cl)c2c(Cl)c[nH]c12)c1ccc(OCCCCc2ccccn2)cc1F ; CN(CCCCCOc1cccc(S(=O)(=O)Nc2ccc(Cl)c3c(Cl)c[nH]c23)c1F)C(=O)OC(C)(C)C]

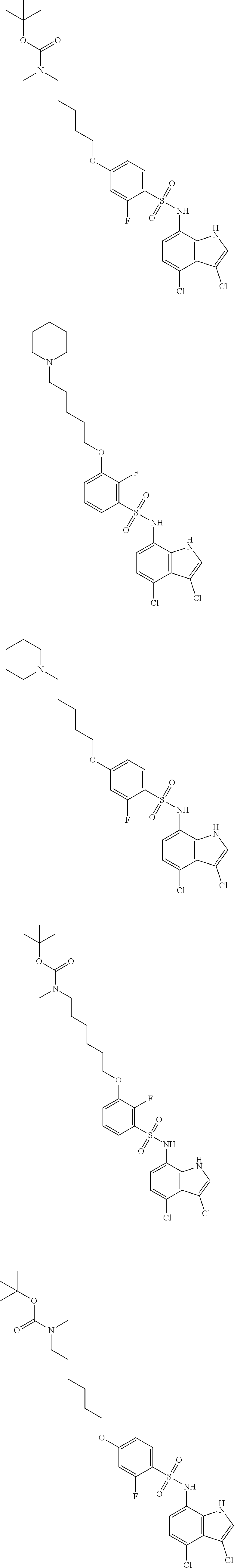 [SMILES: CN(CCCCCOc1ccc(S(=O)(=O)Nc2ccc(Cl)c3c(Cl)c[nH]c23)c(F)c1)C(=O)OC(C)(C)C ; O=S(=O)(Nc1ccc(Cl)c2c(Cl)c[nH]c12)c1cccc(OCCCCCN2CCCCC2)c1F ; O=S(=O)(Nc1ccc(Cl)c2c(Cl)c[nH]c12)c1ccc(OCCCCCN2CCCCC2)cc1F ; CN(CCCCCCOc1cccc(S(=O)(=O)Nc2ccc(Cl)c3c(Cl)c[nH]c23)c1F)C(=O)OC(C)(C)C ; CN(CCCCCCOc1ccc(S(=O)(=O)Nc2ccc(Cl)c3c(Cl)c[nH]c23)c(F)c1)C(=O)OC(C)(C)C]

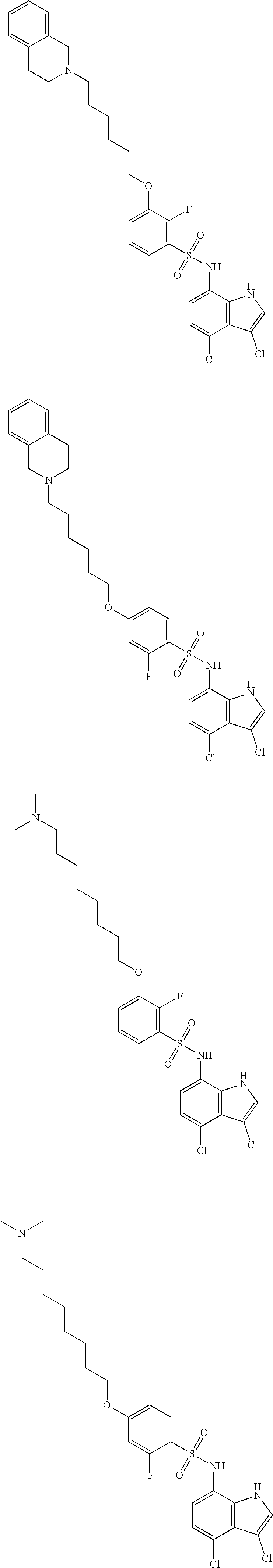 [SMILES: O=S(=O)(Nc1ccc(Cl)c2c(Cl)c[nH]c12)c1cccc(OCCCCCCN2CCc3ccccc3C2)c1F ; O=S(=O)(Nc1ccc(Cl)c2c(Cl)c[nH]c12)c1ccc(OCCCCCCN2CCc3ccccc3C2)cc1F ; CN(C)CCCCCCCCOc1cccc(S(=O)(=O)Nc2ccc(Cl)c3c(Cl)c[nH]c23)c1F ; CN(C)CCCCCCCCOc1ccc(S(=O)(=O)Nc2ccc(Cl)c3c(Cl)c[nH]c23)c(F)c1]

or a pharmaceutically acceptable salt thereof.

In some embodiments the compound of Formula I is a compound of Formula IIc, Formula IId, Formula IIe, Formula IIf or Formula IIg:

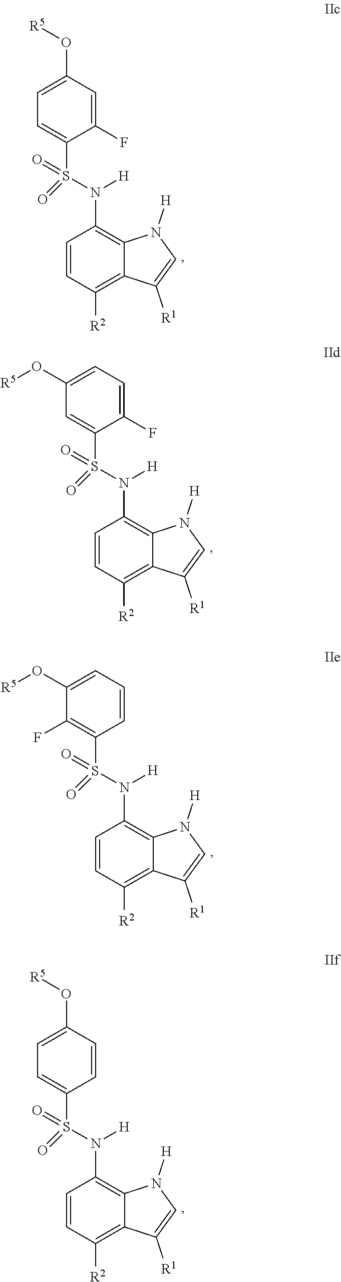

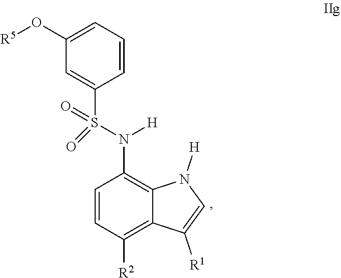

or a pharmaceutically acceptable salt thereof, wherein R¹, R², and R⁵, are as described according to any embodiment of compounds of Formula II.

### Compounds of Formula II

In some embodiments the compound of Formula I is a compound of Formula III

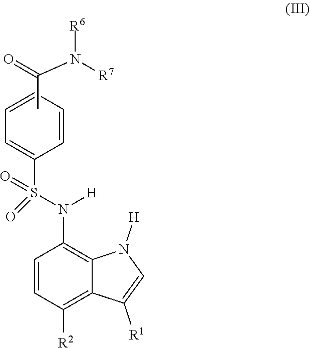

or a pharmaceutically acceptable salt thereof.

Substituent R¹ of Formula II is selected from the group consisting of chloro, bromo, fluoro, and iodo. In some embodiments, R¹ is selected from the group consisting of chloro, bromo, and iodo. In some embodiments, R¹ is chloro.

Substituent R² of Formula III is selected from the group consisting of H, chloro, fluoro, and methyl. In some embodiments R² of Formula II is selected from the group consisting of H and chloro.

Substituent R³ of Formula III is selected from the group consisting of H, chloro, fluoro, cyano and methyl. In some embodiments R³ is selected from the group consisting H, fluoro and cyano. In some embodiments, R³ is H. In some embodiments, R³ is F.

Substituent R⁶ of Formula III is selected from the group consisting of H and CH₃. In some embodiments R⁶ is H.

Substituent R⁷ is selected from the group consisting of optionally substituted C₃-C₇ cycloalkyl, optionally substituted 2,3-dihydro-1H-indenyl, optionally substituted 3 to 7-membered heterocyclyl, and substituted C₁-C₈ alkyl. The C₁-C₈ alkyl is substituted with at least one substituent selected from the group consisting of optionally substituted C₁-C₆ alkoxy, optionally substituted 4 to 8-membered heterocyclyoxy, —C(O)NR⁸R⁹, C₁-C₆ alkylsulfonyl, optionally substituted C₃-C₇ cycloalkyl, optionally substituted bicyclooctatrienyl, optionally substituted 4 to 8-membered heterocyclyl, optionally substituted C₆-C₁₀ aryl, optionally substituted dihydrobenzodioxinyl, and optionally substituted 5 to 10-membered heteroaryl. R⁸ and R⁹ together with the nitrogen atom to which they are attached form an optionally substituted 5 to 6-membered heterocyclyl.

In some embodiments R⁷ is selected from the group consisting of optionally substituted cyclopropyl; optionally substituted 2,3-dihydro-1H-indenyl; optionally substituted 6 to 7-membered heterocyclyl;
C₁ alkyl substituted with at least one substituent selected from the group consisting of optionally substituted cyclopropyl, optionally substituted bicyclooctatrienyl, optionally substituted 6-membered heterocyclyl, optionally substituted phenyl, and optionally substituted 5 to 10-membered heteroaryl;
C₂ alkyl substituted with at least one substituent selected from the group consisting of optionally substituted methoxy, optionally substituted 6-membered heterocyclyoxy, ethylsulfonyl, optionally substituted 4-membered heterocyclyl, optionally substituted phenyl, and optionally substituted 5 to 6 membered heteroaryl;
C₃ alkyl substituted with at least one substituent selected from the group consisting of optionally substituted optionally substituted C₁-C₂ alkoxy; —C(O)NR⁸R⁹, wherein R⁸R⁹ together with the nitrogen atom to which they are attached form an optionally substituted 5 membered heterocyclyl; optionally substituted phenyl, and optionally substituted dihydrobenzodioxinyl;
C₅ alkyl substituted with optionally substituted 9-membered heteroaryl.

In some embodiments R⁷ is selected from the group consisting of optionally substituted cyclopropyl; optionally substituted 2,3-dihydro-1H-indenyl; optionally substituted piperidin-2-only, optionally substituted azepan-2-only,
C₁ alkyl substituted with at least one substituent selected from the group consisting of optionally substituted cyclopropyl, optionally substituted bicyclooctatrienyl, optionally substituted piperidinyl, optionally substituted phenyl, optionally substituted pyrazolyl, optionally substituted oxadiazolyl, optionally substituted pyridinyl, optionally substituted pyrazinyl and optionally substituted pyridinyl;
C₂ alkyl substituted with at least one substituent selected from the group consisting of optionally substituted cyclopropylmethoxy, optionally substituted (tetrahydro-2H-thiopyran 1,1-dioxide)oxy, ethylsulfonyl, optionally substituted optionally substituted azetidinyl, optionally substituted phenyl, optionally substituted furanyl, and optionally substituted pyridinyl;
C₃ alkyl substituted with at least one substituent selected from the group consisting of optionally substituted optionally substituted C₁-C₂ alkoxy; —C(O)NR⁸R⁹, wherein R⁸R⁹ together with the nitrogen atom to which they are attached form an optionally substituted pyrrolidinyl; optionally substituted phenyl, and optionally substituted dihydrobenzodioxinyl; and
C₅ alkyl substituted with optionally substituted 4,5,6,7-tetrahydro-1H-imidazo[4,5-c]pyridinyl.

In some embodiments R⁷ is selected from the group consisting of cyclopropyl, —CH₂CH₂SO₂CH₂CH₃, —CH₂CH₂CH₂OCH₂CH₂OCH₃,

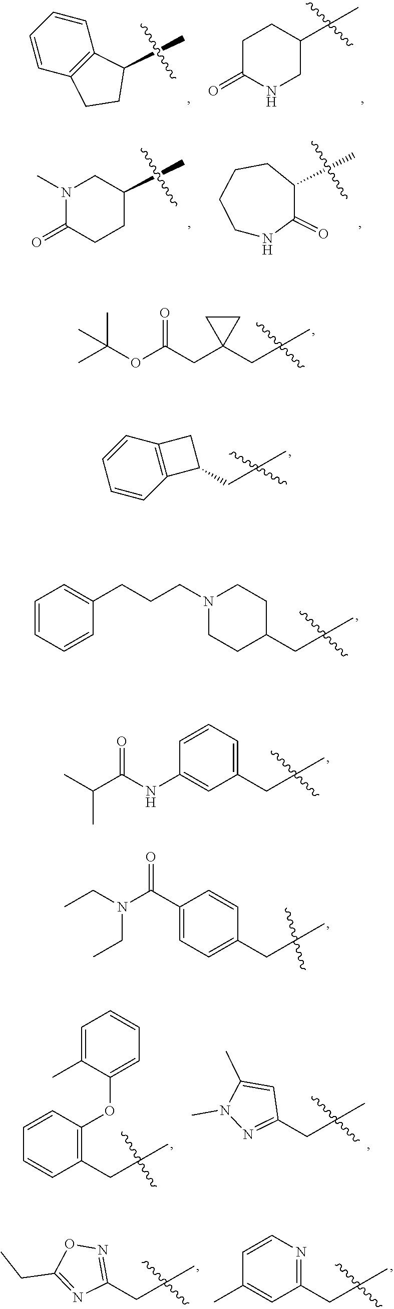

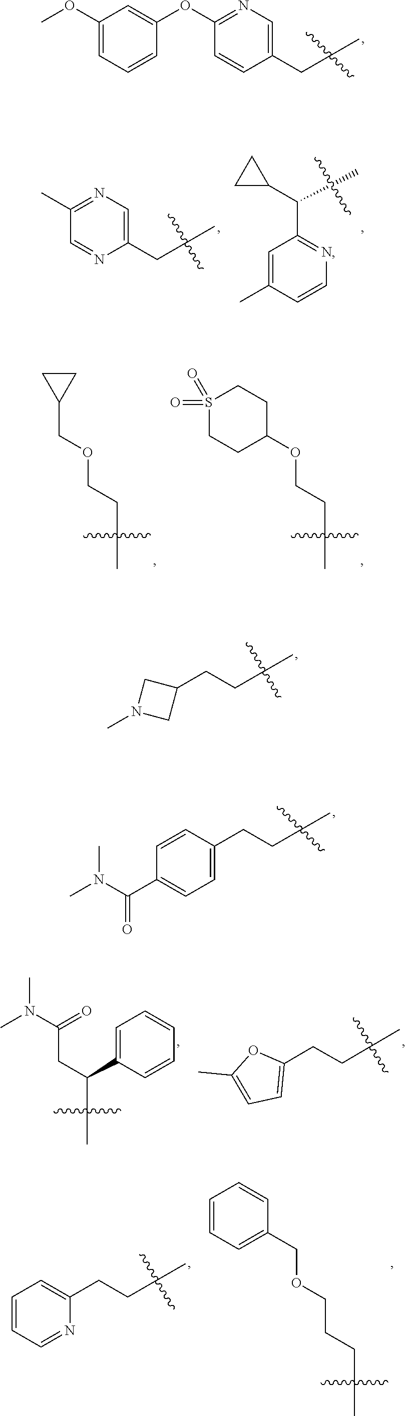

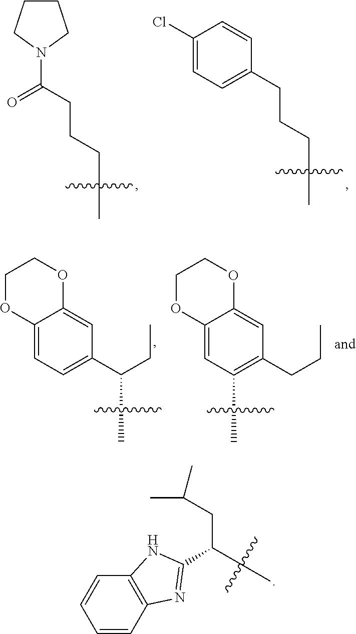

Alternatively R⁶ and R⁷ together with the nitrogen atom to which they are attached form a group selected from optionally substituted 4 to 12-membered optionally substituted heterocyclyl, and optionally substituted 4,5,6,7-tetrahydro-1H-imidazo[4,5-c]pyridinyl. In some embodiments R⁶ and R⁷ together with the nitrogen atom to which they are attached form a group selected from optionally substituted 4 to 11-membered optionally substituted heterocyclyl, optionally substituted 4,5,6,7-tetrahydro-1H-imidazo[4,5-c]pyridinyl and optionally substituted tetrahydroimidazopyridine. In some embodiments R⁶ and R⁷ together with the nitrogen atom to which they are attached form a group selected from optionally substituted azetidinyl, optionally substituted pyrrolidinyl, optionally substituted morpholinyl, optionally substituted piperidinyl, optionally substituted piperazinyl, optionally substituted 1-oxa-4,9-diazaspiro[5.5]undecane, and optionally substituted 4,5,6,7-tetrahydro-1H-imidazo[4,5-c]pyridine. In some embodiments R⁶ and R⁷ together with the nitrogen atom to which they are attached form a group selected from

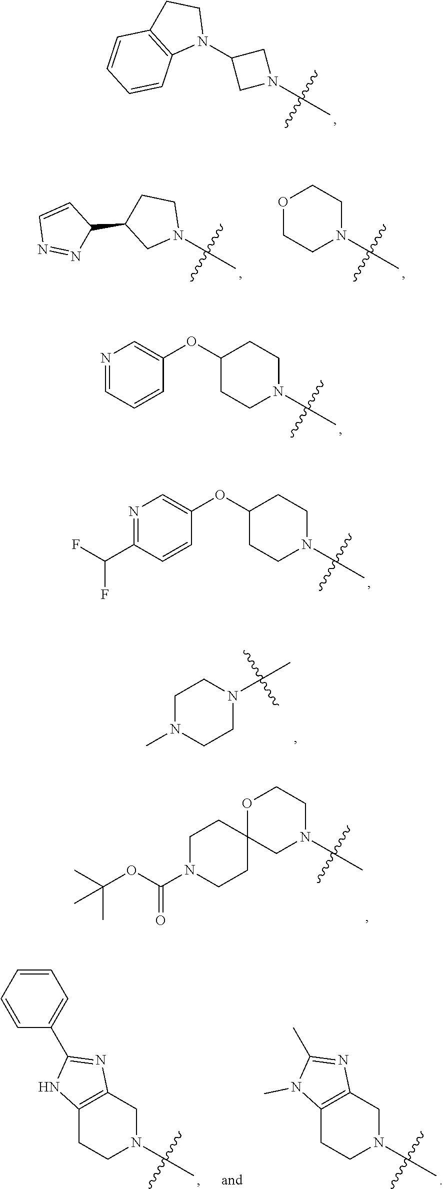

Provided the compound of Formula III is not

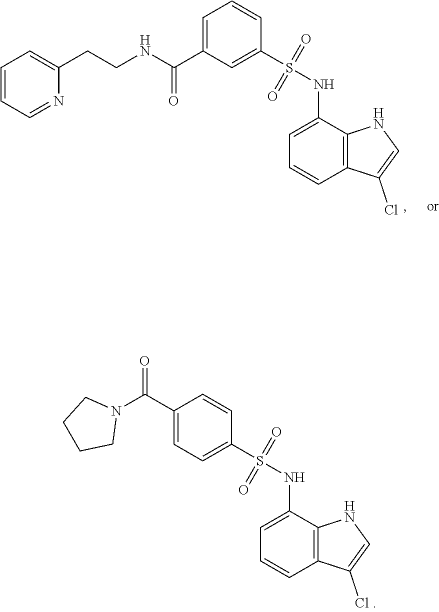 [SMILES: O=C(NCCc1ccccn1)c1cccc(S(=O)(=O)Nc2cccc3c(Cl)c[nH]c23)c1 ; O=C(c1ccc(S(=O)(=O)Nc2cccc3c(Cl)c[nH]c23)cc1)N1CCCC1]

Some embodiments describe a compound of Formula III wherein
R¹ is chloro;
R² is selected from the group consisting of H and chloro;
R³ is selected from the group consisting H, fluoro, and cyano;
R⁶ is selected from the group consisting of H and CH₃;
R⁷ is selected from the group consisting of cyclopropyl, —CH₂CH₂SO₂CH₂CH₃, —CH₂CH₂CH₂OCH₂CH₂OCH₃,

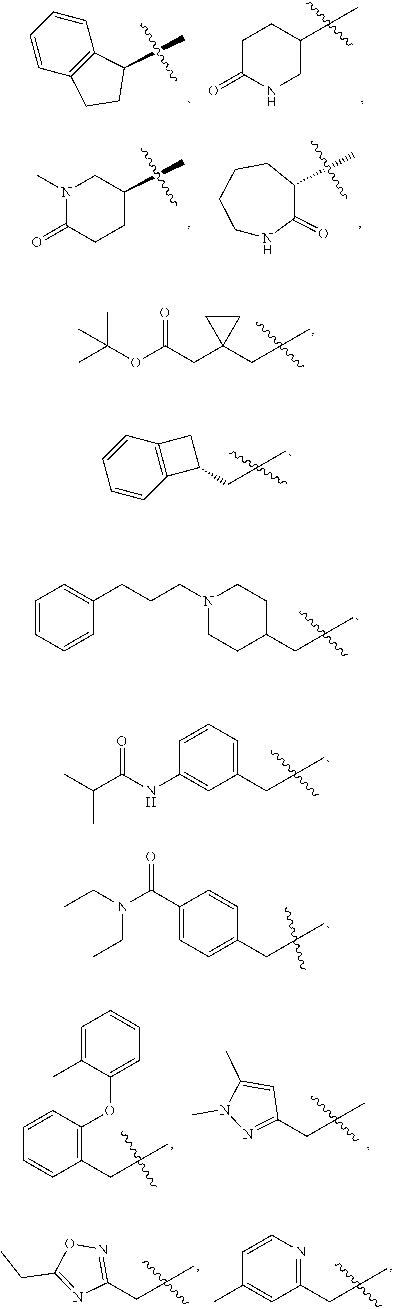

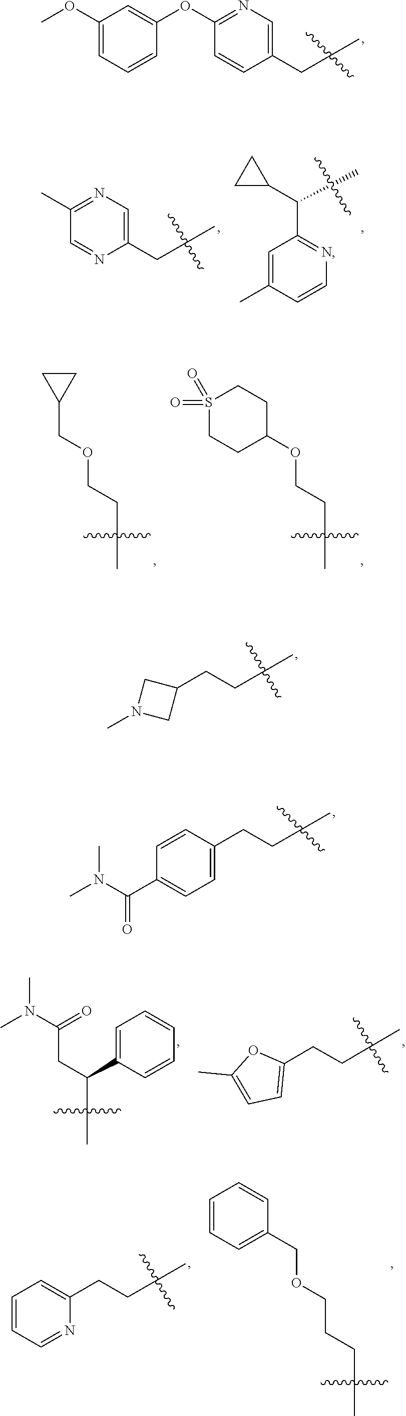

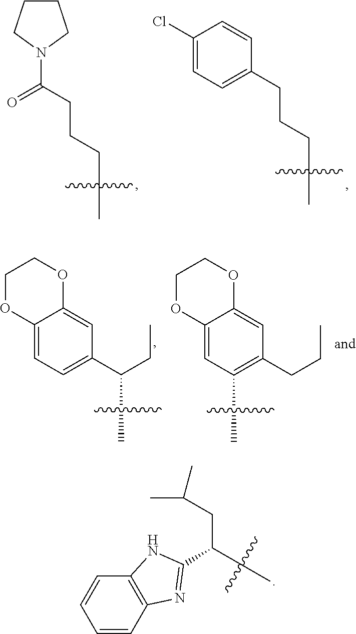

or
alternatively. R⁶ and R⁷ together with the nitrogen atom to which they are attached form a group selected from

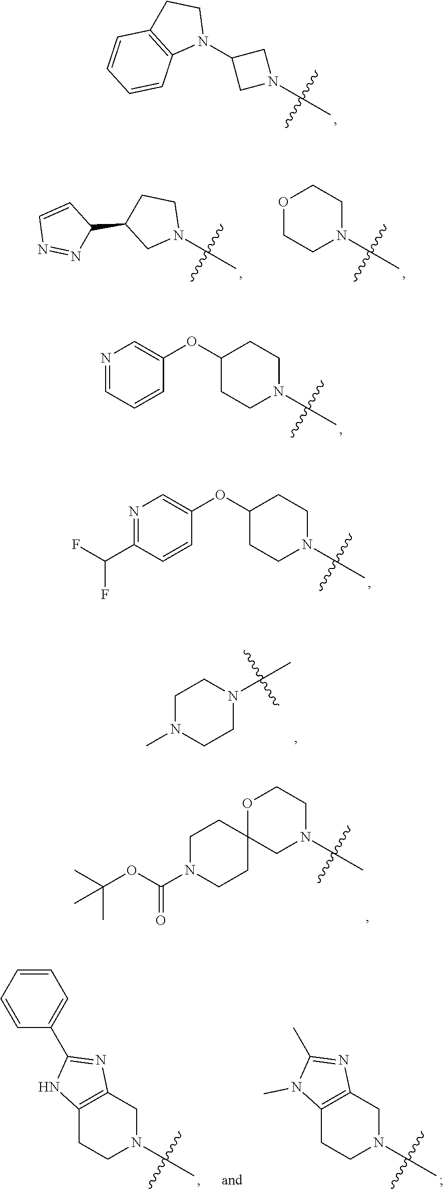

provided the compound of Formula III is not

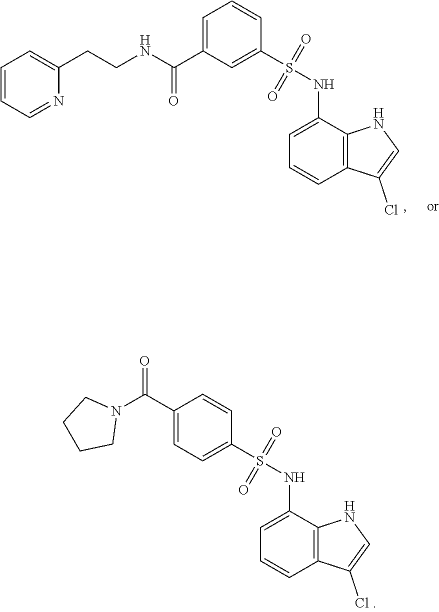 [SMILES: O=C(NCCc1ccccn1)c1cccc(S(=O)(=O)Nc2cccc3c(Cl)c[nH]c23)c1 ; O=C(c1ccc(S(=O)(=O)Nc2cccc3c(Cl)c[nH]c23)cc1)N1CCCC1]

In some embodiments, the compound of Formula III is a compound of Formula IIIa or Formula IIIb:

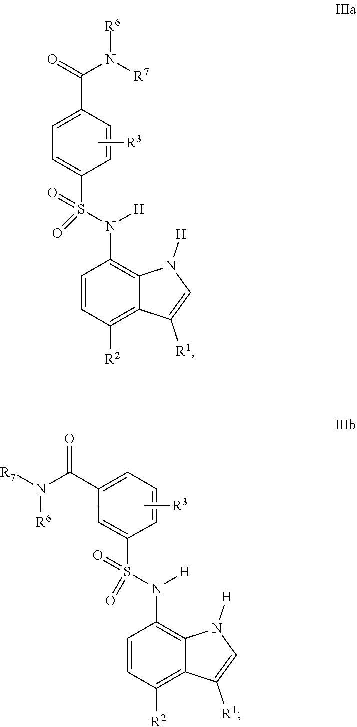

or a pharmaceutically acceptable salt thereof, wherein R¹, R² R³, R⁶, and R⁷ are as described for any embodiment of the compounds of Formula II.

In some embodiments a compound of Formula III is selected from any one of the following:

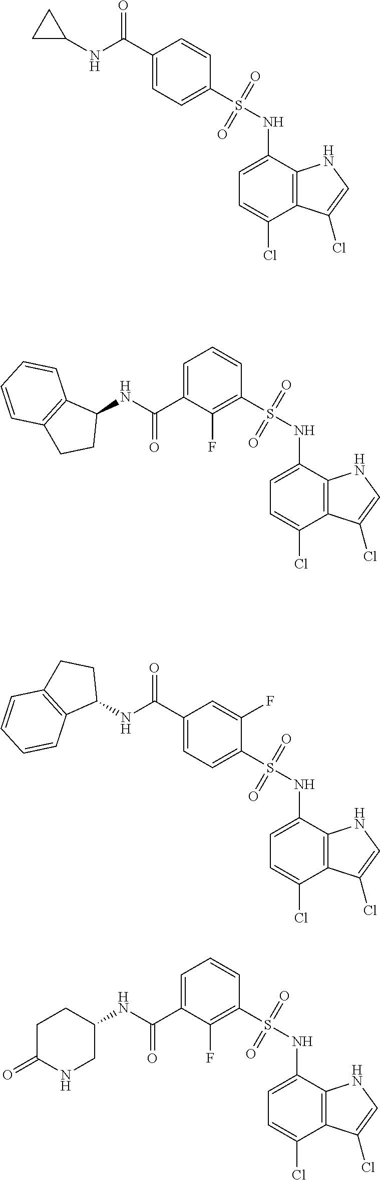 [SMILES: O=C(NC1CC1)c1ccc(S(=O)(=O)Nc2ccc(Cl)c3c(Cl)c[nH]c23)cc1 ; O=C(N[C@H]1CCc2ccccc21)c1cccc(S(=O)(=O)Nc2ccc(Cl)c3c(Cl)c[nH]c23)c1F ; O=C(N[C@H]1CCc2ccccc21)c1ccc(S(=O)(=O)Nc2ccc(Cl)c3c(Cl)c[nH]c23)c(F)c1 ; O=C1CC[C@H](NC(=O)c2cccc(S(=O)(=O)Nc3ccc(Cl)c4c(Cl)c[nH]c34)c2F)CN1]

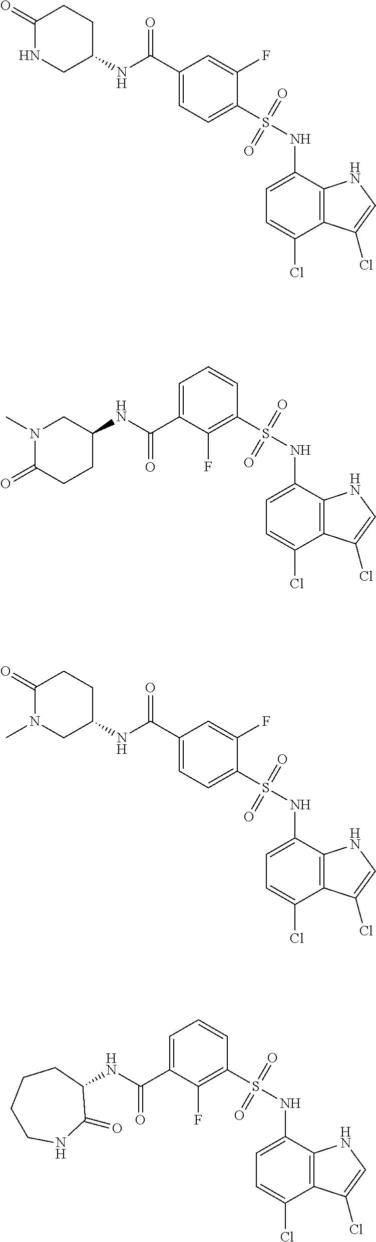 [SMILES: O=C1CC[C@H](NC(=O)c2ccc(S(=O)(=O)Nc3ccc(Cl)c4c(Cl)c[nH]c34)c(F)c2)CN1 ; CN1C[C@@H](NC(=O)c2cccc(S(=O)(=O)Nc3ccc(Cl)c4c(Cl)c[nH]c34)c2F)CCC1=O ; CN1C[C@@H](NC(=O)c2ccc(S(=O)(=O)Nc3ccc(Cl)c4c(Cl)c[nH]c34)c(F)c2)CCC1=O ; O=C(N[C@H]1CCCCNC1=O)c1cccc(S(=O)(=O)Nc2ccc(Cl)c3c(Cl)c[nH]c23)c1F]

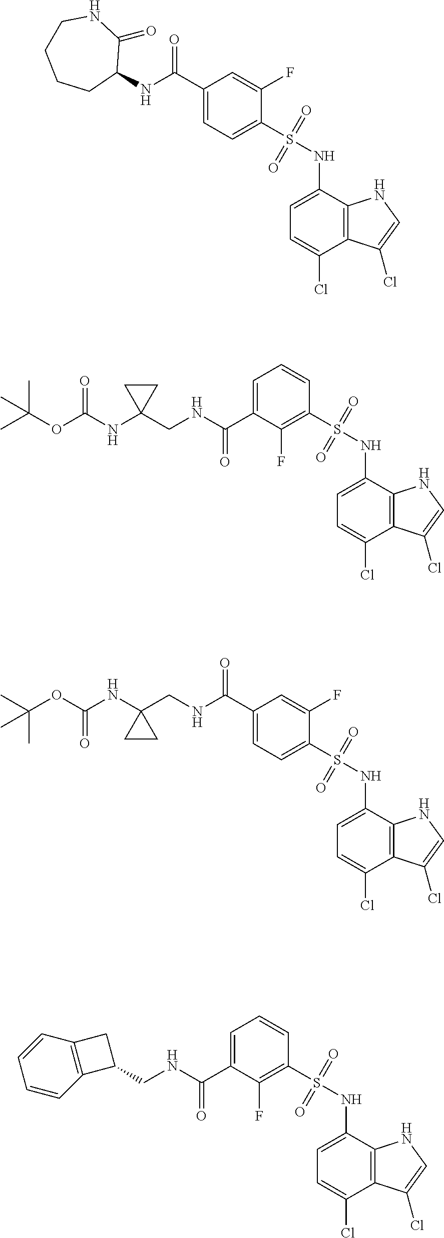 [SMILES: O=C(N[C@H]1CCCCNC1=O)c1ccc(S(=O)(=O)Nc2ccc(Cl)c3c(Cl)c[nH]c23)c(F)c1 ; CC(C)(C)OC(=O)NC1(CNC(=O)c2cccc(S(=O)(=O)Nc3ccc(Cl)c4c(Cl)c[nH]c34)c2F)CC1 ; CC(C)(C)OC(=O)NC1(CNC(=O)c2ccc(S(=O)(=O)Nc3ccc(Cl)c4c(Cl)c[nH]c34)c(F)c2)CC1 ; O=C(NC[C@H]1Cc2ccccc21)c1cccc(S(=O)(=O)Nc2ccc(Cl)c3c(Cl)c[nH]c23)c1F]

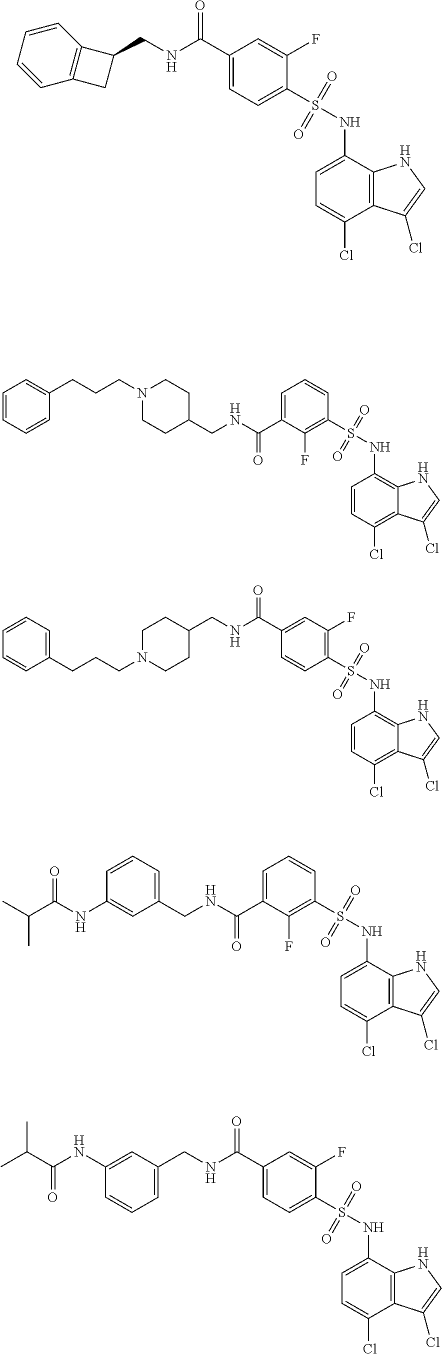 [SMILES: O=C(NC[C@H]1Cc2ccccc21)c1ccc(S(=O)(=O)Nc2ccc(Cl)c3c(Cl)c[nH]c23)c(F)c1 ; O=C(NCC1CCN(CCCc2ccccc2)CC1)c1cccc(S(=O)(=O)Nc2ccc(Cl)c3c(Cl)c[nH]c23)c1F ; O=C(NCC1CCN(CCCc2ccccc2)CC1)c1ccc(S(=O)(=O)Nc2ccc(Cl)c3c(Cl)c[nH]c23)c(F)c1 ; CC(C)C(=O)Nc1cccc(CNC(=O)c2cccc(S(=O)(=O)Nc3ccc(Cl)c4c(Cl)c[nH]c34)c2F)c1 ; CC(C)C(=O)Nc1cccc(CNC(=O)c2ccc(S(=O)(=O)Nc3ccc(Cl)c4c(Cl)c[nH]c34)c(F)c2)c1]

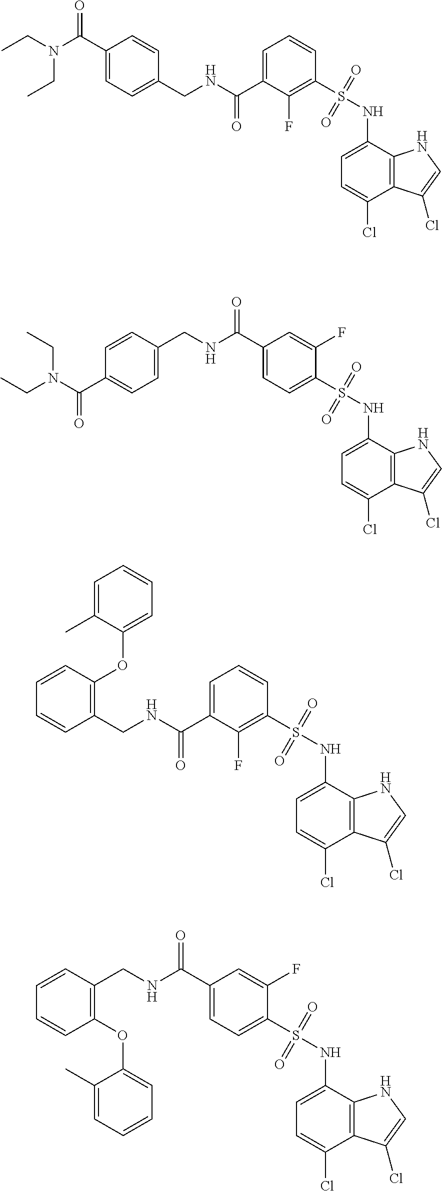 [SMILES: CCN(CC)C(=O)c1ccc(CNC(=O)c2cccc(S(=O)(=O)Nc3ccc(Cl)c4c(Cl)c[nH]c34)c2F)cc1 ; CCN(CC)C(=O)c1ccc(CNC(=O)c2ccc(S(=O)(=O)Nc3ccc(Cl)c4c(Cl)c[nH]c34)c(F)c2)cc1 ; Cc1ccccc1Oc1ccccc1CNC(=O)c1cccc(S(=O)(=O)Nc2ccc(Cl)c3c(Cl)c[nH]c23)c1F ; Cc1ccccc1Oc1ccccc1CNC(=O)c1ccc(S(=O)(=O)Nc2ccc(Cl)c3c(Cl)c[nH]c23)c(F)c1]

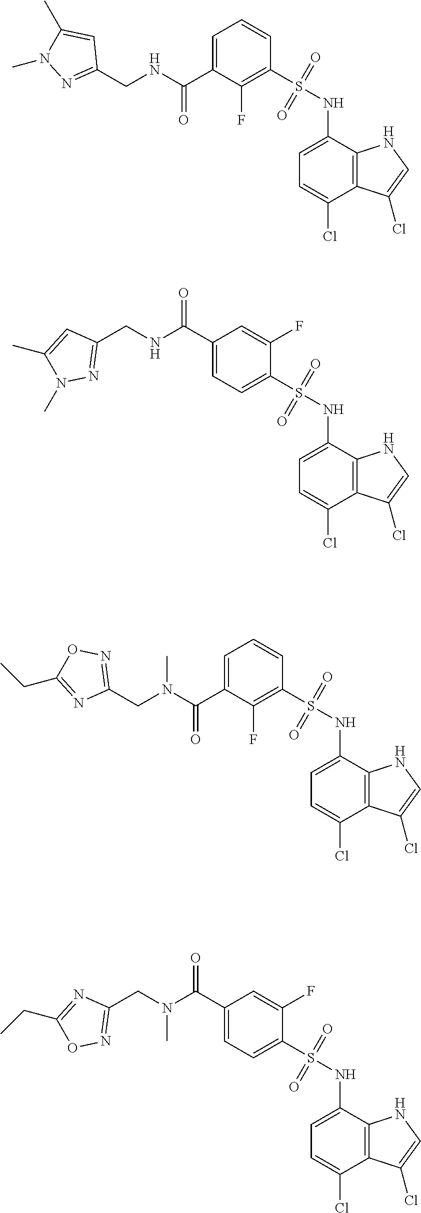 [SMILES: Cc1cc(CNC(=O)c2cccc(S(=O)(=O)Nc3ccc(Cl)c4c(Cl)c[nH]c34)c2F)nn1C ; Cc1cc(CNC(=O)c2ccc(S(=O)(=O)Nc3ccc(Cl)c4c(Cl)c[nH]c34)c(F)c2)nn1C ; CCc1nc(CN(C)C(=O)c2cccc(S(=O)(=O)Nc3ccc(Cl)c4c(Cl)c[nH]c34)c2F)no1 ; CCc1nc(CN(C)C(=O)c2ccc(S(=O)(=O)Nc3ccc(Cl)c4c(Cl)c[nH]c34)c(F)c2)no1]

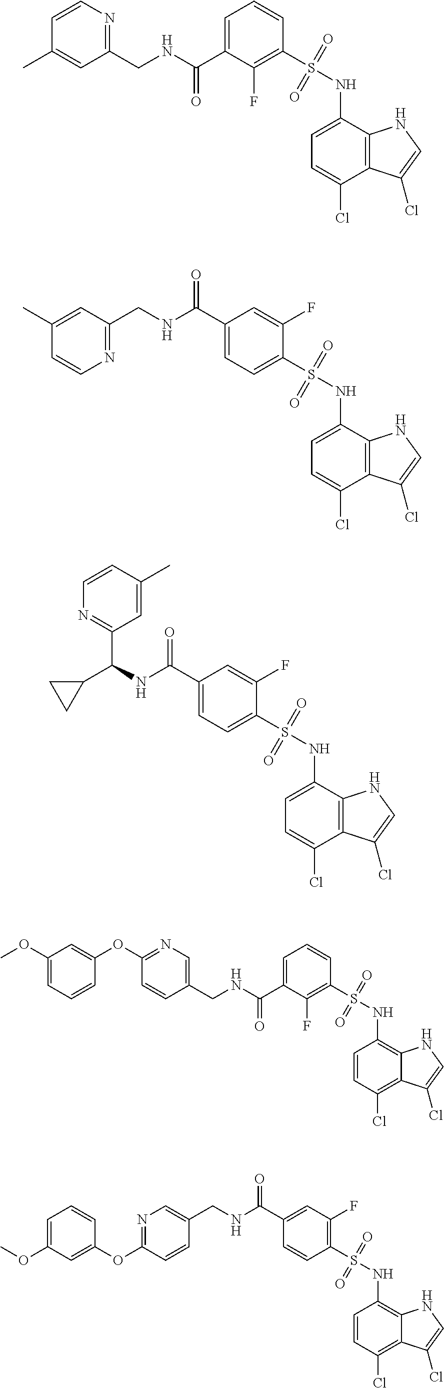 [SMILES: Cc1ccnc(CNC(=O)c2cccc(S(=O)(=O)Nc3ccc(Cl)c4c(Cl)c[nH]c34)c2F)c1 ; Cc1ccnc(CNC(=O)c2ccc(S(=O)(=O)Nc3ccc(Cl)c4c(Cl)c[nH]c34)c(F)c2)c1 ; Cc1ccnc([C@@H](NC(=O)c2ccc(S(=O)(=O)Nc3ccc(Cl)c4c(Cl)c[nH]c34)c(F)c2)C2CC2)c1 ; COc1cccc(Oc2ccc(CNC(=O)c3cccc(S(=O)(=O)Nc4ccc(Cl)c5c(Cl)c[nH]c45)c3F)cn2)c1 ; COc1cccc(Oc2ccc(CNC(=O)c3ccc(S(=O)(=O)Nc4ccc(Cl)c5c(Cl)c[nH]c45)c(F)c3)cn2)c1]

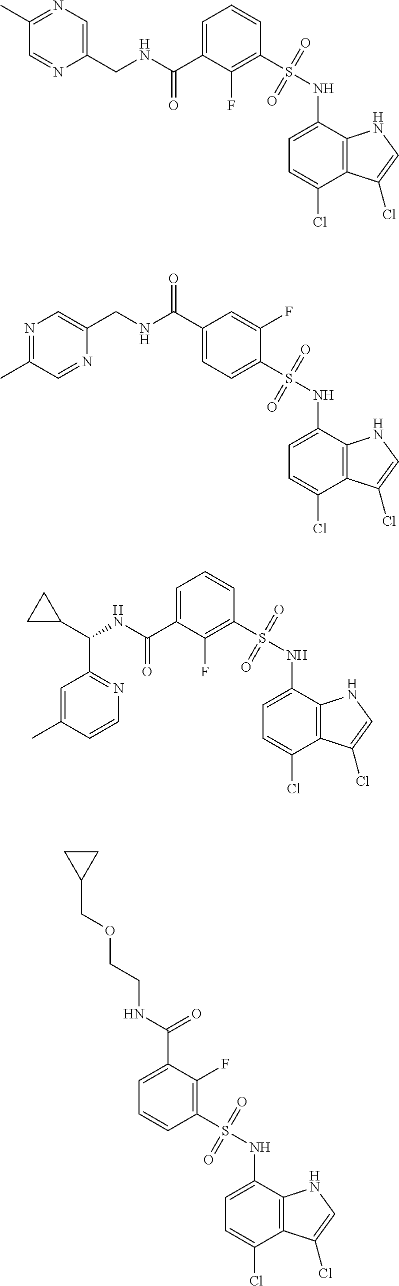 [SMILES: Cc1cnc(CNC(=O)c2cccc(S(=O)(=O)Nc3ccc(Cl)c4c(Cl)c[nH]c34)c2F)cn1 ; Cc1cnc(CNC(=O)c2ccc(S(=O)(=O)Nc3ccc(Cl)c4c(Cl)c[nH]c34)c(F)c2)cn1 ; Cc1ccnc([C@@H](NC(=O)c2cccc(S(=O)(=O)Nc3ccc(Cl)c4c(Cl)c[nH]c34)c2F)C2CC2)c1 ; O=C(NCCOCC1CC1)c1cccc(S(=O)(=O)Nc2ccc(Cl)c3c(Cl)c[nH]c23)c1F]

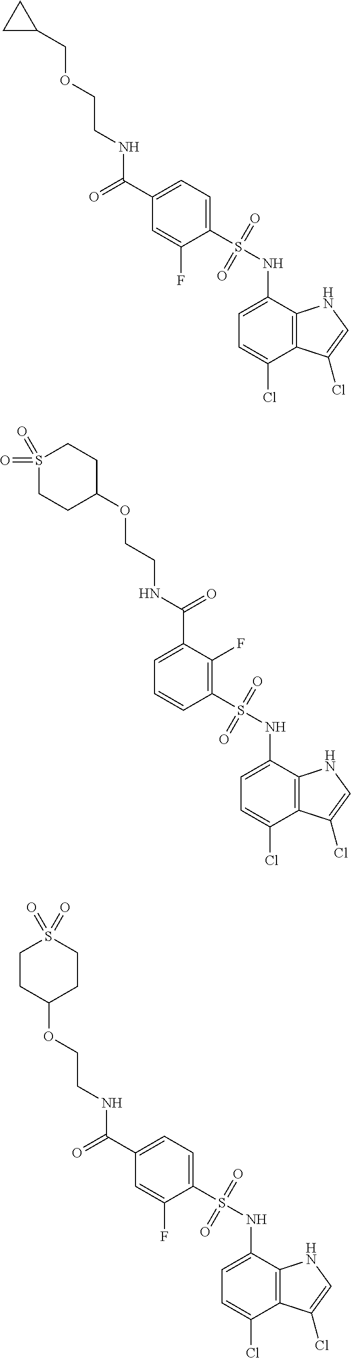 [SMILES: O=C(NCCOCC1CC1)c1ccc(S(=O)(=O)Nc2ccc(Cl)c3c(Cl)c[nH]c23)c(F)c1 ; O=C(NCCOC1CCS(=O)(=O)CC1)c1cccc(S(=O)(=O)Nc2ccc(Cl)c3c(Cl)c[nH]c23)c1F ; O=C(NCCOC1CCS(=O)(=O)CC1)c1ccc(S(=O)(=O)Nc2ccc(Cl)c3c(Cl)c[nH]c23)c(F)c1]

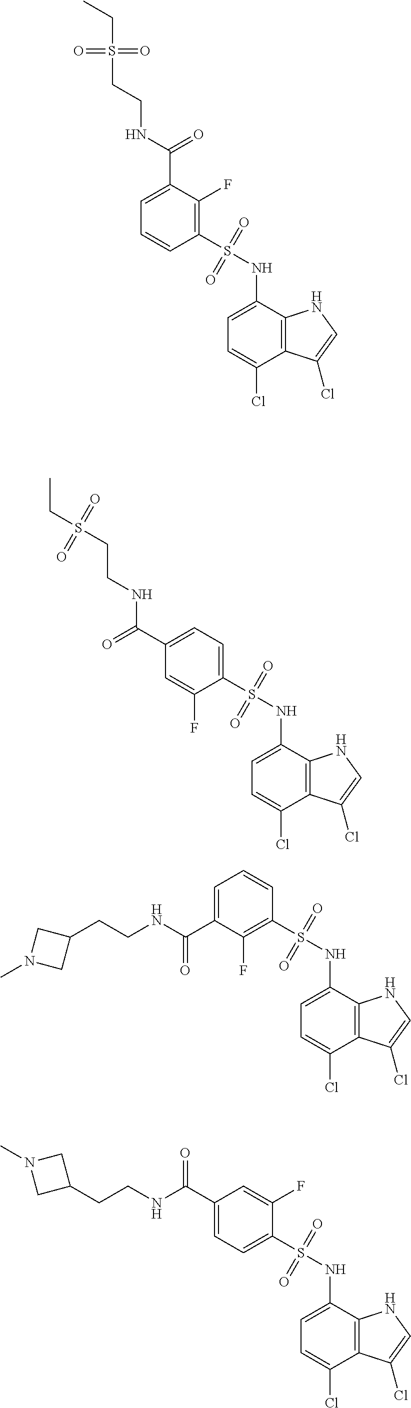

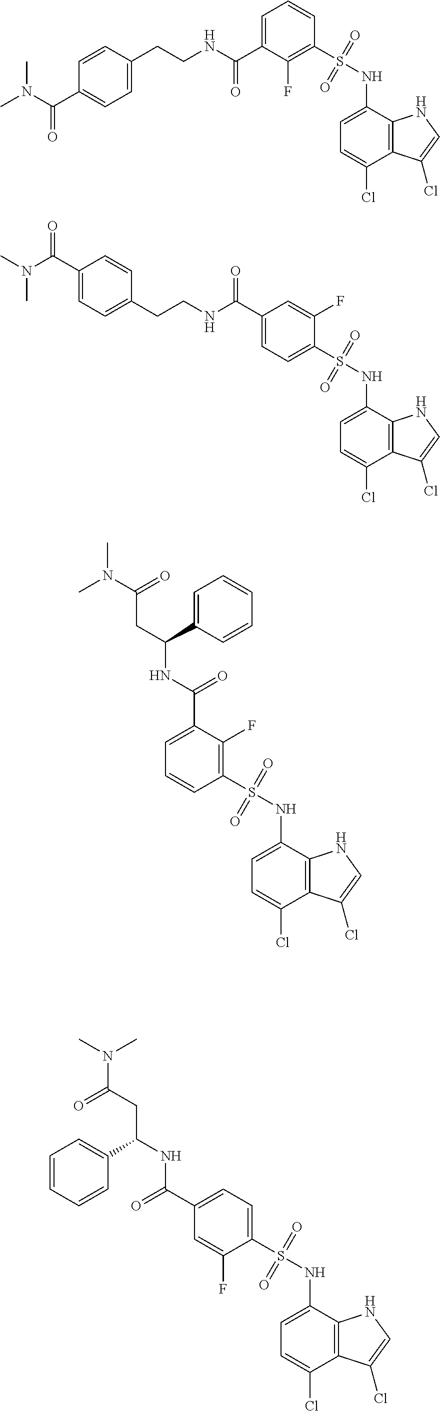 [SMILES: CN(C)C(=O)c1ccc(CCNC(=O)c2cccc(S(=O)(=O)Nc3ccc(Cl)c4c(Cl)c[nH]c34)c2F)cc1 ; CN(C)C(=O)c1ccc(CCNC(=O)c2ccc(S(=O)(=O)Nc3ccc(Cl)c4c(Cl)c[nH]c34)c(F)c2)cc1 ; CN(C)C(=O)C[C@H](NC(=O)c1cccc(S(=O)(=O)Nc2ccc(Cl)c3c(Cl)c[nH]c23)c1F)c1ccccc1 ; CN(C)C(=O)C[C@H](NC(=O)c1ccc(S(=O)(=O)Nc2ccc(Cl)c3c(Cl)c[nH]c23)c(F)c1)c1ccccc1]

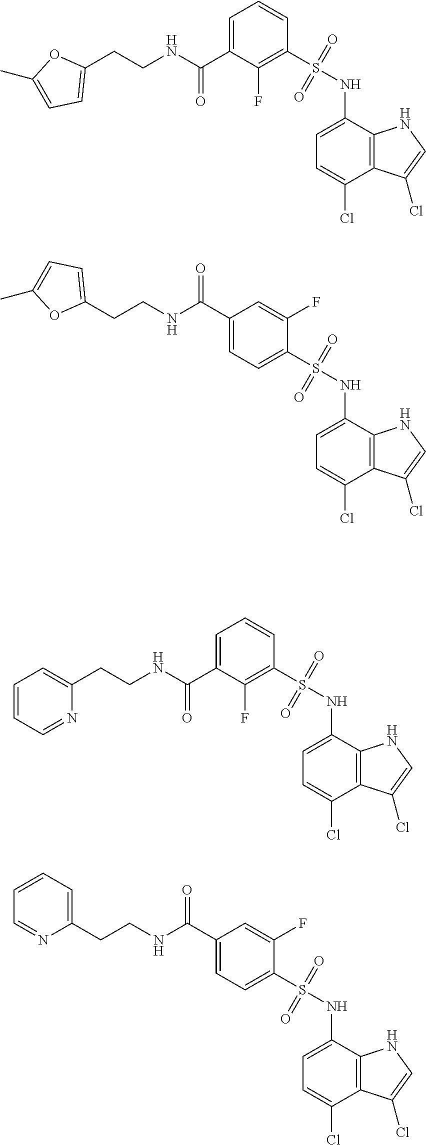 [SMILES: Cc1ccc(CCNC(=O)c2cccc(S(=O)(=O)Nc3ccc(Cl)c4c(Cl)c[nH]c34)c2F)o1 ; Cc1ccc(CCNC(=O)c2ccc(S(=O)(=O)Nc3ccc(Cl)c4c(Cl)c[nH]c34)c(F)c2)o1 ; O=C(NCCc1ccccn1)c1cccc(S(=O)(=O)Nc2ccc(Cl)c3c(Cl)c[nH]c23)c1F ; O=C(NCCc1ccccn1)c1ccc(S(=O)(=O)Nc2ccc(Cl)c3c(Cl)c[nH]c23)c(F)c1]

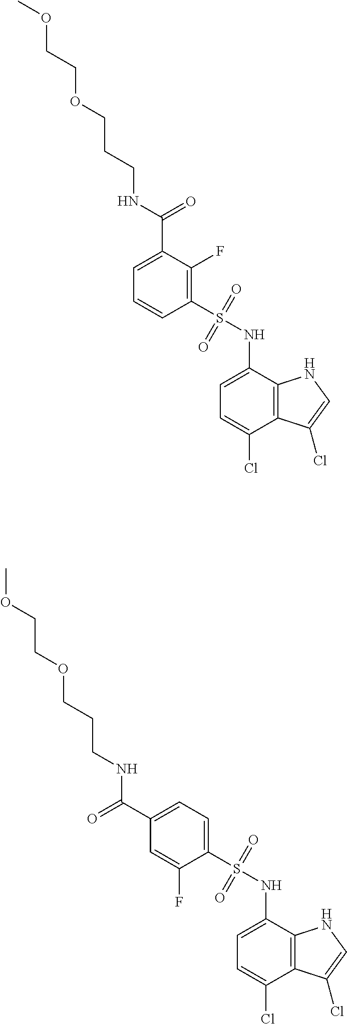 [SMILES: COCCOCCCNC(=O)c1cccc(S(=O)(=O)Nc2ccc(Cl)c3c(Cl)c[nH]c23)c1F ; COCCOCCCNC(=O)c1ccc(S(=O)(=O)Nc2ccc(Cl)c3c(Cl)c[nH]c23)c(F)c1]

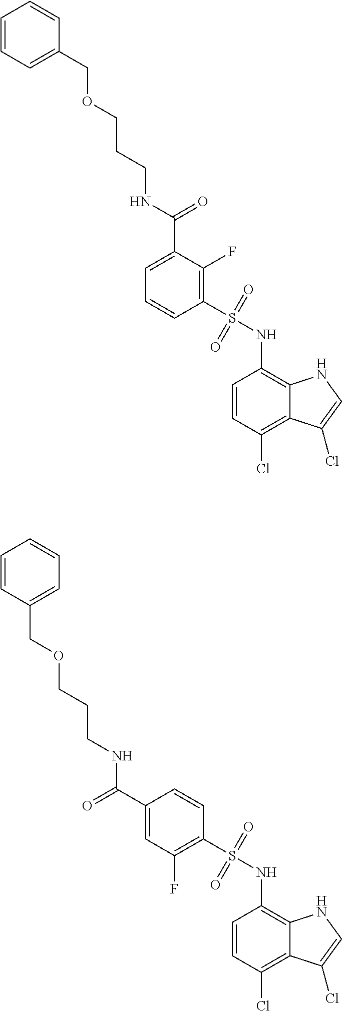 [SMILES: O=C(NCCCOCc1ccccc1)c1cccc(S(=O)(=O)Nc2ccc(Cl)c3c(Cl)c[nH]c23)c1F ; O=C(NCCCOCc1ccccc1)c1ccc(S(=O)(=O)Nc2ccc(Cl)c3c(Cl)c[nH]c23)c(F)c1]

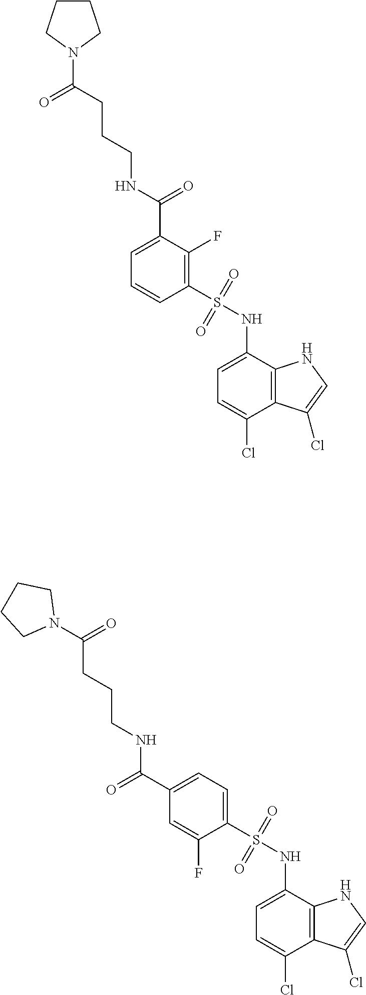 [SMILES: O=C(NCCCC(=O)N1CCCC1)c1cccc(S(=O)(=O)Nc2ccc(Cl)c3c(Cl)c[nH]c23)c1F ; O=C(NCCCC(=O)N1CCCC1)c1ccc(S(=O)(=O)Nc2ccc(Cl)c3c(Cl)c[nH]c23)c(F)c1]

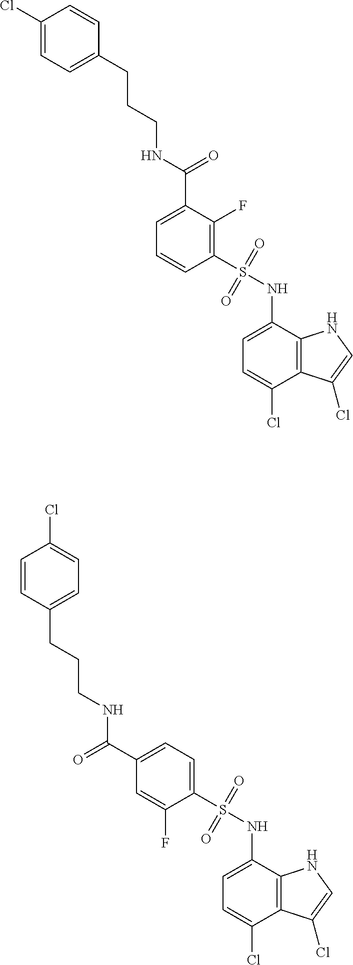 [SMILES: O=C(NCCCc1ccc(Cl)cc1)c1cccc(S(=O)(=O)Nc2ccc(Cl)c3c(Cl)c[nH]c23)c1F ; O=C(NCCCc1ccc(Cl)cc1)c1ccc(S(=O)(=O)Nc2ccc(Cl)c3c(Cl)c[nH]c23)c(F)c1]

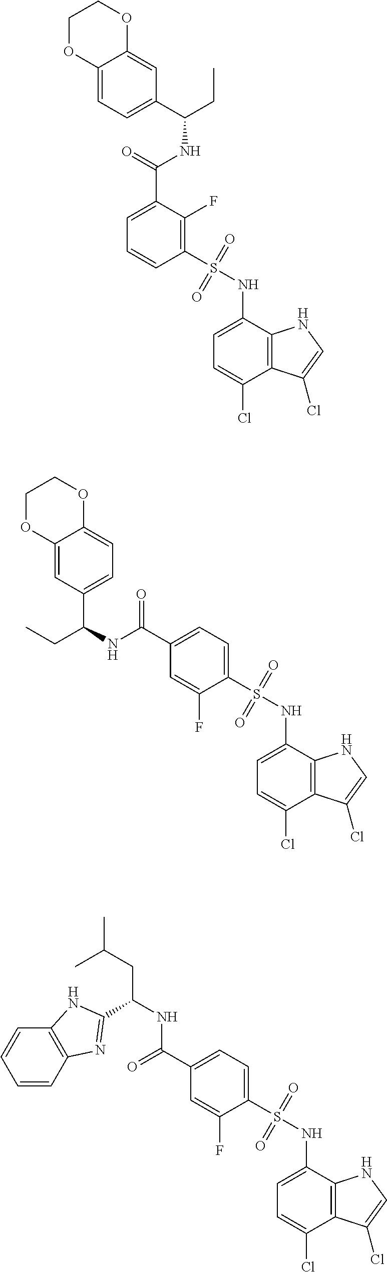 [SMILES: CC[C@H](NC(=O)c1cccc(S(=O)(=O)Nc2ccc(Cl)c3c(Cl)c[nH]c23)c1F)c1ccc2c(c1)OCCO2 ; CC[C@H](NC(=O)c1ccc(S(=O)(=O)Nc2ccc(Cl)c3c(Cl)c[nH]c23)c(F)c1)c1ccc2c(c1)OCCO2 ; CC(C)C[C@H](NC(=O)c1ccc(S(=O)(=O)Nc2ccc(Cl)c3c(Cl)c[nH]c23)c(F)c1)c1nc2ccccc2[nH]1]

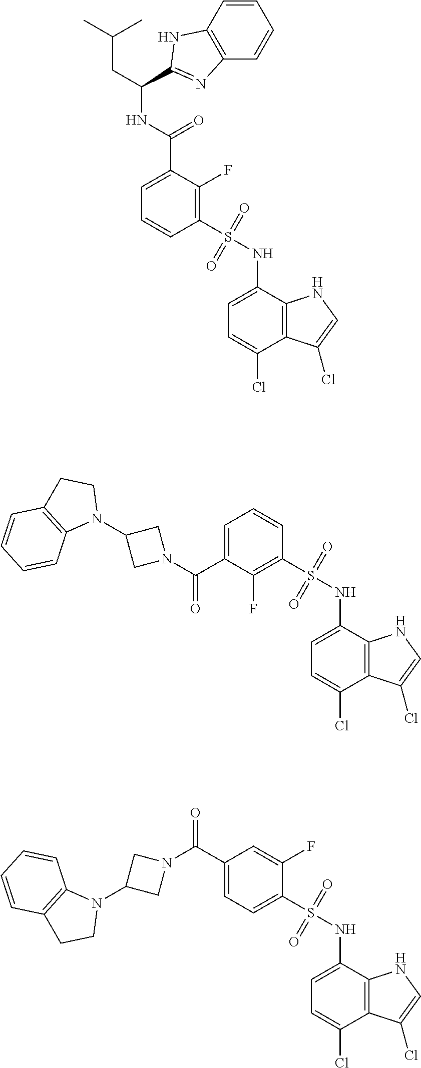 [SMILES: CC(C)C[C@H](NC(=O)c1cccc(S(=O)(=O)Nc2ccc(Cl)c3c(Cl)c[nH]c23)c1F)c1nc2ccccc2[nH]1 ; O=C(c1cccc(S(=O)(=O)Nc2ccc(Cl)c3c(Cl)c[nH]c23)c1F)N1CC(N2CCc3ccccc32)C1 ; O=C(c1ccc(S(=O)(=O)Nc2ccc(Cl)c3c(Cl)c[nH]c23)c(F)c1)N1CC(N2CCc3ccccc32)C1]

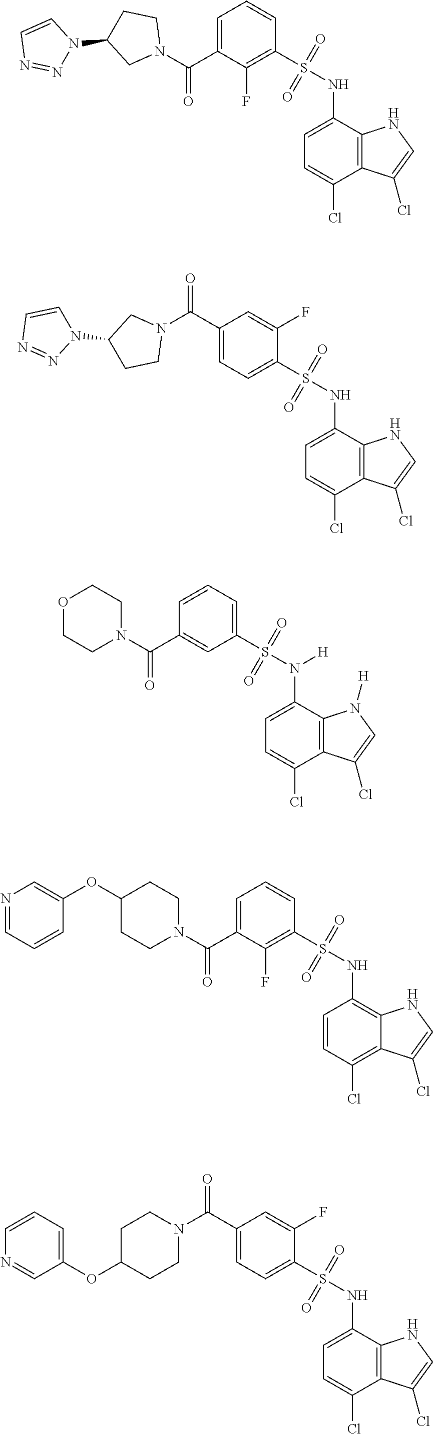 [SMILES: O=C(c1cccc(S(=O)(=O)Nc2ccc(Cl)c3c(Cl)c[nH]c23)c1F)N1CC[C@H](n2ccnn2)C1 ; O=C(c1ccc(S(=O)(=O)Nc2ccc(Cl)c3c(Cl)c[nH]c23)c(F)c1)N1CC[C@H](n2ccnn2)C1 ; O=C(c1cccc(S(=O)(=O)Nc2ccc(Cl)c3c(Cl)c[nH]c23)c1)N1CCOCC1 ; O=C(c1cccc(S(=O)(=O)Nc2ccc(Cl)c3c(Cl)c[nH]c23)c1F)N1CCC(Oc2cccnc2)CC1 ; O=C(c1ccc(S(=O)(=O)Nc2ccc(Cl)c3c(Cl)c[nH]c23)c(F)c1)N1CCC(Oc2cccnc2)CC1]

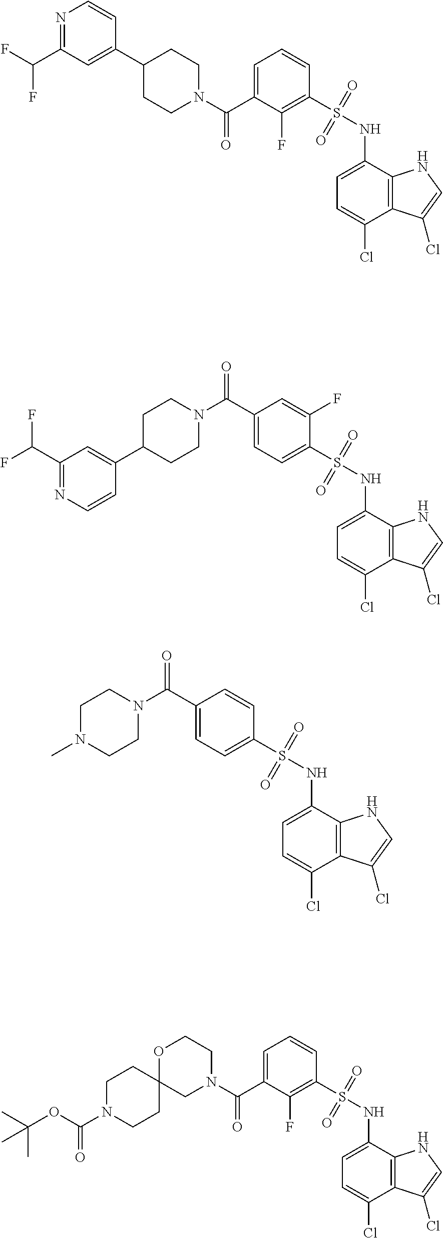 [SMILES: O=C(c1cccc(S(=O)(=O)Nc2ccc(Cl)c3c(Cl)c[nH]c23)c1F)N1CCC(c2ccnc(C(F)F)c2)CC1 ; O=C(c1ccc(S(=O)(=O)Nc2ccc(Cl)c3c(Cl)c[nH]c23)c(F)c1)N1CCC(c2ccnc(C(F)F)c2)CC1 ; CN1CCN(C(=O)c2ccc(S(=O)(=O)Nc3ccc(Cl)c4c(Cl)c[nH]c34)cc2)CC1 ; CC(C)(C)OC(=O)N1CCC2(CC1)CN(C(=O)c1cccc(S(=O)(=O)Nc3ccc(Cl)c4c(Cl)c[nH]c34)c1F)CCO2]

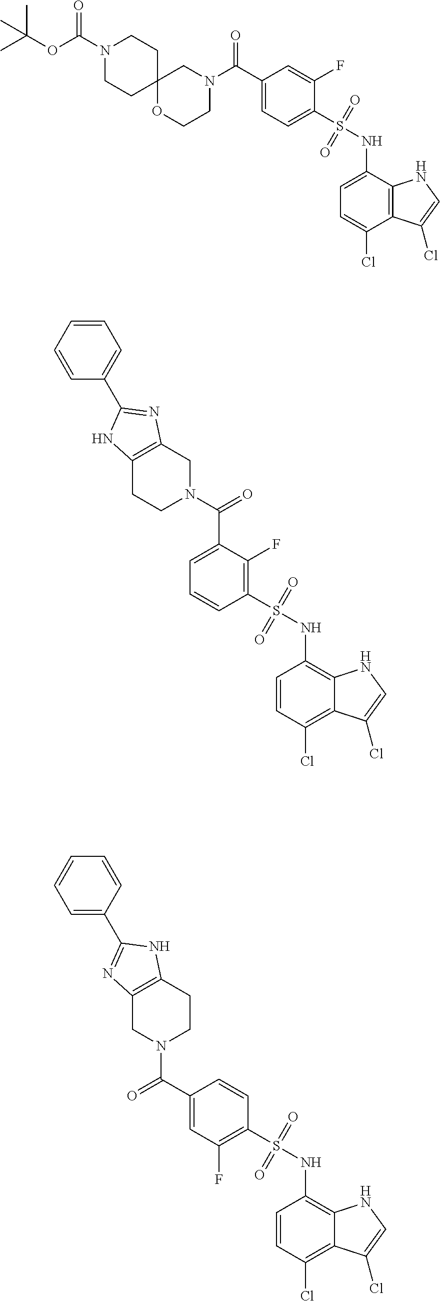 [SMILES: CC(C)(C)OC(=O)N1CCC2(CC1)CN(C(=O)c1ccc(S(=O)(=O)Nc3ccc(Cl)c4c(Cl)c[nH]c34)c(F)c1)CCO2 ; O=C(c1cccc(S(=O)(=O)Nc2ccc(Cl)c3c(Cl)c[nH]c23)c1F)N1CCc2[nH]c(-c3ccccc3)nc2C1 ; O=C(c1ccc(S(=O)(=O)Nc2ccc(Cl)c3c(Cl)c[nH]c23)c(F)c1)N1CCc2[nH]c(-c3ccccc3)nc2C1]

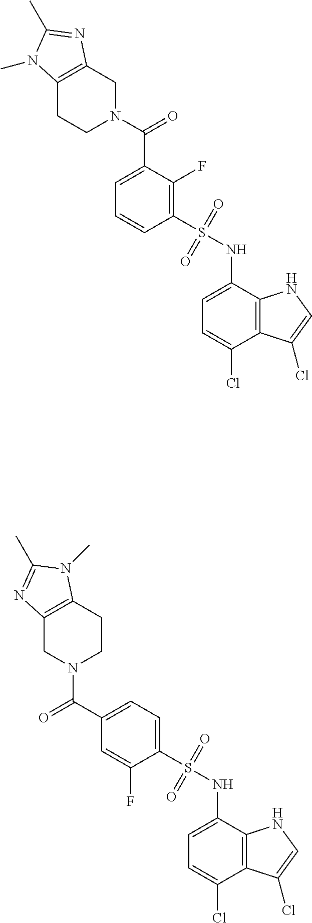 [SMILES: Cc1nc2c(n1C)CCN(C(=O)c1cccc(S(=O)(=O)Nc3ccc(Cl)c4c(Cl)c[nH]c34)c1F)C2 ; Cc1nc2c(n1C)CCN(C(=O)c1ccc(S(=O)(=O)Nc3ccc(Cl)c4c(Cl)c[nH]c34)c(F)c1)C2]

or a pharmaceutically acceptable salt thereof.

In some embodiments the compound of Formula I is a compound of Formula IIIc, Formula IIId, Formula IIIe, Formula IIIf or Formula IIIg:

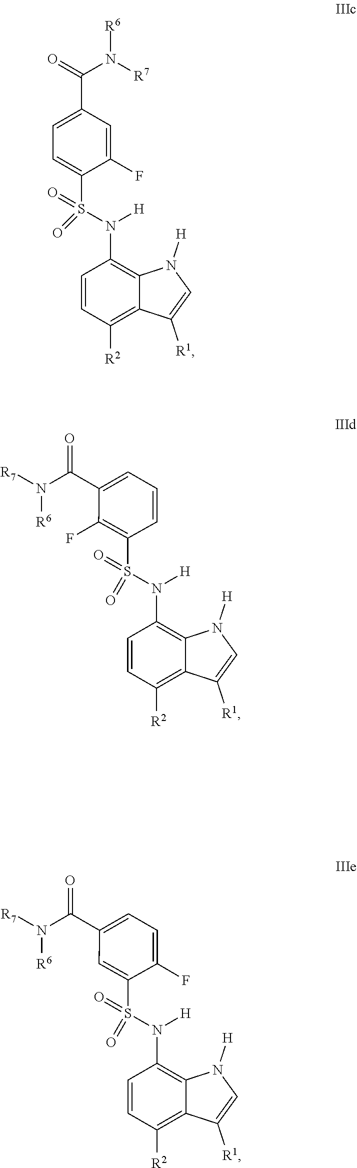

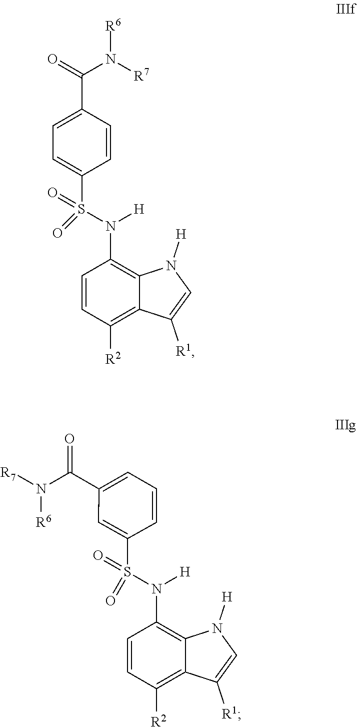

or a pharmaceutically acceptable salt thereof, wherein R¹, R², R⁶, and R⁷, are as described according to any embodiment of compounds of Formula III.

### Additional Compounds

Some embodiments describe a compound selected from any one of the following

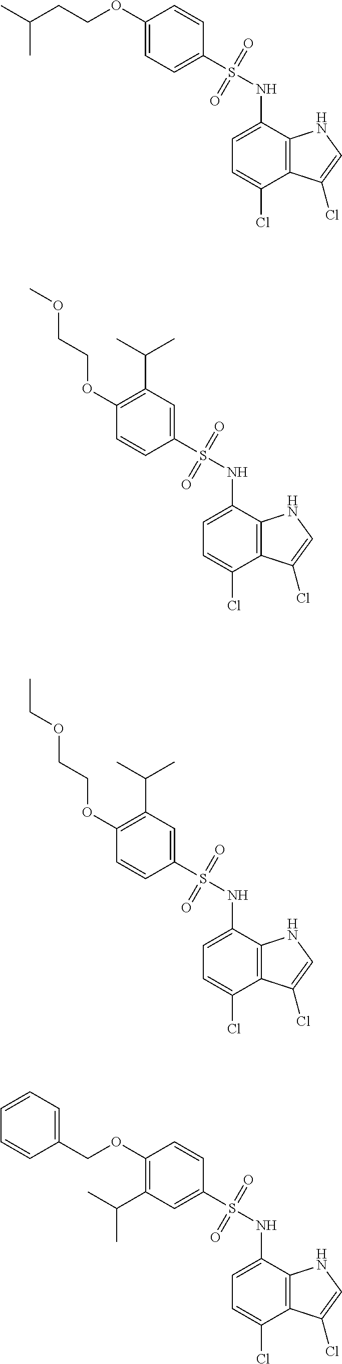 [SMILES: CC(C)CCOc1ccc(S(=O)(=O)Nc2ccc(Cl)c3c(Cl)c[nH]c23)cc1 ; COCCOc1ccc(S(=O)(=O)Nc2ccc(Cl)c3c(Cl)c[nH]c23)cc1C(C)C ; CCOCCOc1ccc(S(=O)(=O)Nc2ccc(Cl)c3c(Cl)c[nH]c23)cc1C(C)C ; CC(C)c1cc(S(=O)(=O)Nc2ccc(Cl)c3c(Cl)c[nH]c23)ccc1OCc1ccccc1]

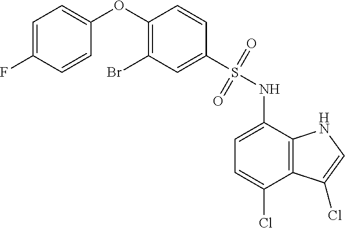 [SMILES: O=S(=O)(Nc1ccc(Cl)c2c(Cl)c[nH]c12)c1ccc(Oc2ccc(F)cc2)c(Br)c1]

or a pharmaceutically acceptable salt thereof.

Some embodiments describe a compound selected from any one of the following

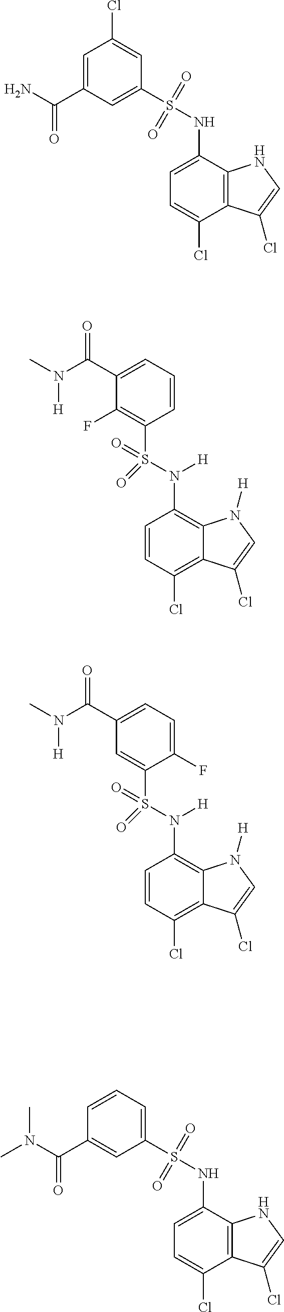 [SMILES: NC(=O)c1cc(Cl)cc(S(=O)(=O)Nc2ccc(Cl)c3c(Cl)c[nH]c23)c1 ; CNC(=O)c1cccc(S(=O)(=O)Nc2ccc(Cl)c3c(Cl)c[nH]c23)c1F ; CNC(=O)c1ccc(F)c(S(=O)(=O)Nc2ccc(Cl)c3c(Cl)c[nH]c23)c1 ; CN(C)C(=O)c1cccc(S(=O)(=O)Nc2ccc(Cl)c3c(Cl)c[nH]c23)c1]

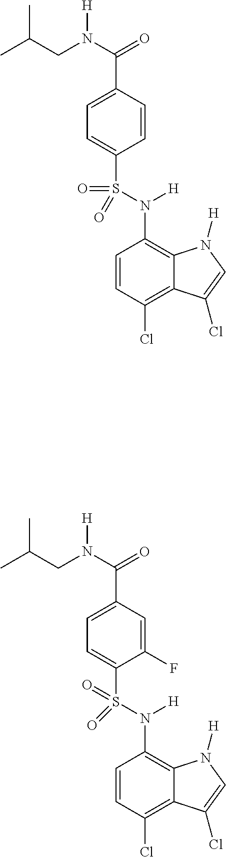 [SMILES: CC(C)CNC(=O)c1ccc(S(=O)(=O)Nc2ccc(Cl)c3c(Cl)c[nH]c23)cc1 ; CC(C)CNC(=O)c1ccc(S(=O)(=O)Nc2ccc(Cl)c3c(Cl)c[nH]c23)c(F)c1]

or a pharmaceutically acceptable salt thereof.

Other compounds as part of the present disclosure are described in the exemplification section. Neutral as well as salt forms of those compounds are included.

Some compounds of embodiments described herein can possess one or more asymmetric carbon atoms and are thus capable of existing in the form of optical isomers as well as in the form of racemic or non-racemic mixtures thereof. The compounds can be utilized in embodiments described herein as a single isomer or as a mixture of stereochemical isomeric forms. Diastereoisomers, i.e., non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of the invention can likewise be obtained by utilizing optically active starting materials. These isomers may be in the form of a free acid, a free base, an ester or a salt.

Compounds according to embodiments described herein may be in the form of pharmaceutically acceptable salts. A pharmaceutically acceptable salt of the compounds described herein includes acid addition salts and base addition salts. Pharmaceutically-acceptable salt embraces salts commonly used to form alkali metal salts and to form addition salts of free acids or free bases The nature of the salt is not critical, provided that it is pharmaceutically-acceptable. Suitable pharmaceutically-acceptable acid addition salts of the compounds described herein may be prepared from an inorganic acid or an organic acid. Examples of such inorganic acids include, without limitation, hydrochloric, hydrobromic, hydroiodic, nitric, carbonic, sulfuric and phosphoric acid. In some embodiments the salt is a hydrochloride salt. Appropriate organic acids may be selected from aliphatic, cycloaliphatic, aromatic, arylaliphatic, heterocyclic, carboxylic and sulfonic classes of organic acids, examples of which include, without limitation, formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, ß-hydroxybutyric, malonic, galactic, and galacturonic acid. Pharmaceutically-acceptable base addition salts for compounds described herein can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cyclo alkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the preparation of pharmaceutically acceptable salts, for example, carboxylic acid amides, including carboxamides, lower alkyl carboxamides, dialkyl carboxamides, and the like.

Acceptable salts may be obtained using standard procedures well known in the art, for example by treating a sufficiently basic compound such as an amine with a suitable acid affording a physiologically acceptable anion. Alkali metal (for example, sodium, potassium or lithium) or alkaline earth metal (for example calcium) salts of organic (e.g., carboxylic) acids can also be made.

In some aspects, compounds of embodiments herein may exist in both unsolvated and solvated forms. The term solvate is used herein to describe a molecular complex comprising a compound of embodiments herein and an amount of one or more pharmaceutically acceptable solvent molecules. The term hydrate is employed when said solvent is water.

Furthermore, it is specifically contemplated that in embodiments herein, more than one solvent molecule may be associated with one molecule of the compounds of embodiments herein, such as a dihydrate. Additionally, it is specifically contemplated that in embodiments herein less than one solvent molecule may be associated with one molecule of the compounds of embodiments herein, such as a hemihydrate. Furthermore, solvates of embodiments herein are contemplated as solvates of compounds of embodiments herein that retain the biological effectiveness of the non-solvate form of the compounds.

In one aspect, embodiments herein also include isotopically-labeled compounds of embodiments herein, wherein one or more atoms is replaced by an atom having the same atomic number, but an atomic mass or mass number different from the atomic mass or mass number usually found in nature. Examples of isotopes suitable for inclusion in the compounds of embodiments herein include isotopes of hydrogen, such as ²H and ³H, carbon, such as ¹¹C, ¹³C and ¹⁴C, chlorine, such as ³¹Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulfur, such as ³⁵S. Certain isotopically-labeled compounds of embodiments herein, for example, those incorporating a radioactive isotope, are useful in drug and/or substrate tissue distribution studies. The radioactive isotopes tritium, ³H, and carbon-14, ¹⁴C, are particularly useful for this purpose in view of their case of incorporation and ready means of detection. Substitution with heavier isotopes such as deuterium, ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of embodiments herein can generally be prepared by conventional techniques known to those skilled in the art or by processes analogous to those described herein, using an appropriate isotopically-labeled reagent in place of the non-labeled reagent otherwise employed.

Preferred isotopically-labeled compounds include deuterated derivatives of the compounds of embodiments herein. As used herein, the term deuterated derivative embraces compounds of embodiments herein where in a particular position at least one hydrogen atom is replaced by deuterium. Deuterium (D or ²H) is a stable isotope of hydrogen which is present at a natural abundance of 0.015 molar %. It is well established that deuteration of physiologically active compounds offer the advantage of retaining the pharmacological profile of their hydrogen counterparts while positively impacting their metabolic outcome. Selective replacement of one or more hydrogen with deuterium, in a compound of the present invention, could improve the safety, tolerability and efficacy of the compound when compared to its all hydrogen counterpart.

Methods for incorporation of deuterium into compounds is well established. Using metabolic studies establish in the art, the compound of the present invention can be tested to identify sites for selective placement of a deuterium isotope, wherein the isotope will not be metabolized. Moreover these studies identify sites of metabolism as the location where a deuterium atom would be placed.

Hydrogen deuterium exchange (deuterium incorporation) is a chemical reaction in which a covalently bonded hydrogen atom is replaced by a deuterium atom. Said exchange (incorporation) reaction can be total or partial.

Typically, a deuterated derivative of a compound of embodiments herein has an isotopic enrichment factor (ratio between the isotopic abundance and the natural abundance of that isotope, i.e. the percentage of incorporation of deuterium at a given position in a molecule in the place of hydrogen) for each deuterium present at a site designated as a potential site of deuteration on the compound of at least 3500 (52.5% deuterium incorporation).

In some embodiments, the isotopic enrichment factor is at least 5000 (75% deuterium). In some embodiments, the isotopic enrichment factor is at least 6333.3 (95% deuterium incorporation). In some embodiments, the isotopic enrichment factor is at least 6633.3 (99.5% deuterium incorporation). It is understood that the isotopic enrichment factor of each deuterium present at a site designated as a site of deuteration is independent from the other deuteration sites.

The isotopic enrichment factor can be determined using conventional analytical methods known to one of ordinary skilled in the art, including mass spectrometry (MS) and nuclear magnetic resonance (NMR).

In certain aspects, prodrugs of the compounds described herein are also within the scope of embodiments herein. Thus, certain derivatives of the compounds of embodiments herein, which derivatives may have little or no pharmacological activity themselves, when administered into or onto the body may be converted into compounds of embodiments herein having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with embodiments herein can, for example, be produced by replacing appropriate functionalities present in the compounds of embodiments herein with certain moieties known to those skilled in the art as ‘pro-moieties’ as described, for example, in Design of Prodrugs by H. Bundgaard (Elsevier, 1985).

In the case of compounds of embodiments herein that are solids, it is understood by those skilled in the art that the inventive compounds and salts may exist in different crystalline or polymorphic forms, or in an amorphous form, all of which are intended to be within the scope of embodiments herein.

The compounds disclosed herein can exist as and therefore include all tautomers, and mixtures thereof in all proportions as well as isotopic forms such as deuterated compounds.

The invention also embraces isolated compounds. An isolated compound refers to a compound which represents at least 10%, preferably at least 20%, more preferably at least 50% and most preferably at least 80% of the compound present in the mixture.

### Pharmaceutical Compositions

Some embodiments describe a pharmaceutical composition comprising: a compound according to any embodiment described herein or a pharmaceutically acceptable salt thereof; and a pharmaceutically acceptable carrier or diluent. The pharmaceutical compositions can be prepared in a manner well known in the pharmaceutical art, and can be administered by a variety of routes, depending upon whether local or systemic treatment is desired and upon the area to be treated.

While it is possible that a compound as described in any embodiment herein, may be administered as the bulk substance, it is preferable to present the compound in a pharmaceutical formulation, e.g., wherein the active agent is in an admixture with a pharmaceutically acceptable carrier selected with regard to the intended route of administration and standard pharmaceutical practice.

In particular, the disclosure provides a pharmaceutical composition comprising a therapeutically effective amount of at least one compound according to any embodiment described herein, and optionally, a pharmaceutically acceptable carrier.

### Combinations

For the pharmaceutical compositions and methods of the disclosure, a compound according to any embodiment described herein, may be used in combination with other therapies and/or active agents.

Accordingly, the disclosure provides, in a further aspect, a pharmaceutical composition comprising at least one compound according to any embodiment described herein, or pharmaceutically acceptable derivative thereof; a second active agent; and, optionally a pharmaceutically acceptable carrier.

When used in combination therapy, the compounds described herein are administered with the second active agent simultaneously or separately. Thus the combination of the two agents can be administered simultaneously in the same dosage form, simultaneously in separate dosage forms or administered separately. When combined in the same formulation it will be appreciated that the two or more compounds must be stable and compatible with each other and the other components of the formulation. When formulated separately they may be provided in any convenient formulation, in such a manner as are known for such compounds in the art. In some embodiments, a compound according to any embodiment described herein is administered first, followed by administration of the second agent. In some embodiments the second agent is administered first, followed by administration of the second. In some embodiments the administration of the two agents can begin simultaneously, but is not completed at the same time. For example, one agent is administered orally, simultaneously with commencement of the administration of a second agent via a thirty minute bolus IV. In some embodiments a compound according to embodiments described herein and a second active agent are administered separately a few minutes apart, a few hours apart or a few days apart.

Preservatives, stabilizers, dyes and flavoring agents may be provided in any pharmaceutical composition described herein. Examples of preservatives include sodium benzoate, ascorbic acid and esters of p-hydroxybenzoic acid. Antioxidants and suspending agents may be also used.

### Routes of Administration and Unit Dosage Forms

Compounds according to any embodiment described herein, or pharmaceutically acceptable salts thereof, a solvate thereof, a stereoisomer thereof, a prodrug thereof, or an active metabolites thereof, can be formulated for any route of administration.

The routes for administration (delivery) include, but are not limited to, one or more of: oral (e.g., as a tablet, capsule, or as an ingestible solution), topical, mucosal (e.g., as a nasal spray or aerosol for inhalation), parenteral (e.g., by an injectable form), gastrointestinal, intraspinal, intraperitoneal, intramuscular, intravenous, intracerebroventricular, or other depot administration etc.

Therefore, the pharmaceutical compositions according to any embodiment described herein, include those in a form especially formulated for the mode of administration. In certain embodiments, the pharmaceutical compositions of the disclosure are formulated in a form that is suitable for oral delivery. In some embodiments, the compound is an orally bioavailable compound, suitable for oral delivery. In other embodiments, the pharmaceutical compositions of the disclosure are formulated in a form that is suitable for parenteral delivery.

The compounds according to any embodiment described herein, may be formulated for administration in any convenient way for use in human or veterinary medicine and the disclosure therefore includes within its scope pharmaceutical compositions comprising a compound according to any embodiment described herein, adapted for use in human or veterinary medicine. Such pharmaceutical compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

There may be different pharmaceutical composition/formulation requirements depending on the different delivery systems. It is to be understood that not all of the compounds need to be administered by the same route. Likewise, if the pharmaceutical composition comprises more than one active component, then those components may be administered by different routes. By way of example, the pharmaceutical composition of the disclosure may be formulated to be delivered using a mini-pump or by a mucosal route, for example, as a nasal spray or aerosol for inhalation or ingestible solution, or parenterally in which the pharmaceutical composition is formulated by an injectable form, for delivery by, for example, an intravenous, intramuscular or subcutaneous route. Alternatively, the formulation may be designed to be delivered by multiple routes.

Where appropriate, the pharmaceutical compositions according to any embodiment described herein, can be administered by inhalation, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the pharmaceutical compositions according to any embodiment described herein, may be administered in the form of tablets or lozenges, which can be formulated in a conventional manner.

Where the pharmaceutical composition according to any embodiment described herein, is to be administered parenterally, such administration includes without limitation: intravenously, intraarterially, intrathecally, intraventricularly, intracranially, intramuscularly or subcutaneously administering the compound of the disclosure; and/or by using infusion techniques.

Pharmaceutical compositions according to any embodiment described herein, suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or ascorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminum monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound according to any embodiment described herein, is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound according to any embodiment described herein, with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the compounds and desired excipients for subsequent preparation of sterile solutions.

The compounds according to any embodiment described herein, may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The pharmaceutical compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds according to any embodiment described herein, can be administered in the form of tablets, capsules, troches, ovules, elixirs, solutions or suspensions, for immediate-, delayed-, modified-, sustained-, pulsed- or controlled-release applications.

The compounds according to any embodiment described herein, may also be presented for human or veterinary use in a form suitable for oral or buccal administration, for example in the form of solutions, gels, syrups, or suspensions, or a dry powder for reconstitution with water or other suitable vehicle before use. Solid pharmaceutical compositions such as tablets, capsules, lozenges, troches, pastilles, pills, boluses, powder, pastes, granules, bullets or premix preparations may also be used. Solid and liquid pharmaceutical compositions for oral use may be prepared according to methods well-known in the art. Such pharmaceutical compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The pharmaceutical compositions according to any embodiment described herein, may be administered orally, in the form of rapid or controlled release tablets, microparticles, mini tablets, capsules, sachets, and oral solutions or suspensions, or powders for the preparation thereof. Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the pharmaceutical compositions, e.g., act as both binders and disintegrants.

Examples of pharmaceutically acceptable disintegrants for oral pharmaceutical compositions according to any embodiment described herein, include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral pharmaceutical compositions according to any embodiment described herein, include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthine resin, alginates, magnesium aluminum silicate, polyethylene glycol or bentonite.

Examples of pharmaceutically acceptable fillers for oral pharmaceutical compositions according to any embodiment described herein, include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the pharmaceutical compositions according to any embodiment described herein, include, but are not limited to, magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odorants for the oral pharmaceutical compositions according to any embodiment described herein, include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral pharmaceutical compositions according to any embodiment described herein, include, but are not limited to, synthetic and natural dyes such as titanium dioxide, beta-carotene and extracts of grapefruit peel.

Examples of useful pharmaceutically acceptable coatings for the oral pharmaceutical compositions according to any embodiment described herein, typically used to facilitate swallowing, modify the release properties, improve the appearance, and/or mask the taste of the pharmaceutical compositions include, but are not limited to, hydroxypropylmethylcellulose, hydroxypropylcellulose and acrylate-methacrylate copolymers.

Suitable examples of pharmaceutically acceptable sweeteners for the oral pharmaceutical compositions according to any embodiment described herein, include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulphate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

As indicated, a compounds according to any embodiment described herein, can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of a compound according to any embodiment described herein, and a suitable powder base such as lactose or starch.

For topical administration by inhalation a compounds according to any embodiment described herein, may be delivered for use in human or veterinary medicine via a nebulizer.

The pharmaceutical compositions of the disclosure may contain from 0.01 to 99% weight per volume of the active material. For topical administration, for example, the pharmaceutical composition will generally contain from 0.01-10%, more preferably 0.01-1% of the active material.

A compound according to any embodiment described herein, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage form, according to any embodiment described herein, may be administered according to a dosage and administration regimen defined by routine testing in the light of the guidelines given above in order to obtain optimal activity while minimizing toxicity or side effects for a particular patient. The dosage of the compounds or unit dosage form may vary according to a variety of factors such as underlying disease conditions, the individual's condition, weight, sex and age, and the mode of administration. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter is within the scope of the disclosure. Clinically significant attenuation or amelioration means perceptible to the patient and/or to the physician.

A pharmaceutical composition for parenteral administration contains from about 0.01% to about 100% by weight of the active compound according to any embodiment described herein, based upon 100% weight of total pharmaceutical composition.

Generally, transdermal dosage forms contain from about 0.01% to about 100% by weight of the active compound according to any embodiment described herein, versus 100% total weight of the dosage form.

The pharmaceutical composition or unit dosage form may be administered in a single daily dose, or the total daily dosage may be administered in divided doses. In addition, co administration or sequential administration of another compound for the treatment of the disorder may be desirable. To this purpose, the combined active principles are formulated into a simple dosage unit.

### Methods of Use

In some embodiments, the present disclosure provides a method of inducing degradation of a protein comprising contacting the protein with an effective amount of a compound or composition according to any embodiment described herein.

Without wishing to be bound by any particular theory, the inventors believe that a compound according to any embodiment herein, can act as a molecular glue and induce an interaction between a ubiquitin E3 ligase and a target protein, leading to degradation of the target protein. Inducing protein degradation results in a decrease in protein levels. In some embodiments, the compounds promotes the formation of a complex between the protein and a substrate recognition subunit of the E3 ligase. In some instances a compound according to any embodiment herein binds to the ubiquitin E3 ligase and recruits proteins for degradation by the ubiquitin-proteasome system. In some embodiments the substrate recognition subunit of the E3 ligase is DCAF15. In some embodiments the substrate recognition subunit of the E3 ligase is human DCAF15. An example of human DCAF15 is Uniprot ID: Q66K64. In some embodiments the compound binds to the protein. In some embodiments the protein is a protein that may lack enzymatic active sites that can be targeted by inhibitors. In some embodiments the protein is RBM39. In some embodiments the substrate recognition subunit of the E3 ligase is DCAF15 and the protein is RBM39.

Some embodiments describe a method of inducing degradation of a protein, in a patient in need thereof, comprising administering to the patient, a therapeutically effective amount of a compound according to any embodiment described herein.

In some embodiments the compounds and compositions according to embodiments of the present invention are useful for treating a disease, disorder or condition associated with a target protein. Any disease, disorder or condition that results directly or indirectly from abnormal activity of the target protein or expression level of the gene that encodes the target protein can be an intended disease condition. Some embodiments describe a method of treating a disease or disorder that results from abnormal activity of a target protein in a subject, comprising administering to the subject, a therapeutically effective amount of a compound according to any embodiment described herein, wherein the compound induces degradation of the protein thereby treating the disease or disorder. In some embodiments the disease or disorder results directly from abnormal activity of a target protein. In some embodiments the disease or disorder results indirectly from the abnormal activity of the target protein. Some embodiments describe a method of treating a disease or disorder that results from abnormal expression of a gene that encodes a target protein in a subject, comprising administering to the subject, a therapeutically effective amount of a compound according to any embodiment described herein, wherein the compound induces degradation of the protein thereby treating the disease or disorder.

Some embodiments of the invention describe use of a compound according to any embodiment described herein, in the preparation of a medicament for promoting the degradation of a protein, in a patient in need thereof. Some embodiments describe a compound according to any embodiment described herein for use in medical therapy.

A number of assay systems can be used to determine if the compounds according to embodiments describe herein, are useful in the methods described above. For example, assays that measure if the compound induces the formation of a complex between the E3-ligase subunit and the target protein; assays that measure if the compound binds the E-3 ligase subunit, assays that measure if protein expression is decreased in a cell upon addition of the compounds and assays that measure cell viability. A non-limiting list of assays includes amplified luminescent proximity homogenous assay, homogeneous time fluorescence competition assays, Western blot assay, and CellTiter-Glo® Luminescent Cell Viability Assay.

These assays can be conducted with one or more positive control compounds-compounds that are known to induce an interaction between a ubiquitin E3 ligase and a target protein, leading to the degradation of the target protein. Examples of such positive control compounds, includes indisulam, E-7820; tasisulam and cloroquinoxaline sulfonamide (CQS).

### EXAMPLES

The following examples are given to illustrate the present subject matter. It should be understood, however, that the subject matter is not limited to the specific conditions or details described in these examples.

### Example 1: Preparation of 3-cyano-N-(3-cyano-4-methyl-1H-indol-7-yl)-5-fluorobenzenesulfonamide; Intermediate A

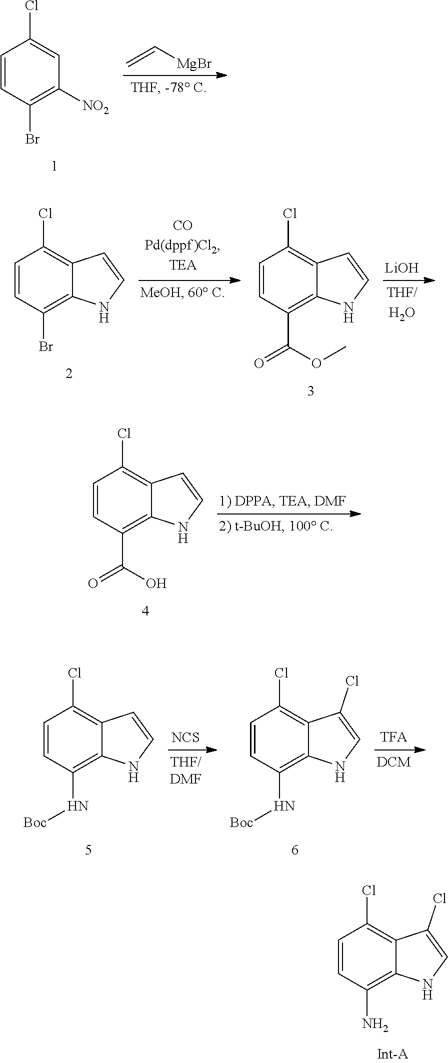

### 1. Preparation of 7-bromo-4-chloro-1H-indole

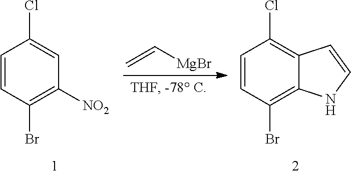

To a solution of 1-bromo-4-chloro-2-nitrobenzene (10.0 g, 42.29 mmol) in THF (100 mL) was added slowly bromo(vinyl)magnesium (211.5 mL, 211.46 mmol) at −78° C. under N₂ atmosphere. The mixture was stirred at −78° C. for 1 h. The mixture was quenched with sat. NH₄Cl and extracted with EA. The organic layer was concentrated and the residue was purified by chromatography column on silica gel to give 7-bromo-4-chloro-1H-indole (4.5 g, 45.8%) as a light yellow solid. LCMS (ESI): 230.0 [M+H]+.

### 2. Preparation of methyl 4-chloro-1H-indole-7-carboxylate

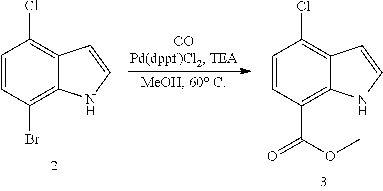

To a solution of 7-bromo-4-chloro-1H-indole (1.0 g, 4.34 mmol) in MeOH (10 mL), were added TEA (2 mL) and Pd(dppf)Cl₂ (320.0 mg, 0.43 mmol). The mixture was stirred at 60° C. under CO atmosphere (200 psi.) overnight. The mixture was filtered and the filtrate was concentrated. The residue was purified by chromatography column on silica gel eluting with PE/EA from 40/1 to 15/1 to afford methyl 4-chloro-1H-indole-7-carboxylate (542.0 mg, 59.6%) as a light-yellow solid. LCMS (ESI): 210 [M+H]⁺.

### 3. Preparation of 4-chloro-1H-indole-7-carboxylic acid

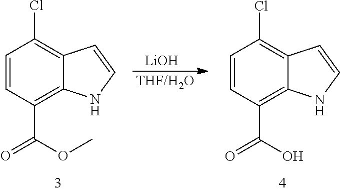

To a solution of methyl 4-chloro-1H-indole-7-carboxylate (200.0 mg, 0.95 mmol) in THF/H₂O (1:1, 4 mL), was added LiOH·H₂O (160.0 mg, 3.82 mmol). The mixture was stirred at rt for 3 h. The mixture was concentrated and diluted with H₂O (15 mL), then the mixture was acidified with 1N HCl aq. to pH˜2. A solid precipitated was collected by filtration. The filtered cake was dissolved in EA, dried over anhydrous Na₂SO₄, then filtered and the filtrate was concentrated to give 4-chloro-1H-indole-7-carboxylic acid (207.0 mg, 98.0%) as a white solid. LCMS (ESI): 194 [M−H]⁻.

### 4. Preparation of tert-butyl (4-chloro-1H-indol-7-yl)carbamate

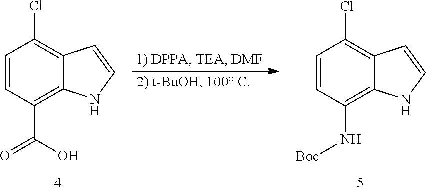

To a solution of 4-chloro-1H-indole-7-carboxylic acid (100.0 mg, 0.51 mmol) in t-Butanol (5 mL) was added TEA (207.6 mg, 207.6 mmol) and DPPA (169 mg, 0.61 mmol) under N₂ atmosphere, the mixture was stirred at 100° C. overnight. The reaction mixture was diluted with EA (10 mL). The mixture was washed with 1N HCl aq. (10 mL), water (10 mL) and brine (10 mL). The organic phase was dried over Na₂SO₄ and concentrated to give a residue, which was purified by column chromatography on silica gel eluting with PE/EA from 20/1 to 8/1 to afford tert-butyl N-(4-chloro-1H-indol-7-yl)carbamate (41.0 mg, 0.15 mmol, yield: 29.41%) as a white solid. LCMS (ESI): 267 [M+H]⁺.

### 5. Preparation of tert-butyl (3,4-dichloro-1H-indol-7-yl)carbamate

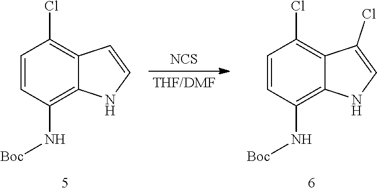 [SMILES: CC(C)(C)OC(=O)Nc1ccc(Cl)c2cc[nH]c12 ; "NCS" ; "THF/DMF" ; CC(C)(C)OC(=O)Nc1ccc(Cl)c2c(Cl)c[nH]c12]

To a solution of tert-butyl N-(4-chloro-1H-indol-7-yl)carbamate (500.0 mg, 1.88 mmol) in THF/DMF (10:1, 10 mL) was added NCS (262.9 mg, 1.97 mmol) at 0° C. The mixture was stirred at rt for 1 h. The reaction was quenched with saturated Na₂SO₃ solution (5 mL). The mixture was extracted with EA (20 mL). The organic layer was concentrated and purified by column chromatography on silica gel eluting with PE/EA from 20/1 to 8/1 to afford tert-butyl N-(3,4-dichloro-1H-indol-7-yl)carbamate (352.0 mg, 62.4%) as a white solid. LCMS (ESI): 301 [M+H]⁺.

### 6. Preparation of 3,4-dichloro-1H-indol-7-amine

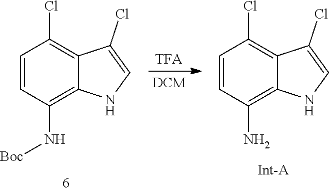

To a solution of tert-butyl N-(3,4-dichloro-1H-indol-7-yl)carbamate (2.4 g, 7.97 mmol) in DCM (20 mL) was added TFA (4 mL). The mixture was stirred at rt for 5 h. The mixture was concentrated and diluted with EA. The mixture was washed with sat. NaHCO₃. The organic layer was washed with brine, dried over Na₂SO₄, filtered, concentrated. The residue was recrystallized from DCM/hexane to afford 3,4-dichloro-1H-indol-7-amine (Int-A, 1.0 g, 65.6%) as a brown solid. LCMS (ESI): 200 [M+H]⁺. ¹H NMR (400 MHZ, DMSO-d6) δ 11.20 (s, 1H), 7.50 (d, J=4.0 Hz, 1H), 6.78 (d, J=8.0 Hz, 1H), 6.32 (d, J=8.0 Hz, 1H), 5.28 (s, 2H).

### Example 2: Preparation of N-(3,4-dichloro-1H-indol-7-yl)-4-((4-methylpiperidin-4-yl)oxy)benzenesulfonamide; Compound 22

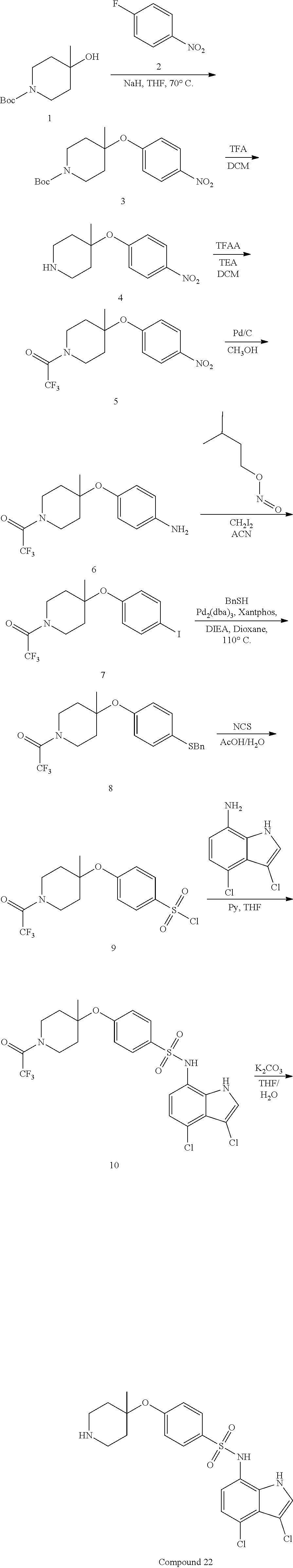

### 1. Preparation of tert-butyl 4-methyl-4-(4-nitrophenoxy)piperidine-1-carboxylate

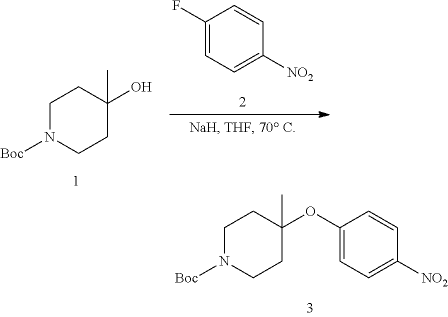 [SMILES: CC1(O)CCN(C(=O)OC(C)(C)C)CC1 ; O=[N+]([O-])c1ccc(F)cc1 ; CC(C)(C)OC(=O)N1CCC(C)(Oc2ccc([N+](=O)[O-])cc2)CC1]

To a solution of tert-butyl 4-hydroxy-4-methylpiperidine-1-carboxylate (10.0 g, 46.45 mmol) in THF (150 mL) was added NaH (60% in mineral oil, 3.7 g, 92.90 mmol). The mixture was stirred at rt for 30 min. 1-fluoro-4-nitrobenzene (13.1 g, 92.90 mmol) was added. The mixture was stirred at 70° C. for 18 h. After completion, the reaction was quenched with sat. NH₄Cl (200 mL) and extracted with EA (150 mL×3). The combined organic layer was dried over MgSO₄, filtered and concentrated to give a crude product, which was purified by column chromatography on silica gel eluting with PE/EA from 25/1 to 5/1 to give tert-butyl 4-methyl-4-(4-nitrophenoxy)piperidine-1-carboxylate (7.6 g, yield: 48.5%) as a white solid. LCMS (ESI) found: 337 [M+H]⁺.

### 2. Preparation of 4-methyl-4-(4-nitrophenoxy)piperidine

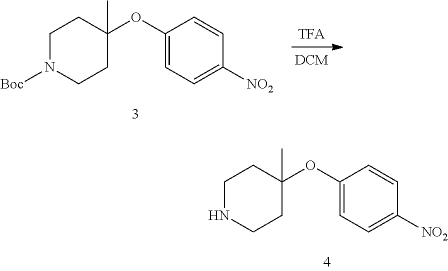 [SMILES: CC(C)(C)OC(=O)N1CCC(C)(Oc2ccc([N+](=O)[O-])cc2)CC1 ; CC1(Oc2ccc([N+](=O)[O-])cc2)CCNCC1]

To a solution of tert-butyl 4-methyl-4-(4-nitrophenoxy)piperidine-1-carboxylate (7.6 g, 22.51 mmol) in DCM (50 mL) was added TFA (12.0 mL) slowly. The mixture was stirred for at rt for 2 h until the starting material was consumed completely. The reaction mixture was concentrated and basified NaHCO₃ solution (100 mL). The mixture was extracted with DCM (150 mL×3). The combined organic layer was dried over MgSO₄, filtered and concentrated to give a crude product, which was purified by column chromatography on silica gel eluting with DCM/MeOH from 25/1 to 20/1 to give 4-methyl-4-(4-nitrophenoxy)piperidine (4.2 g, yield: 79.7%) as a white solid. LCMS (ESI) found: 237 [M+H]⁺.

### 3. Preparation of 2,2,2-trifluoro-1-(4-methyl-4-(4-nitrophenoxy)piperidin-1-yl)ethan-1-one

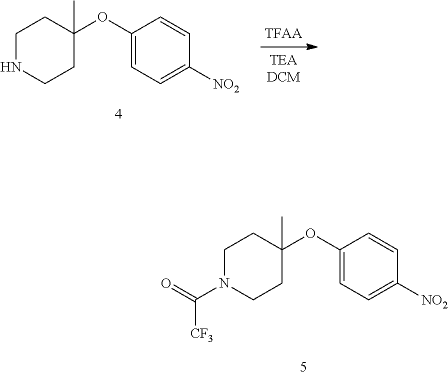 [SMILES: CC1(Oc2ccc([N+](=O)[O-])cc2)CCNCC1 ; "TFAA" ; CC1(Oc2ccc([N+](=O)[O-])cc2)CCN(C(=O)C(F)(F)F)CC1]

To a solution of 4-methyl-4-(4-nitrophenoxy)piperidine (4.2 g, 17.95 mmol) in DCM (50 mL) were added TEA (3.6 g, 35.90 mmol) and TFAA (7.5 g, 35.90 mmol) at 0° C. The mixture was stirred at rt for 2 h. The mixture was concentrated and diluted with water (100 ml). The mixture was extracted with DCM (150 mL×3). The combined organic layer was dried over MgSO₄, filtered and concentrated to give a crude product, which was purified by column chromatography on silica gel eluting with DCM/MeOH from 35/1 to 25/1 to give 2,2,2-trifluoro-1-(4-methyl-4-(4-nitrophenoxy) piperidin-1-yl)ethan-1-one (3.8 g, yield: 64.0%) as a yellow solid. LCMS (ESI) found: 333 [M+H]⁺.

### 4. Preparation of 1-(4-(4-aminophenoxy)-4-methylpiperidin-1-yl)-2,2,2-trifluoro ethan-1-one

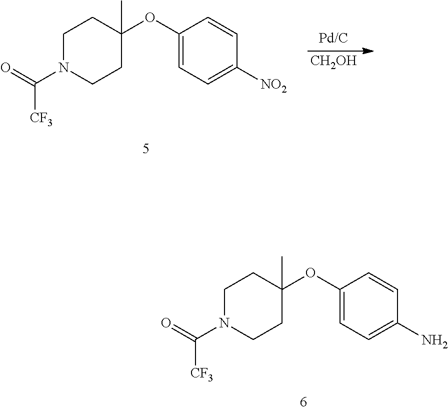 [SMILES: CC1(Oc2ccc([N+](=O)[O-])cc2)CCN(C(=O)C(F)(F)F)CC1 ; "CH2OH" ; "Pd/C" ; CC1(Oc2ccc(N)cc2)CCN(C(=O)C(F)(F)F)CC1]

To a solution of 2,2,2-trifluoro-1-(4-methyl-4-(4-nitrophenoxy) piperidin-1-yl)ethan-1-one (3.8 g, 11.49 mmol) in CH₃OH (50 mL) was added Pd/C. The mixture was charged with H₂ for three times and stirred at rt under H₂ atmosphere for 1 hour until the starting material was consumed completely. The reaction mixture was filtered. The filtrate was concentrated to give a crude product, which was purified by column chromatography on silica gel eluting with PE/EA from 10/1 to 3/1 to give 1-(4-(4-aminophenoxy)-4-methylpiperidin-1-yl)-2,2,2-trifluoroethan-1-one (2.6 g, yield: 75.0%) as a white solid. LCMS (ESI) found: 303 [M+H]⁺.

### 5. Preparation of 2,2,2-trifluoro-1-(4-(4-iodophenoxy)-4-methylpiperidin-1-yl)ethan-1-one

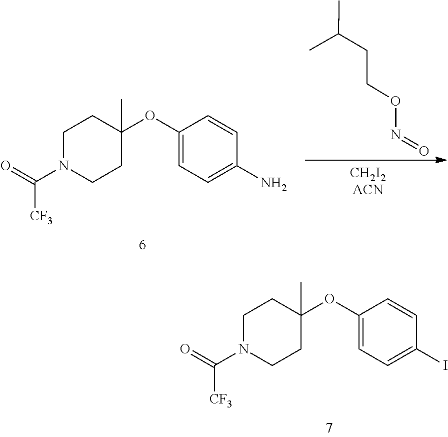

To a solution of 1-(4-(4-aminophenoxy)-4-methylpiperidin-1-yl)-2,2,2-trifluoroethan-1-one (2.6 g, 8.61 mmol) in ACN (25 mL) were added 3-methylbutyl nitrite (2.0 g, 17.24 mmol) and diiodomethane (4.6 g, 17.24 mmol). The mixture was stirred at rt for 2 h until the starting material was consumed completely. The mixture was quenched with sat. NaHCO₃ (20 mL) and extracted with EA (50 mL×3). The combined organic layer was dried over MgSO₄, filtered and concentrated to give a crude product, which was purified by column chromatography on silica gel eluting with PE/EA from 25/1 to 10/1 to give 2,2,2-trifluoro-1-(4-(4-iodophenoxy)-4-methyl piperidin-1-yl)ethan-1-one (2.2 g, yield: 60.8%) as a white solid. LCMS (ESI) found: 414 [M+H]⁺.

### 6. Preparation of 1-(4-(4-(benzylthio)phenoxy)-4-methylpiperidin-1-yl)-2,2,2-trifluoroethan-1-one

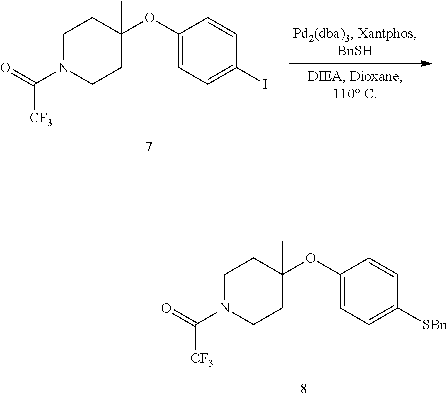

To a solution of 2,2,2-trifluoro-1-[4-(4-iodophenoxy)-4-methylpiperidin-1-yl]ethan-1-one (2.2 g, 5.23 mmol) and BnSH (1.3 g, 10.46 mmol) in dioxane (25 mL) were added DIEA (1.7 g, 13.07 mmol), Pd₂(dba)₃ (478.9 mg, 0.52 mmol) and Xantphos (300.8 mg, 0.52 mmol). The mixture was charged with N₂ for three times and stirred at 110° C. for 2 h under N₂ atmosphere. LCMS showed the reaction was completed. The mixture was cooled to room temperature, filtered and concentrated. The residue was poured into water (60 mL) and extracted with EA (60 mL×3). The combined organic layer was dried over MgSO₄, filtered and concentrated to give a crude product, which was purified by column chromatography on silica gel eluting with PE/EA from 25/1 to 10/1 to give 1-(4-(4-(benzylthio)phenoxy)-4-methylpiperidin-1-yl)-2,2,2-trifluoroethan-1-one (1.6 g, yield: 72.6%) as a yellow solid. LCMS (ESI) found: 410 [M+H]⁺.

### 7. Preparation of 4-((4-methyl-1-(2,2,2-trifluoroacetyl)piperidin-4-yl)oxy)benzenesulfonyl chloride

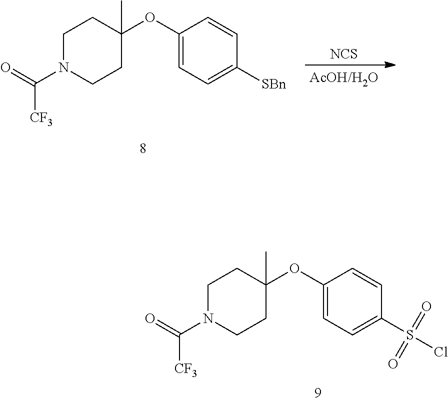

To a solution of 1-(4-(4-(benzylthio)phenoxy)-4-methylpiperidin-1-yl)-2,2,2-trifluoro ethan-1-one (200.0 mg, 0.48 mmol) in AcOH/H₂O (9/1, 4 mL) was added NCS (111.5 mg, 1.92 mmol). The mixture was stirred at rt for 1 hour until the starting material was consumed completely. The reaction was quenched with aqueous Na₂S203 (15 mL) and extracted with DCM (15 mL×3). The combined organic layer was concentrated to give a crude product, which was purified by column chromatography on silica gel eluting with PE/EA from 15/1 to 3/1 to give 4-((4-methyl-1-(2,2,2-trifluoroacetyl)piperidin-4-yl)oxy)benzenesulfonyl chloride (160.0 mg, yield: 86.4%) as a white solid. LCMS (ESI) found: 386 [M+H]⁺.

### 8. Preparation of N-(3,4-dichloro-1H-indol-7-yl)-4-((4-methyl-1-(2,2,2-trifluoroacetyl) piperidin-4-yl)oxy)benzenesulfonamide

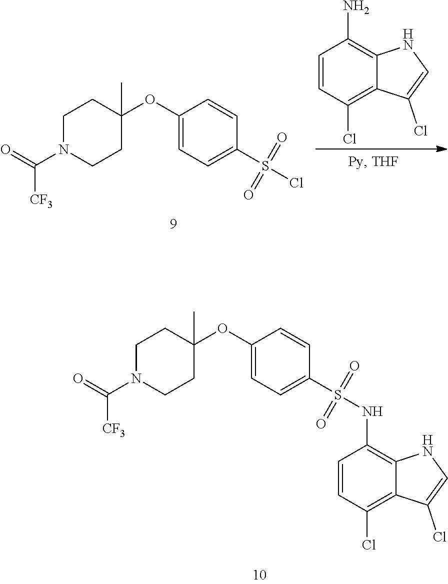 [SMILES: CC1(Oc2ccc(S(=O)(=O)Cl)cc2)CCN(C(=O)C(F)(F)F)CC1 ; Nc1ccc(Cl)c2c(Cl)c[nH]c12 ; CC1(Oc2ccc(S(=O)(=O)Nc3ccc(Cl)c4c(Cl)c[nH]c34)cc2)CCN(C(=O)C(F)(F)F)CC1]

To a solution of 3,4-dichloro-1H-indol-7-amine (52.1 mg, 0.26 mmol) and Pyridine (61.5 mg, 0.78 mmol) in THF (5 mL) was added 4-((4-methyl-1-(2,2,2-trifluoroacetyl)piperidin-4-yl)oxy)benzenesulfonyl chloride (100.0 mg, 0.26 mmol). The mixture was stirred at rt for 18 h until the starting material was consumed completely. The mixture was concentrated to give a crude product, which was purified by flash column to give N-(3,4-dichloro-1H-indol-7-yl)-4-((4-methyl-1-(2,2,2-trifluoroacetyl)piperidin-4-yl)oxy)benzenesulfonamide (80.0 mg, yield: 55.9%) as a white solid. LCMS (ESI) found: 551 [M+H]⁺.

### 9. Preparation of N-(3,4-dichloro-1H-indol-7-yl)-4-((4-methylpiperidin-4-yl)oxy)benzenesulfonamide

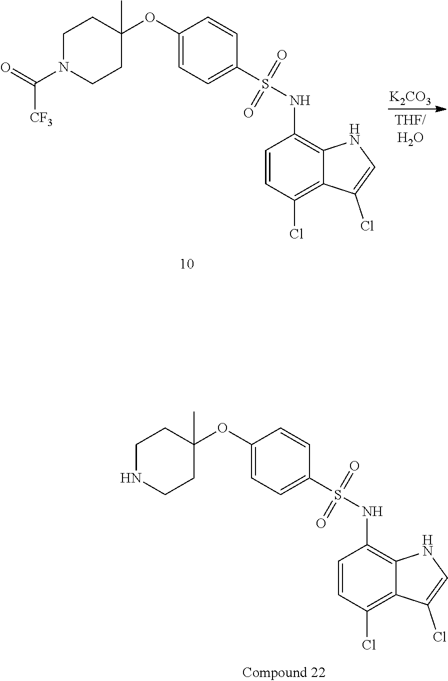 [SMILES: CC1(Oc2ccc(S(=O)(=O)Nc3ccc(Cl)c4c(Cl)c[nH]c34)cc2)CCN(C(=O)C(F)(F)F)CC1 ; "K2CO3" ; "THF/H2O" ; CC1(Oc2ccc(S(=O)(=O)Nc3ccc(Cl)c4c(Cl)c[nH]c34)cc2)CCNCC1]

To a solution of N-(3,4-dichloro-1H-indol-7-yl)-4-((4-methyl-1-(2,2,2-trifluoroacetyl) piperidin-4-yl)oxy)benzenesulfonamide (50.0 mg, 0.091 mmol) in THF/H₂O (4/1, 5 mL) was added K₂CO₃ (37.7 mg, 0.23 mmol). The mixture was stirred at 50° C. for 2 h until the starting material was consumed completely. The reaction mixture was filtered and the filtrated was concentrated to give a crude product, which was purified by prep-HPLC to give N-(3,4-dichloro-1H-indol-7-yl)-4-((4-methylpiperidin-4-yl)oxy)benzenesulfonamide (HCOOH salt) (Compound 22, 33.0 mg, yield: 79.8%) as a white solid. LCMS (ESI) found: 456 [M+H]⁺. ¹H NMR (400 MHZ, DMSO-d6) δ 11.42 (s, 1H), 8.27 (s, 0.32H), 7.63 (d, J=8.8 Hz, 2H), 7.48 (s, 1H), 7.12 (d, J=8.8 Hz, 2H), 6.90 (d, J=8.2 Hz, 1H), 6.71 (d, J=8.2 Hz, 1H), 3.17-3.05 (m, 4H), 2.07-2.01 (m, 2H), 1.88-1.71 (m, 2H), 1.30 (s, 3H).

### Example 3: Preparation of N-(3,4-dichloro-1H-indol-7-yl)-4-((1-(2-hydroxyethyl)-4-methylpiperidin-4-yl)oxy)benzenesulfonamide; Compound 23

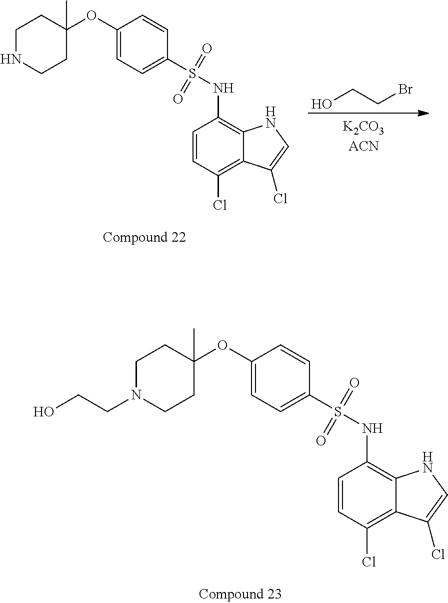

### Preparation of N-(3,4-dichloro-1H-indol-7-yl)-4-((1-(2-hydroxyethyl)-4-methylpiperidin-4-yl)oxy)benzenesulfonamide

To a solution of N-(3,4-dichloro-1H-indol-7-yl)-4-((4-methylpiperidin-4-yl)oxy)benzene sulfonamide (15.0 mg, 0.033 mmol) in CH₃CN (5 mL) was added 2-bromoethan-1-ol (4.1 mg, 0.033 mmol) and K₂CO₃ (13.7 mg, 0.099 mmol). The mixture was stirred for 2 h. The solid was filtered off and the filtrated was concentrated to give a residue, which was purified by prep-HPLC to give N-(3,4-dichloro-1H-indol-7-yl)-4-((1-(2-hydroxyethyl)-4-methyl piperidin-4-yl)oxy)benzenesulfonamide (HCOOH salt) (Compound 23, 5.8 mg, 35.3%) as a white solid. LCMS (ESI): 499.4 [M+H]⁺. 1H NMR (400 MHZ, DMSO-d6) δ 11.32 (s, 1H), 8.16 (s, 0.35H), 7.59 (d, J=8.8 Hz, 2H), 7.50 (s, 1H), 7.07 (d, J=8.8 Hz, 2H), 6.92 (d, J=8.2 Hz, 1H), 6.69 (d, J=8.2 Hz, 1H), 4.47 (s, 1H), 3.50 (t, J=6.2 Hz, 2H), 2.62-2.54 (m, 2H), 2.48-2.42 (m, 2H), 2.48-2.42 (m, 2H), 1.97-1.85 (m, 2H), 1.74-1.61 (m, 2H), 1.29 (s, 3H).

### Example 4: Preparation of N-(3,4-dichloro-1H-indol-7-yl)-4-((2-methyl-2-azaspiro[3.3]heptan-6-yl)oxy)benzenesulfonamide; Compound 24

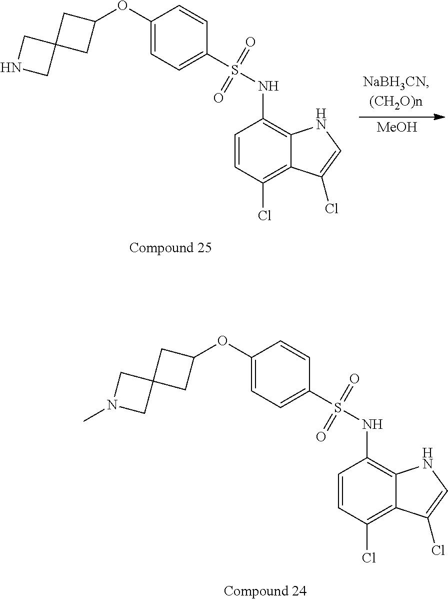 [SMILES: "MeOH" ; O=S(=O)(Nc1ccc(Cl)c2c(Cl)c[nH]c12)c1ccc(OC2CC3(CNC3)C2)cc1 ; CN1CC2(CC(Oc3ccc(S(=O)(=O)Nc4ccc(Cl)c5c(Cl)c[nH]c45)cc3)C2)C1]

### Preparation of N-(3,4-dichloro-1H-indol-7-yl)-4-((2-methyl-2-azaspiro[3.3]heptan-6-yl)oxy)benzenesulfonamide

To a solution of 4-((2-azaspiro[3.3]heptan-6-yl)oxy)-N-(3,4-dichloro-1H-indol-7-yl)benzenesulfonamide (30.0 mg, 0.07 mmol) and (CH₂O)ₙ (6.3 mg, 0.21 mmol) in MeOH (2 mL) were added a drop of AcOH and NaBH₃CN (13.2 mg, 0.27 mmol). The mixture was stirred at rt for 4 h. The mixture was washed with 10% aqueous NH₄Cl (20 mL) and extracted with DCM (20 mL×3). The combined organic layer was concentrated to give a crude product, which was purified by prep-HPLC eluting with CH₃CN/H₂O (0.5% HCOOH) to give N-(3,4-dichloro-1H-indol-7-yl)-4-((2-methyl-2-azaspiro[3.3]heptan-6-yl)oxy)benzenesulfonamide (HCOOH salt) (Compound 24, 4.7 mg, 0.010 mmol, 14.41%) as a white solid. LCMS (ESI) found: 466 [M+H]⁺ 1H NMR (400 MHZ, DMSO-d6) δ 11.38 (s, 1H), 8.18 (s, 0.74H), 7.62 (d, J=8.9 Hz, 2H), 7.49 (s, 1H), 6.93-6.85 (m, 3H), 6.71 (d, J=8.2 Hz, 1H), 4.63 (p, J=6.9 Hz, 1H), 3.45 (s, 4H), 2.70-2.62 (m, 2H), 2.36 (s, 3H), 2.20-2.11 (m, 2H).

### Example 5: Preparation of 4-((2-azaspiro[3.3]heptan-6-yl)oxy)-N-(3,4-dichloro-1H-indol-7-yl)benzenesulfonamide; Compound 25

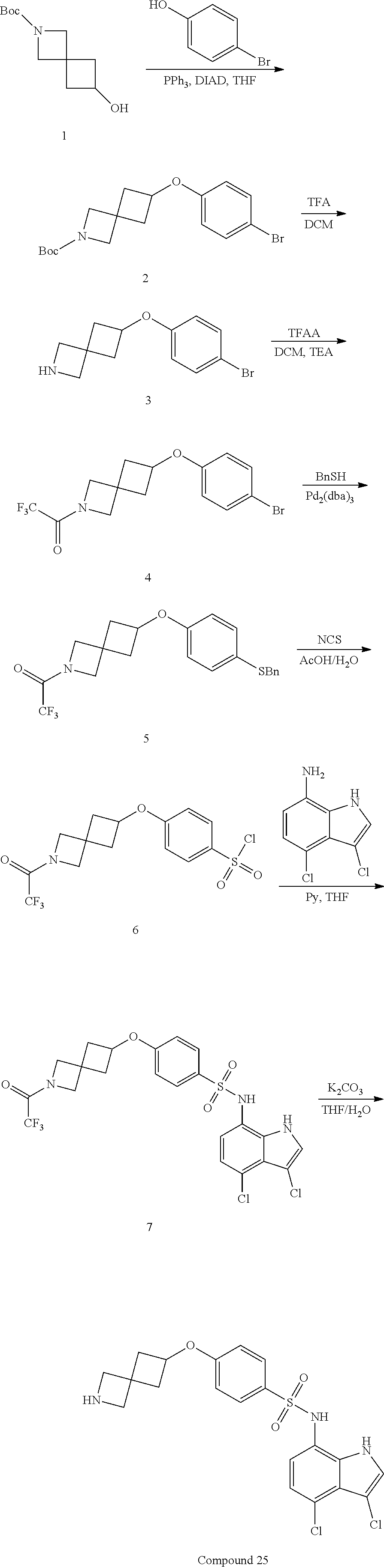

### 1. Preparation of tert-butyl 6-(4-bromophenoxy)-2-azaspiro[3.3]heptane-2-carboxylate

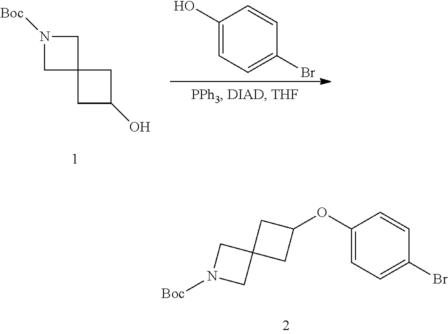 [SMILES: CC(C)(C)OC(=O)N1CC2(CC(O)C2)C1 ; Oc1ccc(Br)cc1 ; CC(C)(C)OC(=O)N1CC2(CC(Oc3ccc(Br)cc3)C2)C1]

To a solution of tert-butyl 6-hydroxy-2-azaspiro[3.3]heptane-2-carboxylate (1.0 g, 4.69 mmol), 4-bromophenol (1.0 g, 5.63 mmol) and triphenylphosphine (1.8 g, 7.03 mmol) in dry THF (10 mL) was added DIAD (1.4 g, 7.03 mmol) under N₂. The reaction mixture was stirred at rt for 18 h. TLC showed the reaction was completed. The mixture was concentrated under reduced pressure to give a crude product, which was purified by column to give tert-butyl 6-(4-bromophenoxy)-2-azaspiro[3.3]heptane-2-carboxylate (1.3 g, 75.3%) as a solid. LCMS (ESI) found: 368 [M+H]⁺.

### 2. Preparation of 6-(4-bromophenoxy)-2-azaspiro[3.3]heptane

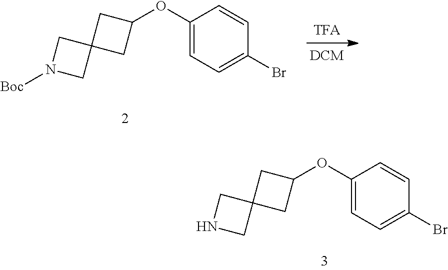 [SMILES: CC(C)(C)OC(=O)N1CC2(CC(Oc3ccc(Br)cc3)C2)C1 ; Brc1ccc(OC2CC3(CNC3)C2)cc1]

A solution of tert-butyl 6-(4-bromophenoxy)-2-azaspiro[3.3]heptane-2-carboxylate (1.3 g, 3.53 mmol) in DCM (5 mL) and TFA (5 mL) was stirred at rt for 1.5 h. LCMS showed the reaction was completed. The mixture was concentrated under reduced pressure to give a residue, which was washed with aqueous NaHCO₃ (20 mL) and extracted with EA (20 mL×3). The combined organic layer was washed with brine, dried over anhydrous Na₂SO₄ and concentrated under reduced pressure to give a crude product, which was purified by column to give 6-(4-bromophenoxy)-2-azaspiro[3.3]heptane (820.0 mg, 86.6%) as a solid. LCMS (ESI) found: 268 [M+H]⁺.

### 3. Preparation of 6-(4-bromophenoxy)-2-azaspiro[3.3]heptane

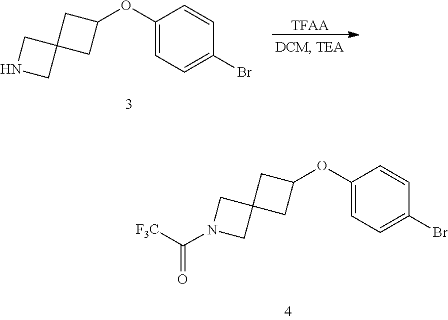 [SMILES: Brc1ccc(OC2CC3(CNC3)C2)cc1 ; "TFAA" ; O=C(N1CC2(CC(Oc3ccc(Br)cc3)C2)C1)C(F)(F)F]

To a solution of 6-(4-bromophenoxy)-2-azaspiro[3.3]heptane (820.0 mg, 3.06 mmol) and TEA (926.2 mg, 9.17 mmol) in DCM (15 mL) was added TFAA (770.6 mg, 3.67 mmol) at 0° C. The reaction mixture was stirred at rt for 1 h. The reaction mixture was poured into water (50 mL) and extracted with DCM (50 mL×3). The combined organic layer was washed with brine, dried over anhydrous Na₂SO₄ and concentrated under reduced pressure to give a crude product, which was purified by column to give 1-(6-(4-bromophenoxy)-2-azaspiro[3.3]heptan-2-yl)-2,2,2-trifluoroethan-1-one (780.0 mg, 70.0%) as a solid. LCMS (ESI) found: 364 [M+H]⁺.

### 4. Preparation of 1-(6-(4-(benzylthio)phenoxy)-2-azaspiro[3.3]heptan-2-yl)-2,2,2-trifluoroethan-1-one

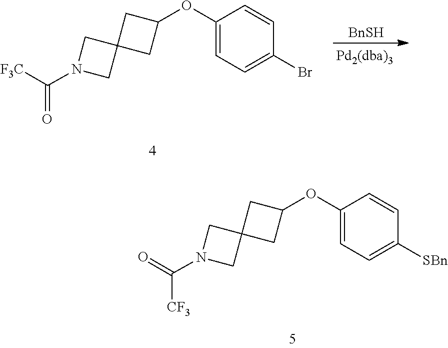

To a solution of 1-(6-(4-bromophenoxy)-2-azaspiro[3.3]heptan-2-yl)-2,2,2-trifluoroethan-1-one (680.0 mg, 1.87 mmol), BnSH (463.1 mg, 3.73 mmol) and DIEA (565.6 mg, 5.602 mmol) in dioxane (5 mL) were added Pd₂(dba)₃ (171.0 mg, 0.19 mmol) and Xantphos (324.1 mg, 0.56 mmol). The mixture was evaporated and backfilled with N₂ for three times. The reaction mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 1-(6-(4-(benzylthio)phenoxy)-2-azaspiro[3.3]heptan-2-yl)-2,2,2-trifluoroethan-1-one (660.0 mg, 86.7%) as a solid. LCMS (ESI) found: 408 [M+H]⁺.

### 5. Preparation of 4-((2-(2,2,2-trifluoroacetyl)-2-azaspiro[3.3]heptan-6-yl)oxy)benzenesulfonyl chloride

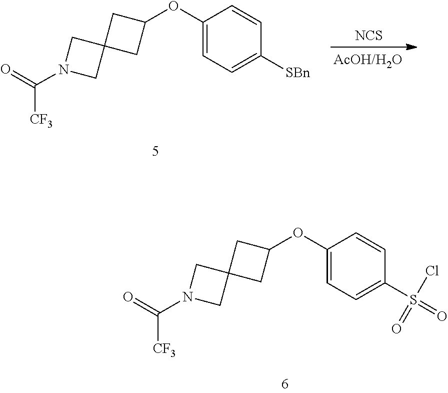

To a solution of 1-(6-(4-(benzylthio)phenoxy)-2-azaspiro[3.3]heptan-2-yl)-2,2,2-trifluoroethan-1-one (317.0 mg, 0.78 mmol) in AcOH/H₂O (9/1, 5 mL) was added NCS (11.6 mg, 0.20 mmol). The reaction mixture was stirred at rt for 2 h. The mixture was stirred at rt for 2 h. The reaction was quenched with aqueous Na₂S203 (15 mL) and extracted with DCM (15 mL×3). The combined organic layer was concentrated to give a crude product, which was purified by column chromatography on silica gel eluting with PE/EA from 15/1 to 5/1 to afford 4-((2-(2,2,2-trifluoroacetyl)-2-azaspiro[3.3]heptan-6-yl)oxy)benzenesulfonyl chloride (260.0 mg, 87.1%) as a solid. LCMS (ESI) found: 384 [M+H]⁺.

### 6. Preparation of 4-((2-(2,2,2-trifluoroacetyl)-2-azaspiro[3.3]heptan-6-yl)oxy)benzenesulfonyl chloride

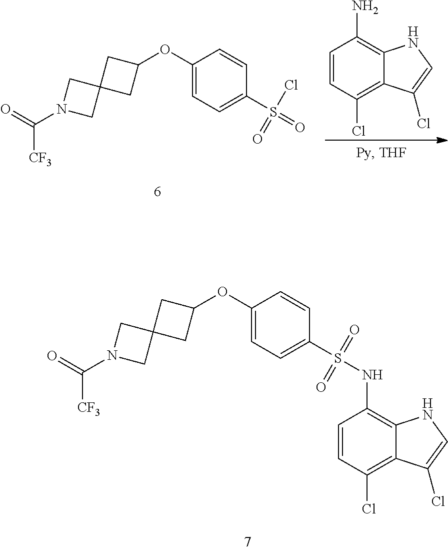

To a solution of 3,4-dichloro-1H-indol-7-amine (54.5 mg, 0.27 mmol) in THF (2 mL), were added pyridine (0.07 mL, 0.81 mmol) and 4-((2-(2,2,2-trifluoroacetyl)-2-azaspiro[3.3]heptan-6-yl)oxy)benzenesulfonyl chloride (42.6 mg, 0.22 mmol). The mixture was stirred at rt for 2 h. The mixture was concentrated and purified by flash column eluting with DCM/MeOH from 100/0 to 90/10 to give N-(3,4-dichloro-1H-indol-7-yl)-4-((2-(2,2,2-trifluoroacetyl)-2-azaspiro[3.3]heptan-6-yl)oxy)benzenesulfonamide (132.3 mg, 89.6%) as a light brown solid. LCMS (ESI) found: 548 [M+H]⁺.

### 7. Preparation of 4-((2-azaspiro[3.3]heptan-6-yl)oxy)-N-(3,4-dichloro-1H-indol-7-yl)benzenesulfonamide

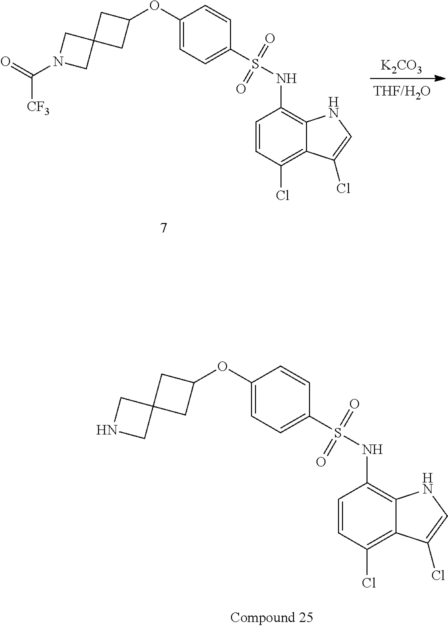 [SMILES: O=C(N1CC2(CC(Oc3ccc(S(=O)(=O)Nc4ccc(Cl)c5c(Cl)c[nH]c45)cc3)C2)C1)C(F)(F)F ; "K2CO3" ; O=S(=O)(Nc1ccc(Cl)c2c(Cl)c[nH]c12)c1ccc(OC2CC3(CNC3)C2)cc1]

To a mixture of N-(3,4-dichloro-1H-indol-7-yl)-4-((2-(2,2,2-trifluoroacetyl)-2-azaspiro[3.3]heptan-6-yl)oxy)benzenesulfonamide (120.0 mg, 0.22 mmol) in THF/H₂O (4/1, 2 mL) was added K₂CO₃ (90.6 mg, 0.66 mmol). The mixture was stirred at rt for 4 h. The mixture was concentrated and purified by flash eluting with DCM/MeOH from 95/5 to 50/50 to give 4-((2-azaspiro[3.3]heptan-6-yl)oxy)-N-(3,4-dichloro-1H-indol-7-yl)benzenesulfonamide (60.0 mg, 60.6%) as a white solid. 30 mg DP was used for next step (for Compound 24). The other 30 mg DP was further purified by prep-HPLC (0.5% NH₄OH) to give pure DP (Compound 25, 2.1 mg+1.9 mg), submitted as Compound 25. LCMS (ESI) found: 452 [M+H]⁺. 1H NMR (400 MHZ, DMSO-d6) δ 7.62 (d, J=8.8 Hz, 2H), 7.25 (s, 1H), 6.78 (d, J=8.8 Hz, 2H), 6.67 (s, 2H), 4.62-4.55 (m, 1H), 3.92 (s, 2H), 3.85 (s, 2H), 2.77-2.66 (m, 2H), 2.23-2.14 (m, 2H).

### Synthesis of Ether Analogs

Treatment of 7-amino-3,4-dichloroindole with 4-hydroxyphenylsulfonyl chloride yielded intermediate E2. Treatment of E2 with tributylsilyl chloride afforded the silyl ether E3. Treatment of E3 with (Boc₂)O led to intermediate E4, which was treated with TBAF to yield intermediate E5. Intermediate E5 was treated with primary and secondary alcohols as indicated to yield ethers E6. Removal of the Boc protecting groups with TFA in DCM then yielded the final products E7. The final products were purified by HPLC giving the pure materials with purity ≥95% as confirmed by LC-MS.

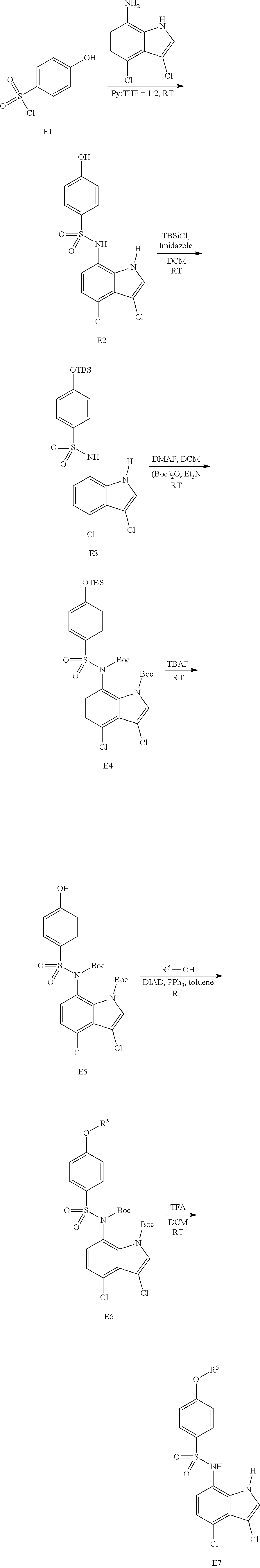

Starting with E8 (2-Fluoro-4-hydroxybenzenesulfonyl chloride), E9 (3-Fluoro-4-hydroxybenzenesulfonyl chloride and E10 (2-Fluoro-3-hydroxybenzenesulfonyl chloride), and using procedures similar to those described above, sulfonamide analogs E11, E12 and E13 were synthesized. The final products were purified by HPLC giving the pure materials with purity ≥95% as confirmed by LC-MS.

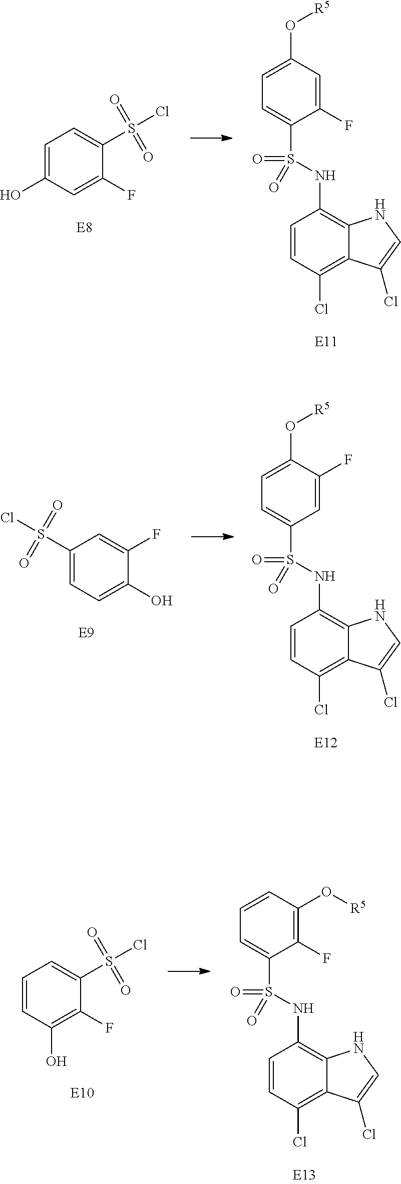

### Synthesis of 3-Carboxamidophenylsulfonamide Analogs

Treatment of methyl 3-amino-2-fluorobenzoate with sodium nitrite, followed by thionyl chloride and cuprous chloride yielded sulfonyl chloride A2. Coupling of A1 with 7-amino-3,4, -dichloroindole afforded intermediate A3, which was treated with LiOH to yield intermediate A4. Coupling of A4 with primary and secondary amines yielded amide analogs A5. The final products were purified by HPLC giving the pure materials with purity ≥95% as confirmed by LC-MS.

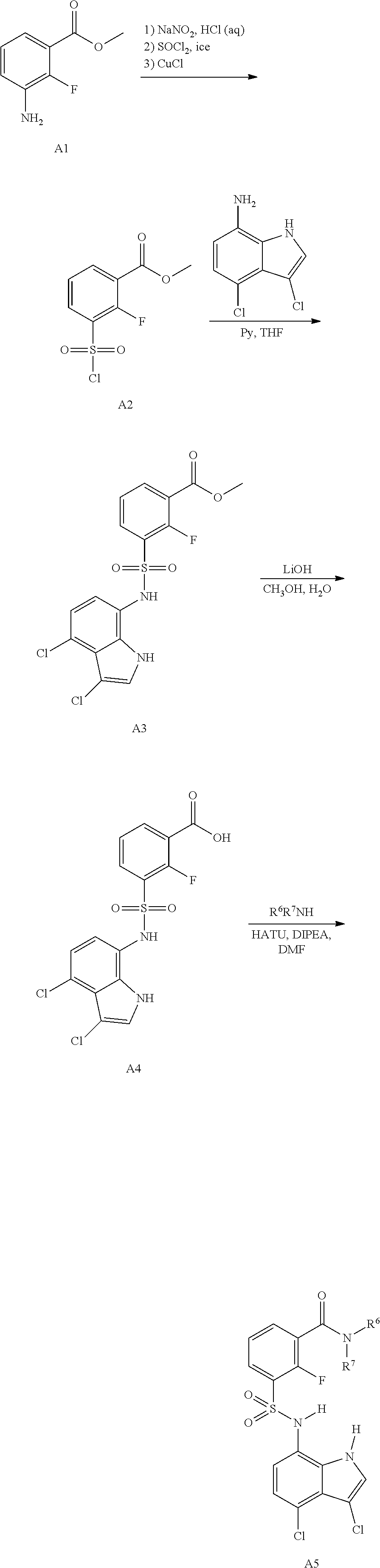

### Synthesis of 4-Carboxamidophenylsulfonamide Analogs

Using a similar synthetic route, coupling of 4-carbomethoxyl-2-fluorophenylsulfonyl chloride (A6) with 7-amino-3,4, -dichloroindole afforded intermediate A7, which was treated with LiOH to yield intermediate A8. Coupling of A8 with primary and secondary amines yielded amide analogs A9. The final products were purified by HPLC giving the pure materials with purity ≥95% as confirmed by LC-MS.

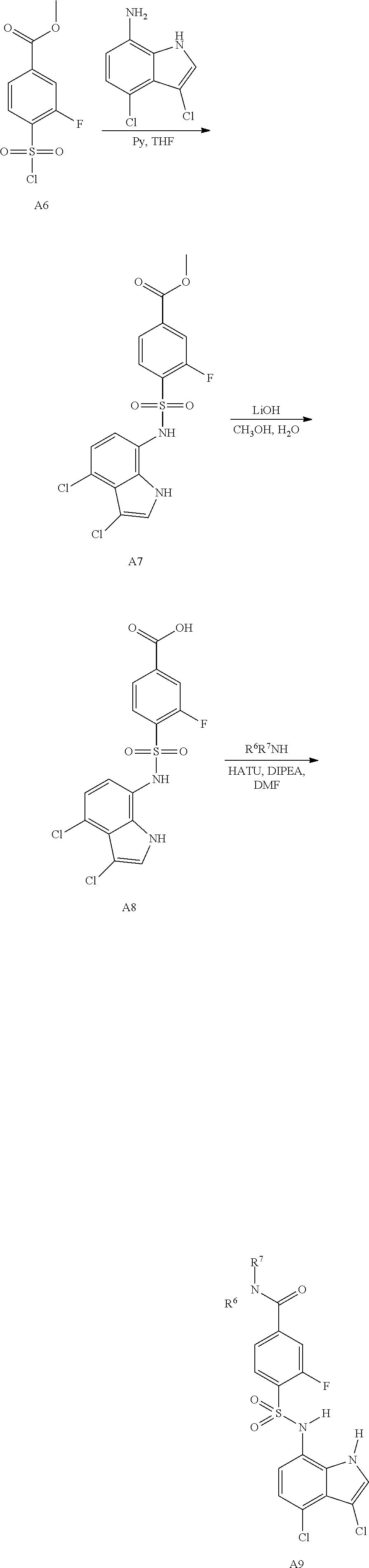

Starting with A10 (Methyl 4-(chlorosulfonyl)benzoate) and A11 (Methyl 4-(chlorosulfonyl)benzoate, and using procedures similar to those described above, sulfonamide analogs A12 and A13 were synthesized. The final products were purified by HPLC giving the pure materials with purity ≥95% as confirmed by LC-MS.

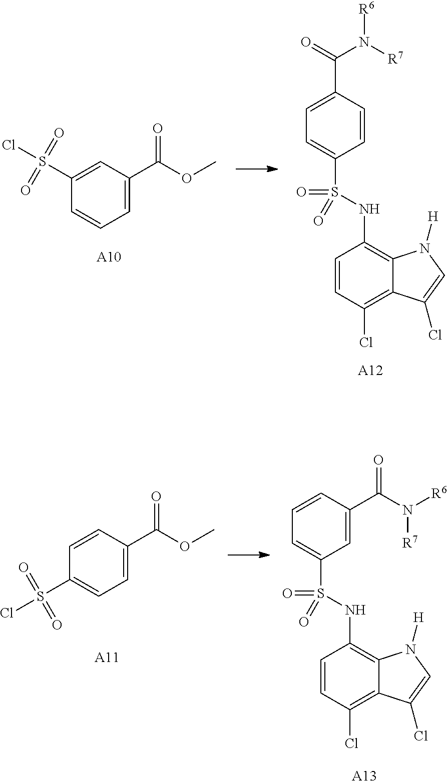

The following additional compounds were made according to the procedures described above using the appropriate starting materials.

TABLE A

| | | MS |
|---|---|---|
| | | Measured |
| Cmp# | Structure | (m/e + 1) |
| 1 | 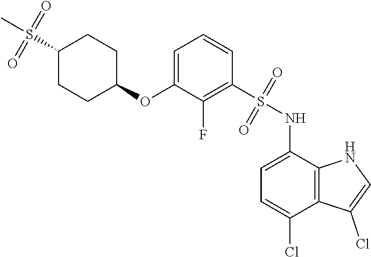 [SMILES: CS(=O)(=O)[C@H]1CC[C@H](Oc2cccc(S(=O)(=O)Nc3ccc(Cl)c4c(Cl)c[nH]c34)c2F)CC1] | NA |
| 2 | 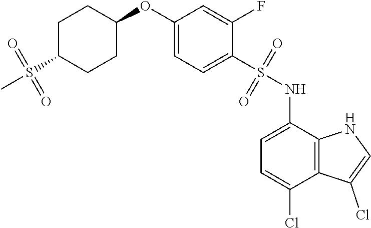 [SMILES: CS(=O)(=O)[C@H]1CC[C@H](Oc2ccc(S(=O)(=O)Nc3ccc(Cl)c4c(Cl)c[nH]c34)c(F)c2)CC1] | NA |
| 3 | 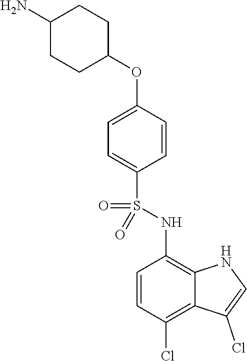 [SMILES: NC1CCC(Oc2ccc(S(=O)(=O)Nc3ccc(Cl)c4c(Cl)c[nH]c34)cc2)CC1] | 454.4 |
| 4 | 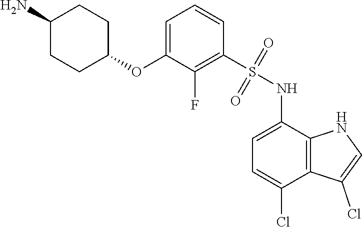 [SMILES: N[C@H]1CC[C@H](Oc2cccc(S(=O)(=O)Nc3ccc(Cl)c4c(Cl)c[nH]c34)c2F)CC1] | 454.4 |
| 5 | 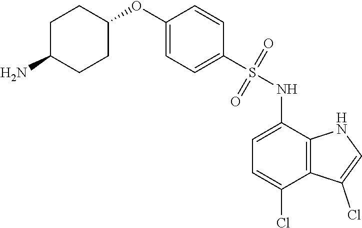 [SMILES: N[C@H]1CC[C@H](Oc2ccc(S(=O)(=O)Nc3ccc(Cl)c4c(Cl)c[nH]c34)cc2)CC1] | 454.4 |
| 6 | 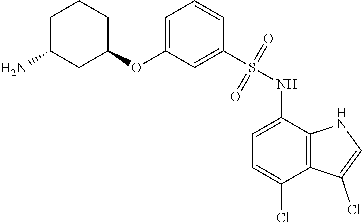 [SMILES: N[C@@H]1CCC[C@@H](Oc2cccc(S(=O)(=O)Nc3ccc(Cl)c4c(Cl)c[nH]c34)c2)C1] | 454.4 |
| 7 | 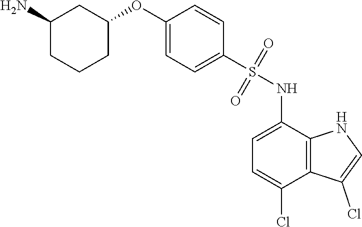 [SMILES: N[C@@H]1CCC[C@@H](Oc2ccc(S(=O)(=O)Nc3ccc(Cl)c4c(Cl)c[nH]c34)cc2)C1] | 454.4 |
| 8 | 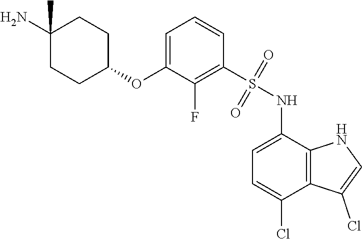 [SMILES: C[C@]1(N)CC[C@@H](Oc2cccc(S(=O)(=O)Nc3ccc(Cl)c4c(Cl)c[nH]c34)c2F)CC1] | 468.4 |
| 9 | 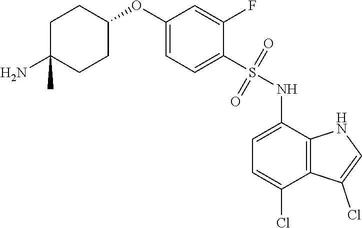 [SMILES: C[C@]1(N)CC[C@@H](Oc2ccc(S(=O)(=O)Nc3ccc(Cl)c4c(Cl)c[nH]c34)c(F)c2)CC1] | 468.5 |
| 12 | 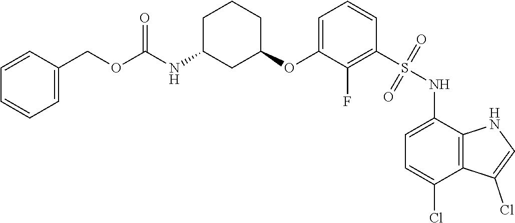 [SMILES: O=C(N[C@@H]1CCC[C@@H](Oc2cccc(S(=O)(=O)Nc3ccc(Cl)c4c(Cl)c[nH]c34)c2F)C1)OCc1ccccc1] | 587.4 |
| 13 | 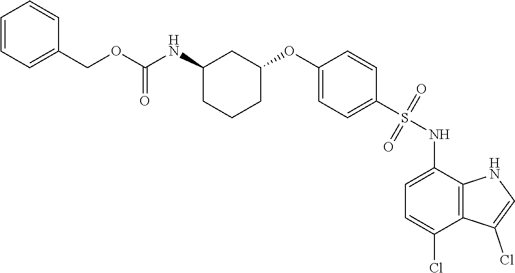 [SMILES: O=C(N[C@@H]1CCC[C@@H](Oc2ccc(S(=O)(=O)Nc3ccc(Cl)c4c(Cl)c[nH]c34)cc2)C1)OCc1ccccc1] | 588 |
| 14 | 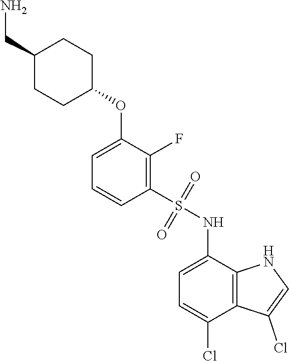 [SMILES: NC[C@H]1CC[C@H](Oc2cccc(S(=O)(=O)Nc3ccc(Cl)c4c(Cl)c[nH]c34)c2F)CC1] | 468.4 |
| 15 | 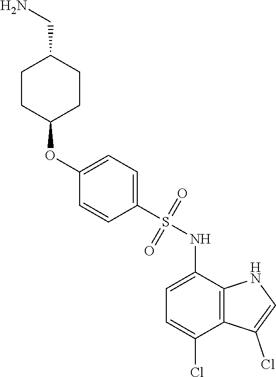 [SMILES: NC[C@H]1CC[C@H](Oc2ccc(S(=O)(=O)Nc3ccc(Cl)c4c(Cl)c[nH]c34)cc2)CC1] | 468 |
| 16 | 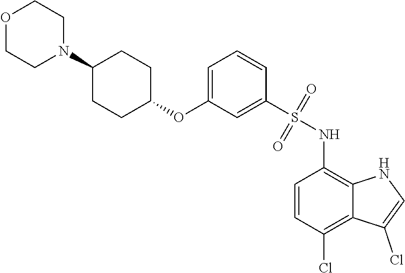 [SMILES: O=S(=O)(Nc1ccc(Cl)c2c(Cl)c[nH]c12)c1cccc(O[C@H]2CC[C@H](N3CCOCC3)CC2)c1] | 524 |
| 17 | 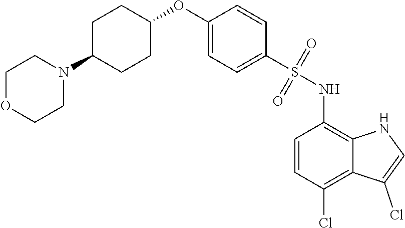 [SMILES: O=S(=O)(Nc1ccc(Cl)c2c(Cl)c[nH]c12)c1ccc(O[C@H]2CC[C@H](N3CCOCC3)CC2)cc1] | 524.5 |
| 18 | 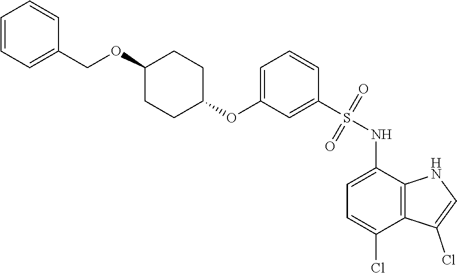 [SMILES: O=S(=O)(Nc1ccc(Cl)c2c(Cl)c[nH]c12)c1cccc(O[C@H]2CC[C@H](OCc3ccccc3)CC2)c1] | 545.5 |
| 19 | 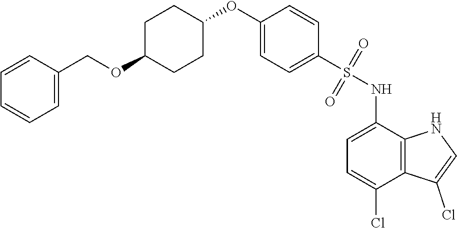 [SMILES: O=S(=O)(Nc1ccc(Cl)c2c(Cl)c[nH]c12)c1ccc(O[C@H]2CC[C@H](OCc3ccccc3)CC2)cc1] | 545.5 |
| 20 | 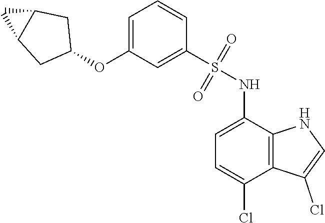 [SMILES: O=S(=O)(Nc1ccc(Cl)c2c(Cl)c[nH]c12)c1cccc(O[C@@H]2C[C@@H]3C[C@@H]3C2)c1] | 437.9 |
| 21 | 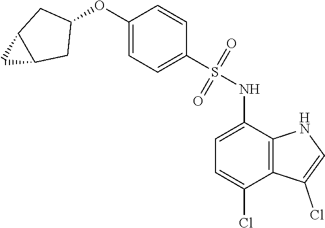 [SMILES: O=S(=O)(Nc1ccc(Cl)c2c(Cl)c[nH]c12)c1ccc(O[C@@H]2C[C@@H]3C[C@@H]3C2)cc1] | 437 |
| 26 | 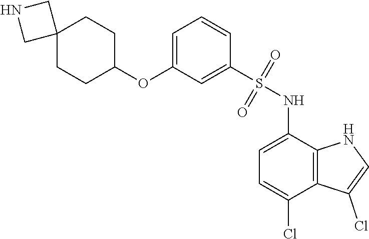 [SMILES: O=S(=O)(Nc1ccc(Cl)c2c(Cl)c[nH]c12)c1cccc(OC2CCC3(CC2)CNC3)c1] | 480 |
| 27 | 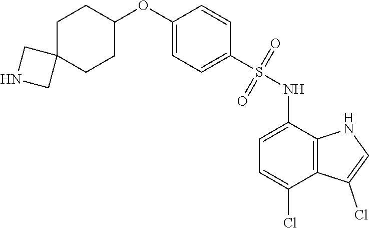 [SMILES: O=S(=O)(Nc1ccc(Cl)c2c(Cl)c[nH]c12)c1ccc(OC2CCC3(CC2)CNC3)cc1] | 480 |
| 32 | 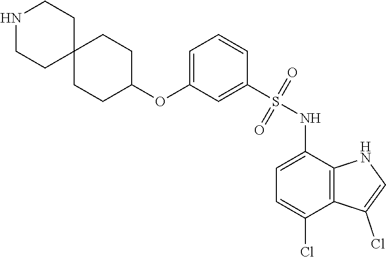 [SMILES: O=S(=O)(Nc1ccc(Cl)c2c(Cl)c[nH]c12)c1cccc(OC2CCC3(CCNCC3)CC2)c1] | 508 |
| 34 | 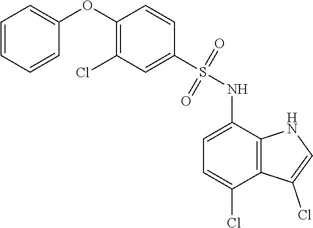 [SMILES: O=S(=O)(Nc1ccc(Cl)c2c(Cl)c[nH]c12)c1ccc(Oc2ccccc2)c(Cl)c1] | NA |
| 35 | 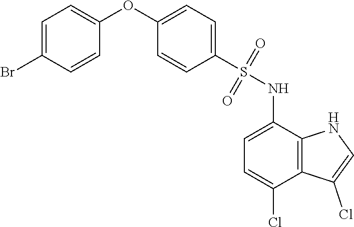 [SMILES: O=S(=O)(Nc1ccc(Cl)c2c(Cl)c[nH]c12)c1ccc(Oc2ccc(Br)cc2)cc1] | NA |
| 36 | 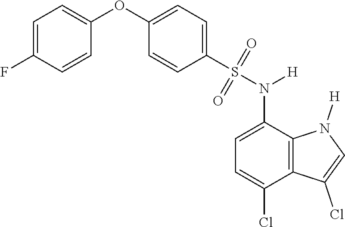 [SMILES: O=S(=O)(Nc1ccc(Cl)c2c(Cl)c[nH]c12)c1ccc(Oc2ccc(F)cc2)cc1] | NA |
| 37 | 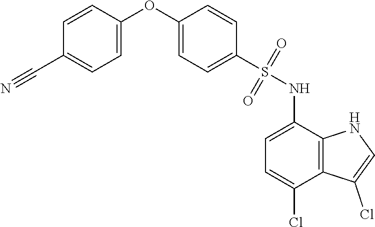 [SMILES: N#Cc1ccc(Oc2ccc(S(=O)(=O)Nc3ccc(Cl)c4c(Cl)c[nH]c34)cc2)cc1] | NA |
| 38 | 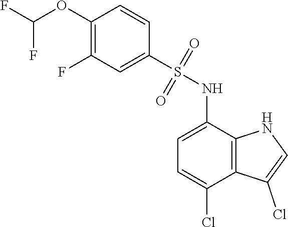 [SMILES: O=S(=O)(Nc1ccc(Cl)c2c(Cl)c[nH]c12)c1ccc(OC(F)F)c(F)c1] | NA |
| 39 | 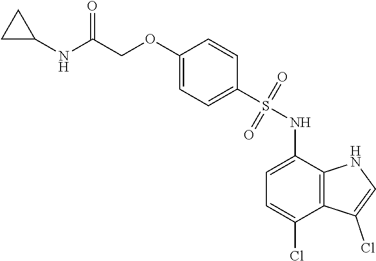 [SMILES: O=C(COc1ccc(S(=O)(=O)Nc2ccc(Cl)c3c(Cl)c[nH]c23)cc1)NC1CC1] | NA |
| 40 | 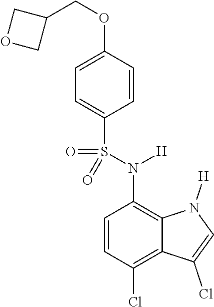 [SMILES: O=S(=O)(Nc1ccc(Cl)c2c(Cl)c[nH]c12)c1ccc(OCC2COC2)cc1] | 427.3 |
| 41 | 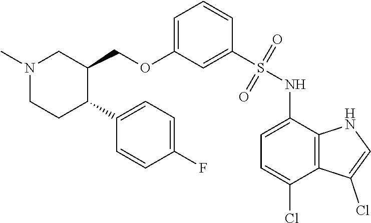 [SMILES: CN1CC[C@@H](c2ccc(F)cc2)[C@H](COc2cccc(S(=O)(=O)Nc3ccc(Cl)c4c(Cl)c[nH]c34)c2)C1] | 562.4 |
| 42 | 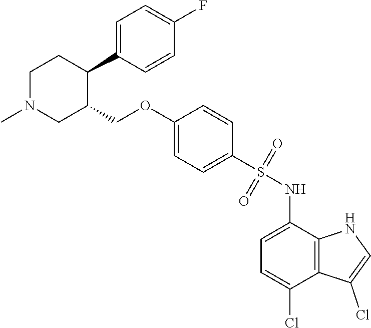 [SMILES: CN1CC[C@@H](c2ccc(F)cc2)[C@H](COc2ccc(S(=O)(=O)Nc3ccc(Cl)c4c(Cl)c[nH]c34)cc2)C1] | 562.4 |
| 43 | 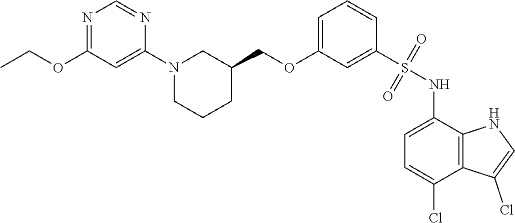 [SMILES: CCOc1cc(N2CCC[C@H](COc3cccc(S(=O)(=O)Nc4ccc(Cl)c5c(Cl)c[nH]c45)c3)C2)ncn1] | 576.5 |
| 44 | 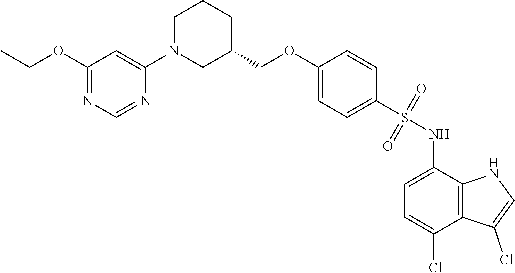 [SMILES: CCOc1cc(N2CCC[C@H](COc3ccc(S(=O)(=O)Nc4ccc(Cl)c5c(Cl)c[nH]c45)cc3)C2)ncn1] | 576 |
| 45 | 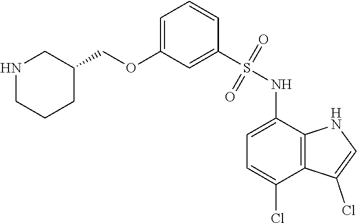 [SMILES: O=S(=O)(Nc1ccc(Cl)c2c(Cl)c[nH]c12)c1cccc(OC[C@@H]2CCCNC2)c1] | 454 |
| 46 | 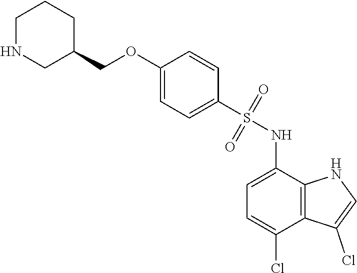 [SMILES: O=S(=O)(Nc1ccc(Cl)c2c(Cl)c[nH]c12)c1ccc(OC[C@@H]2CCCNC2)cc1] | 453.4 |
| 47 | 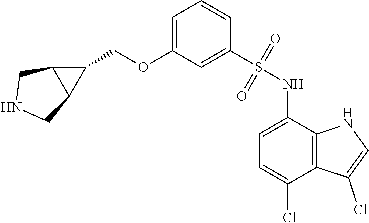 [SMILES: O=S(=O)(Nc1ccc(Cl)c2c(Cl)c[nH]c12)c1cccc(OC[C@H]2[C@@H]3CNC[C@@H]32)c1] | 452.4 |
| 48 | 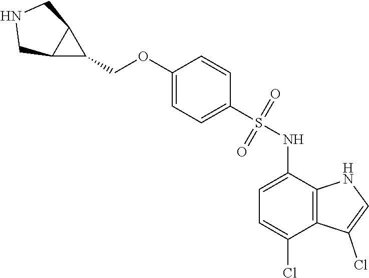 [SMILES: O=S(=O)(Nc1ccc(Cl)c2c(Cl)c[nH]c12)c1ccc(OC[C@H]2[C@@H]3CNC[C@@H]32)cc1] | 452.4 |
| 49 | 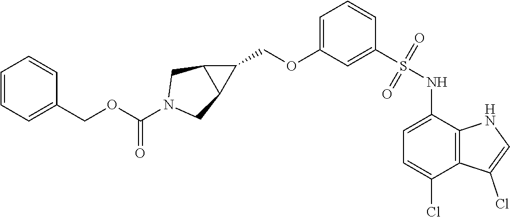 [SMILES: O=C(OCc1ccccc1)N1C[C@@H]2[C@@H](COc3cccc(S(=O)(=O)Nc4ccc(Cl)c5c(Cl)c[nH]c45)c3)[C@@H]2C1] | 586.4 |
| 50 | 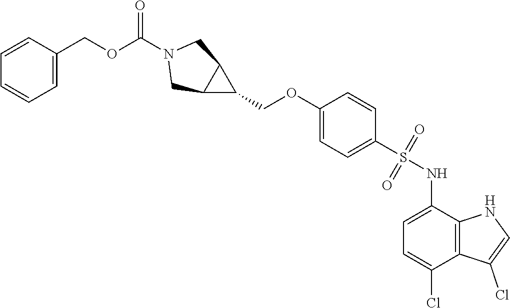 [SMILES: O=C(OCc1ccccc1)N1C[C@@H]2[C@@H](COc3ccc(S(=O)(=O)Nc4ccc(Cl)c5c(Cl)c[nH]c45)cc3)[C@@H]2C1] | 586.5 |
| 53 | 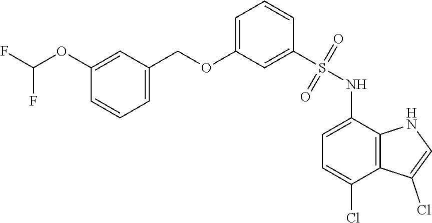 [SMILES: O=S(=O)(Nc1ccc(Cl)c2c(Cl)c[nH]c12)c1cccc(OCc2cccc(OC(F)F)c2)c1] | 513 |
| 54 | 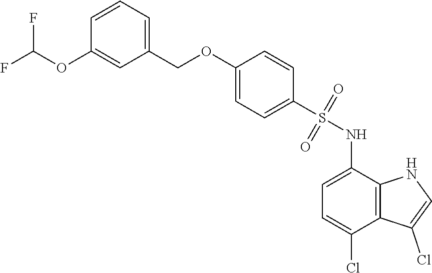 [SMILES: O=S(=O)(Nc1ccc(Cl)c2c(Cl)c[nH]c12)c1ccc(OCc2cccc(OC(F)F)c2)cc1] | 513 |
| 59 | 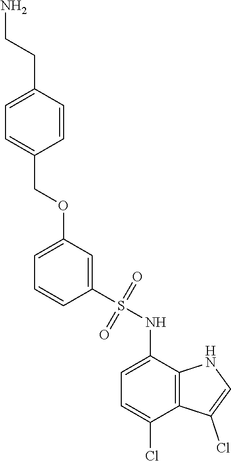 [SMILES: NCCc1ccc(COc2cccc(S(=O)(=O)Nc3ccc(Cl)c4c(Cl)c[nH]c34)c2)cc1] | 489.9 |
| 60 | 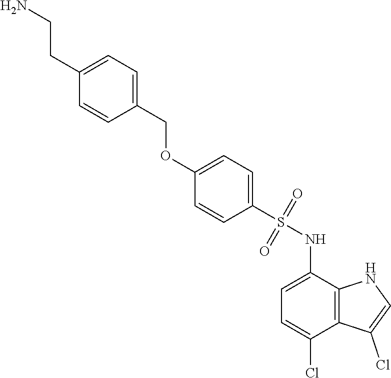 [SMILES: NCCc1ccc(COc2ccc(S(=O)(=O)Nc3ccc(Cl)c4c(Cl)c[nH]c34)cc2)cc1] | 490.4 |
| 63 | 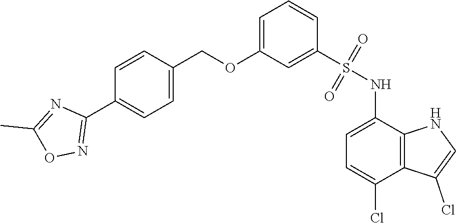 [SMILES: Cc1nc(-c2ccc(COc3cccc(S(=O)(=O)Nc4ccc(Cl)c5c(Cl)c[nH]c45)c3)cc2)no1] | 529 |
| 64 | 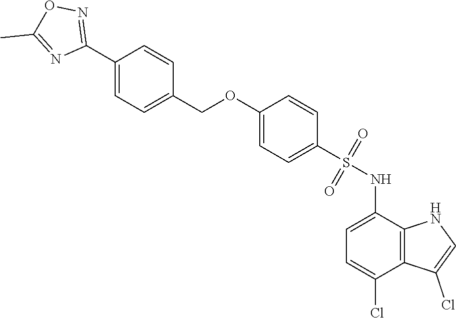 [SMILES: Cc1nc(-c2ccc(COc3ccc(S(=O)(=O)Nc4ccc(Cl)c5c(Cl)c[nH]c45)cc3)cc2)no1] | 529 |
| 65 | 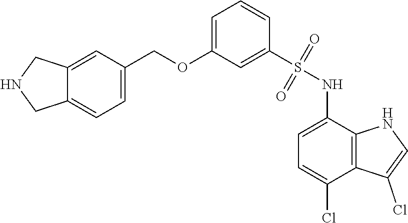 [SMILES: O=S(=O)(Nc1ccc(Cl)c2c(Cl)c[nH]c12)c1cccc(OCc2ccc3c(c2)CNC3)c1] | 488.4 |
| 66 | 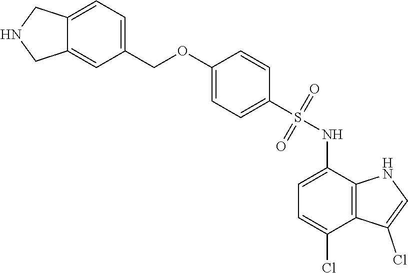 [SMILES: O=S(=O)(Nc1ccc(Cl)c2c(Cl)c[nH]c12)c1ccc(OCc2ccc3c(c2)CNC3)cc1] | 488.4 |
| 67 | 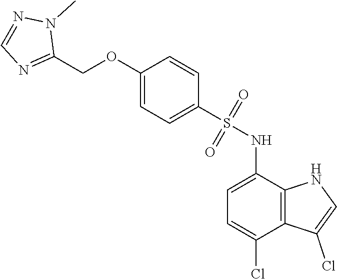 [SMILES: Cn1ncnc1COc1ccc(S(=O)(=O)Nc2ccc(Cl)c3c(Cl)c[nH]c23)cc1] | NA |
| 68 | 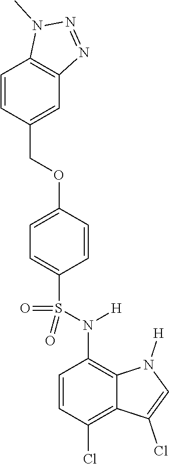 [SMILES: Cn1nnc2cc(COc3ccc(S(=O)(=O)Nc4ccc(Cl)c5c(Cl)c[nH]c45)cc3)ccc21] | 502.0 |
| 71 | 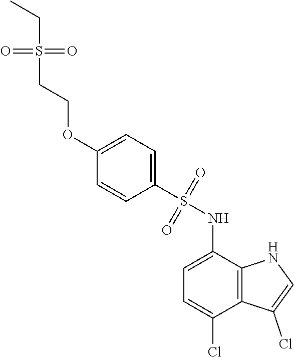 [SMILES: CCS(=O)(=O)CCOc1ccc(S(=O)(=O)Nc2ccc(Cl)c3c(Cl)c[nH]c23)cc1] | NA |
| 74 | 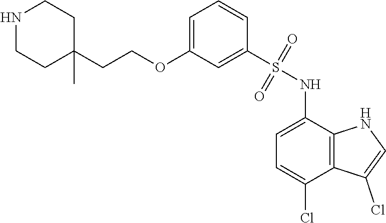 [SMILES: CC1(CCOc2cccc(S(=O)(=O)Nc3ccc(Cl)c4c(Cl)c[nH]c34)c2)CCNCC1] | 482 |
| 75 | 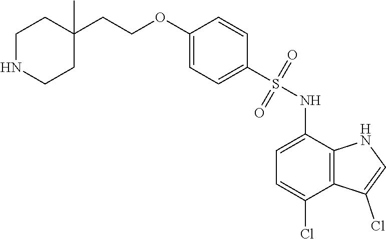 [SMILES: CC1(CCOc2ccc(S(=O)(=O)Nc3ccc(Cl)c4c(Cl)c[nH]c34)cc2)CCNCC1] | 482.5 |
| 76 | 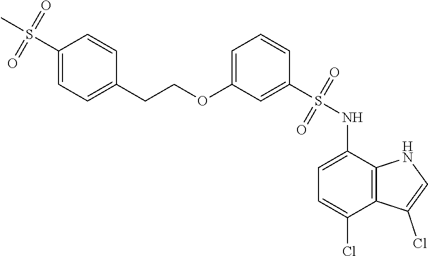 [SMILES: CS(=O)(=O)c1ccc(CCOc2cccc(S(=O)(=O)Nc3ccc(Cl)c4c(Cl)c[nH]c34)c2)cc1] | 539 |
| 77 | 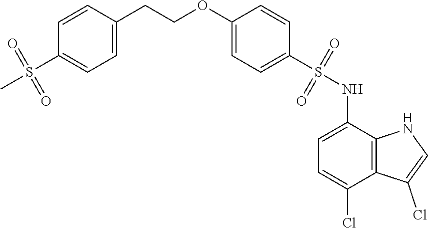 [SMILES: CS(=O)(=O)c1ccc(CCOc2ccc(S(=O)(=O)Nc3ccc(Cl)c4c(Cl)c[nH]c34)cc2)cc1] | 539.4 |
| 92 | 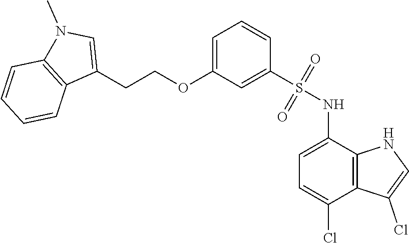 [SMILES: Cn1cc(CCOc2cccc(S(=O)(=O)Nc3ccc(Cl)c4c(Cl)c[nH]c34)c2)c2ccccc21] | 514 |
| 93 | 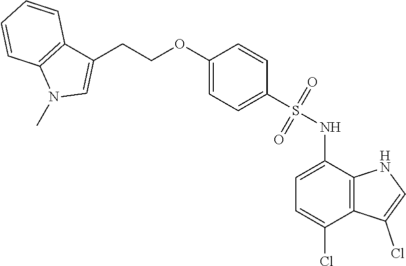 [SMILES: Cn1cc(CCOc2ccc(S(=O)(=O)Nc3ccc(Cl)c4c(Cl)c[nH]c34)cc2)c2ccccc21] | 514.4 |
| 96 | 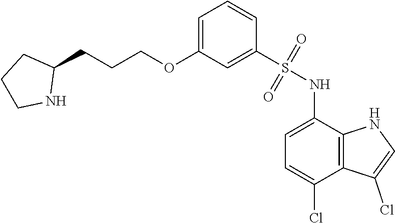 [SMILES: O=S(=O)(Nc1ccc(Cl)c2c(Cl)c[nH]c12)c1cccc(OCCC[C@@H]2CCCN2)c1] | 468.4 |
| 97 | 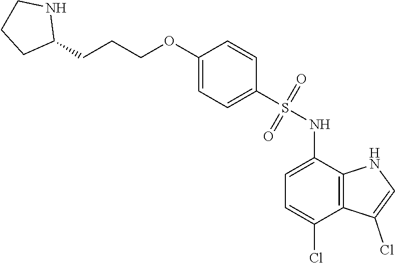 [SMILES: O=S(=O)(Nc1ccc(Cl)c2c(Cl)c[nH]c12)c1ccc(OCCC[C@@H]2CCCN2)cc1] | 468.5 |
| 98 | 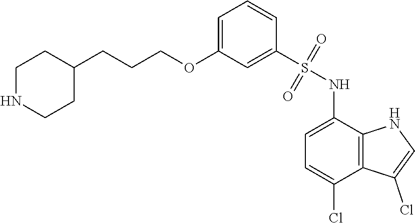 [SMILES: O=S(=O)(Nc1ccc(Cl)c2c(Cl)c[nH]c12)c1cccc(OCCCC2CCNCC2)c1] | 482.5 |
| 99 | 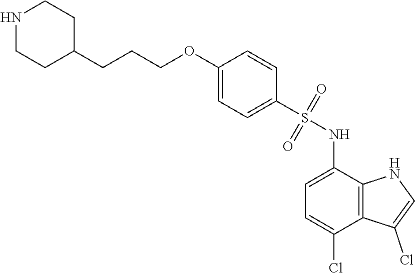 [SMILES: O=S(=O)(Nc1ccc(Cl)c2c(Cl)c[nH]c12)c1ccc(OCCCC2CCNCC2)cc1] | 482.4 |
| 100 | 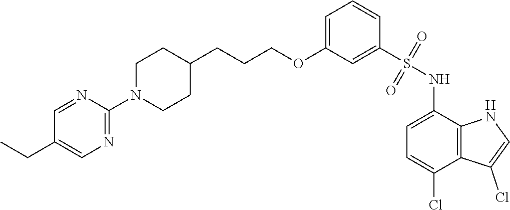 [SMILES: CCc1cnc(N2CCC(CCCOc3cccc(S(=O)(=O)Nc4ccc(Cl)c5c(Cl)c[nH]c45)c3)CC2)nc1] | 588 |
| 101 | 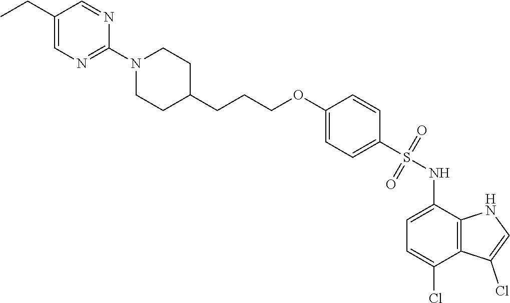 [SMILES: CCc1cnc(N2CCC(CCCOc3ccc(S(=O)(=O)Nc4ccc(Cl)c5c(Cl)c[nH]c45)cc3)CC2)nc1] | 588.5 |
| 104 | 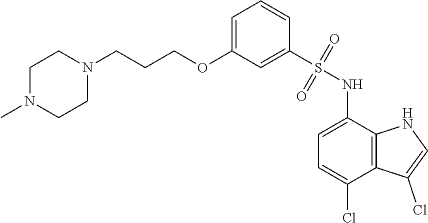 [SMILES: CN1CCN(CCCOc2cccc(S(=O)(=O)Nc3ccc(Cl)c4c(Cl)c[nH]c34)c2)CC1] | 497 |
| 105 | 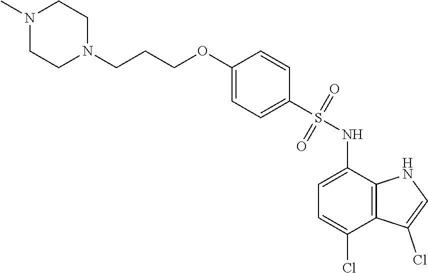 [SMILES: CN1CCN(CCCOc2ccc(S(=O)(=O)Nc3ccc(Cl)c4c(Cl)c[nH]c34)cc2)CC1] | 497 |
| 108 | 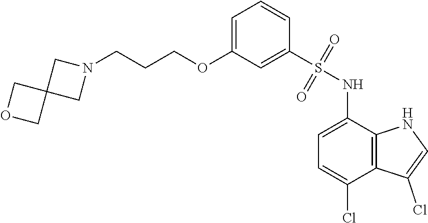 [SMILES: O=S(=O)(Nc1ccc(Cl)c2c(Cl)c[nH]c12)c1cccc(OCCCN2CC3(COC3)C2)c1] | 496 |
| 109 | 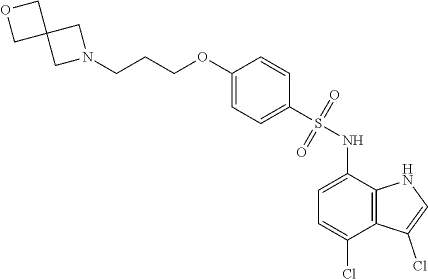 [SMILES: O=S(=O)(Nc1ccc(Cl)c2c(Cl)c[nH]c12)c1ccc(OCCCN2CC3(COC3)C2)cc1] | 496 |
| 110 | 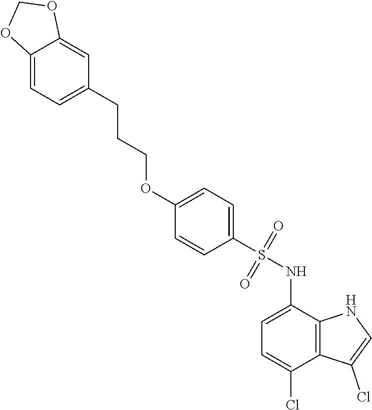 [SMILES: O=S(=O)(Nc1ccc(Cl)c2c(Cl)c[nH]c12)c1ccc(OCCCc2ccc3c(c2)OCO3)cc1] | 519 |
| 111 | 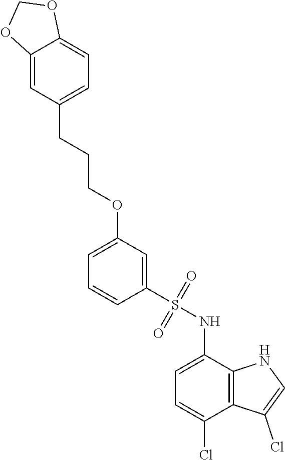 [SMILES: O=S(=O)(Nc1ccc(Cl)c2c(Cl)c[nH]c12)c1cccc(OCCCc2ccc3c(c2)OCO3)c1] | 519.4 |
| 112 | 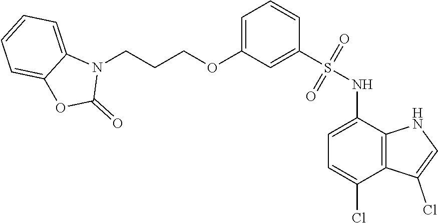 [SMILES: O=c1oc2ccccc2n1CCCOc1cccc(S(=O)(=O)Nc2ccc(Cl)c3c(Cl)c[nH]c23)c1] | 532 |
| 113 | 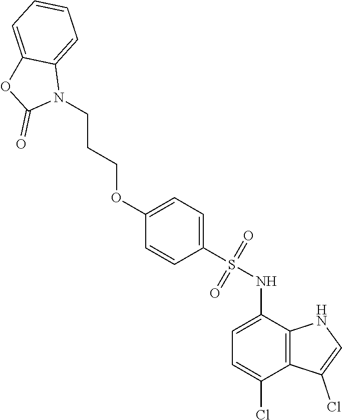 [SMILES: O=c1oc2ccccc2n1CCCOc1ccc(S(=O)(=O)Nc2ccc(Cl)c3c(Cl)c[nH]c23)cc1] | 532 |
| 114 | 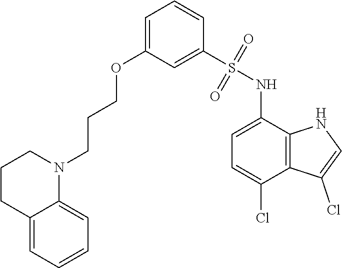 [SMILES: O=S(=O)(Nc1ccc(Cl)c2c(Cl)c[nH]c12)c1cccc(OCCCN2CCCc3ccccc32)c1] | 530 |
| 115 | 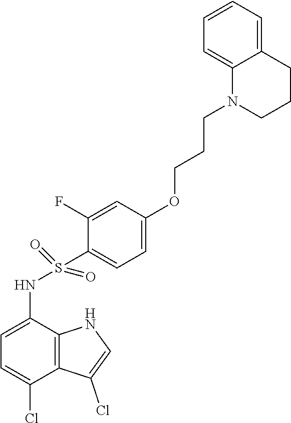 [SMILES: O=S(=O)(Nc1ccc(Cl)c2c(Cl)c[nH]c12)c1ccc(OCCCN2CCCc3ccccc32)cc1F] | 530 |
| 116 | 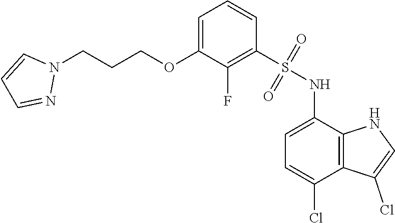 [SMILES: O=S(=O)(Nc1ccc(Cl)c2c(Cl)c[nH]c12)c1cccc(OCCCn2cccn2)c1F] | 465 |
| 117 | 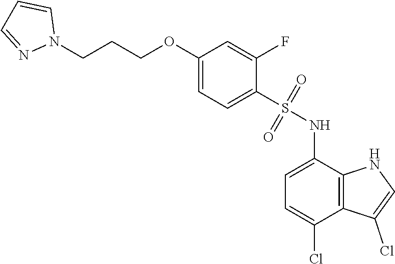 [SMILES: O=S(=O)(Nc1ccc(Cl)c2c(Cl)c[nH]c12)c1ccc(OCCCn2cccn2)cc1F] | 465 |
| 120 | 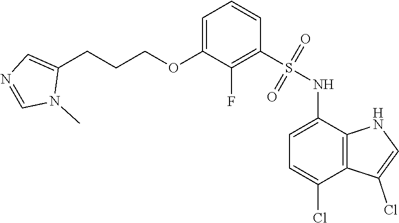 [SMILES: Cn1cncc1CCCOc1cccc(S(=O)(=O)Nc2ccc(Cl)c3c(Cl)c[nH]c23)c1F] | 479 |
| 121 | 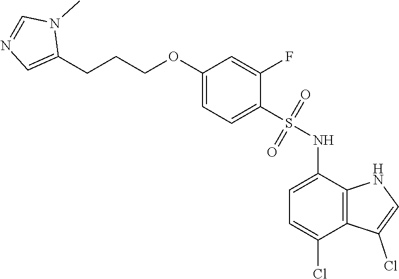 [SMILES: Cn1cncc1CCCOc1ccc(S(=O)(=O)Nc2ccc(Cl)c3c(Cl)c[nH]c23)c(F)c1] | 479.4 |
| 122 | 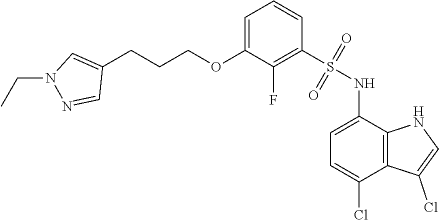 [SMILES: CCn1cc(CCCOc2cccc(S(=O)(=O)Nc3ccc(Cl)c4c(Cl)c[nH]c34)c2F)cn1] | 493.4 |
| 123 | 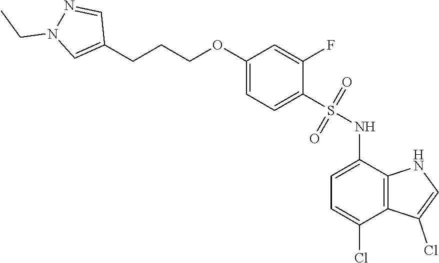 [SMILES: CCn1cc(CCCOc2ccc(S(=O)(=O)Nc3ccc(Cl)c4c(Cl)c[nH]c34)c(F)c2)cn1] | 493 |
| 124 | 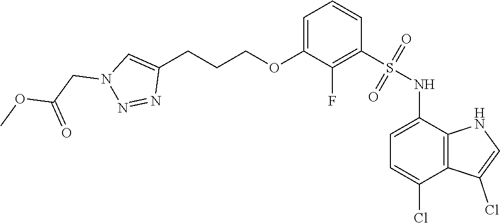 [SMILES: COC(=O)Cn1cc(CCCOc2cccc(S(=O)(=O)Nc3ccc(Cl)c4c(Cl)c[nH]c34)c2F)nn1] | 538 |
| 128 | 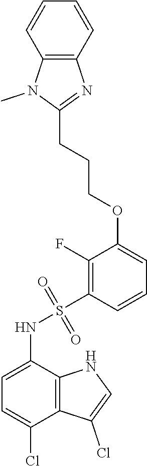 [SMILES: Cn1c(CCCOc2cccc(S(=O)(=O)Nc3ccc(Cl)c4c(Cl)c[nH]c34)c2F)nc2ccccc21] | 529 |
| 129 | 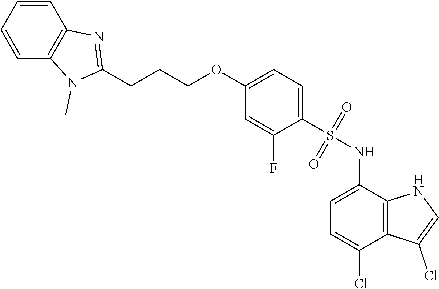 [SMILES: Cn1c(CCCOc2ccc(S(=O)(=O)Nc3ccc(Cl)c4c(Cl)c[nH]c34)c(F)c2)nc2ccccc21] | 529.4 |
| 140 | 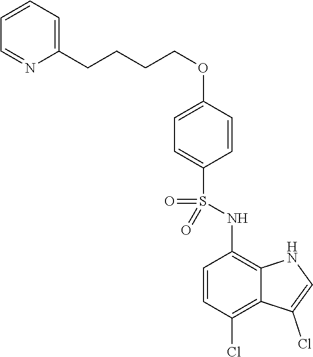 [SMILES: O=S(=O)(Nc1ccc(Cl)c2c(Cl)c[nH]c12)c1ccc(OCCCCc2ccccn2)cc1] | 490.3 |
| 143 | 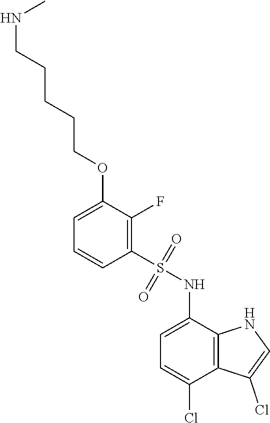 [SMILES: CNCCCCCOc1cccc(S(=O)(=O)Nc2ccc(Cl)c3c(Cl)c[nH]c23)c1F] | 456 |
| 144 | 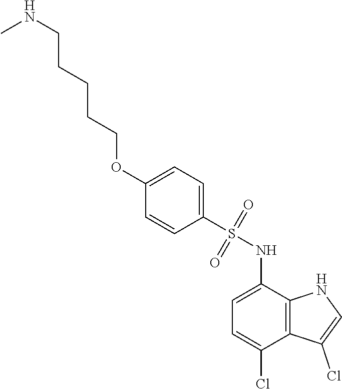 [SMILES: CNCCCCCOc1ccc(S(=O)(=O)Nc2ccc(Cl)c3c(Cl)c[nH]c23)cc1] | 456 |
| 148 | 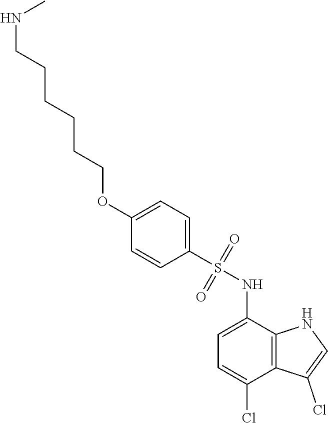 [SMILES: CNCCCCCCOc1ccc(S(=O)(=O)Nc2ccc(Cl)c3c(Cl)c[nH]c23)cc1] | 470 |
| 153 | 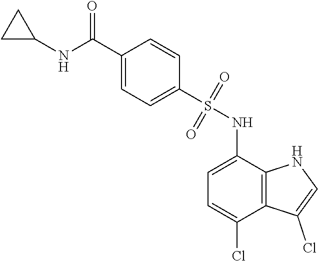 [SMILES: O=C(NC1CC1)c1ccc(S(=O)(=O)Nc2ccc(Cl)c3c(Cl)c[nH]c23)cc1] | NA |
| 154 | 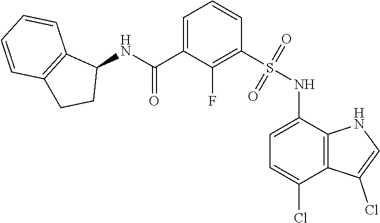 [SMILES: O=C(N[C@H]1CCc2ccccc21)c1cccc(S(=O)(=O)Nc2ccc(Cl)c3c(Cl)c[nH]c23)c1F] | 598.5 |
| 155 | 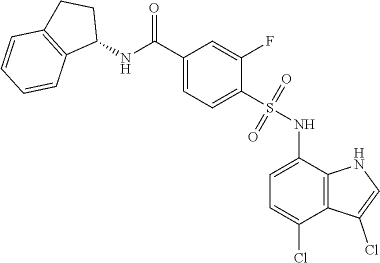 [SMILES: O=C(N[C@H]1CCc2ccccc21)c1ccc(S(=O)(=O)Nc2ccc(Cl)c3c(Cl)c[nH]c23)c(F)c1] | 518.4 |
| 156 | 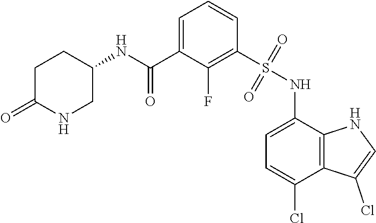 [SMILES: O=C1CC[C@H](NC(=O)c2cccc(S(=O)(=O)Nc3ccc(Cl)c4c(Cl)c[nH]c34)c2F)CN1] | 499.1 |
| 157 | 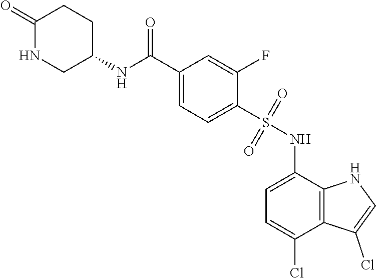 [SMILES: O=C1CC[C@H](NC(=O)c2ccc(S(=O)(=O)Nc3ccc(Cl)c4c(Cl)c[nH]c34)c(F)c2)CN1] | 499 |
| 158 | 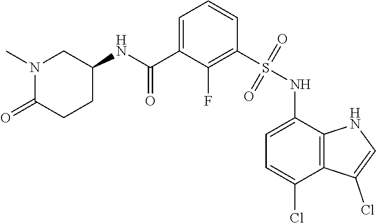 [SMILES: CN1C[C@@H](NC(=O)c2cccc(S(=O)(=O)Nc3ccc(Cl)c4c(Cl)c[nH]c34)c2F)CCC1=O] | 513 |
| 159 | 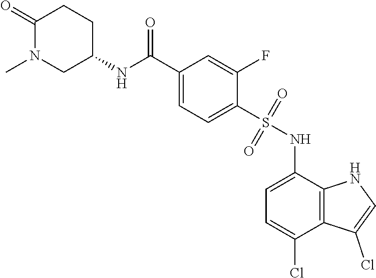 [SMILES: CN1C[C@@H](NC(=O)c2ccc(S(=O)(=O)Nc3ccc(Cl)c4c(Cl)c[nH]c34)c(F)c2)CCC1=O] | 513 |
| 160 | 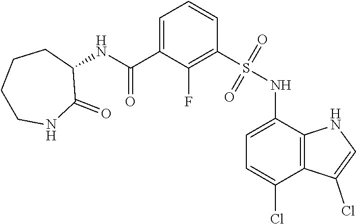 [SMILES: O=C(N[C@H]1CCCCNC1=O)c1cccc(S(=O)(=O)Nc2ccc(Cl)c3c(Cl)c[nH]c23)c1F] | 513 |
| 161 | 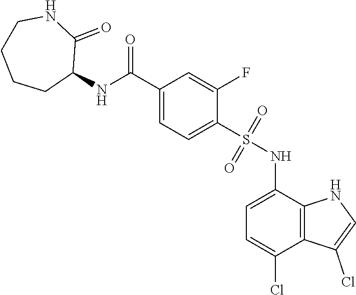 [SMILES: O=C(N[C@H]1CCCCNC1=O)c1ccc(S(=O)(=O)Nc2ccc(Cl)c3c(Cl)c[nH]c23)c(F)c1] | 513 |
| 162 | 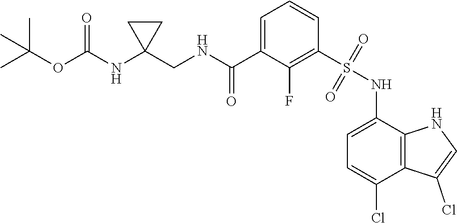 [SMILES: CC(C)(C)OC(=O)NC1(CNC(=O)c2cccc(S(=O)(=O)Nc3ccc(Cl)c4c(Cl)c[nH]c34)c2F)CC1] | 571.3 |
| 163 | 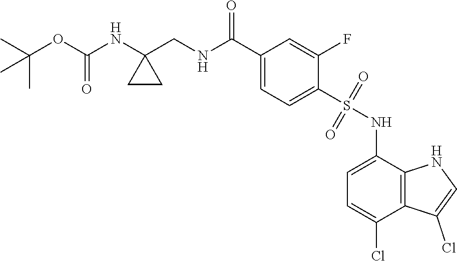 [SMILES: CC(C)(C)OC(=O)NC1(CNC(=O)c2ccc(S(=O)(=O)Nc3ccc(Cl)c4c(Cl)c[nH]c34)c(F)c2)CC1] | 571 |
| 164 | 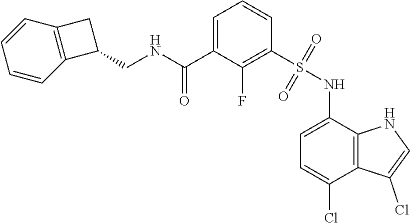 [SMILES: O=C(NC[C@H]1Cc2ccccc21)c1cccc(S(=O)(=O)Nc2ccc(Cl)c3c(Cl)c[nH]c23)c1F] | 518.4 |
| 165 | 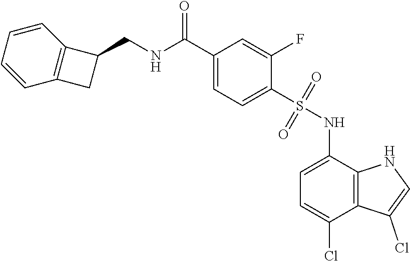 [SMILES: O=C(NC[C@H]1Cc2ccccc21)c1ccc(S(=O)(=O)Nc2ccc(Cl)c3c(Cl)c[nH]c23)c(F)c1] | 518.4 |
| 166 | 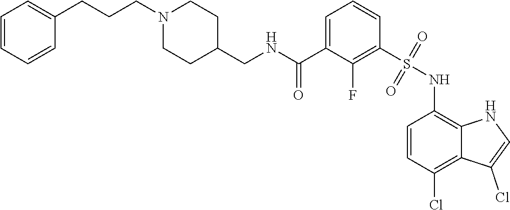 [SMILES: O=C(NCC1CCN(CCCc2ccccc2)CC1)c1cccc(S(=O)(=O)Nc2ccc(Cl)c3c(Cl)c[nH]c23)c1F] | 617.5 |
| 167 | 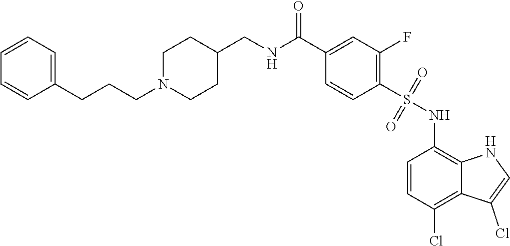 [SMILES: O=C(NCC1CCN(CCCc2ccccc2)CC1)c1ccc(S(=O)(=O)Nc2ccc(Cl)c3c(Cl)c[nH]c23)c(F)c1] | 617 |
| 168 | 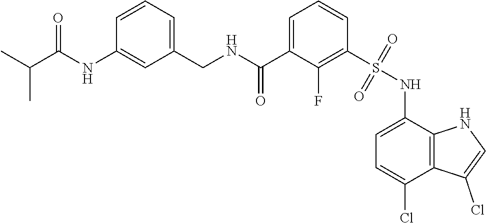 [SMILES: CC(C)C(=O)Nc1cccc(CNC(=O)c2cccc(S(=O)(=O)Nc3ccc(Cl)c4c(Cl)c[nH]c34)c2F)c1] | 577.4 |
| 169 | 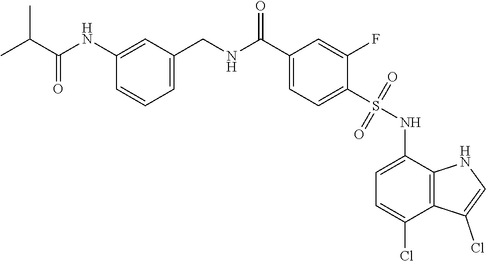 [SMILES: CC(C)C(=O)Nc1cccc(CNC(=O)c2ccc(S(=O)(=O)Nc3ccc(Cl)c4c(Cl)c[nH]c34)c(F)c2)c1] | 577.1 |
| 170 | 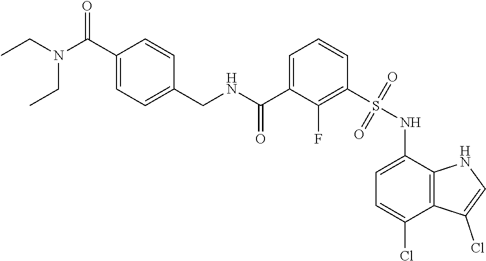 [SMILES: CCN(CC)C(=O)c1ccc(CNC(=O)c2cccc(S(=O)(=O)Nc3ccc(Cl)c4c(Cl)c[nH]c34)c2F)cc1] | 591.4 |
| 171 | 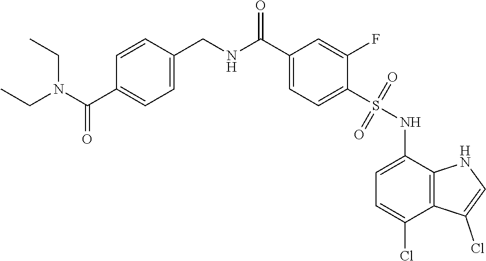 [SMILES: CCN(CC)C(=O)c1ccc(CNC(=O)c2ccc(S(=O)(=O)Nc3ccc(Cl)c4c(Cl)c[nH]c34)c(F)c2)cc1] | 591 |
| 172 | 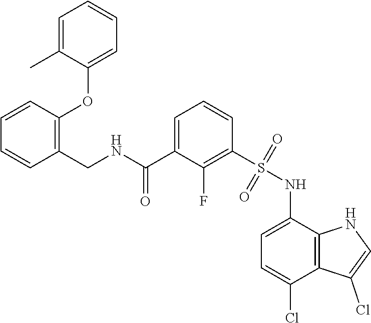 [SMILES: Cc1ccccc1Oc1ccccc1CNC(=O)c1cccc(S(=O)(=O)Nc2ccc(Cl)c3c(Cl)c[nH]c23)c1F] | 598.5 |
| 173 | 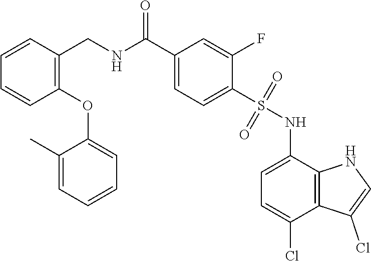 [SMILES: Cc1ccccc1Oc1ccccc1CNC(=O)c1ccc(S(=O)(=O)Nc2ccc(Cl)c3c(Cl)c[nH]c23)c(F)c1] | 598.4 |
| 174 | 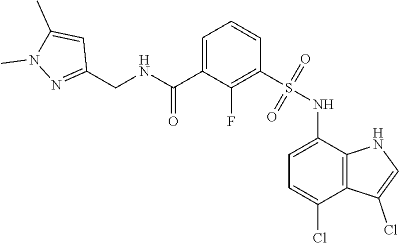 [SMILES: Cc1cc(CNC(=O)c2cccc(S(=O)(=O)Nc3ccc(Cl)c4c(Cl)c[nH]c34)c2F)nn1C] | 510 |
| 175 | 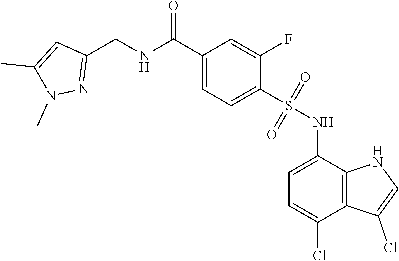 [SMILES: Cc1cc(CNC(=O)c2ccc(S(=O)(=O)Nc3ccc(Cl)c4c(Cl)c[nH]c34)c(F)c2)nn1C] | 510 |
| 176 | 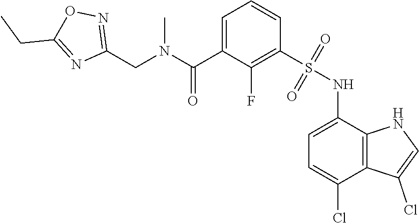 [SMILES: CCc1nc(CN(C)C(=O)c2cccc(S(=O)(=O)Nc3ccc(Cl)c4c(Cl)c[nH]c34)c2F)no1] | 526 |
| 177 | 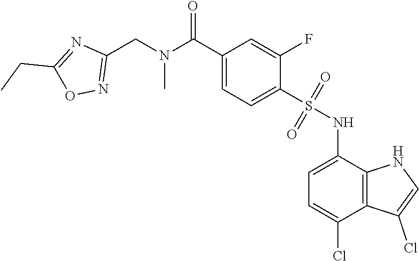 [SMILES: CCc1nc(CN(C)C(=O)c2ccc(S(=O)(=O)Nc3ccc(Cl)c4c(Cl)c[nH]c34)c(F)c2)no1] | 526 |
| 178 | 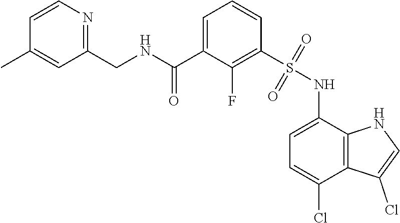 [SMILES: Cc1ccnc(CNC(=O)c2cccc(S(=O)(=O)Nc3ccc(Cl)c4c(Cl)c[nH]c34)c2F)c1] | 507.4 |
| 179 | 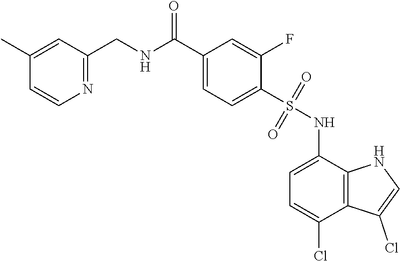 [SMILES: Cc1ccnc(CNC(=O)c2ccc(S(=O)(=O)Nc3ccc(Cl)c4c(Cl)c[nH]c34)c(F)c2)c1] | 507.4 |
| 180 | 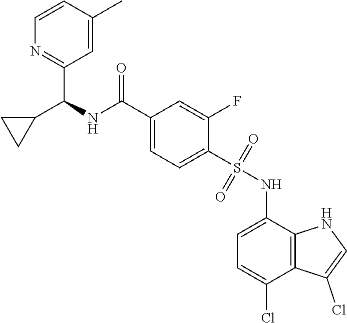 [SMILES: Cc1ccnc([C@@H](NC(=O)c2ccc(S(=O)(=O)Nc3ccc(Cl)c4c(Cl)c[nH]c34)c(F)c2)C2CC2)c1] | 546.4 |
| 181 | 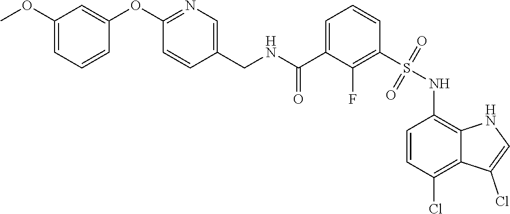 [SMILES: COc1cccc(Oc2ccc(CNC(=O)c3cccc(S(=O)(=O)Nc4ccc(Cl)c5c(Cl)c[nH]c45)c3F)cn2)c1] | 615 |
| 182 | 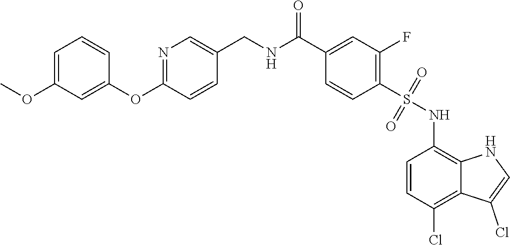 [SMILES: COc1cccc(Oc2ccc(CNC(=O)c3ccc(S(=O)(=O)Nc4ccc(Cl)c5c(Cl)c[nH]c45)c(F)c3)cn2)c1] | 615.4 |
| 183 | 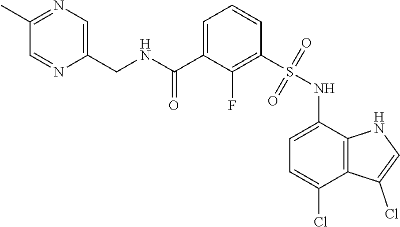 [SMILES: Cc1cnc(CNC(=O)c2cccc(S(=O)(=O)Nc3ccc(Cl)c4c(Cl)c[nH]c34)c2F)cn1] | 508 |
| 184 | 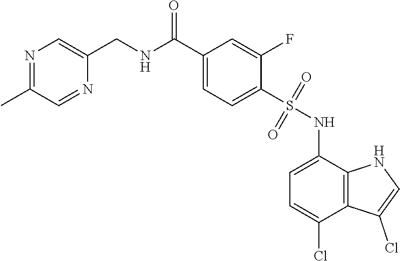 [SMILES: Cc1cnc(CNC(=O)c2ccc(S(=O)(=O)Nc3ccc(Cl)c4c(Cl)c[nH]c34)c(F)c2)cn1] | 508.3 |
| 185 | 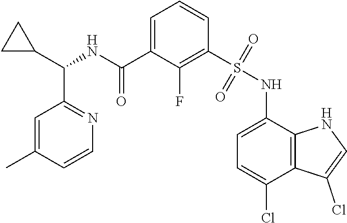 [SMILES: Cc1ccnc([C@@H](NC(=O)c2cccc(S(=O)(=O)Nc3ccc(Cl)c4c(Cl)c[nH]c34)c2F)C2CC2)c1] | 547 |
| 186 | 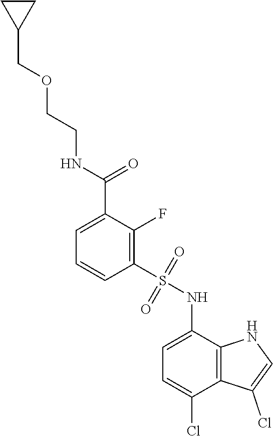 [SMILES: O=C(NCCOCC1CC1)c1cccc(S(=O)(=O)Nc2ccc(Cl)c3c(Cl)c[nH]c23)c1F] | 500 |
| 187 | 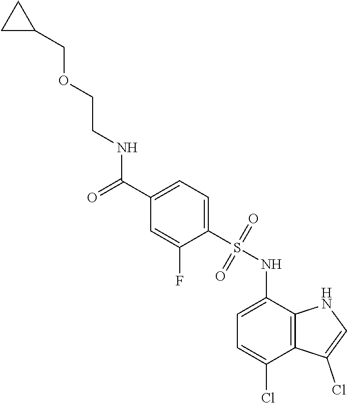 [SMILES: O=C(NCCOCC1CC1)c1ccc(S(=O)(=O)Nc2ccc(Cl)c3c(Cl)c[nH]c23)c(F)c1] | 500 |
| 188 | 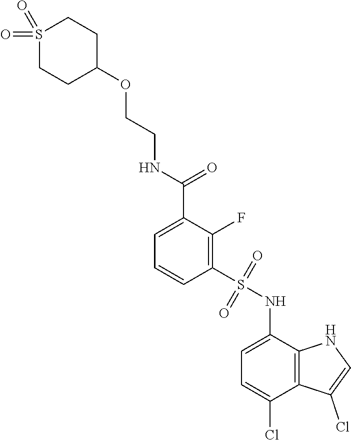 [SMILES: O=C(NCCOC1CCS(=O)(=O)CC1)c1cccc(S(=O)(=O)Nc2ccc(Cl)c3c(Cl)c[nH]c23)c1F] | 578.4 |
| 189 | 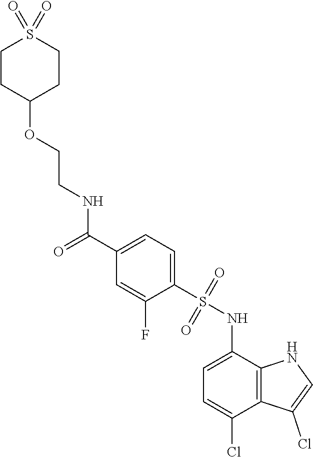 [SMILES: O=C(NCCOC1CCS(=O)(=O)CC1)c1ccc(S(=O)(=O)Nc2ccc(Cl)c3c(Cl)c[nH]c23)c(F)c1] | 578.3 |
| 190 | 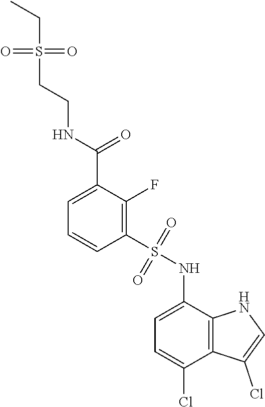 [SMILES: CCS(=O)(=O)CCNC(=O)c1cccc(S(=O)(=O)Nc2ccc(Cl)c3c(Cl)c[nH]c23)c1F] | 522 |
| 191 | 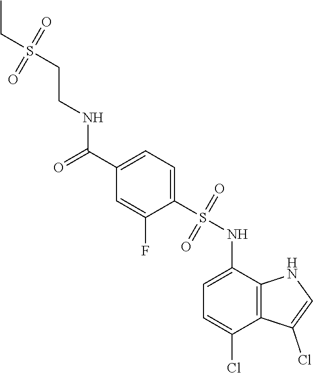 [SMILES: CCS(=O)(=O)CCNC(=O)c1ccc(S(=O)(=O)Nc2ccc(Cl)c3c(Cl)c[nH]c23)c(F)c1] | 522 |
| 192 | 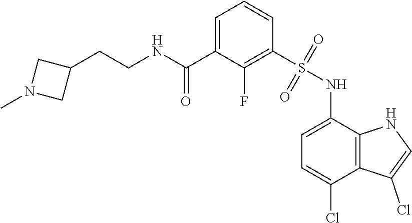 [SMILES: CN1CC(CCNC(=O)c2cccc(S(=O)(=O)Nc3ccc(Cl)c4c(Cl)c[nH]c34)c2F)C1] | 499.1 |
| 193 | 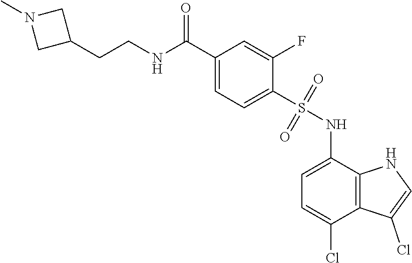 [SMILES: CN1CC(CCNC(=O)c2ccc(S(=O)(=O)Nc3ccc(Cl)c4c(Cl)c[nH]c34)c(F)c2)C1] | 499 |
| 194 | 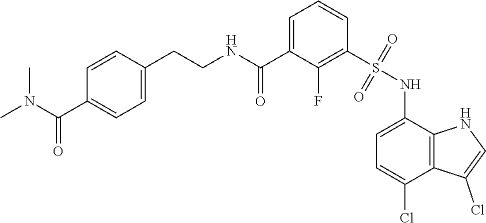 [SMILES: CN(C)C(=O)c1ccc(CCNC(=O)c2cccc(S(=O)(=O)Nc3ccc(Cl)c4c(Cl)c[nH]c34)c2F)cc1] | 577.4 |
| 195 | 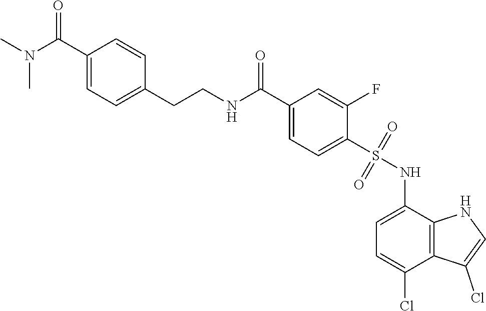 [SMILES: CN(C)C(=O)c1ccc(CCNC(=O)c2ccc(S(=O)(=O)Nc3ccc(Cl)c4c(Cl)c[nH]c34)c(F)c2)cc1] | 577.1 |
| 196 | 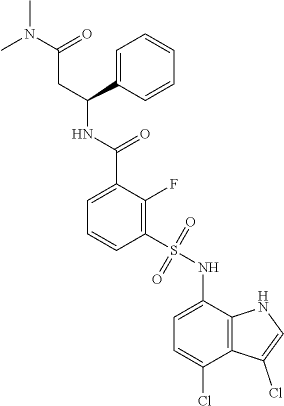 [SMILES: CN(C)C(=O)C[C@H](NC(=O)c1cccc(S(=O)(=O)Nc2ccc(Cl)c3c(Cl)c[nH]c23)c1F)c1ccccc1] | 577.4 |
| 197 | 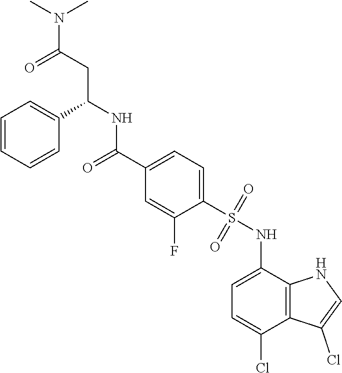 [SMILES: CN(C)C(=O)C[C@H](NC(=O)c1ccc(S(=O)(=O)Nc2ccc(Cl)c3c(Cl)c[nH]c23)c(F)c1)c1ccccc1] | 577.1 |
| 198 | 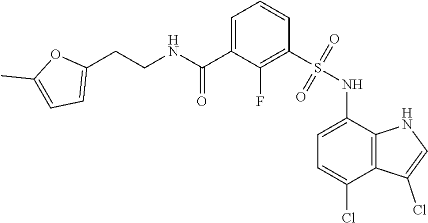 [SMILES: Cc1ccc(CCNC(=O)c2cccc(S(=O)(=O)Nc3ccc(Cl)c4c(Cl)c[nH]c34)c2F)o1] | 510 |
| 199 | 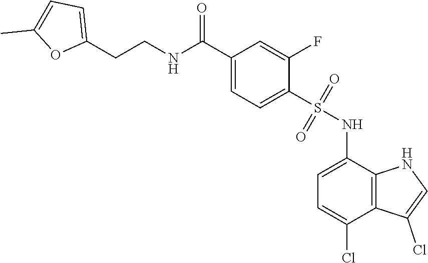 [SMILES: Cc1ccc(CCNC(=O)c2ccc(S(=O)(=O)Nc3ccc(Cl)c4c(Cl)c[nH]c34)c(F)c2)o1] | 510 |
| 200 | 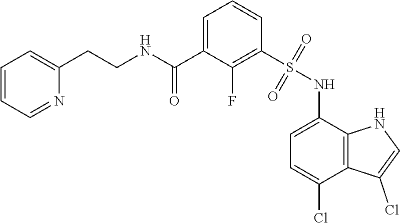 [SMILES: O=C(NCCc1ccccn1)c1cccc(S(=O)(=O)Nc2ccc(Cl)c3c(Cl)c[nH]c23)c1F] | 507 |
| 201 | 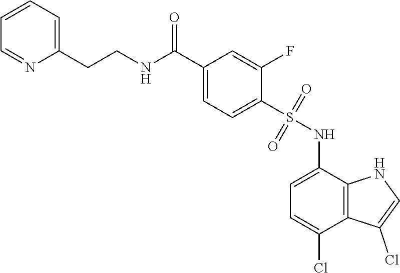 [SMILES: O=C(NCCc1ccccn1)c1ccc(S(=O)(=O)Nc2ccc(Cl)c3c(Cl)c[nH]c23)c(F)c1] | 507.4 |
| 202 | 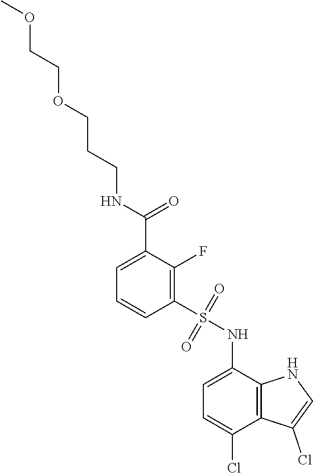 [SMILES: COCCOCCCNC(=O)c1cccc(S(=O)(=O)Nc2ccc(Cl)c3c(Cl)c[nH]c23)c1F] | 518.4 |
| 203 | 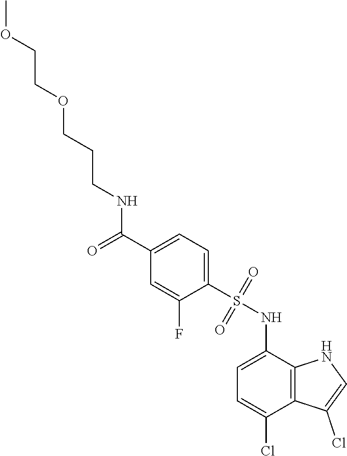 [SMILES: COCCOCCCNC(=O)c1ccc(S(=O)(=O)Nc2ccc(Cl)c3c(Cl)c[nH]c23)c(F)c1] | 518 |
| 204 | 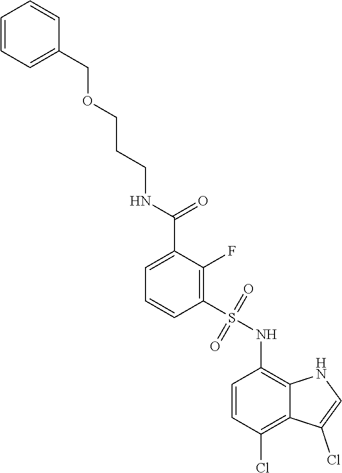 [SMILES: O=C(NCCCOCc1ccccc1)c1cccc(S(=O)(=O)Nc2ccc(Cl)c3c(Cl)c[nH]c23)c1F] | 550 |
| 205 | 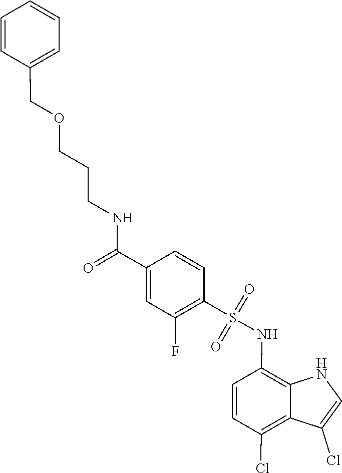 [SMILES: O=C(NCCCOCc1ccccc1)c1ccc(S(=O)(=O)Nc2ccc(Cl)c3c(Cl)c[nH]c23)c(F)c1] | 550 |
| 206 | 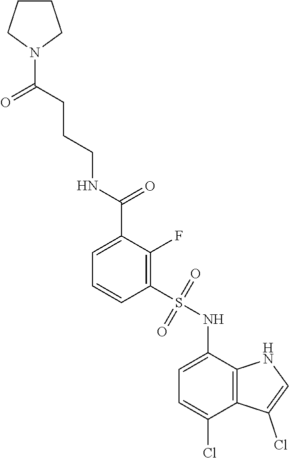 [SMILES: O=C(NCCCC(=O)N1CCCC1)c1cccc(S(=O)(=O)Nc2ccc(Cl)c3c(Cl)c[nH]c23)c1F] | 541 |
| 207 | 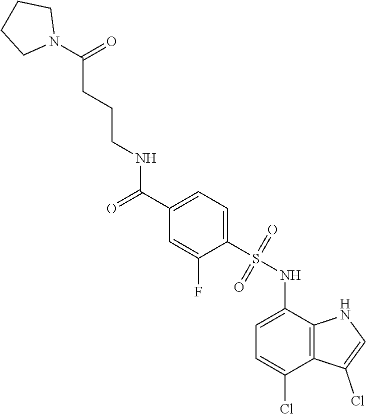 [SMILES: O=C(NCCCC(=O)N1CCCC1)c1ccc(S(=O)(=O)Nc2ccc(Cl)c3c(Cl)c[nH]c23)c(F)c1] | 541 |
| 208 | 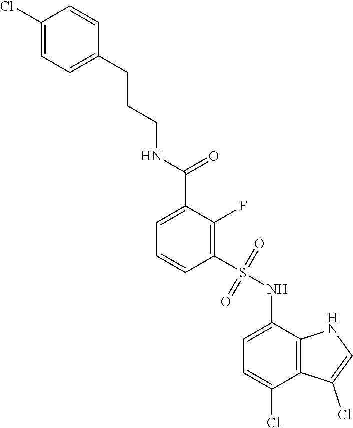 [SMILES: O=C(NCCCc1ccc(Cl)cc1)c1cccc(S(=O)(=O)Nc2ccc(Cl)c3c(Cl)c[nH]c23)c1F] | 554 |
| 209 | 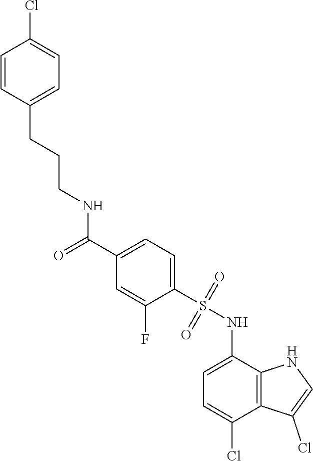 [SMILES: O=C(NCCCc1ccc(Cl)cc1)c1ccc(S(=O)(=O)Nc2ccc(Cl)c3c(Cl)c[nH]c23)c(F)c1] | 554.4 |
| 210 | 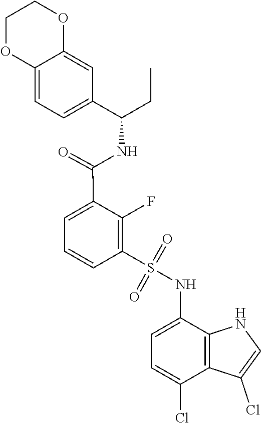 [SMILES: CC[C@H](NC(=O)c1cccc(S(=O)(=O)Nc2ccc(Cl)c3c(Cl)c[nH]c23)c1F)c1ccc2c(c1)OCCO2] | 578.4 |
| 211 | 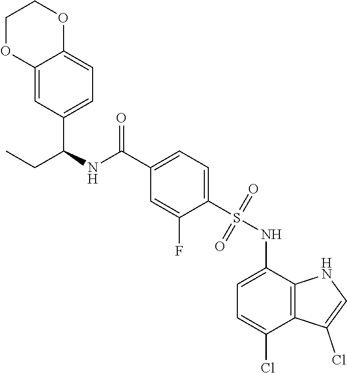 [SMILES: CC[C@H](NC(=O)c1ccc(S(=O)(=O)Nc2ccc(Cl)c3c(Cl)c[nH]c23)c(F)c1)c1ccc2c(c1)OCCO2] | 578.3 |
| 212 | 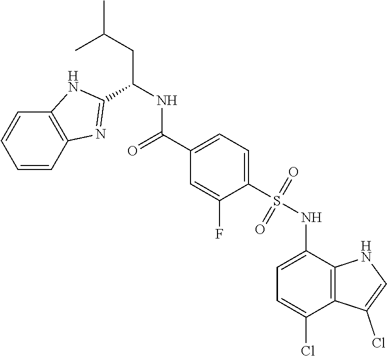 [SMILES: CC(C)C[C@H](NC(=O)c1ccc(S(=O)(=O)Nc2ccc(Cl)c3c(Cl)c[nH]c23)c(F)c1)c1nc2ccccc2[nH]1] | 588.5 |
| 213 | 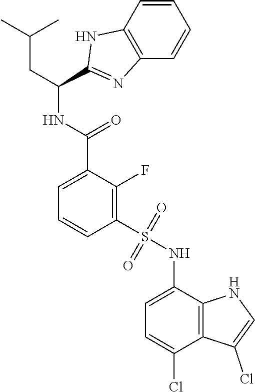 [SMILES: CC(C)C[C@H](NC(=O)c1cccc(S(=O)(=O)Nc2ccc(Cl)c3c(Cl)c[nH]c23)c1F)c1nc2ccccc2[nH]1] | 588 |
| 214 | 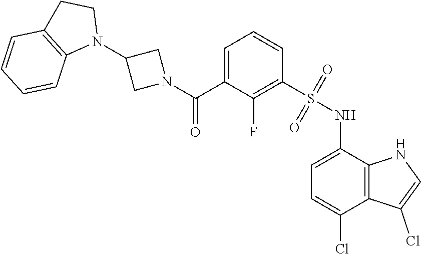 [SMILES: O=C(c1cccc(S(=O)(=O)Nc2ccc(Cl)c3c(Cl)c[nH]c23)c1F)N1CC(N2CCc3ccccc32)C1] | 559 |
| 215 | 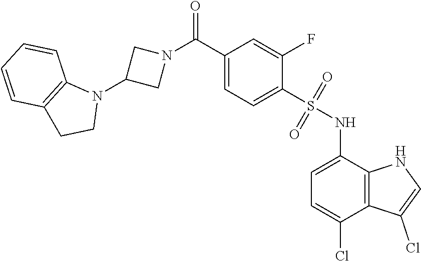 [SMILES: O=C(c1ccc(S(=O)(=O)Nc2ccc(Cl)c3c(Cl)c[nH]c23)c(F)c1)N1CC(N2CCc3ccccc32)C1] | 559 |
| 216 | 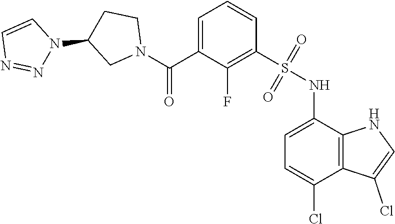 [SMILES: O=C(c1cccc(S(=O)(=O)Nc2ccc(Cl)c3c(Cl)c[nH]c23)c1F)N1CC[C@H](n2ccnn2)C1] | 523 |
| 217 | 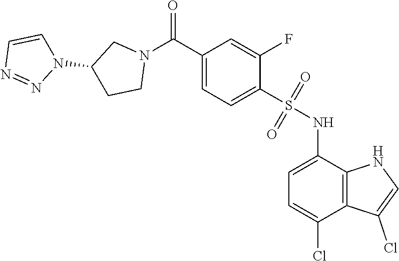 [SMILES: O=C(c1ccc(S(=O)(=O)Nc2ccc(Cl)c3c(Cl)c[nH]c23)c(F)c1)N1CC[C@H](n2ccnn2)C1] | 523.4 |
| 218 | 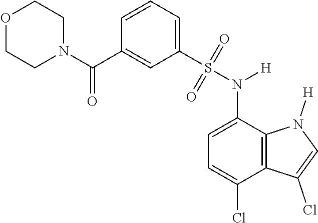 [SMILES: O=C(c1cccc(S(=O)(=O)Nc2ccc(Cl)c3c(Cl)c[nH]c23)c1)N1CCOCC1] | NA |
| 219 | 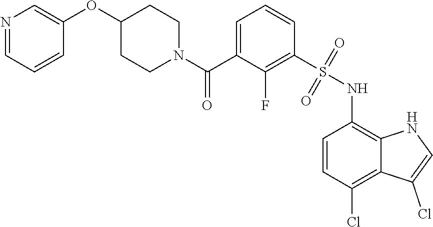 [SMILES: O=C(c1cccc(S(=O)(=O)Nc2ccc(Cl)c3c(Cl)c[nH]c23)c1F)N1CCC(Oc2cccnc2)CC1] | 563.4 |
| 220 | 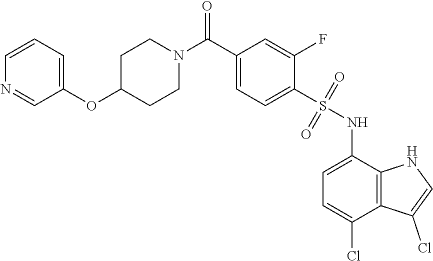 [SMILES: O=C(c1ccc(S(=O)(=O)Nc2ccc(Cl)c3c(Cl)c[nH]c23)c(F)c1)N1CCC(Oc2cccnc2)CC1] | 563.4 |
| 221 | 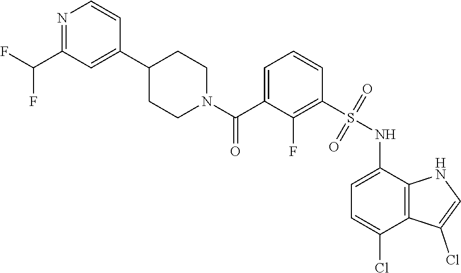 [SMILES: O=C(c1cccc(S(=O)(=O)Nc2ccc(Cl)c3c(Cl)c[nH]c23)c1F)N1CCC(c2ccnc(C(F)F)c2)CC1] | 597 |
| 222 | 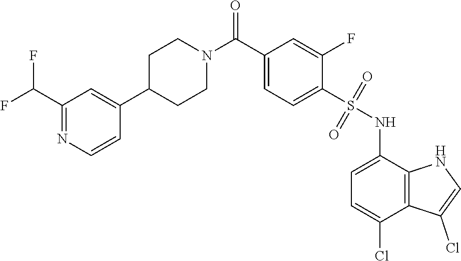 [SMILES: O=C(c1ccc(S(=O)(=O)Nc2ccc(Cl)c3c(Cl)c[nH]c23)c(F)c1)N1CCC(c2ccnc(C(F)F)c2)CC1] | 597.4 |
| 223 | 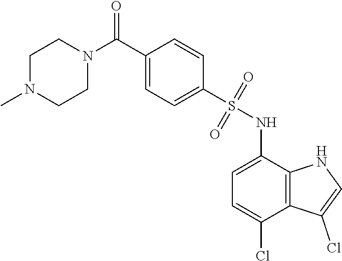 [SMILES: CN1CCN(C(=O)c2ccc(S(=O)(=O)Nc3ccc(Cl)c4c(Cl)c[nH]c34)cc2)CC1] | 467 |
| 224 | 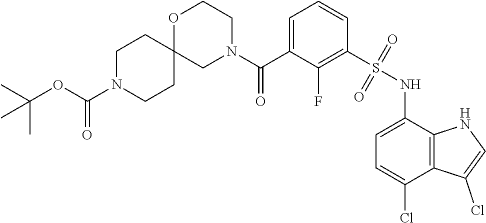 [SMILES: CC(C)(C)OC(=O)N1CCC2(CC1)CN(C(=O)c1cccc(S(=O)(=O)Nc3ccc(Cl)c4c(Cl)c[nH]c34)c1F)CCO2] | 641 |
| 225 | 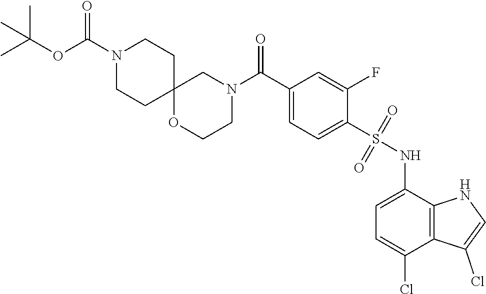 [SMILES: CC(C)(C)OC(=O)N1CCC2(CC1)CN(C(=O)c1ccc(S(=O)(=O)Nc3ccc(Cl)c4c(Cl)c[nH]c34)c(F)c1)CCO2] | 641 |
| 226 | 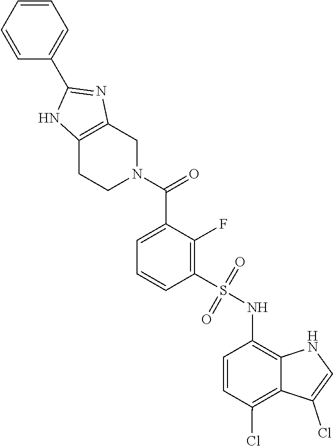 [SMILES: O=C(c1cccc(S(=O)(=O)Nc2ccc(Cl)c3c(Cl)c[nH]c23)c1F)N1CCc2[nH]c(-c3ccccc3)nc2C1] | 584 |
| 227 | 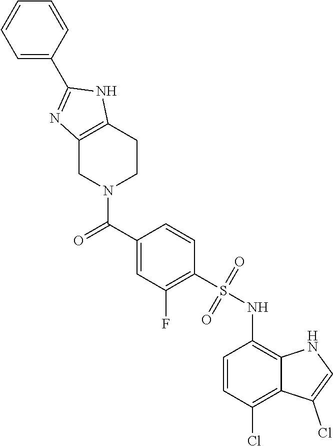 [SMILES: O=C(c1ccc(S(=O)(=O)Nc2ccc(Cl)c3c(Cl)c[nH]c23)c(F)c1)N1CCc2[nH]c(-c3ccccc3)nc2C1] | 584 |
| 228 | 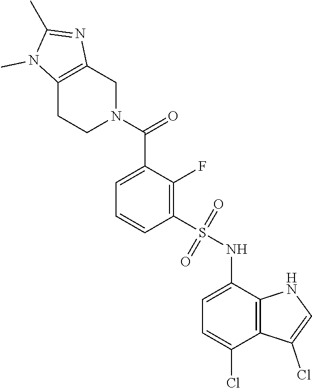 [SMILES: Cc1nc2c(n1C)CCN(C(=O)c1cccc(S(=O)(=O)Nc3ccc(Cl)c4c(Cl)c[nH]c34)c1F)C2] | 536.4 |
| 229 | 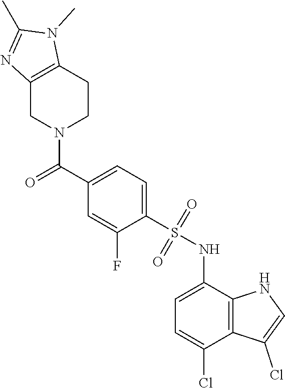 [SMILES: Cc1nc2c(n1C)CCN(C(=O)c1ccc(S(=O)(=O)Nc3ccc(Cl)c4c(Cl)c[nH]c34)c(F)c1)C2] | 536 |
| 230 | 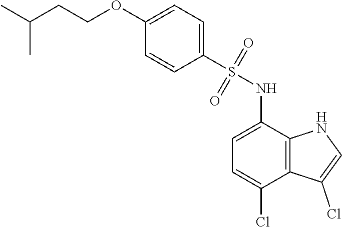 [SMILES: CC(C)CCOc1ccc(S(=O)(=O)Nc2ccc(Cl)c3c(Cl)c[nH]c23)cc1] | NA |
| 231 | 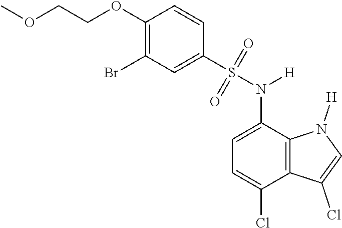 [SMILES: COCCOc1ccc(S(=O)(=O)Nc2ccc(Cl)c3c(Cl)c[nH]c23)cc1Br] | NA |
| 232 | 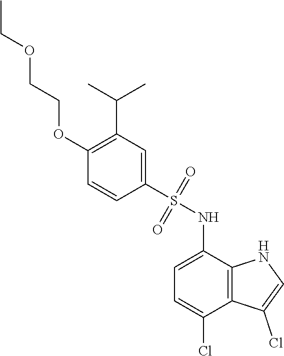 [SMILES: CCOCCOc1ccc(S(=O)(=O)Nc2ccc(Cl)c3c(Cl)c[nH]c23)cc1C(C)C] | NA |
| 233 | 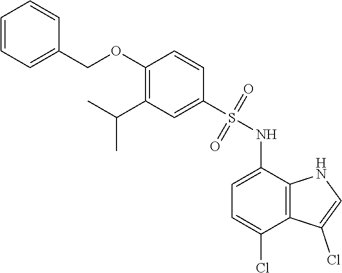 [SMILES: CC(C)c1cc(S(=O)(=O)Nc2ccc(Cl)c3c(Cl)c[nH]c23)ccc1OCc1ccccc1] | NA |
| 234 | 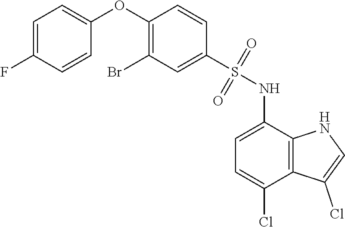 [SMILES: O=S(=O)(Nc1ccc(Cl)c2c(Cl)c[nH]c12)c1ccc(Oc2ccc(F)cc2)c(Br)c1] | NA |
| 235 | 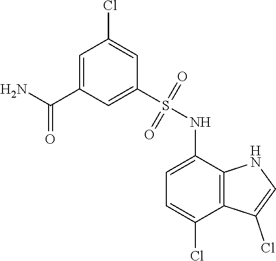 [SMILES: NC(=O)c1cc(Cl)cc(S(=O)(=O)Nc2ccc(Cl)c3c(Cl)c[nH]c23)c1] | NA |
| 236 | 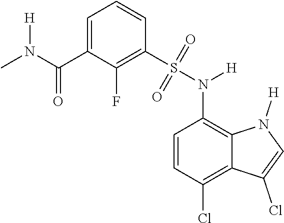 [SMILES: CNC(=O)c1cccc(S(=O)(=O)Nc2ccc(Cl)c3c(Cl)c[nH]c23)c1F] | 416.3 |
| 237 | 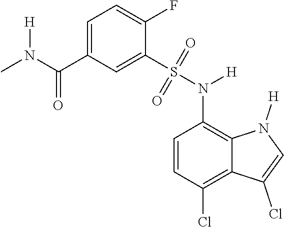 [SMILES: CNC(=O)c1ccc(F)c(S(=O)(=O)Nc2ccc(Cl)c3c(Cl)c[nH]c23)c1] | 416.2 |
| 238 | 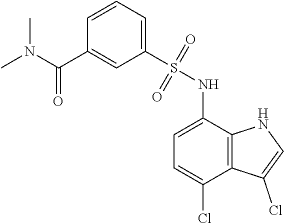 [SMILES: CN(C)C(=O)c1cccc(S(=O)(=O)Nc2ccc(Cl)c3c(Cl)c[nH]c23)c1] | NA |
| 239 | 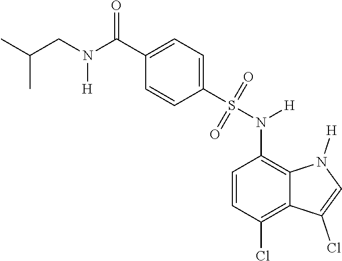 [SMILES: CC(C)CNC(=O)c1ccc(S(=O)(=O)Nc2ccc(Cl)c3c(Cl)c[nH]c23)cc1] | 440 |
| 240 | 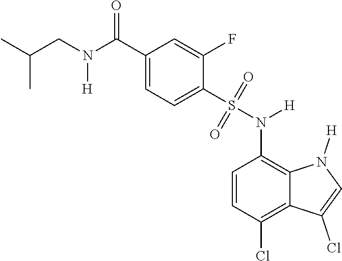 [SMILES: CC(C)CNC(=O)c1ccc(S(=O)(=O)Nc2ccc(Cl)c3c(Cl)c[nH]c23)c(F)c1] | 458.4 |

### Example 6: Quantitative Evaluation of Compound-Induced Ternary Complex Formation Using Amplified Luminescent Proximity Homogeneous Assay

Compound-induced DCAF15-RBM39 ternary complex formation was monitored using AlphaLISA® assays (Perkin Elmer, Waltham, MA). Test compounds were first collected in a 384-well microplate (cat. #781280, Grenier Bio-One, Monroe, NC) as 2 mM stocks in 100% dimethyl sulfoxide (DMSO). These stocks were then serially diluted in DMSO by 3-fold into a new plate (cat. #781280, Grenier Bio-One, Monroe, NC) using a Mosquito HTS dispenser (mosquito@ HTS, SPT Labtech, Boston, MA). 0.2 ul of these diluted stocks were then dispensed into a 384-well Optiplate (cat. #6007290, Perkin Elmer, Waltham, MA) in duplicate wells, to assemble the final assay plate consisting of duplicated dose ranges. Separately, a protein mixture consisting of His-tagged DCAF15 complex and biotin-carrying RBM39, each at 408 nM (2.04×), was prepared in 50 mM HEPES, pH 7.5, 100 mM NaCl, 2 mM MgCl₂, 0.1% BSA, 2 mM TCEP, 0.05% Tween-20 (dilution buffer). 9.8 ul of this protein mixture was then mixed with the compounds in the assay plate and incubated for one hour at room temperature. Reaction mixtures with no compounds added were included as negative controls, while a His-tagged and biotinylated GST protein was included to track general assay performance. Nickel Chelate AlphaLISA Acceptor beads (cat. #AL108M, Perkin Elmer, MA) and AlphaScreen Streptavidin Donor beads (cat. #6760002, Perkin Elmer, Waltham, MA) were prepared separately in the dilution buffer as 4× stocks (80 ug/ml). The beads were then added to the assay plate sequentially with incubation at room temperature for 1-2 hours after each addition. Signals were recorded using EnVision 2104 Multilabel Plate Reader (Perkin Elmer, Waltham, MA) and titration curves were generated in GraphPad Prism (7.00, GraphPad, La Jolla, CA). Results are summarized in Table 2.

### Example 7: Measurement of Binary Interaction Between Compounds And DCAF15 Using Homogeneous Time Resolved Fluorescence (HTRF)

DCAF15 interactions with test compounds were monitored using HTRF competition assays (Cisbio, Bedford, MA). Test compounds were first collected in a 384-well microplate (cat. #781280, Grenier Bio-One, Monroe, NC) as 10 mM stocks in 100% dimethyl sulfoxide (DMSO). A second stock plate (cat. #781280, Grenier Bio-One, Monroe, NC) was prepared through 3-fold serial dilution in DMSO of the afore-mentioned stocks using a Mosquito HTS dispenser (mosquito@ HTS, SPT Labtech, Boston, MA). 0.2 ul of these diluted stocks were then dispensed into a 384-well Optiplate (cat. #6007290, Perkin Elmer, Waltham, MA) in duplicate wells, to assemble the assay plate. The tracer molecule containing an Alexa Fluor 647 probe was prepared in 25 mM HEPES pH 7.5, 100 mM NaCl, 0.1 mg/ml BSA, 0.005% Tween 20, 0.5 mM TCEP (dilution buffer) to 612 nM (2.04×). Separately, His-tagged DCAF15 complex was prepared at 32 nM (4×) in the dilution buffer, and mAb Anti-6His-Eu cryptate Gold (cat. #61H12KLA, Cisbio, Bedford, MA) at 4× dilution in the detection buffer (cat. #61 DB9RDF, Cisbio, Bedford, MA). These two solutions were mixed and incubated for 15 min at room temperature. 9.8 ul of the tracer solution and 10 ul of the DCAF15/mAb Anti-6His-Eu cryptate Gold mixture were added sequentially to each well in the assay plate. Reaction mixtures with no DCAF15 added were included as positive controls. The final mixture was incubated for an hour at room temperature and spun down briefly before data collection at 615 nm and 666 nm using En Vision 2104 Multilabel Plate Reader (Perkin Elmer, Bedford, MA). Binding was estimated based on ratiometric analysis and titration curves were generated in GraphPad Prism (7.00, GraphPad, La Jolla, CA). Results are summarized in Table 2.

### Example 8: Western Blot Assay Procedure for Assessing Degradation of RBM39 in HCT116 Cells

HCT116 cells were used with passage numbers ranging from 13 to 25. Cells were seeded in 12-well plated at a density of 1 million cells per well in 1 mL cell culture medium containing McCoy's 5A medium (Cat. #16600082, ThermoFisher, Waltham, MA), 10% Fetal Bovine Serum (Cat. #26140, ThermoFisher, Waltham, MA) and 1% Pen-Strep (Cat. #. 15070063, ThermoFisher, Waltham, MA) and incubated overnight. The next morning, compounds were serially diluted (1:3, 9 points) in the medium separately and were prepared 2× the final assay concentration. Subsequently, 1.0 mL medium containing DMSO or the appropriate concentration of each compound was added to respective wells. The final DMSO concentration in each well was 0.1%. After 24 hour incubation with compounds, cell were harvested (approximately 90-100% confluency at the time of harvest). Cells were then washed once with 1 mL of ice cold Dulbecco's phosphate-buffered saline (Cat. #28374, ThermoFisher, Waltham, MA) and then lysed with 100 μl of lysis buffer containing 1×RIPA (Cat. #. 89900, ThermoFisher, Waltham, MA) and HALT protease and phosphatase inhibitor (Cat. #78442, ThermoFisher, Waltham, MA) on ice for 10 minutes with intermittent rocking. Lysates were then sonicated in a sonication bath (3 pulses of 10 sec each with 1 min incubation on ice between each pulse). Samples were then centrifuged for 20 min at 14,000 rpm for 20 min. Supernatants were then transferred to fresh microfuge tubes and stored frozen at −80° C. until use. Total protein quantitation in lysate samples was done using the BCA protein quantitation kit following the manufacturer's instructions. For polyacrylamide gel electrophoresis, each sample (15-30 uL total protein), was diluted with Laemmli sample buffer (Cat. #. 1610747, BioRad, Hercules, CA) by adding 9 ul of lysate to 3 ul of sample buffer containing 10% β-mercaptoethanol. Samples were then denatured at 95° C. for 5 minutes in a heat block, centrifuged at 14,000 rpm for 5 min, and placed on ice. Subsequently, 12 ul of each sample was loaded on 4-20% Tris/Glycine gels and electrophoresed in 1×Tris/Glycine SDS buffer at 80 (constant) volts for 2 h. Resolved samples were then transferred from gels to PVDF membrane using the BioRad Turbo-transfer unit with the Turbo default program (Cat. #. 1704150EDU, BioRad, Hercules, CA). For immunoblotting, membranes were first blocked for 1 hour at room temperature using Odyssey Blocking Buffer (Cat. #: 927-60001, Li-cor, Lincoln, NE) with continuous rocking. Membrane was then incubated with primary antibodies, anti-RBM39 (Sigma Cat. #: HPA001591, St. Louis, MO) and anti-β-actin (Cell Signaling Cat. #3700S, Danvers, MA), diluted 1000 fold each, at 4° C. for overnight. Subsequently, membrane was washed three times for at room temperature with continuous rocking (5 minutes each wash) using TBS buffer containing 0.1% Tween-20. After the final wash, secondary antibodies, IRDye 680 RD Goat anti Mouse IgG H+L (Cat. #: 926-68070, Li-cor, Lincoln, NE), and IRDye 800CW Goat anti Rabbit IgG H+L (Cat. #: 926-32211, Li-cor, Lincoln, NE), each diluted 10,000-fold, were added to the membranes and incubated at room temperature for 1 h with continuous rocking. Membranes were then washed 3 times with 1×TBS and scanned using Odessey CLx imaging system (Li-cor, Lincoln, NE) using default settings. Band intensity in images was quantified using the Image Studio Lite software (Version 5.2). Signal intensity for RBM-39 specific bands were normalized using the signal intensity for β-actin-specific band, and percent degradation was assessed by comparing RBM39 signal in compound-treated vs. DMSO-treated samples. Results are summarized in Table 3.

TABLE 2

| | AlphaLISA | HTRF | | AlphaLISA | HTRF |
|---|---|---|---|---|---|
| | ternary | binary | | ternary | binary |
| | DCAF15/RBM39: | DCAF15 | | DCAF15/RBM39: | DCAF15 |
| | Mean EC₅₀ | Mean IC50 | | Mean EC₅₀ | Mean IC50 |
| | (M) | (M) | | (M) | (M) |
| | “− >100 uM” | “− >100 uM” | | “− >100 uM” | “− >100 uM” |
| | “+ <100 uM” | “+ <100 uM” | | “+ <100 uM” | “+ <100 uM” |
| | “++ <10 uM” | “++ <10 uM” | | “++ <10 uM” | “++ <10 uM” |
| Cmp# | “+++ <1 uM” | “+++ <1 uM” | Cmp# | “+++ <1 uM” | “+++ <1 uM” |
| Indisulam | +++ | | Indisulam | +++ | |
| E-7820 | +++ | ++ | E-7820 | +++ | ++ |
| Tasisulam | ++ | | Tasisulam | ++ | |
| CQS | ++ | | CQS | ++ | |
| 3 | +++ | ++ | 153 | +++ | ++ |
| 4 | +++ | ++ | 154 | ++ | ++ |
| 5 | +++ | ++ | 155 | − | − |
| 6 | +++ | + | 156 | +++ | ++ |
| 7 | +++ | ++ | 157 | +++ | +++ |
| 8 | +++ | ++ | 158 | +++ | +++ |
| 9 | ++ | ++ | 159 | +++ | +++ |
| 12 | − | − | 160 | +++ | +++ |
| 13 | − | − | 161 | +++ | +++ |
| 14 | +++ | ++ | 162 | ++ | ++ |
| 15 | ++ | ++ | 163 | ++ | ++ |
| 16 | +++ | ++ | 164 | ++ | ++ |
| 17 | +++ | + | 165 | − | − |
| 18 | − | − | 166 | +++ | ++ |
| 19 | − | − | 167 | +++ | +++ |
| 20 | ++ | − | 168 | ++ | ++ |
| 21 | ++ | − | 169 | ++ | ++ |
| 22 | +++ | ++ | 170 | ++ | ++ |
| 23 | +++ | ++ | 171 | ++ | ++ |
| 24 | +++ | ++ | 172 | ++ | ++ |
| 25 | +++ | ++ | 173 | − | − |
| 26 | +++ | + | 174 | +++ | ++ |
| 27 | ++ | ++ | 175 | +++ | ++ |
| 32 | ++ | + | 176 | +++ | ++ |
| 34 | ++ | − | 177 | +++ | ++ |
| 35 | + | − | 178 | ++ | ++ |
| 36 | ++ | + | 179 | +++ | ++ |
| 37 | ++ | − | 180 | ++ | ++ |
| 38 | +++ | ++ | 181 | ++ | ++ |
| 39 | +++ | − | 182 | − | − |
| 40 | +++ | − | 183 | +++ | ++ |
| 41 | +++ | ++ | 184 | − | +++ |
| 42 | ++ | ++ | 185 | ++ | ++ |
| 43 | ++ | − | 186 | ++ | ++ |
| 44 | ++ | − | 187 | +++ | ++ |
| 45 | +++ | + | 188 | +++ | ++ |
| 46 | ++ | + | 189 | +++ | +++ |
| 47 | +++ | + | 190 | +++ | ++ |
| 48 | +++ | + | 191 | +++ | +++ |
| 49 | ++ | − | 192 | +++ | +++ |
| 50 | − | − | 193 | +++ | ++ |
| 53 | ++ | − | 194 | ++ | ++ |
| 54 | ++ | − | 195 | ++ | ++ |
| 59 | ++ | + | 196 | ++ | ++ |
| 60 | ++ | ++ | 197 | +++ | ++ |
| 63 | ++ | − | 198 | ++ | ++ |
| 64 | − | − | 199 | ++ | ++ |
| 65 | +++ | − | 200 | +++ | +++ |
| 66 | +++ | ++ | 201 | +++ | ++ |
| 67 | +++ | ++ | 202 | +++ | ++ |
| 68 | +++ | − | 203 | +++ | ++ |
| 71 | ++ | ++ | 204 | ++ | ++ |
| 74 | +++ | + | 205 | +++ | − |
| 75 | +++ | ++ | 206 | +++ | +++ |
| 76 | ++ | − | 207 | +++ | +++ |
| 77 | ++ | − | 208 | − | − |
| 92 | + | − | 209 | ++ | − |
| 93 | − | − | 210 | ++ | ++ |
| 96 | +++ | − | 211 | ++ | ++ |
| 97 | +++ | ++ | 212 | ++ | ++ |
| 98 | ++ | + | 213 | +++ | ++ |
| 99 | +++ | ++ | 214 | ++ | ++ |
| 100 | − | − | 215 | ++ | + |
| 101 | | | 216 | +++ | +++ |
| 104 | +++ | + | 217 | +++ | +++ |
| 105 | | | 218 | +++ | ++ |
| 108 | +++ | − | 219 | +++ | +++ |
| 109 | | | 220 | +++ | ++ |
| 110 | − | − | 221 | +++ | +++ |
| 111 | − | − | 222 | − | − |
| 112 | ++ | − | 223 | +++ | ++ |
| 113 | ++ | − | 224 | +++ | ++ |
| 114 | ++ | − | 225 | ++ | ++ |
| 115 | − | − | 226 | +++ | ++ |
| 116 | ++ | − | 227 | ++ | ++ |
| 117 | ++ | − | 228 | +++ | +++ |
| 120 | +++ | − | 229 | +++ | +++ |
| 121 | +++ | + | 230 | ++ | ++ |
| 122 | ++ | − | 231 | ++ | ++ |
| 123 | − | − | 232 | ++ | − |
| 124 | + | − | 233 | + | + |
| 128 | + | − | 234 | ++ | − |
| 129 | ++ | − | 235 | ++ | ++ |
| 140 | ++ | ++ | 236 | +++ | ++ |
| 141 | ++ | − | 237 | ++ | + |
| 143 | +++ | − | 238 | ++ | ++ |
| 144 | +++ | ++ | 239 | +++ | − |
| 148 | +++ | ++ | 240 | +++ | ++ |

TABLE 3

| | RBM39 | RBM39 |
|---|---|---|
| | 4-Hour DC₅₀ | DegradationDₘₐₓ |
| Cmp# | (M) | (%) |
| E-7820 | +++ | ++ |
| Tasisulam | ++ | |
| CQS | ++ | |
| E-7820 | +++ | ++ |
| 39 | 0.0931 | 97.6 |
| 71 | 0.112 | 100 |
| 153 | 0.174 | 94.8 |
| 218 | 0.1 | 100 |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) that may be cited throughout this application are hereby expressly incorporated herein in their entireties by reference. Unless otherwise defined, all technical and scientific terms used herein are accorded the meaning commonly known to one with ordinary skill in the art.

1. A compound Formula I 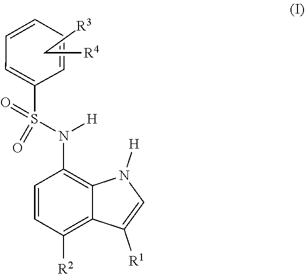
or a pharmaceutically acceptable salt thereof, wherein:
R¹ is selected from the group consisting of chloro, bromo, fluoro, and iodo;
R² is selected from the group consisting of H, chloro, fluoro, and methyl;
R³ is selected from the group consisting of H, chloro, fluoro, cyano, and methyl;
R⁴ is selected from the group consisting of
(a) 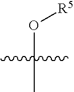
wherein:
R⁵ is selected from the group consisting of optionally substituted C₃ to C₇ cycloalkyl, optionally substituted 4 to 12-membered heterocyclyl, optionally substituted C₆-C₁₀ aryl, and substituted C₁-C₈ alkyl;
wherein the C₁-C₈ alkyl is substituted with at least one substituent selected from the group consisting of halo, C₁-C₆ alkoxy, (optionally substituted C₃-C₇cycloalkyl amino)carbonyl, (C₁-C₆ alkoxycarbonyl)(C₁-C₆ alkyl)amino, (C₁-C₆ alkyl)(C₁-C₆ alkyl)N—, C₁-C₆ alkylsulfonyl, optionally substituted 4 to 8-membered heterocyclyl, optionally substituted benzo[d][1,3]dioxolyl, optionally substituted benzooxazolonyl, optionally substituted tetrahydroquinolinyl, optionally substituted tetrahydroisoquinolinyl, optionally substituted C₆-C₁₀ aryl, optionally substituted isoindolinyl, and optionally substituted 5 to 10-membered heteroaryl; and
(b) 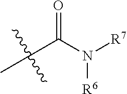
wherein
R⁶ is selected from the group consisting of H and CH₃;
R⁷ is selected from the group consisting of optionally substituted C₃-C₇ cycloalkyl, optionally substituted 2,3-dihydro-1H-indenyl, optionally substituted 3 to 7-membered heterocyclyl, and substituted C₁-C₅ alkyl;
wherein the C₁-C₅ alkyl is substituted with at least one substituent selected from the group consisting of optionally substituted C₁-C₆ alkoxy, optionally substituted 4 to 8-membered heterocyclyoxy, —C(O)NR⁸R⁹, C₁-C₆ alkylsulfonyl, optionally substituted C₃-C₇ cycloalkyl, optionally substituted bicyclooctatrienyl, optionally substituted 4 to 8-membered heterocyclyl, optionally substituted C₆-C₁₀ aryl, optionally substituted dihydrobenzodioxinyl, and optionally substituted 5 to 10-membered heteroaryl;
wherein R⁸ and R⁹ together with the nitrogen atom to which they are attached form an optionally substituted 5-6 membered heterocyclyl; or alternatively,
R⁶ and R⁷ together with the nitrogen atom to which they are attached form a group selected from optionally substituted 4 to 12-membered optionally substituted heterocyclyl, and optionally substituted 4,5,6,7-tetrahydro-1H-imidazo[4,5-c]pyridinyl;
with the proviso that the compound is not

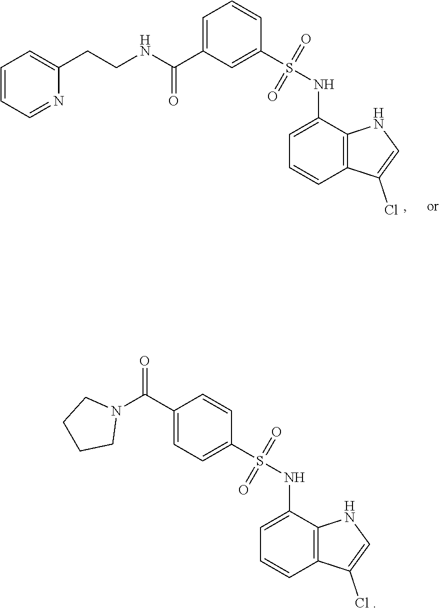 [SMILES: O=C(NCCc1ccccn1)c1cccc(S(=O)(=O)Nc2cccc3c(Cl)c[nH]c23)c1 ; O=C(c1ccc(S(=O)(=O)Nc2cccc3c(Cl)c[nH]c23)cc1)N1CCCC1]

2. A compound of Formula II 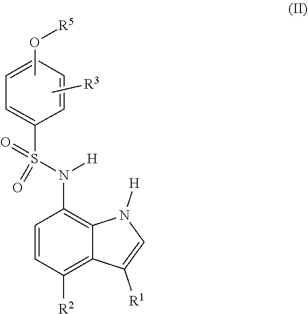
or a pharmaceutically acceptable salt thereof, wherein
R¹ is selected from the group consisting of chloro, bromo, fluoro, and iodo;
R² is selected from the group consisting of H, chloro, fluoro, and methyl;
R³ is selected from the group consisting of H, chloro, fluoro, cyano, and methyl;
R⁵ is selected from the group consisting of optionally substituted C₃ to C₇ cycloalkyl, optionally substituted 4 to 12-membered heterocyclyl, optionally substituted C₆-C₁₀ aryl, and substituted C₁-C₈ alkyl;
wherein, the C₁-C₈ alkyl is substituted with at least one substituent selected from the group consisting of halo, C₁-C₆ alkoxy, (optionally substituted C₃-C₇cycloalkyl amino)carbonyl, (C₁-C₆ alkoxycarbonyl)(C₁-C₆ alkyl)amino, —N(C₁-C₆ alkyl)(C₁-C₆ alkyl), C₁-C₆ alkylsulfonyl, optionally substituted 4 to 8-membered heterocyclyl, optionally substituted benzo[d][1,3]dioxolyl, optionally substituted benzooxazolonyl, optionally substituted tetrahydroquinolinyl, optionally substituted tetrahydroisoquinolinyl, optionally substituted C₆-C₁₀ aryl, optionally substituted isoindolinyl, and optionally substituted 5 to 10-membered heteroaryl.

3. The compound according to claim 2, or a pharmaceutically acceptable salt thereof, wherein the substituted C₁-C₈ alkyl is selected from the group consisting of:
C₁ alkyl substituted with at least one substituent selected from the group consisting of halo, (optionally substituted cyclopropylamino)carbonyl, optionally substituted oxetanyl, optionally substituted piperidinyl, optionally substituted azabicyclo[3.1.0]hexyl, optionally substituted octahydrocyclopenta[c]pyrrolyl, optionally substituted phenyl, optionally substituted isoindolinyl, optionally substituted triazolyl, and optionally substituted benzotriazolyl;
C₂ alkyl substituted with at least one substituent selected from the group consisting of methoxy, ethoxy, ethylsulfonyl, optionally substituted pyrrolidinyl, optionally substituted piperidinyl, optionally substituted phenyl, optionally substituted indolyl, optionally substituted isoindolinyl;
C₃ alkyl substituted with at least one substituent selected from the group consisting of optionally substituted pyrrolidinyl, optionally substituted piperidinyl, optionally substituted piperazinyl, 2-oxa-6-azaspiro[3.3]heptyl, optionally substituted benzo[d][1,3]dioxolyl, optionally substituted benzooxazolonyl, optionally substituted tetrahydroquinolinyl, optionally substituted pyrazolyl, optionally substituted triazolyl, optionally substituted pyridinyl, and optionally substituted benzoimidazolyl;
C₄ alkyl substituted with at least one substituent selected from the group consisting of optionally substituted piperidinyl, optionally substituted piperazinyl, optionally substituted thiomorpholinyl 1,1-dioxidyl, optionally substituted tetrahydroquinolinyl, optionally substituted pyrazolyl, and optionally substituted pyridinyl;
C₅ alkyl substituted with at least one substituent selected from the group consisting of (C₁-C₄ alkoxycarbonyl)(methyl)amino, and optionally substituted piperidinyl;
C₆ alkyl substituted with at least one substituent selected from the group consisting of (C₁-C₆ alkoxycarbonyl)(methyl)amino, and optionally substituted tetrahydroisoquinolinyl; and
C₈ alkyl substituted with —N(CH₃)₂.

4. The compound according to claim 2, or a pharmaceutically acceptable salt thereof wherein:
R¹ is chloro;
R² is selected from the group consisting of H and chloro;
R³ is selected from the group consisting H and F; and
R⁵ is selected from the group consisting of phenyl, —CHF₂, benzoyl, —CH₂CH₂OCH₃, —CH₂CH₂OCH₂CH₃, —CH₂CH₂S(O)₂CH₂CH₃,

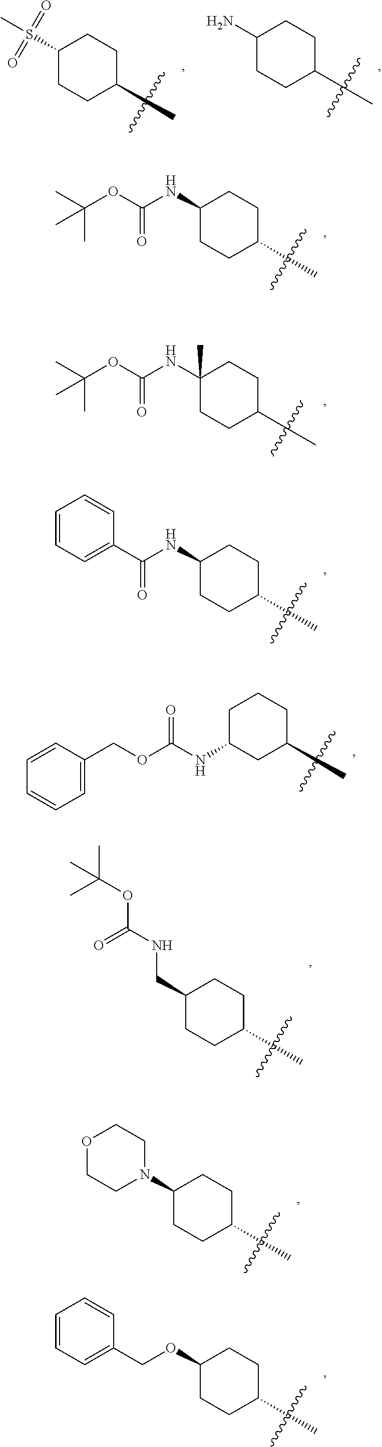

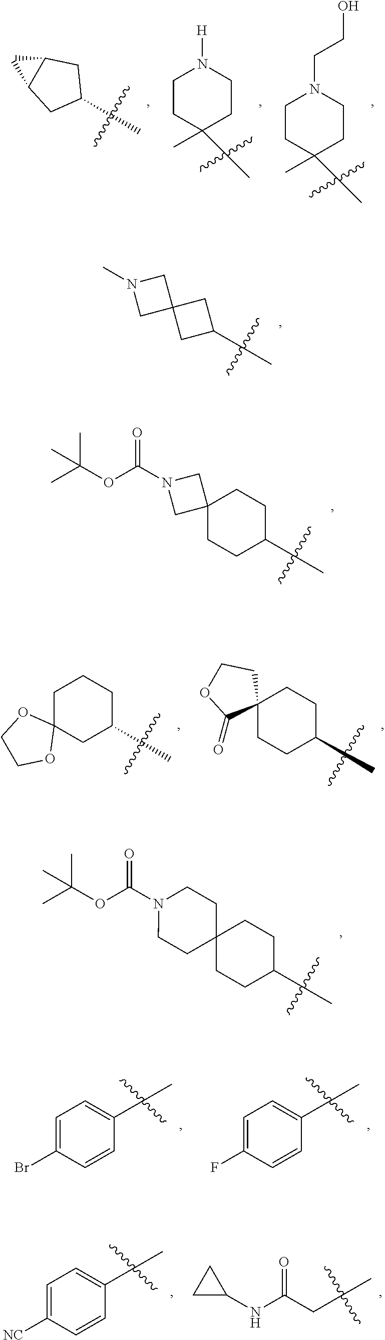

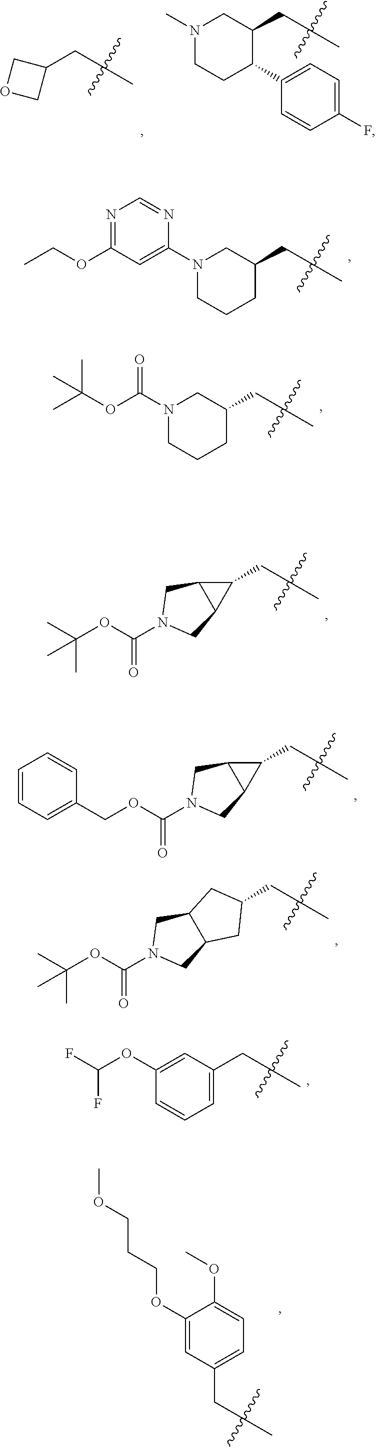

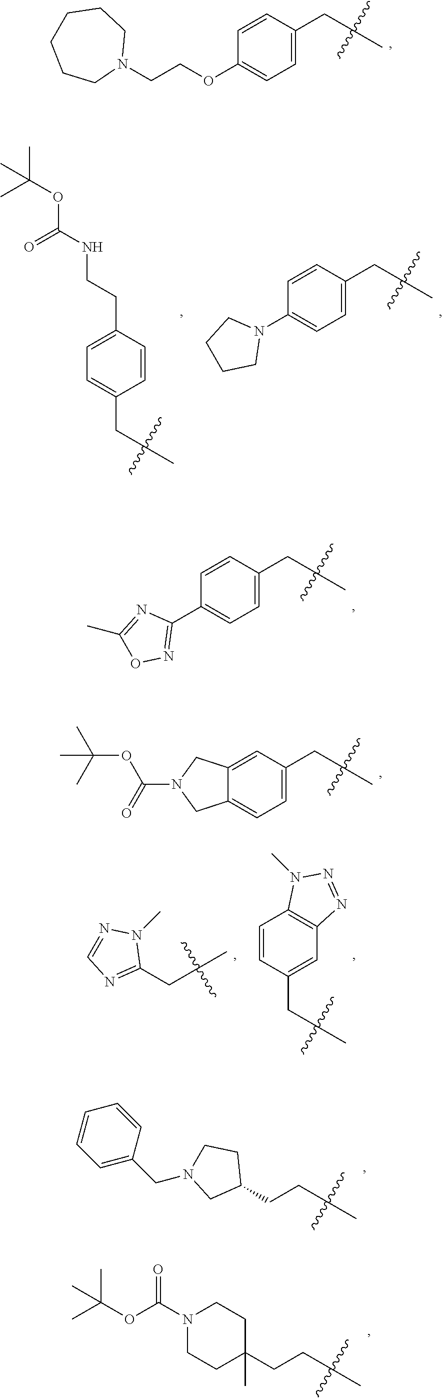

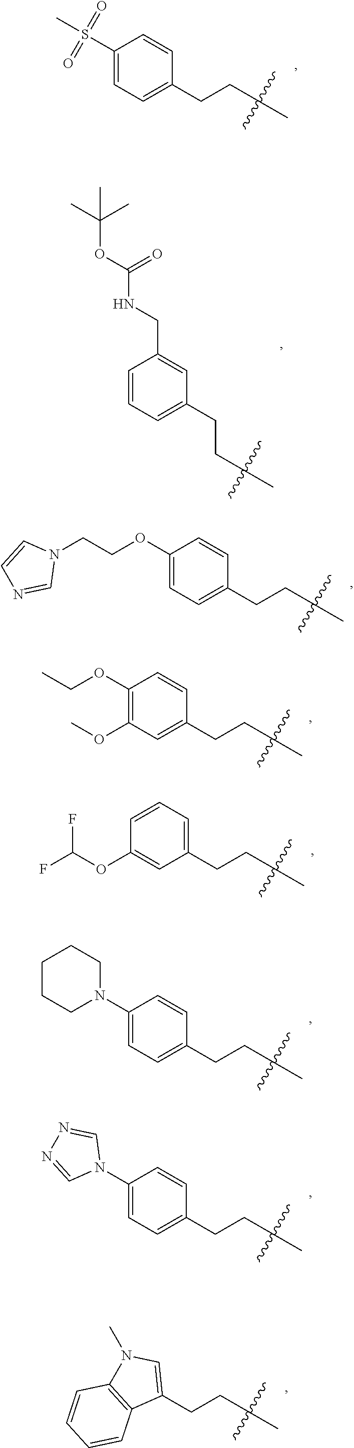

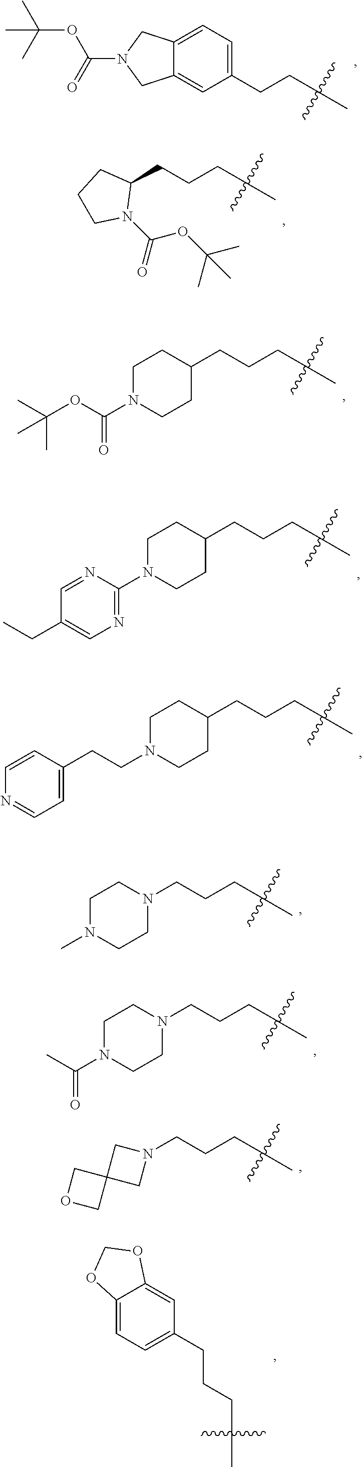

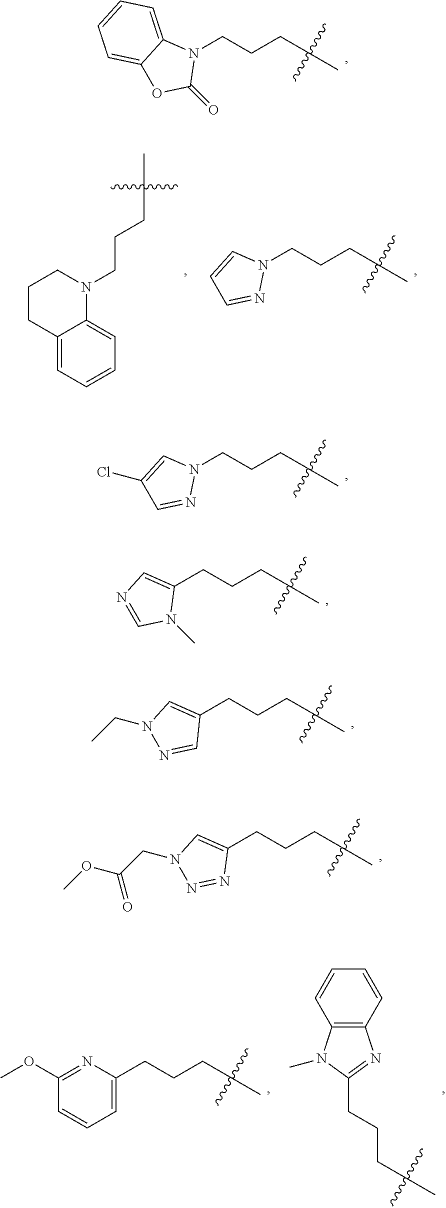

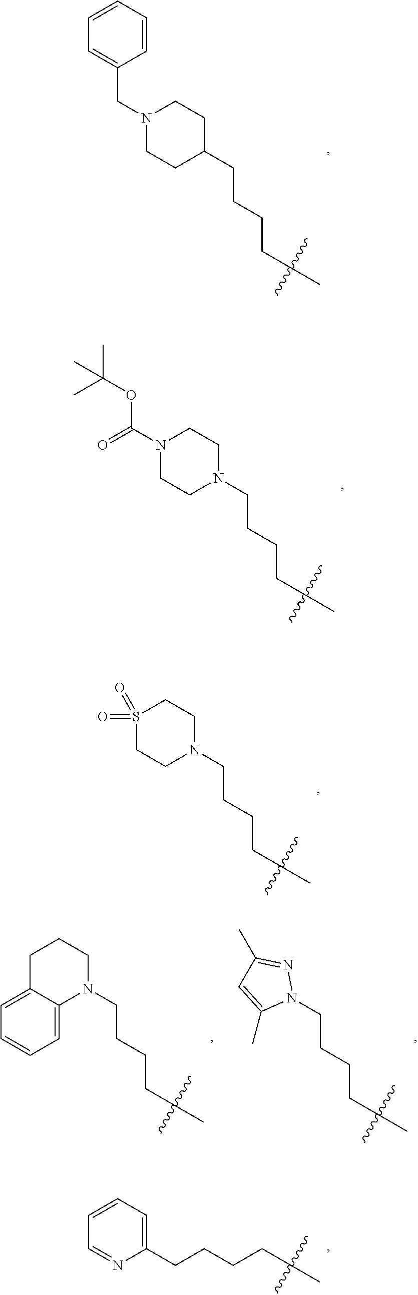 [SMILES: *CCCCC1CCN(Cc2ccccc2)CC1 ; *CCCCN1CCN(C(=O)OC(C)(C)C)CC1 ; *CCCCN1CCS(=O)(=O)CC1 ; *CCCCN1CCCc2ccccc21 ; *CCCCn1nc(C)cc1C ; *CCCCc1ccccn1]

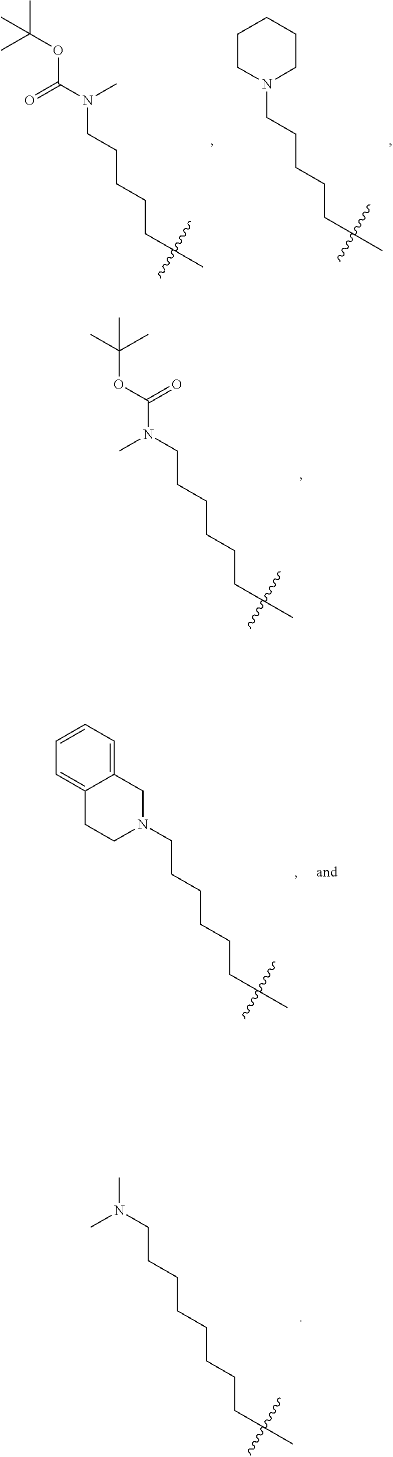 [SMILES: *CCCCCN(C)C(=O)OC(C)(C)C ; *CCCCCN1CCCCC1 ; *CCCCCCN(C)C(=O)OC(C)(C)C ; *CCCCCCN1CCc2ccccc2C1 ; *CCCCCCCCN(C)C]

5. The compound according to claim 2, or a pharmaceutically acceptable salt thereof wherein:
R¹ is chloro;
R² is selected from the group consisting of H and chloro;
R³ is selected from the group consisting H and F; and
R⁵ is selected from the group consisting of phenyl, —CHF₂, benzoyl, —CH₂CH₂OCH₃, —CH₂CH₂OCH₂CH₃, —CH₂CH₂S(O)₂CH₂CH₃,

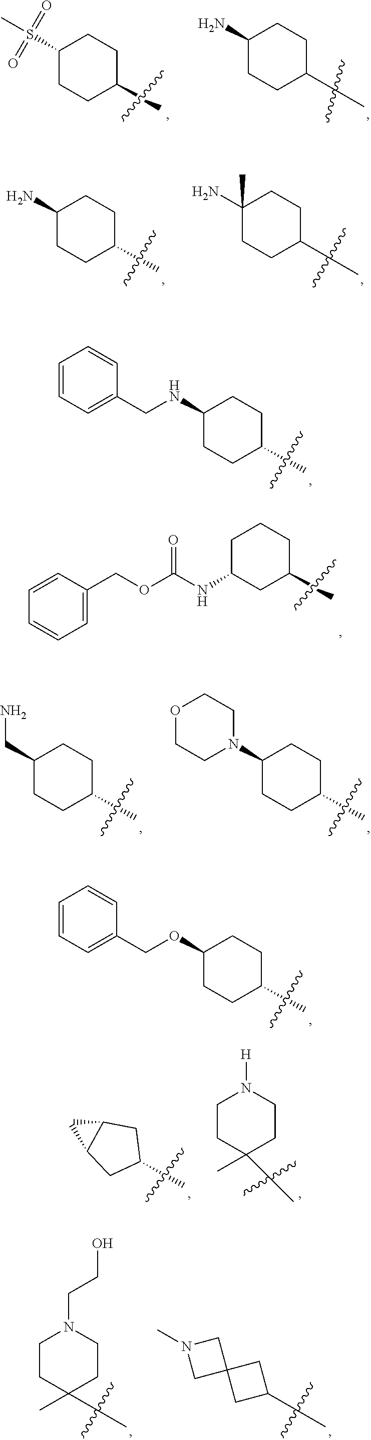

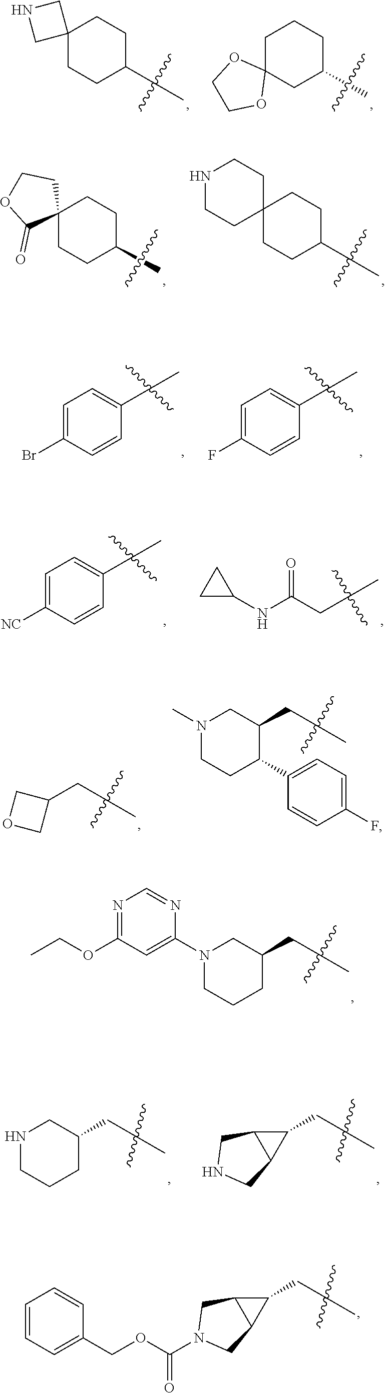

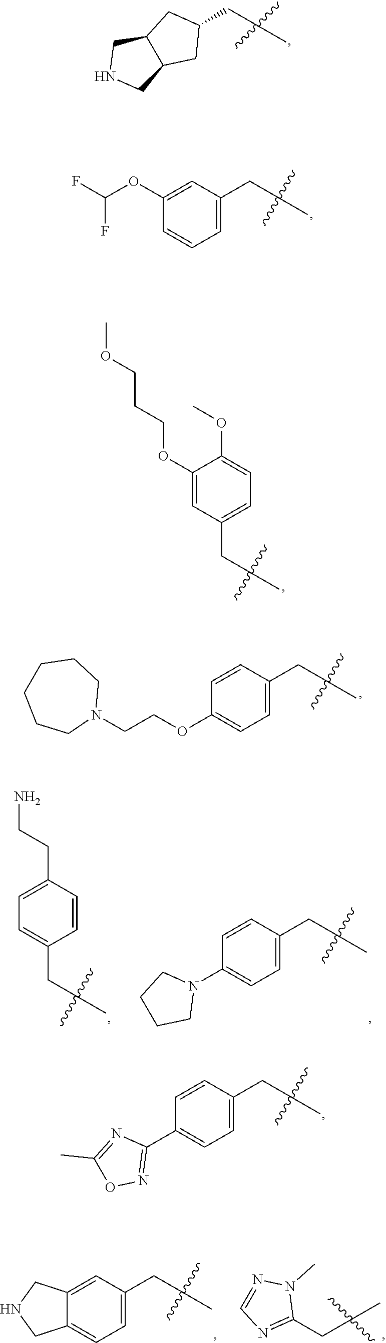

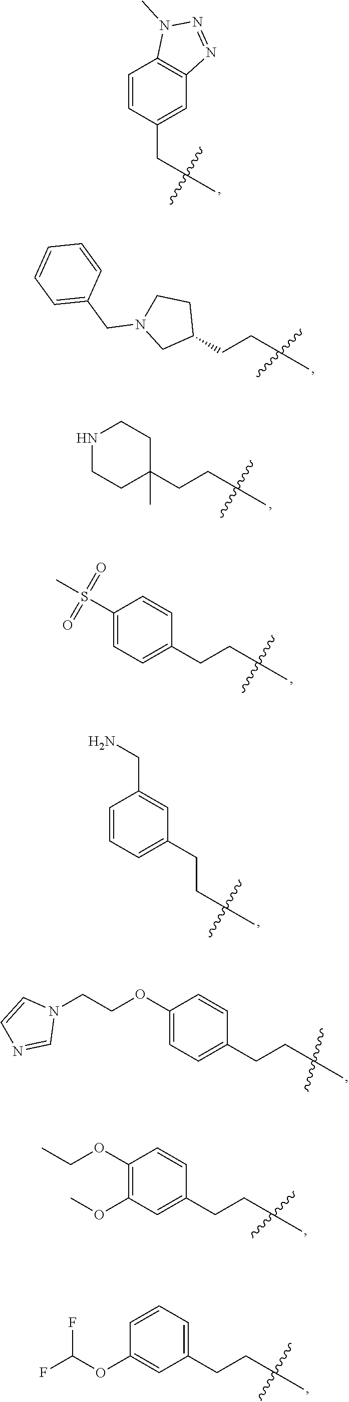

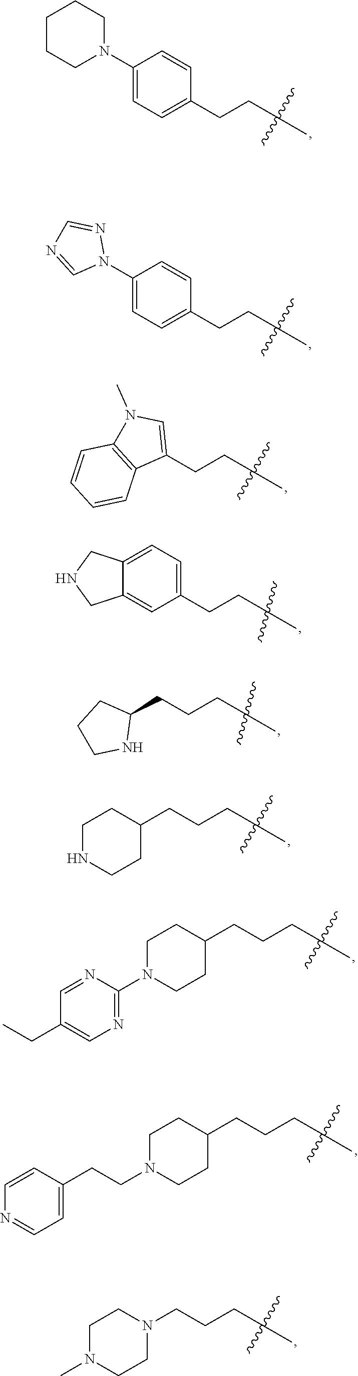

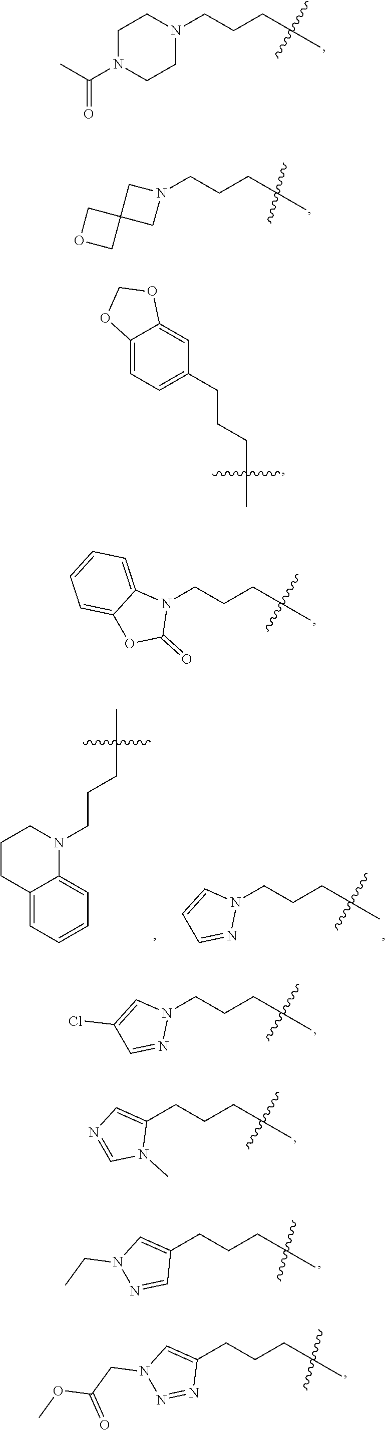

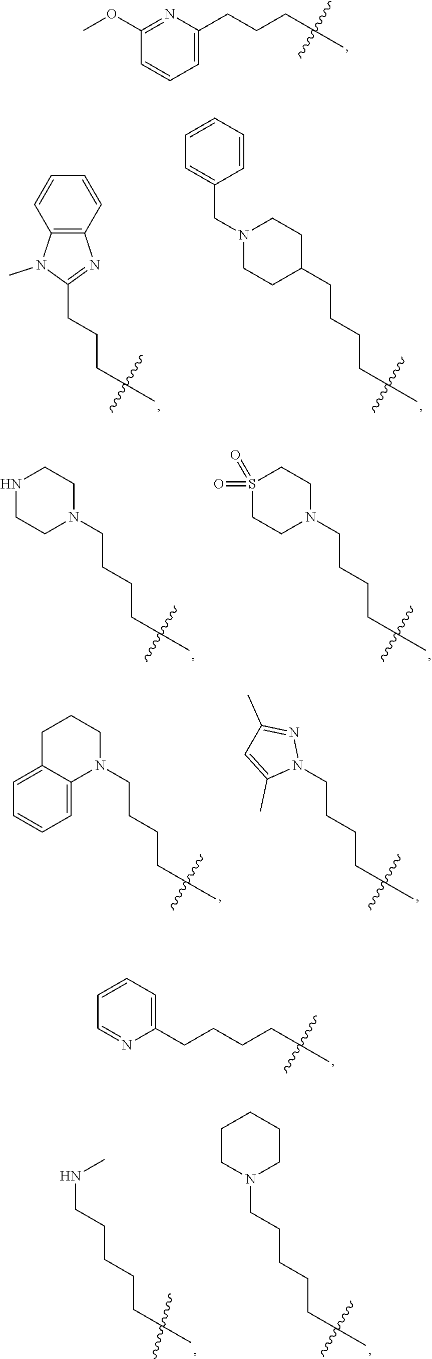

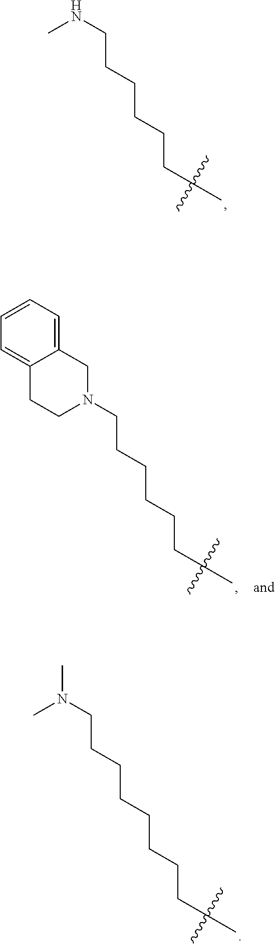

6. A compound according to claim 2 selected from the group consisting of 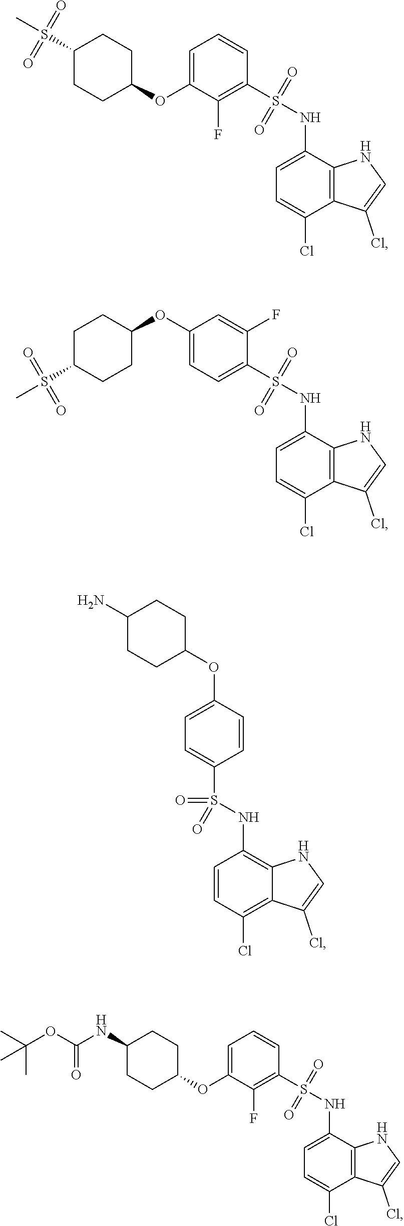 [SMILES: CS(=O)(=O)[C@H]1CC[C@H](Oc2cccc(S(=O)(=O)Nc3ccc(Cl)c4c(Cl)c[nH]c34)c2F)CC1 ; CS(=O)(=O)[C@H]1CC[C@H](Oc2ccc(S(=O)(=O)Nc3ccc(Cl)c4c(Cl)c[nH]c34)c(F)c2)CC1 ; NC1CCC(Oc2ccc(S(=O)(=O)Nc3ccc(Cl)c4c(Cl)c[nH]c34)cc2)CC1 ; CC(C)(C)OC(=O)N[C@H]1CC[C@H](Oc2cccc(S(=O)(=O)Nc3ccc(Cl)c4c(Cl)c[nH]c34)c2F)CC1] 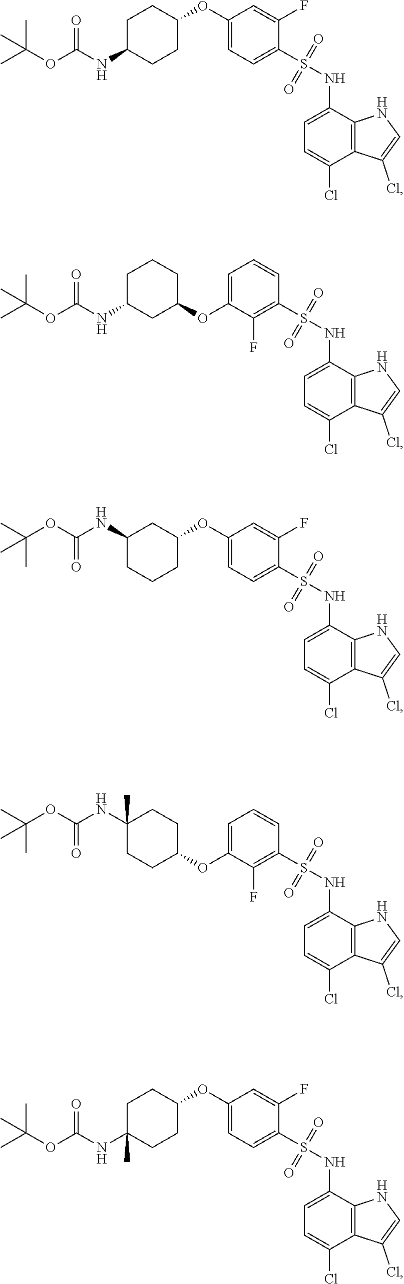 [SMILES: CC(C)(C)OC(=O)N[C@H]1CC[C@H](Oc2ccc(S(=O)(=O)Nc3ccc(Cl)c4c(Cl)c[nH]c34)c(F)c2)CC1 ; CC(C)(C)OC(=O)N[C@@H]1CCC[C@@H](Oc2cccc(S(=O)(=O)Nc3ccc(Cl)c4c(Cl)c[nH]c34)c2F)C1 ; CC(C)(C)OC(=O)N[C@@H]1CCC[C@@H](Oc2ccc(S(=O)(=O)Nc3ccc(Cl)c4c(Cl)c[nH]c34)c(F)c2)C1 ; CC(C)(C)OC(=O)N[C@]1(C)CC[C@H](Oc2cccc(S(=O)(=O)Nc3ccc(Cl)c4c(Cl)c[nH]c34)c2F)CC1 ; CC(C)(C)OC(=O)N[C@]1(C)CC[C@H](Oc2ccc(S(=O)(=O)Nc3ccc(Cl)c4c(Cl)c[nH]c34)c(F)c2)CC1] 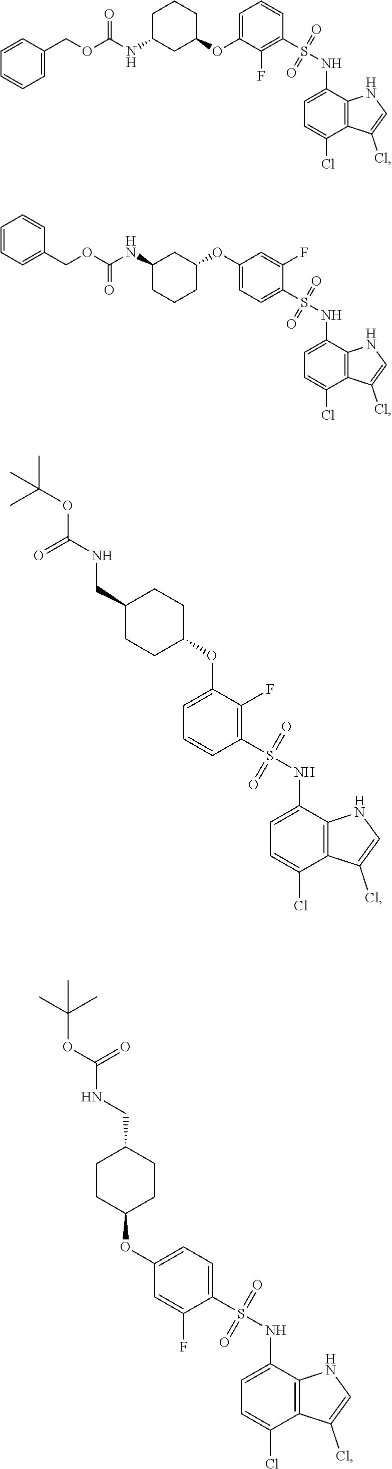 [SMILES: O=C(N[C@@H]1CCC[C@@H](Oc2cccc(S(=O)(=O)Nc3ccc(Cl)c4c(Cl)c[nH]c34)c2F)C1)OCc1ccccc1 ; O=C(N[C@@H]1CCC[C@@H](Oc2ccc(S(=O)(=O)Nc3ccc(Cl)c4c(Cl)c[nH]c34)c(F)c2)C1)OCc1ccccc1 ; CC(C)(C)OC(=O)NC[C@H]1CC[C@H](Oc2cccc(S(=O)(=O)Nc3ccc(Cl)c4c(Cl)c[nH]c34)c2F)CC1 ; CC(C)(C)OC(=O)NC[C@H]1CC[C@H](Oc2ccc(S(=O)(=O)Nc3ccc(Cl)c4c(Cl)c[nH]c34)c(F)c2)CC1] 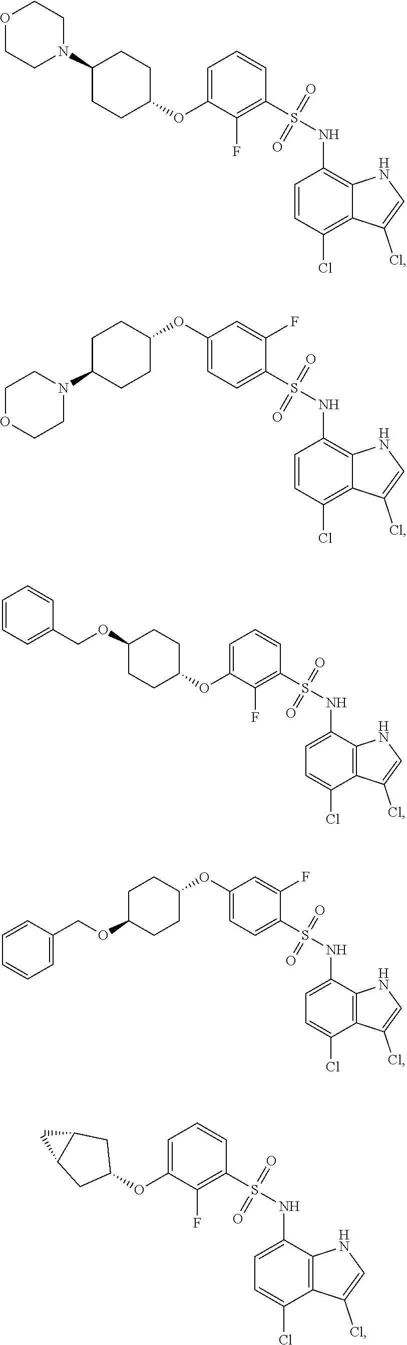 [SMILES: O=S(=O)(Nc1ccc(Cl)c2c(Cl)c[nH]c12)c1cccc(O[C@H]2CC[C@H](N3CCOCC3)CC2)c1F ; O=S(=O)(Nc1ccc(Cl)c2c(Cl)c[nH]c12)c1ccc(O[C@H]2CC[C@H](N3CCOCC3)CC2)cc1F ; O=S(=O)(Nc1ccc(Cl)c2c(Cl)c[nH]c12)c1cccc(O[C@H]2CC[C@H](OCc3ccccc3)CC2)c1F ; O=S(=O)(Nc1ccc(Cl)c2c(Cl)c[nH]c12)c1ccc(O[C@H]2CC[C@H](OCc3ccccc3)CC2)cc1F ; O=S(=O)(Nc1ccc(Cl)c2c(Cl)c[nH]c12)c1cccc(O[C@@H]2C[C@@H]3C[C@@H]3C2)c1F] 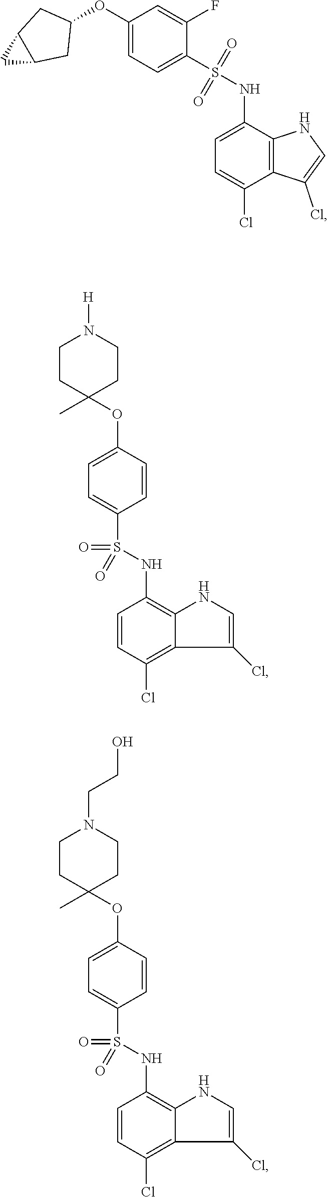 [SMILES: O=S(=O)(Nc1ccc(Cl)c2c(Cl)c[nH]c12)c1ccc(O[C@@H]2C[C@@H]3C[C@@H]3C2)cc1F ; CC1(Oc2ccc(S(=O)(=O)Nc3ccc(Cl)c4c(Cl)c[nH]c34)cc2)CCNCC1 ; CC1(Oc2ccc(S(=O)(=O)Nc3ccc(Cl)c4c(Cl)c[nH]c34)cc2)CCN(CCO)CC1] 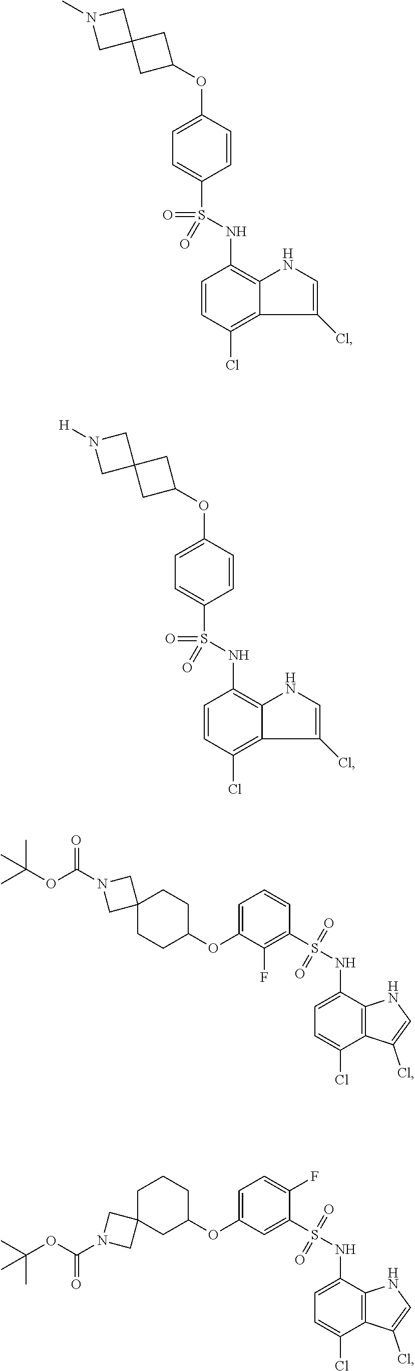 [SMILES: CN1CC2(CC(Oc3ccc(S(=O)(=O)Nc4ccc(Cl)c5c(Cl)c[nH]c45)cc3)C2)C1 ; O=S(=O)(Nc1ccc(Cl)c2c(Cl)c[nH]c12)c1ccc(OC2CC3(CNC3)C2)cc1 ; CC(C)(C)OC(=O)N1CC2(CCC(Oc3cccc(S(=O)(=O)Nc4ccc(Cl)c5c(Cl)c[nH]c45)c3F)CC2)C1 ; CC(C)(C)OC(=O)N1CC2(CCCC(Oc3ccc(F)c(S(=O)(=O)Nc4ccc(Cl)c5c(Cl)c[nH]c45)c3)C2)C1] 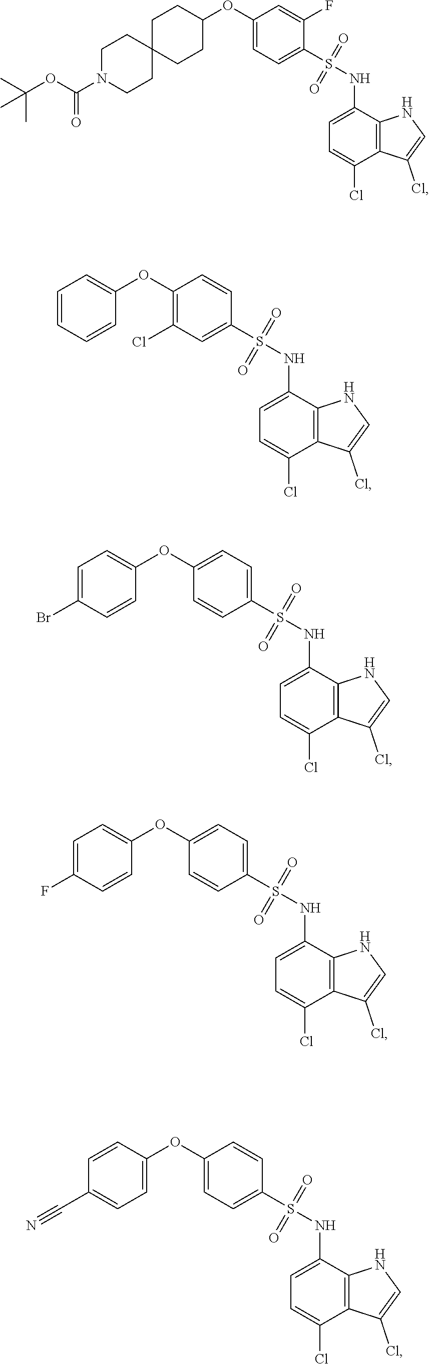 [SMILES: CC(C)(C)OC(=O)N1CCC2(CCC(Oc3ccc(S(=O)(=O)Nc4ccc(Cl)c5c(Cl)c[nH]c45)c(F)c3)CC2)CC1 ; O=S(=O)(Nc1ccc(Cl)c2c(Cl)c[nH]c12)c1ccc(Oc2ccccc2)c(Cl)c1 ; O=S(=O)(Nc1ccc(Cl)c2c(Cl)c[nH]c12)c1ccc(Oc2ccc(Br)cc2)cc1 ; O=S(=O)(Nc1ccc(Cl)c2c(Cl)c[nH]c12)c1ccc(Oc2ccc(F)cc2)cc1 ; N#Cc1ccc(Oc2ccc(S(=O)(=O)Nc3ccc(Cl)c4c(Cl)c[nH]c34)cc2)cc1] 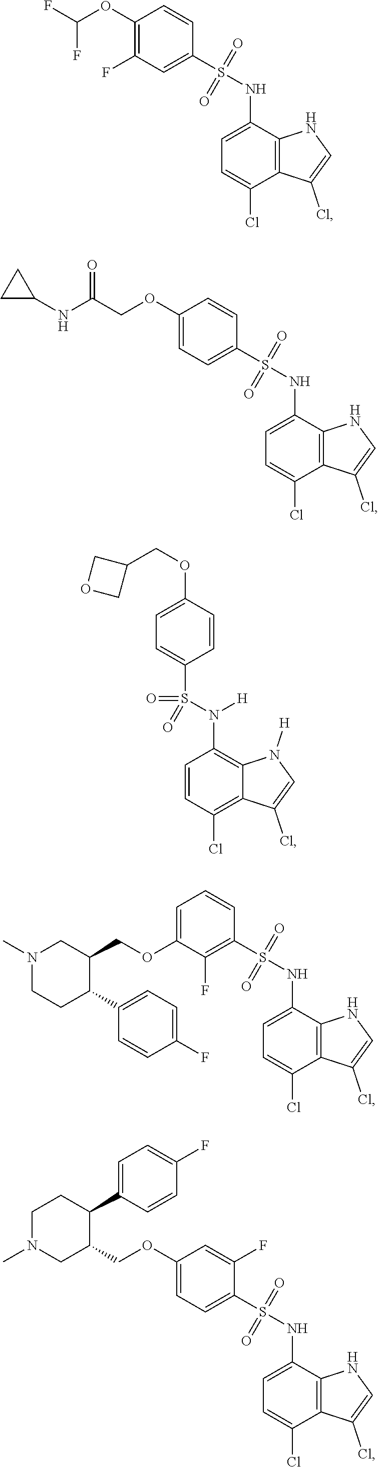 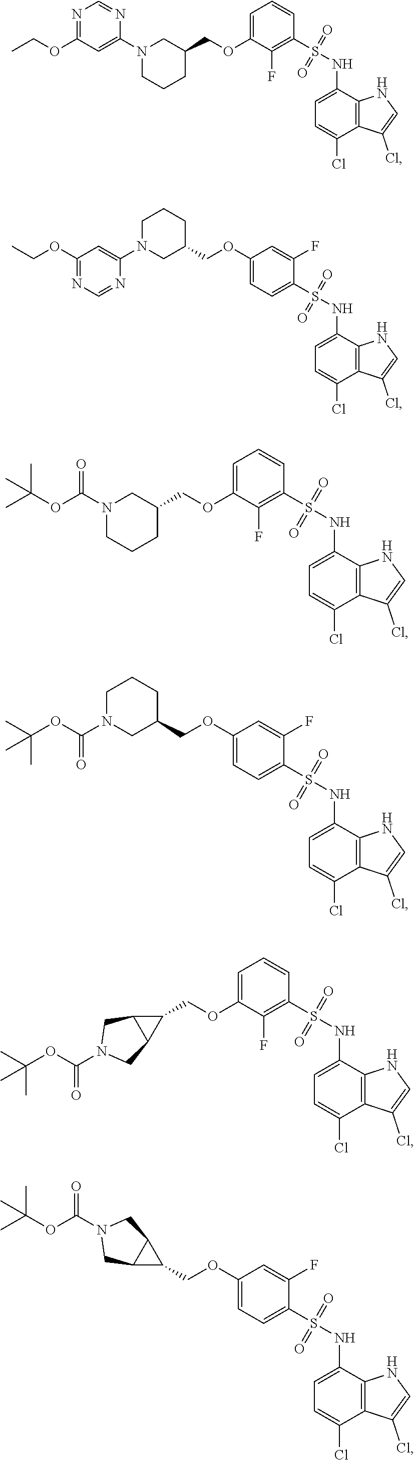 [SMILES: CCOc1cc(N2CCC[C@H](COc3cccc(S(=O)(=O)Nc4ccc(Cl)c5c(Cl)c[nH]c45)c3F)C2)ncn1 ; CCOc1cc(N2CCC[C@H](COc3ccc(S(=O)(=O)Nc4ccc(Cl)c5c(Cl)c[nH]c45)c(F)c3)C2)ncn1 ; CC(C)(C)OC(=O)N1CCC[C@@H](COc2cccc(S(=O)(=O)Nc3ccc(Cl)c4c(Cl)c[nH]c34)c2F)C1 ; CC(C)(C)OC(=O)N1CCC[C@@H](COc2ccc(S(=O)(=O)Nc3ccc(Cl)c4c(Cl)c[nH]c34)c(F)c2)C1 ; CC(C)(C)OC(=O)N1C[C@@H]2[C@@H](COc3cccc(S(=O)(=O)Nc4ccc(Cl)c5c(Cl)c[nH]c45)c3F)[C@@H]2C1 ; CC(C)(C)OC(=O)N1C[C@@H]2[C@@H](COc3ccc(S(=O)(=O)Nc4ccc(Cl)c5c(Cl)c[nH]c45)c(F)c3)[C@@H]2C1] 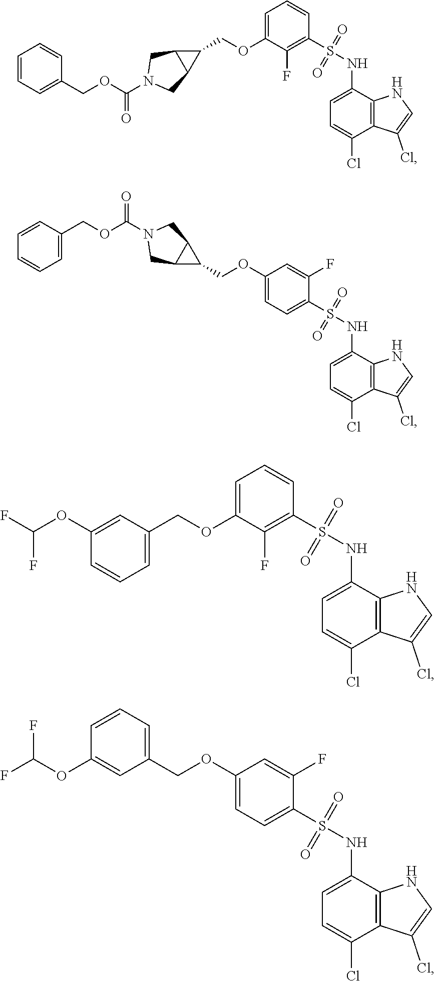 [SMILES: O=C(OCc1ccccc1)N1C[C@@H]2[C@@H](COc3cccc(S(=O)(=O)Nc4ccc(Cl)c5c(Cl)c[nH]c45)c3F)[C@@H]2C1 ; O=C(OCc1ccccc1)N1C[C@@H]2[C@@H](COc3ccc(S(=O)(=O)Nc4ccc(Cl)c5c(Cl)c[nH]c45)c(F)c3)[C@@H]2C1 ; O=S(=O)(Nc1ccc(Cl)c2c(Cl)c[nH]c12)c1cccc(OCc2cccc(OC(F)F)c2)c1F ; O=S(=O)(Nc1ccc(Cl)c2c(Cl)c[nH]c12)c1ccc(OCc2cccc(OC(F)F)c2)cc1F] 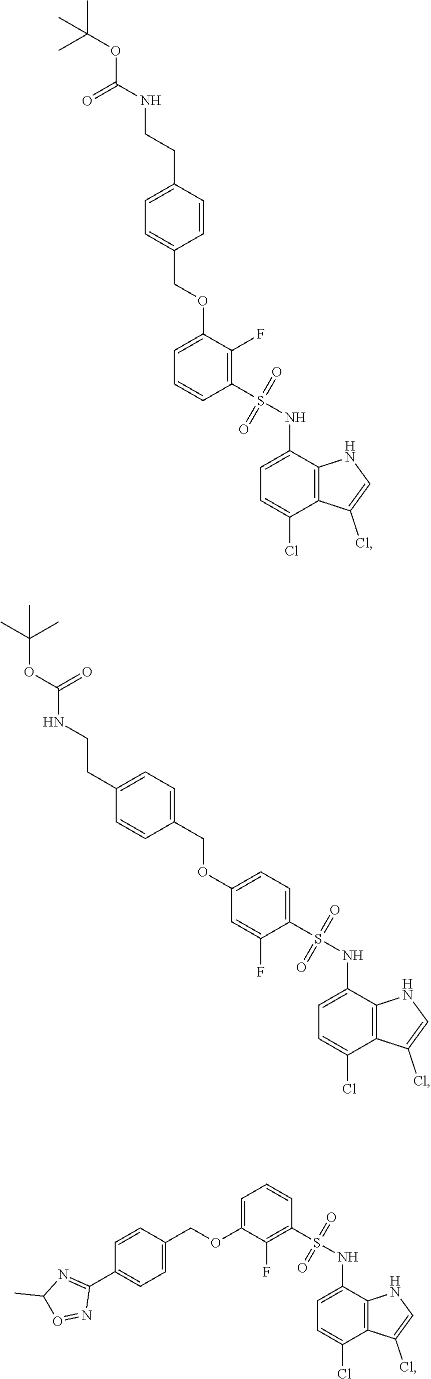 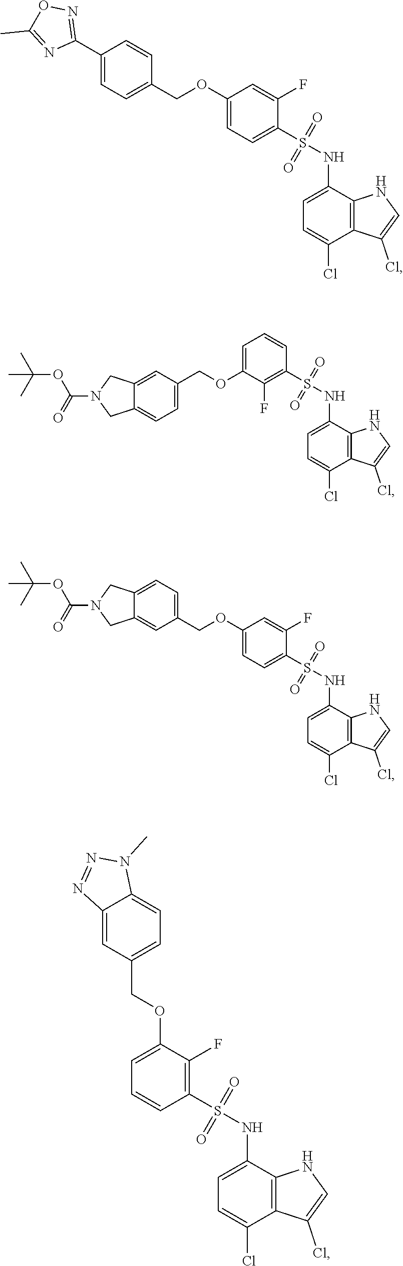 [SMILES: Cc1nc(-c2ccc(COc3ccc(S(=O)(=O)Nc4ccc(Cl)c5c(Cl)c[nH]c45)c(F)c3)cc2)no1 ; CC(C)(C)OC(=O)N1Cc2ccc(COc3cccc(S(=O)(=O)Nc4ccc(Cl)c5c(Cl)c[nH]c45)c3F)cc2C1 ; CC(C)(C)OC(=O)N1Cc2ccc(COc3ccc(S(=O)(=O)Nc4ccc(Cl)c5c(Cl)c[nH]c45)c(F)c3)cc2C1 ; Cn1nnc2cc(COc3cccc(S(=O)(=O)Nc4ccc(Cl)c5c(Cl)c[nH]c45)c3F)ccc21] 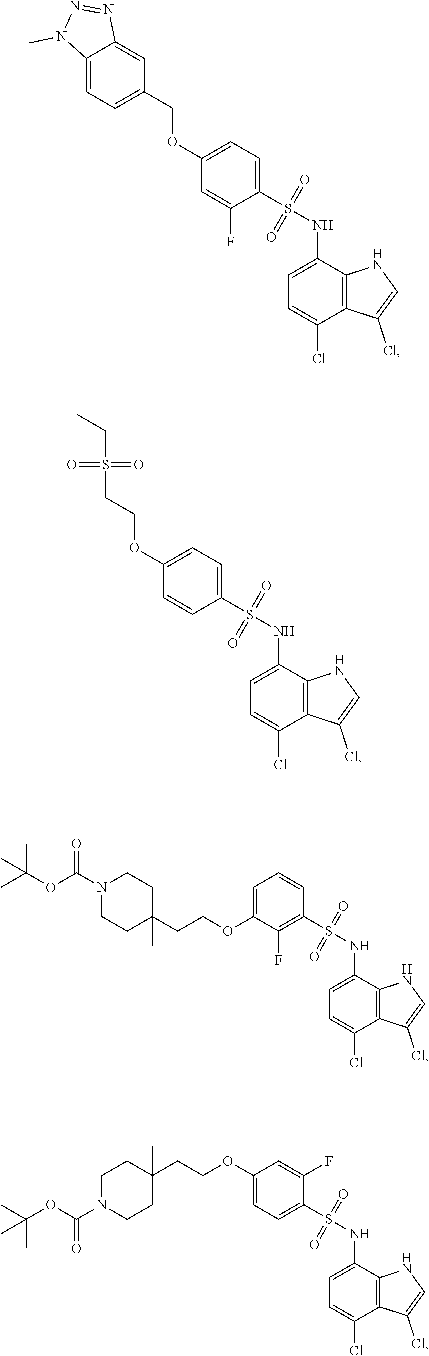 [SMILES: Cn1nnc2cc(COc3ccc(S(=O)(=O)Nc4ccc(Cl)c5c(Cl)c[nH]c45)c(F)c3)ccc21 ; CCS(=O)(=O)CCOc1ccc(S(=O)(=O)Nc2ccc(Cl)c3c(Cl)c[nH]c23)cc1 ; CC1(CCOc2cccc(S(=O)(=O)Nc3ccc(Cl)c4c(Cl)c[nH]c34)c2F)CCN(C(=O)OC(C)(C)C)CC1 ; CC1(CCOc2ccc(S(=O)(=O)Nc3ccc(Cl)c4c(Cl)c[nH]c34)c(F)c2)CCN(C(=O)OC(C)(C)C)CC1] 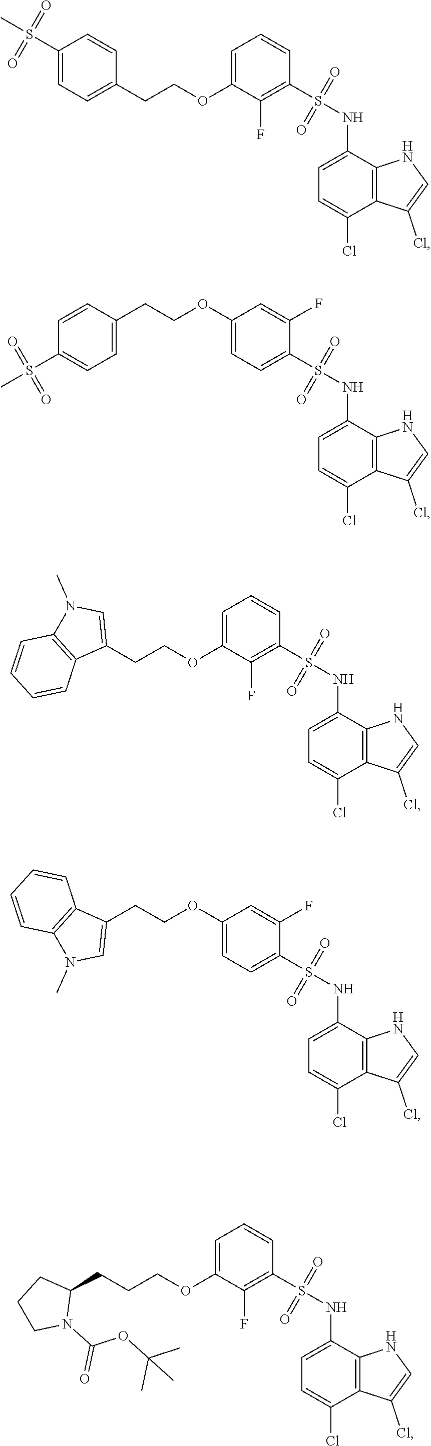 [SMILES: CS(=O)(=O)c1ccc(CCOc2cccc(S(=O)(=O)Nc3ccc(Cl)c4c(Cl)c[nH]c34)c2F)cc1 ; CS(=O)(=O)c1ccc(CCOc2ccc(S(=O)(=O)Nc3ccc(Cl)c4c(Cl)c[nH]c34)c(F)c2)cc1 ; Cn1cc(CCOc2cccc(S(=O)(=O)Nc3ccc(Cl)c4c(Cl)c[nH]c34)c2F)c2ccccc21 ; Cn1cc(CCOc2ccc(S(=O)(=O)Nc3ccc(Cl)c4c(Cl)c[nH]c34)c(F)c2)c2ccccc21 ; CC(C)(C)OC(=O)N1CCC[C@H]1CCCOc1cccc(S(=O)(=O)Nc2ccc(Cl)c3c(Cl)c[nH]c23)c1F] 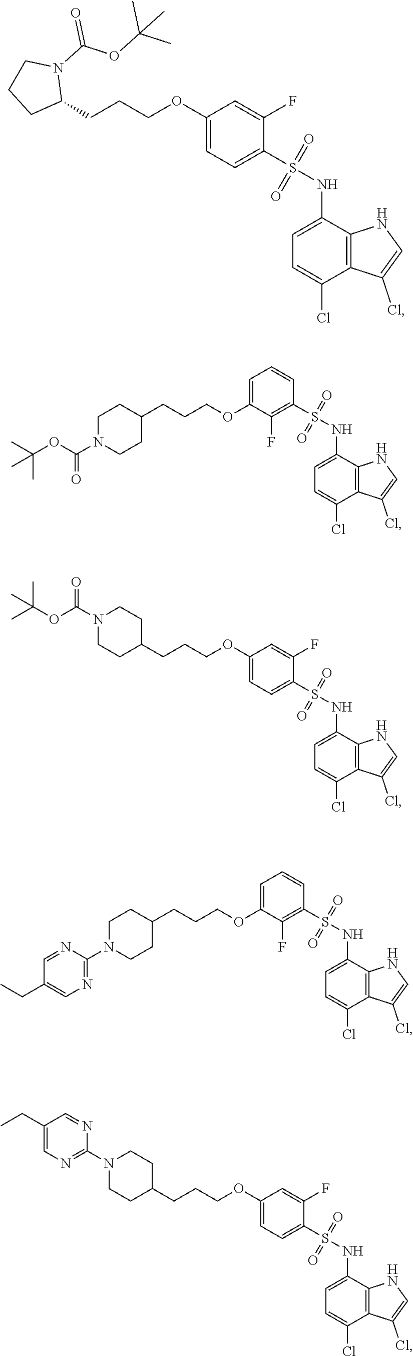 [SMILES: CC(C)(C)OC(=O)N1CCC[C@H]1CCCOc1ccc(S(=O)(=O)Nc2ccc(Cl)c3c(Cl)c[nH]c23)c(F)c1 ; CC(C)(C)OC(=O)N1CCC(CCCOc2cccc(S(=O)(=O)Nc3ccc(Cl)c4c(Cl)c[nH]c34)c2F)CC1 ; CC(C)(C)OC(=O)N1CCC(CCCOc2ccc(S(=O)(=O)Nc3ccc(Cl)c4c(Cl)c[nH]c34)c(F)c2)CC1 ; CCc1cnc(N2CCC(CCCOc3cccc(S(=O)(=O)Nc4ccc(Cl)c5c(Cl)c[nH]c45)c3F)CC2)nc1 ; CCc1cnc(N2CCC(CCCOc3ccc(S(=O)(=O)Nc4ccc(Cl)c5c(Cl)c[nH]c45)c(F)c3)CC2)nc1] 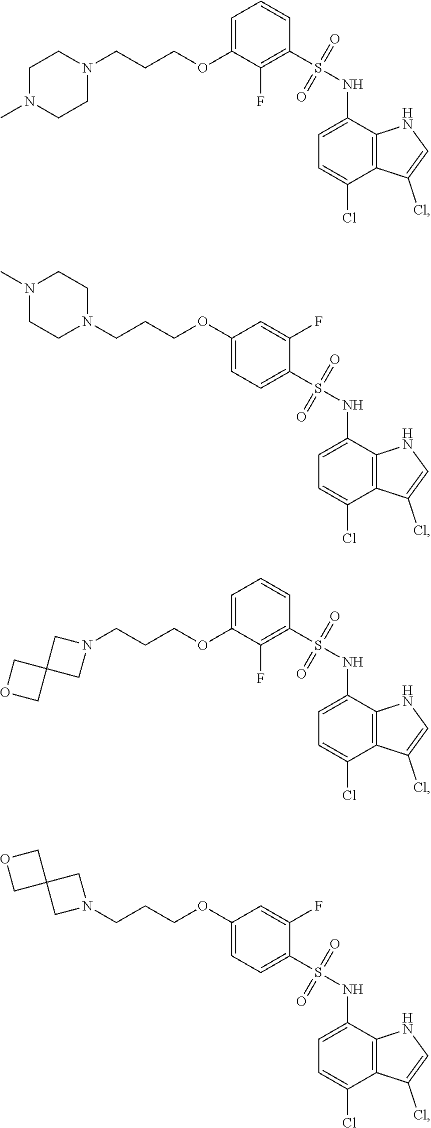 [SMILES: CN1CCN(CCCOc2cccc(S(=O)(=O)Nc3ccc(Cl)c4c(Cl)c[nH]c34)c2F)CC1 ; CN1CCN(CCCOc2ccc(S(=O)(=O)Nc3ccc(Cl)c4c(Cl)c[nH]c34)c(F)c2)CC1 ; O=S(=O)(Nc1ccc(Cl)c2c(Cl)c[nH]c12)c1cccc(OCCCN2CC3(COC3)C2)c1F ; O=S(=O)(Nc1ccc(Cl)c2c(Cl)c[nH]c12)c1ccc(OCCCN2CC3(COC3)C2)cc1F] 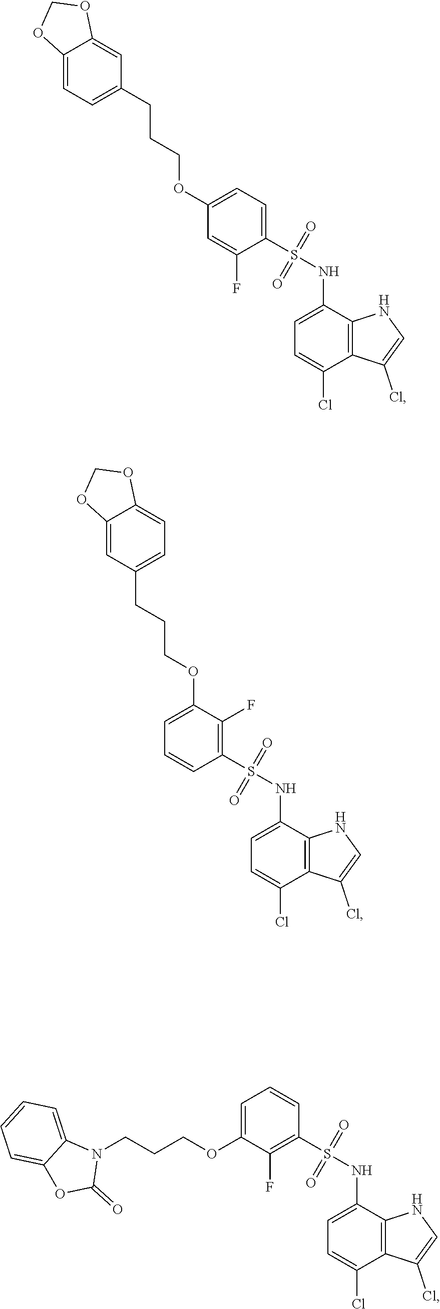 [SMILES: O=S(=O)(Nc1ccc(Cl)c2c(Cl)c[nH]c12)c1ccc(OCCCc2ccc3c(c2)OCO3)cc1F ; O=S(=O)(Nc1ccc(Cl)c2c(Cl)c[nH]c12)c1cccc(OCCCc2ccc3c(c2)OCO3)c1F ; O=c1oc2ccccc2n1CCCOc1cccc(S(=O)(=O)Nc2ccc(Cl)c3c(Cl)c[nH]c23)c1F] 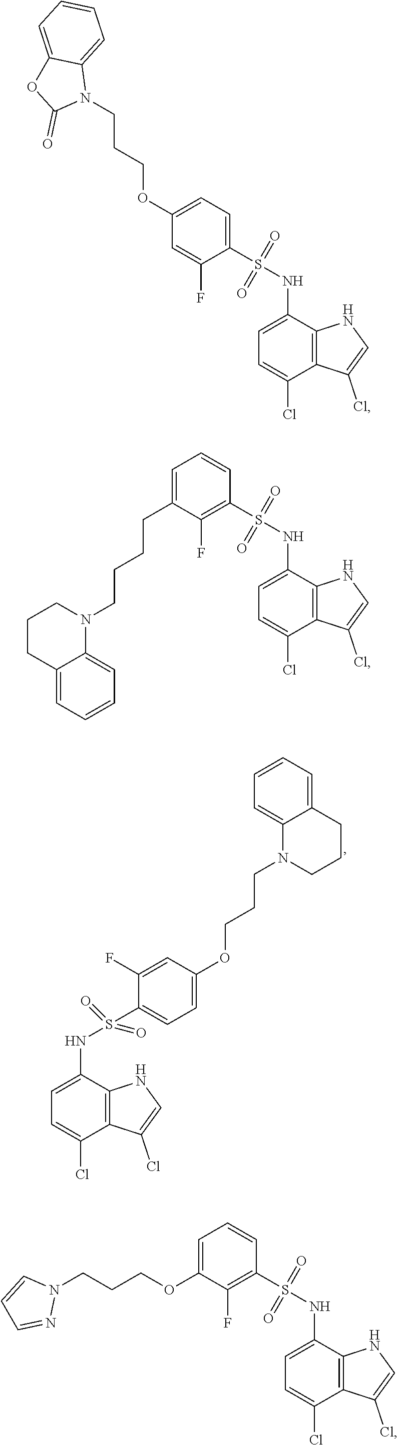 [SMILES: O=c1oc2ccccc2n1CCCOc1ccc(S(=O)(=O)Nc2ccc(Cl)c3c(Cl)c[nH]c23)c(F)c1 ; O=S(=O)(Nc1ccc(Cl)c2c(Cl)c[nH]c12)c1cccc(CCCCN2CCCc3ccccc32)c1F ; O=S(=O)(Nc1ccc(Cl)c2c(Cl)c[nH]c12)c1ccc(OCCCN2CCCc3ccccc32)cc1F ; O=S(=O)(Nc1ccc(Cl)c2c(Cl)c[nH]c12)c1cccc(OCCCn2cccn2)c1F] 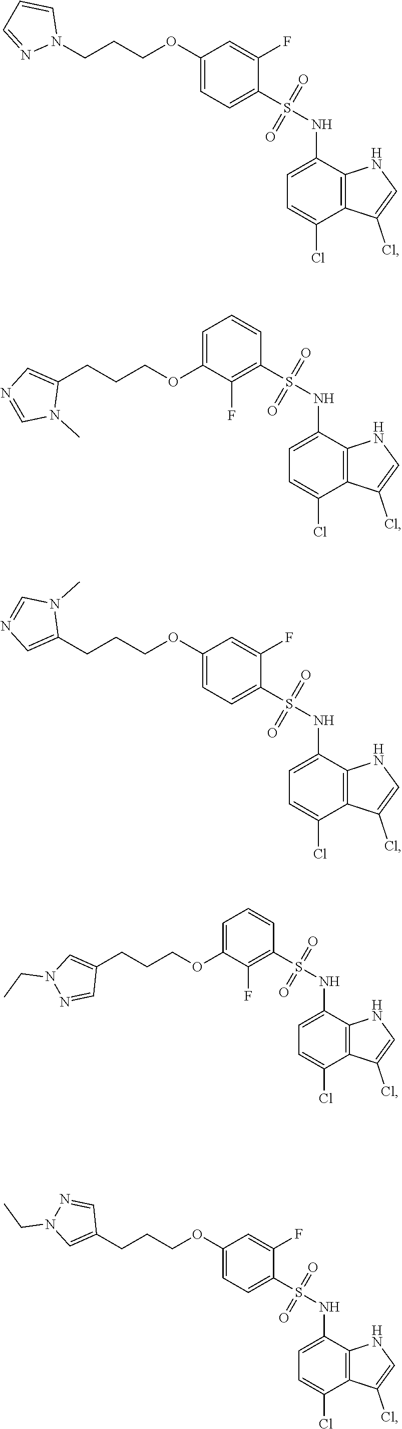 [SMILES: O=S(=O)(Nc1ccc(Cl)c2c(Cl)c[nH]c12)c1ccc(OCCCn2cccn2)cc1F ; Cn1cncc1CCCOc1cccc(S(=O)(=O)Nc2ccc(Cl)c3c(Cl)c[nH]c23)c1F ; Cn1cncc1CCCOc1ccc(S(=O)(=O)Nc2ccc(Cl)c3c(Cl)c[nH]c23)c(F)c1 ; CCn1cc(CCCOc2cccc(S(=O)(=O)Nc3ccc(Cl)c4c(Cl)c[nH]c34)c2F)cn1 ; CCn1cc(CCCOc2ccc(S(=O)(=O)Nc3ccc(Cl)c4c(Cl)c[nH]c34)c(F)c2)cn1] 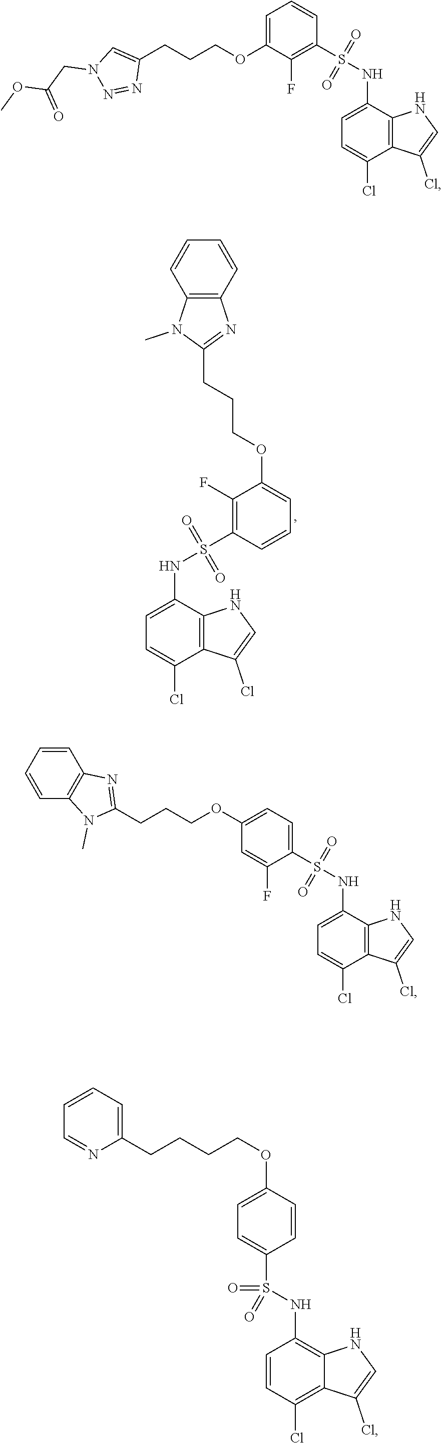 [SMILES: COC(=O)Cn1cc(CCCOc2cccc(S(=O)(=O)Nc3ccc(Cl)c4c(Cl)c[nH]c34)c2F)nn1 ; Cn1c(CCCOc2cccc(S(=O)(=O)Nc3ccc(Cl)c4c(Cl)c[nH]c34)c2F)nc2ccccc21 ; Cn1c(CCCOc2ccc(S(=O)(=O)Nc3ccc(Cl)c4c(Cl)c[nH]c34)c(F)c2)nc2ccccc21 ; O=S(=O)(Nc1ccc(Cl)c2c(Cl)c[nH]c12)c1ccc(OCCCCc2ccccn2)cc1] 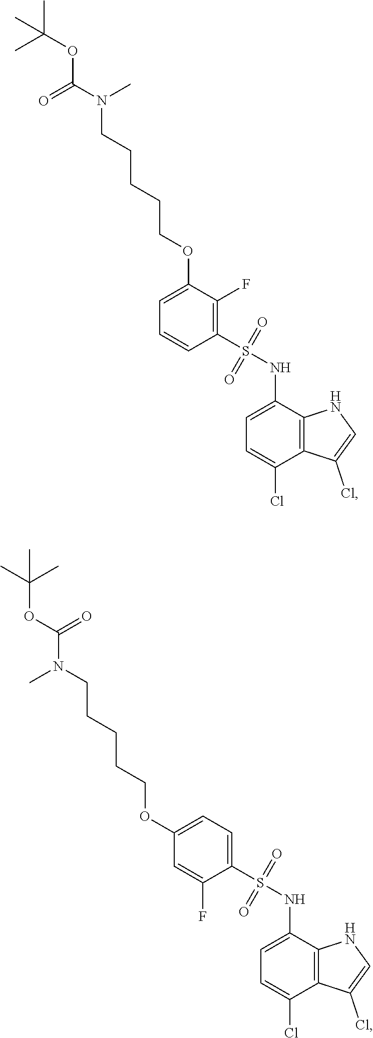 [SMILES: CN(CCCCCOc1cccc(S(=O)(=O)Nc2ccc(Cl)c3c(Cl)c[nH]c23)c1F)C(=O)OC(C)(C)C ; CN(CCCCCOc1ccc(S(=O)(=O)Nc2ccc(Cl)c3c(Cl)c[nH]c23)c(F)c1)C(=O)OC(C)(C)C] 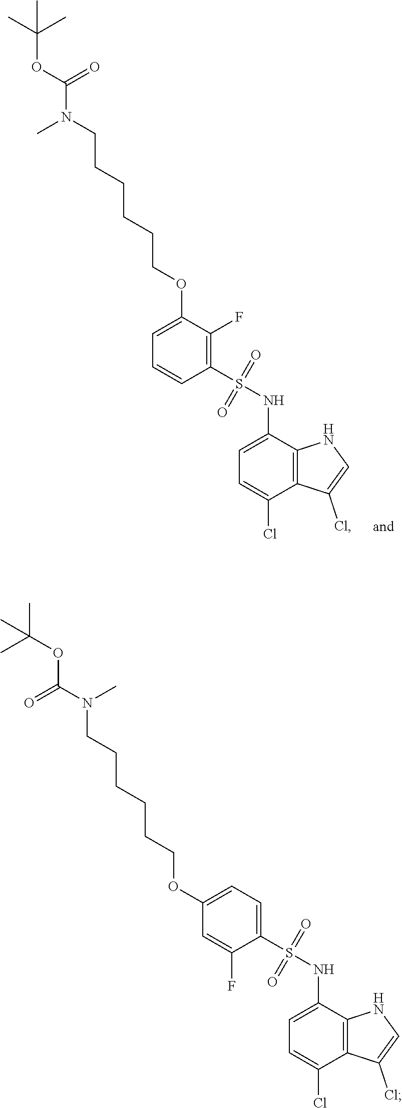 [SMILES: CN(CCCCCCOc1cccc(S(=O)(=O)Nc2ccc(Cl)c3c(Cl)c[nH]c23)c1F)C(=O)OC(C)(C)C ; CN(CCCCCCOc1ccc(S(=O)(=O)Nc2ccc(Cl)c3c(Cl)c[nH]c23)c(F)c1)C(=O)OC(C)(C)C]
or a pharmaceutically acceptable salt thereof.

7. A compound of Formula III 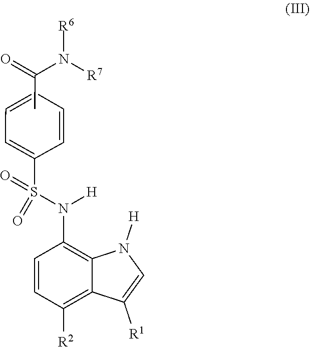
or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl;
R⁶ is selected from the group consisting of H and CH₃;
R⁷ is selected from the group consisting of optionally substituted C₃-C₇ cycloalkyl, optionally substituted 2,3-dihydro-1H-indenyl, optionally substituted 3 to 7-membered heterocyclyl, and substituted C₁-C₈ alkyl;
wherein the C₁-C₈ alkyl is substituted with at least one substituent selected from the group consisting of optionally substituted C₁-C₆ alkoxy, optionally substituted 4 to 8-membered heterocyclyoxy, —C(O)NR⁸R⁹, C₁-C₆ alkylsulfonyl, optionally substituted C₃-C₇ cycloalkyl, optionally substituted bicyclooctatrienyl, optionally substituted 4 to 8-membered heterocyclyl, optionally substituted C₆-C₁₀ aryl, optionally substituted dihydrobenzodioxinyl, and optionally substituted 5 to 10 membered heteroaryl;
wherein R⁸ and R⁹ together with the nitrogen atom to which they are attached form an optionally substituted 5-6 membered heterocyclyl; or alternatively,
R⁶ and R⁷ together with the nitrogen atom to which they are attached form a group selected from optionally substituted 4 to 12-membered optionally substituted heterocyclyl, and optionally substituted 4,5,6,7-tetrahydro-1H-imidazo[4,5-c]pyridinyl; with the proviso that the compound is not

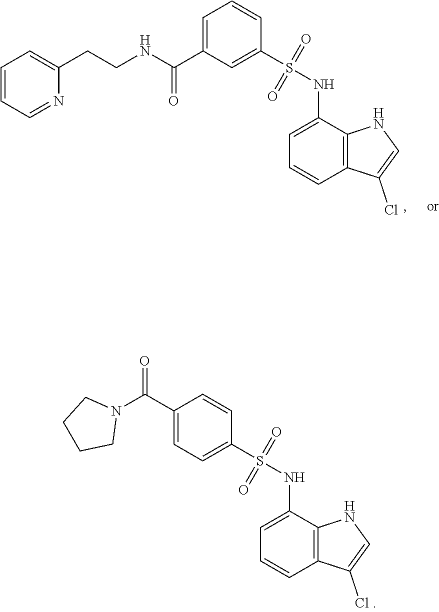 [SMILES: O=C(NCCc1ccccn1)c1cccc(S(=O)(=O)Nc2cccc3c(Cl)c[nH]c23)c1 ; O=C(c1ccc(S(=O)(=O)Nc2cccc3c(Cl)c[nH]c23)cc1)N1CCCC1]

8. The compound according to claim 7, or a pharmaceutically acceptable salt thereof, wherein the substituted C₁-C₈ alkyl is selected from the group consisting of:
C₁ alkyl substituted with at least one substituent selected from the group consisting of optionally substituted cyclopropyl, optionally substituted bicyclooctatrienyl, optionally substituted piperidinyl, optionally substituted phenyl, optionally substituted pyrazolyl, optionally substituted oxadiazolyl, optionally substituted pyridinyl, optionally substituted pyrazinyl and optionally substituted pyridinyl;
C₂ alkyl substituted with at least one substituent selected from the group consisting of optionally substituted cyclopropylmethoxy, optionally substituted (tetrahydro-2H-thiopyran 1,1-dioxide)oxy, ethylsulfonyl, optionally substituted azetidinyl, optionally substituted phenyl, optionally substituted furanyl, and optionally substituted pyridinyl;
C₃ alkyl substituted with at least one substituent selected from the group consisting of optionally substituted C₁-C₂ alkoxy; —C(O)NR⁸R⁹, wherein R⁸ and R⁹ together with the nitrogen atom to which they are attached form an optionally substituted pyrrolidinyl; optionally substituted phenyl, and optionally substituted dihydrobenzodioxinyl; and
C₅ alkyl substituted with optionally substituted 4,5,6,7-tetrahydro-1H-imidazo[4,5-c]pyridinyl.

9. The compound according to claim 7, or a pharmaceutically acceptable salt thereof, wherein R⁶ and R⁷ together with the nitrogen atom to which they are attached form a group selected from optionally substituted azetidinyl, optionally substituted pyrrolidinyl, optionally substituted morpholinyl, optionally substituted piperidinyl, optionally substituted piperazinyl, optionally substituted 1-oxa-4,9-diazaspiro[5.5]undecane, and optionally substituted 4,5,6,7-tetrahydro-1H-imidazo[4,5-c]pyridine.

10. The compound of claim 7 wherein
R¹ is chloro;
R² is selected from the group consisting of H and chloro;
R³ is selected from the group consisting H, F and cyano; and
R⁶ is selected from the group consisting of H and CH₃;
R⁷ is selected from the group consisting of cyclopropyl, —CH₂CH₂SO₂CH₂CH₃, —CH₂CH₂CH₂OCH₂CH₂OCH₃, 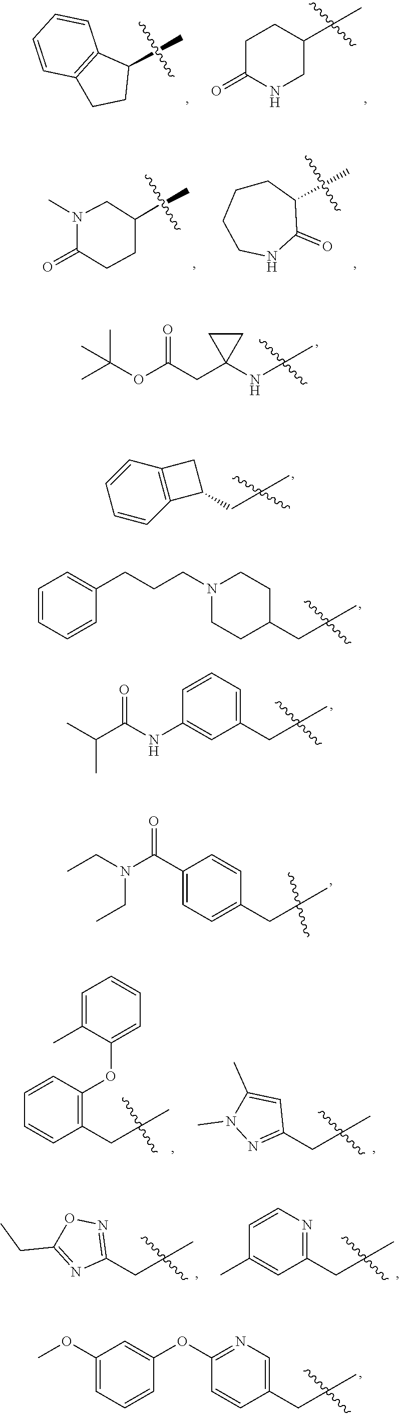 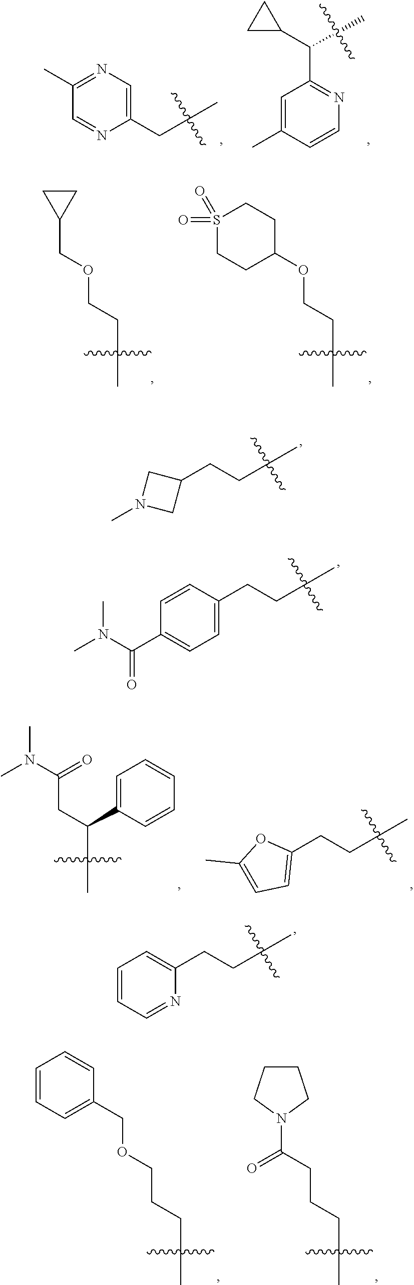 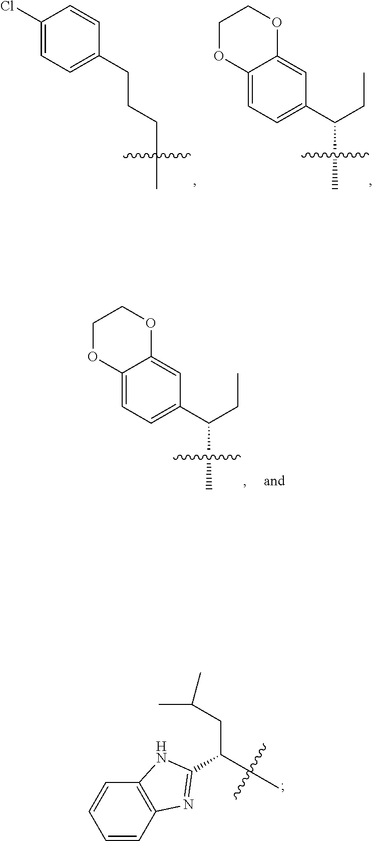
or alternatively
R⁶ and R⁷ together with the nitrogen atom to which they are attached form a group selected from

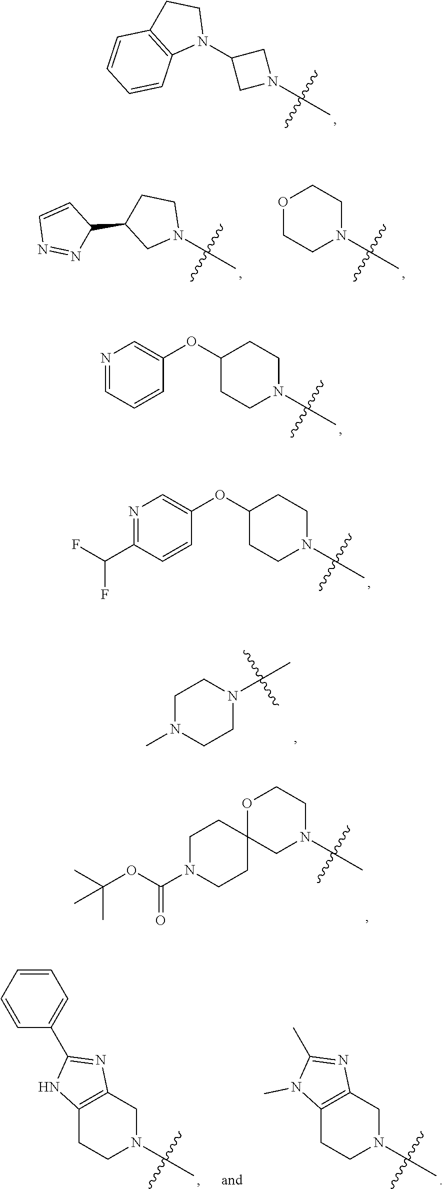

11. A compound according to claim 7 selected from the group consisting of 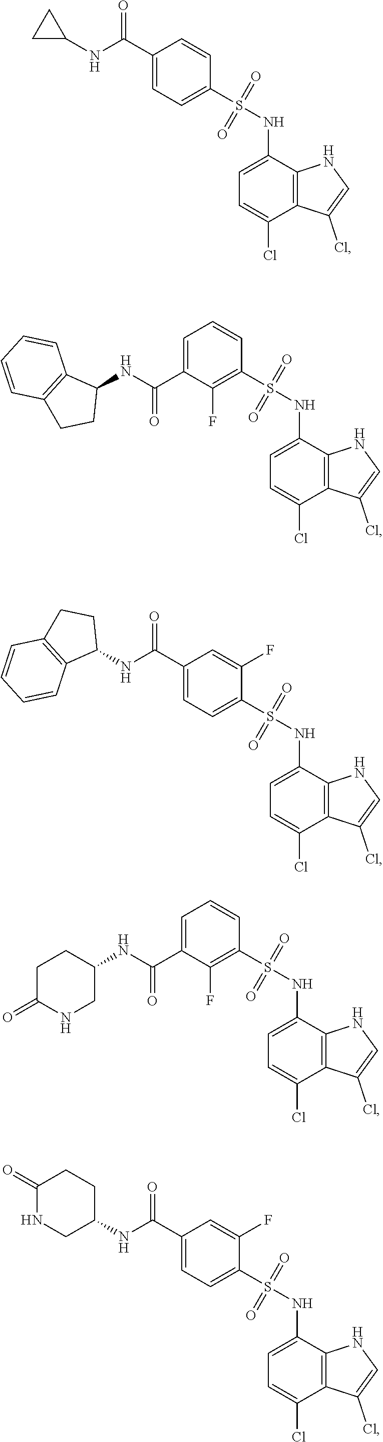 [SMILES: O=C(NC1CC1)c1ccc(S(=O)(=O)Nc2ccc(Cl)c3c(Cl)c[nH]c23)cc1 ; O=C(N[C@H]1CCc2ccccc21)c1cccc(S(=O)(=O)Nc2ccc(Cl)c3c(Cl)c[nH]c23)c1F ; O=C(N[C@H]1CCc2ccccc21)c1ccc(S(=O)(=O)Nc2ccc(Cl)c3c(Cl)c[nH]c23)c(F)c1 ; O=C1CC[C@H](NC(=O)c2cccc(S(=O)(=O)Nc3ccc(Cl)c4c(Cl)c[nH]c34)c2F)CN1 ; O=C1CC[C@H](NC(=O)c2ccc(S(=O)(=O)Nc3ccc(Cl)c4c(Cl)c[nH]c34)c(F)c2)CN1] 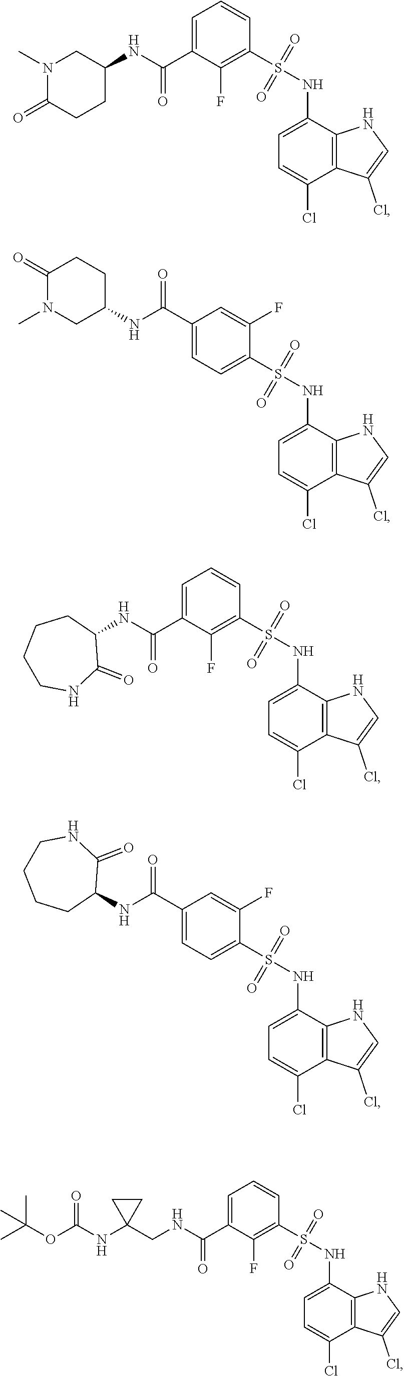 [SMILES: CN1C[C@@H](NC(=O)c2cccc(S(=O)(=O)Nc3ccc(Cl)c4c(Cl)c[nH]c34)c2F)CCC1=O ; CN1C[C@@H](NC(=O)c2ccc(S(=O)(=O)Nc3ccc(Cl)c4c(Cl)c[nH]c34)c(F)c2)CCC1=O ; O=C(N[C@H]1CCCCNC1=O)c1cccc(S(=O)(=O)Nc2ccc(Cl)c3c(Cl)c[nH]c23)c1F ; O=C(N[C@H]1CCCCNC1=O)c1ccc(S(=O)(=O)Nc2ccc(Cl)c3c(Cl)c[nH]c23)c(F)c1 ; CC(C)(C)OC(=O)NC1(CNC(=O)c2cccc(S(=O)(=O)Nc3ccc(Cl)c4c(Cl)c[nH]c34)c2F)CC1] 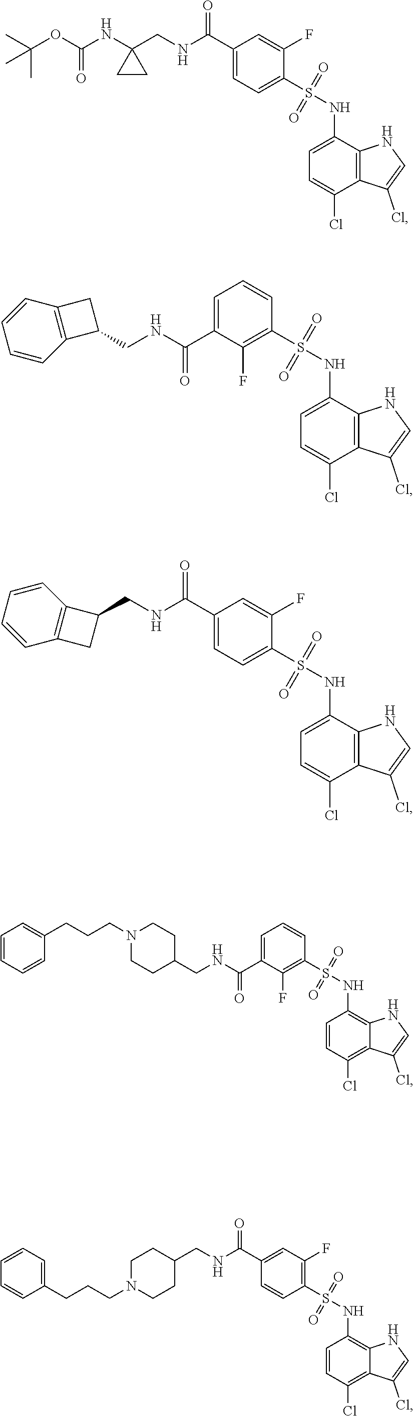 [SMILES: CC(C)(C)OC(=O)NC1(CNC(=O)c2ccc(S(=O)(=O)Nc3ccc(Cl)c4c(Cl)c[nH]c34)c(F)c2)CC1 ; O=C(NC[C@H]1Cc2ccccc21)c1cccc(S(=O)(=O)Nc2ccc(Cl)c3c(Cl)c[nH]c23)c1F ; O=C(NC[C@H]1Cc2ccccc21)c1ccc(S(=O)(=O)Nc2ccc(Cl)c3c(Cl)c[nH]c23)c(F)c1 ; O=C(NCC1CCN(CCCc2ccccc2)CC1)c1cccc(S(=O)(=O)Nc2ccc(Cl)c3c(Cl)c[nH]c23)c1F ; O=C(NCC1CCN(CCCc2ccccc2)CC1)c1ccc(S(=O)(=O)Nc2ccc(Cl)c3c(Cl)c[nH]c23)c(F)c1] 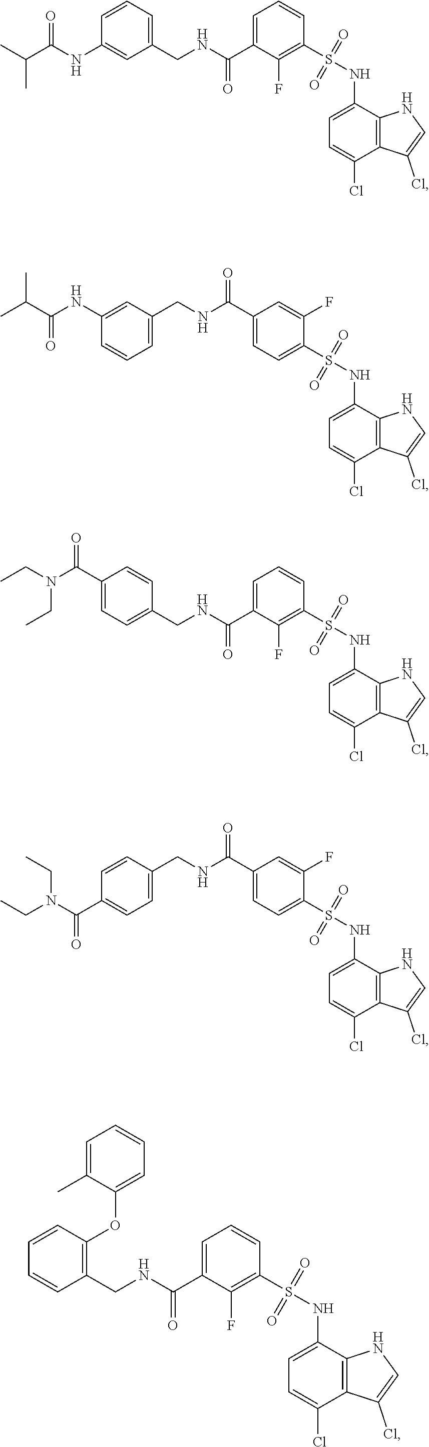 [SMILES: CC(C)C(=O)Nc1cccc(CNC(=O)c2cccc(S(=O)(=O)Nc3ccc(Cl)c4c(Cl)c[nH]c34)c2F)c1 ; CC(C)C(=O)Nc1cccc(CNC(=O)c2ccc(S(=O)(=O)Nc3ccc(Cl)c4c(Cl)c[nH]c34)c(F)c2)c1 ; CCN(CC)C(=O)c1ccc(CNC(=O)c2cccc(S(=O)(=O)Nc3ccc(Cl)c4c(Cl)c[nH]c34)c2F)cc1 ; CCN(CC)C(=O)c1ccc(CNC(=O)c2ccc(S(=O)(=O)Nc3ccc(Cl)c4c(Cl)c[nH]c34)c(F)c2)cc1 ; Cc1ccccc1Oc1ccccc1CNC(=O)c1cccc(S(=O)(=O)Nc2ccc(Cl)c3c(Cl)c[nH]c23)c1F] 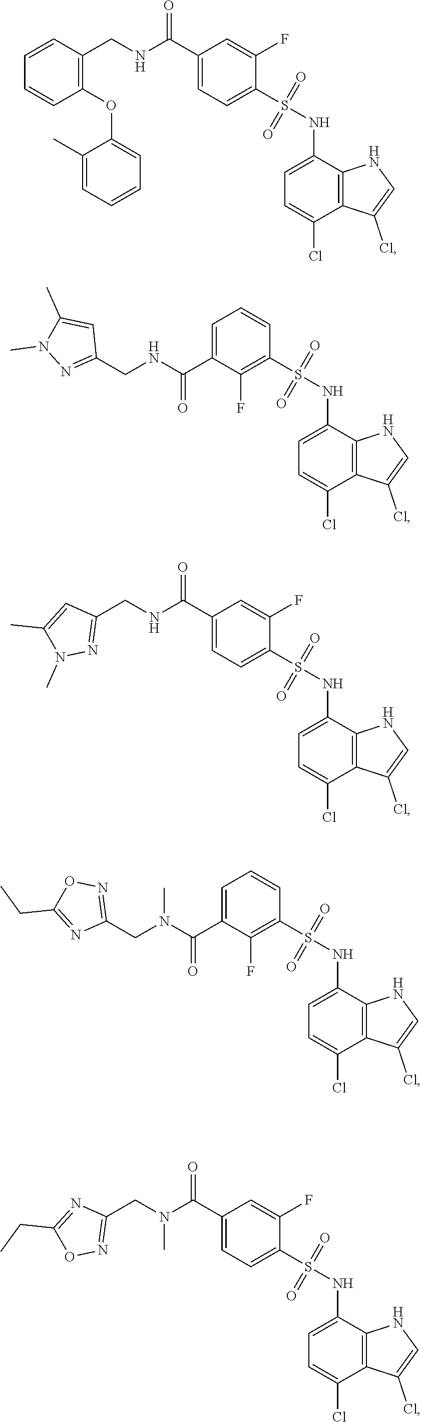 [SMILES: Cc1ccccc1Oc1ccccc1CNC(=O)c1ccc(S(=O)(=O)Nc2ccc(Cl)c3c(Cl)c[nH]c23)c(F)c1 ; Cc1cc(CNC(=O)c2cccc(S(=O)(=O)Nc3ccc(Cl)c4c(Cl)c[nH]c34)c2F)nn1C ; Cc1cc(CNC(=O)c2ccc(S(=O)(=O)Nc3ccc(Cl)c4c(Cl)c[nH]c34)c(F)c2)nn1C ; CCc1nc(CN(C)C(=O)c2cccc(S(=O)(=O)Nc3ccc(Cl)c4c(Cl)c[nH]c34)c2F)no1 ; CCc1nc(CN(C)C(=O)c2ccc(S(=O)(=O)Nc3ccc(Cl)c4c(Cl)c[nH]c34)c(F)c2)no1] 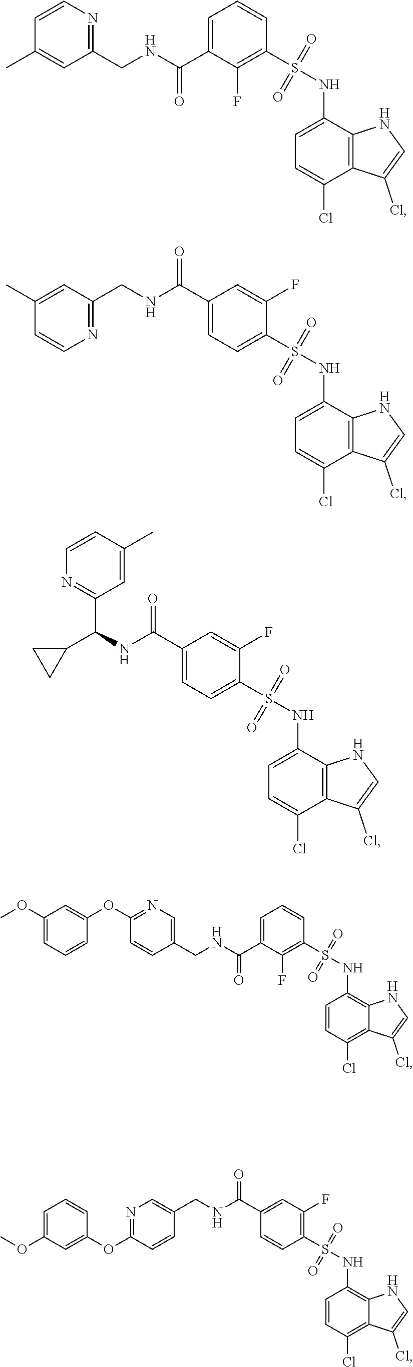 [SMILES: Cc1ccnc(CNC(=O)c2cccc(S(=O)(=O)Nc3ccc(Cl)c4c(Cl)c[nH]c34)c2F)c1 ; Cc1ccnc(CNC(=O)c2ccc(S(=O)(=O)Nc3ccc(Cl)c4c(Cl)c[nH]c34)c(F)c2)c1 ; Cc1ccnc([C@@H](NC(=O)c2ccc(S(=O)(=O)Nc3ccc(Cl)c4c(Cl)c[nH]c34)c(F)c2)C2CC2)c1 ; COc1cccc(Oc2ccc(CNC(=O)c3cccc(S(=O)(=O)Nc4ccc(Cl)c5c(Cl)c[nH]c45)c3F)cn2)c1 ; COc1cccc(Oc2ccc(CNC(=O)c3ccc(S(=O)(=O)Nc4ccc(Cl)c5c(Cl)c[nH]c45)c(F)c3)cn2)c1] 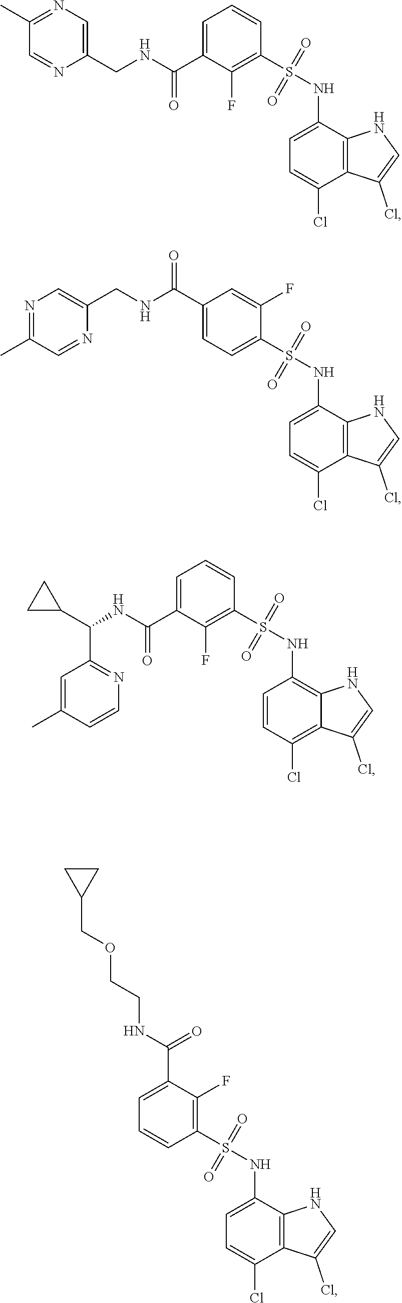 [SMILES: Cc1cnc(CNC(=O)c2cccc(S(=O)(=O)Nc3ccc(Cl)c4c(Cl)c[nH]c34)c2F)cn1 ; Cc1cnc(CNC(=O)c2ccc(S(=O)(=O)Nc3ccc(Cl)c4c(Cl)c[nH]c34)c(F)c2)cn1 ; Cc1ccnc([C@@H](NC(=O)c2cccc(S(=O)(=O)Nc3ccc(Cl)c4c(Cl)c[nH]c34)c2F)C2CC2)c1 ; O=C(NCCOCC1CC1)c1cccc(S(=O)(=O)Nc2ccc(Cl)c3c(Cl)c[nH]c23)c1F] 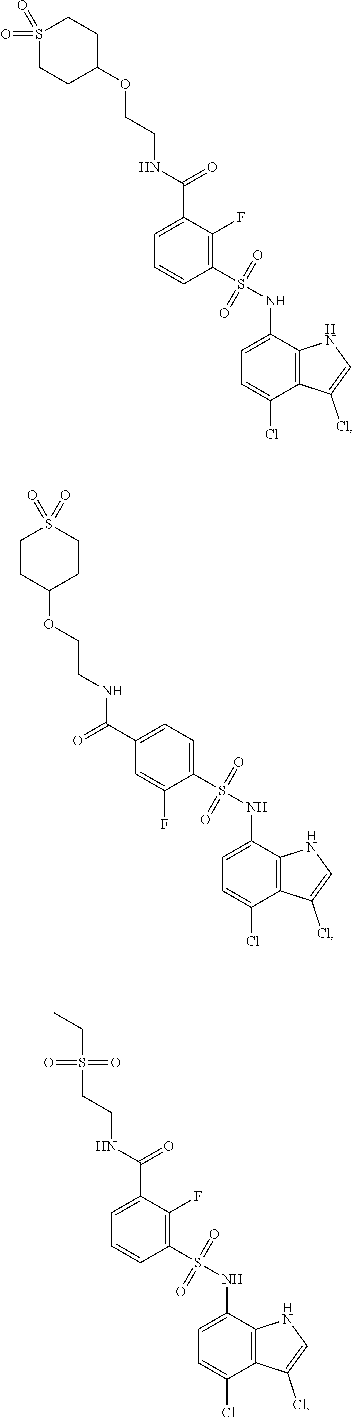 [SMILES: O=C(NCCOC1CCS(=O)(=O)CC1)c1cccc(S(=O)(=O)Nc2ccc(Cl)c3c(Cl)c[nH]c23)c1F ; O=C(NCCOC1CCS(=O)(=O)CC1)c1ccc(S(=O)(=O)Nc2ccc(Cl)c3c(Cl)c[nH]c23)c(F)c1 ; CCS(=O)(=O)CCNC(=O)c1cccc(S(=O)(=O)Nc2ccc(Cl)c3c(Cl)c[nH]c23)c1F] 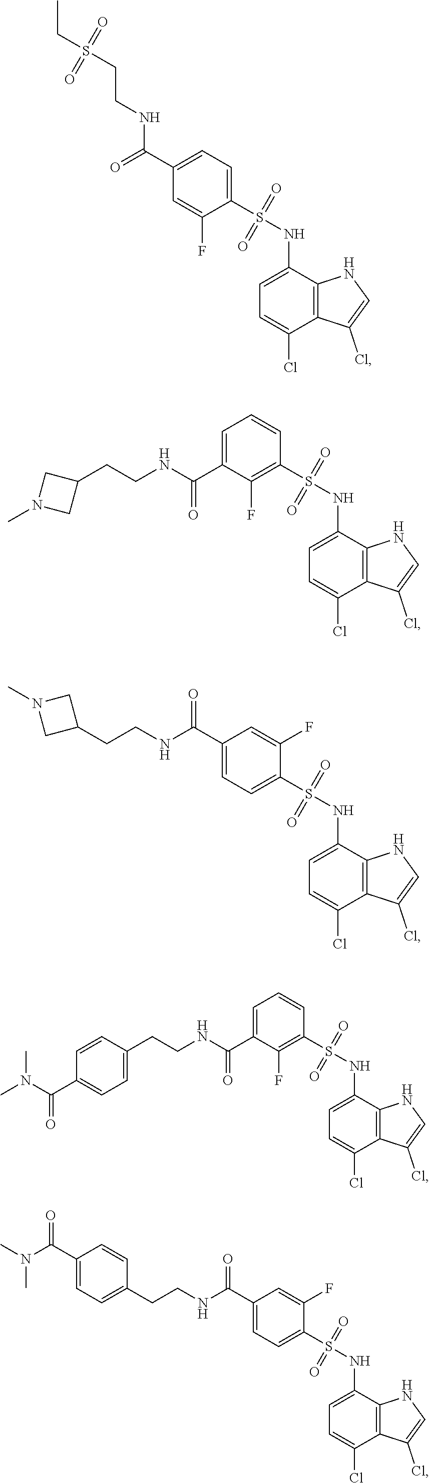 [SMILES: CCS(=O)(=O)CCNC(=O)c1ccc(S(=O)(=O)Nc2ccc(Cl)c3c(Cl)c[nH]c23)c(F)c1 ; CN1CC(CCNC(=O)c2cccc(S(=O)(=O)Nc3ccc(Cl)c4c(Cl)c[nH]c34)c2F)C1 ; CN1CC(CCNC(=O)c2ccc(S(=O)(=O)Nc3ccc(Cl)c4c(Cl)c[nH]c34)c(F)c2)C1 ; CN(C)C(=O)c1ccc(CCNC(=O)c2cccc(S(=O)(=O)Nc3ccc(Cl)c4c(Cl)c[nH]c34)c2F)cc1 ; CN(C)C(=O)c1ccc(CCNC(=O)c2ccc(S(=O)(=O)Nc3ccc(Cl)c4c(Cl)c[nH]c34)c(F)c2)cc1] 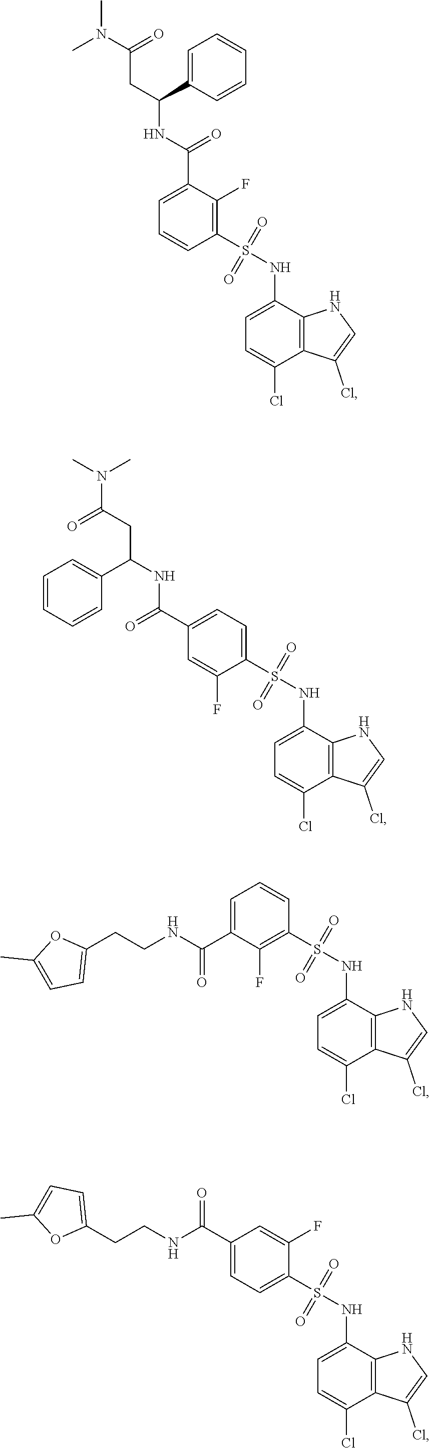 [SMILES: CN(C)C(=O)C[C@H](NC(=O)c1cccc(S(=O)(=O)Nc2ccc(Cl)c3c(Cl)c[nH]c23)c1F)c1ccccc1 ; CN(C)C(=O)CC(NC(=O)c1ccc(S(=O)(=O)Nc2ccc(Cl)c3c(Cl)c[nH]c23)c(F)c1)c1ccccc1 ; Cc1ccc(CCNC(=O)c2cccc(S(=O)(=O)Nc3ccc(Cl)c4c(Cl)c[nH]c34)c2F)o1 ; Cc1ccc(CCNC(=O)c2ccc(S(=O)(=O)Nc3ccc(Cl)c4c(Cl)c[nH]c34)c(F)c2)o1] 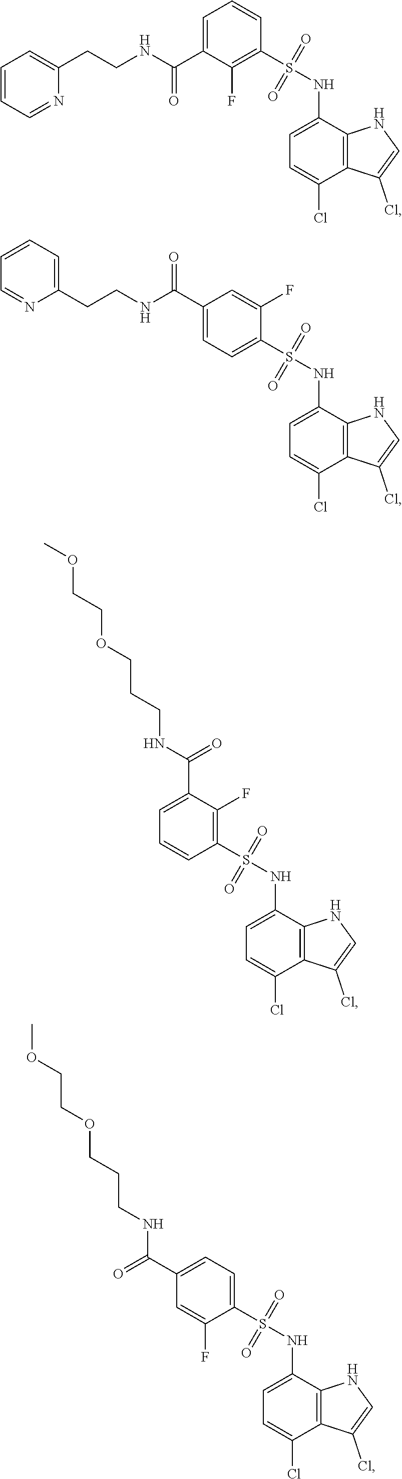 [SMILES: O=C(NCCc1ccccn1)c1cccc(S(=O)(=O)Nc2ccc(Cl)c3c(Cl)c[nH]c23)c1F ; O=C(NCCc1ccccn1)c1ccc(S(=O)(=O)Nc2ccc(Cl)c3c(Cl)c[nH]c23)c(F)c1 ; COCCOCCCNC(=O)c1cccc(S(=O)(=O)Nc2ccc(Cl)c3c(Cl)c[nH]c23)c1F ; COCCOCCCNC(=O)c1ccc(S(=O)(=O)Nc2ccc(Cl)c3c(Cl)c[nH]c23)c(F)c1] 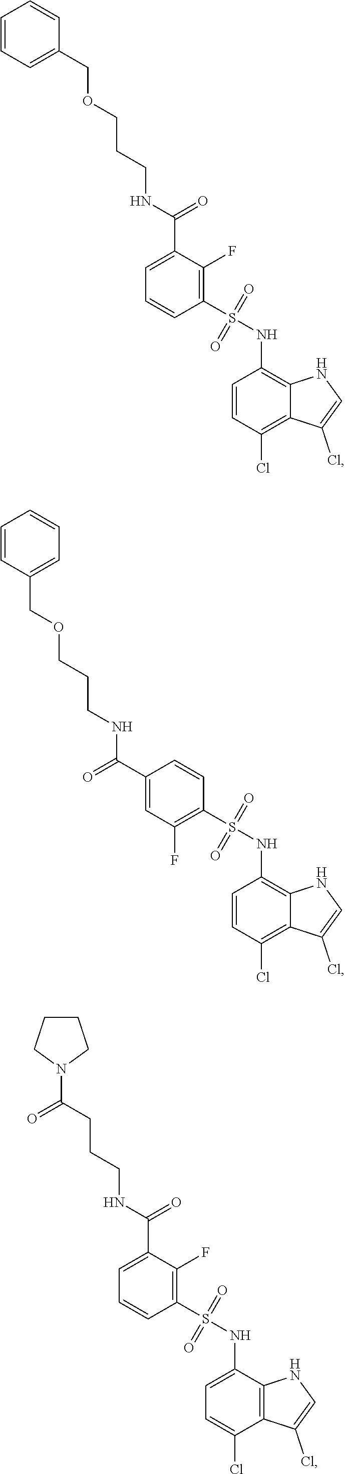 [SMILES: O=C(NCCCOCc1ccccc1)c1cccc(S(=O)(=O)Nc2ccc(Cl)c3c(Cl)c[nH]c23)c1F ; O=C(NCCCOCc1ccccc1)c1ccc(S(=O)(=O)Nc2ccc(Cl)c3c(Cl)c[nH]c23)c(F)c1 ; O=C(NCCCC(=O)N1CCCC1)c1cccc(S(=O)(=O)Nc2ccc(Cl)c3c(Cl)c[nH]c23)c1F] 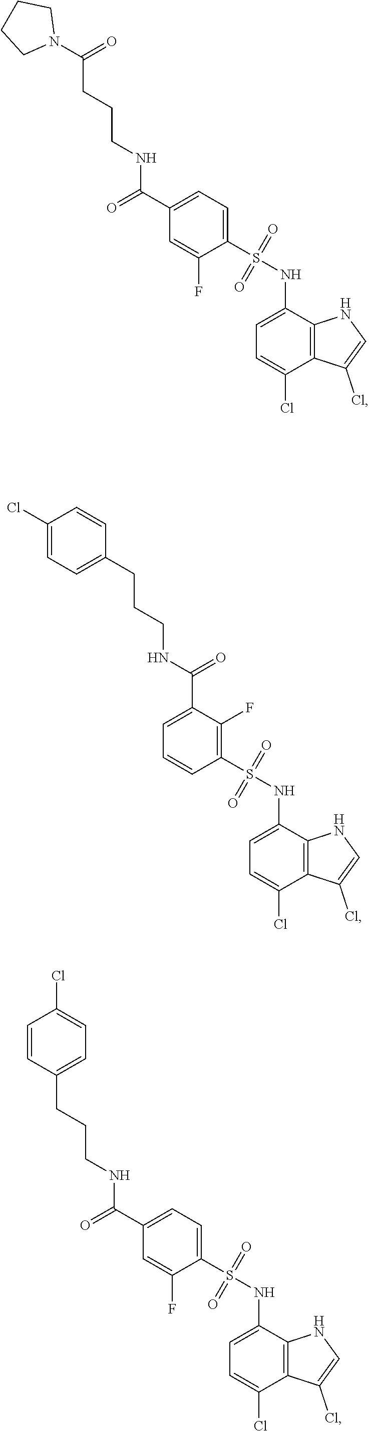 [SMILES: O=C(NCCCC(=O)N1CCCC1)c1ccc(S(=O)(=O)Nc2ccc(Cl)c3c(Cl)c[nH]c23)c(F)c1 ; O=C(NCCCc1ccc(Cl)cc1)c1cccc(S(=O)(=O)Nc2ccc(Cl)c3c(Cl)c[nH]c23)c1F ; O=C(NCCCc1ccc(Cl)cc1)c1ccc(S(=O)(=O)Nc2ccc(Cl)c3c(Cl)c[nH]c23)c(F)c1] 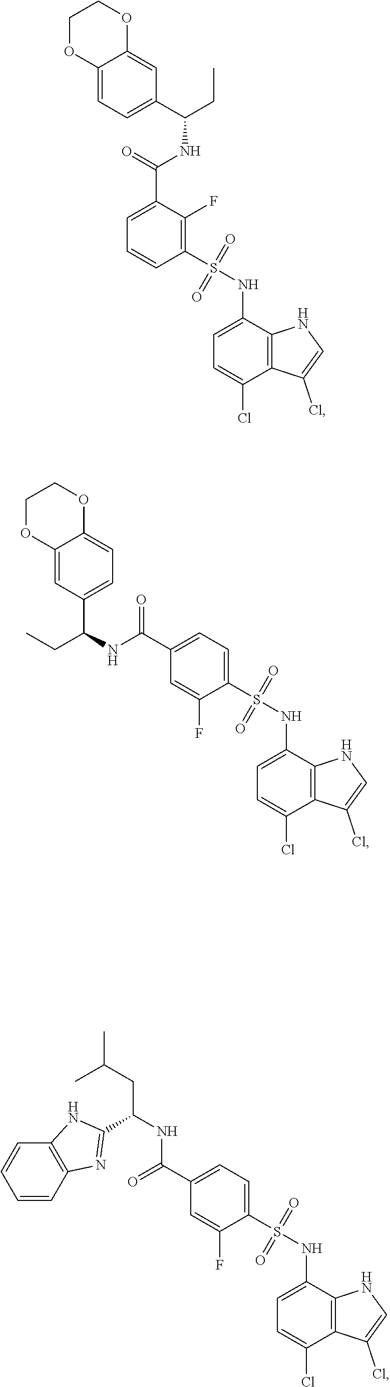 [SMILES: CC[C@H](NC(=O)c1cccc(S(=O)(=O)Nc2ccc(Cl)c3c(Cl)c[nH]c23)c1F)c1ccc2c(c1)OCCO2 ; CC[C@H](NC(=O)c1ccc(S(=O)(=O)Nc2ccc(Cl)c3c(Cl)c[nH]c23)c(F)c1)c1ccc2c(c1)OCCO2 ; CC(C)C[C@H](NC(=O)c1ccc(S(=O)(=O)Nc2ccc(Cl)c3c(Cl)c[nH]c23)c(F)c1)c1nc2ccccc2[nH]1] 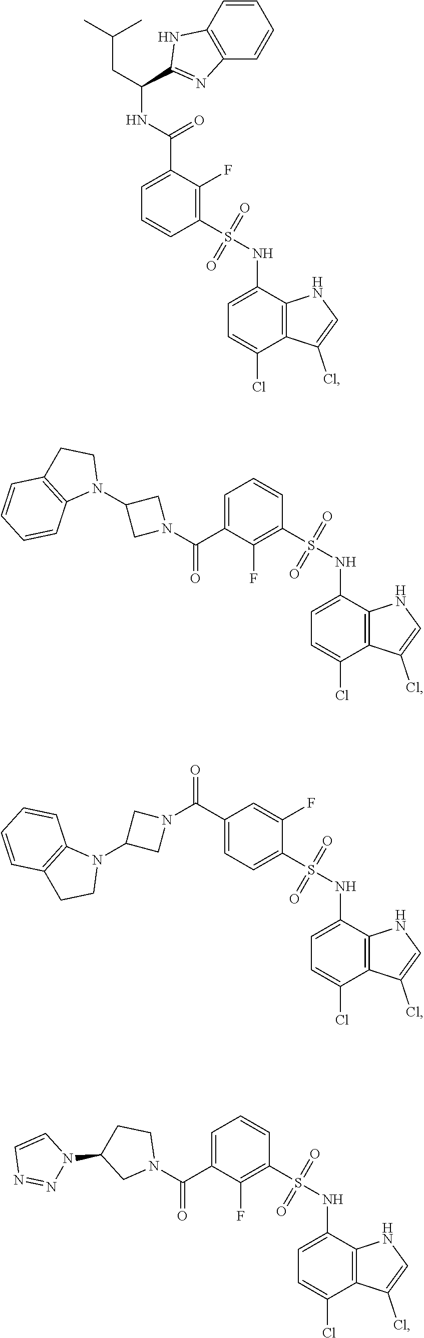 [SMILES: CC(C)C[C@H](NC(=O)c1cccc(S(=O)(=O)Nc2ccc(Cl)c3c(Cl)c[nH]c23)c1F)c1nc2ccccc2[nH]1 ; O=C(c1cccc(S(=O)(=O)Nc2ccc(Cl)c3c(Cl)c[nH]c23)c1F)N1CC(N2CCc3ccccc32)C1 ; O=C(c1ccc(S(=O)(=O)Nc2ccc(Cl)c3c(Cl)c[nH]c23)c(F)c1)N1CC(N2CCc3ccccc32)C1 ; O=C(c1cccc(S(=O)(=O)Nc2ccc(Cl)c3c(Cl)c[nH]c23)c1F)N1CC[C@H](n2ccnn2)C1] 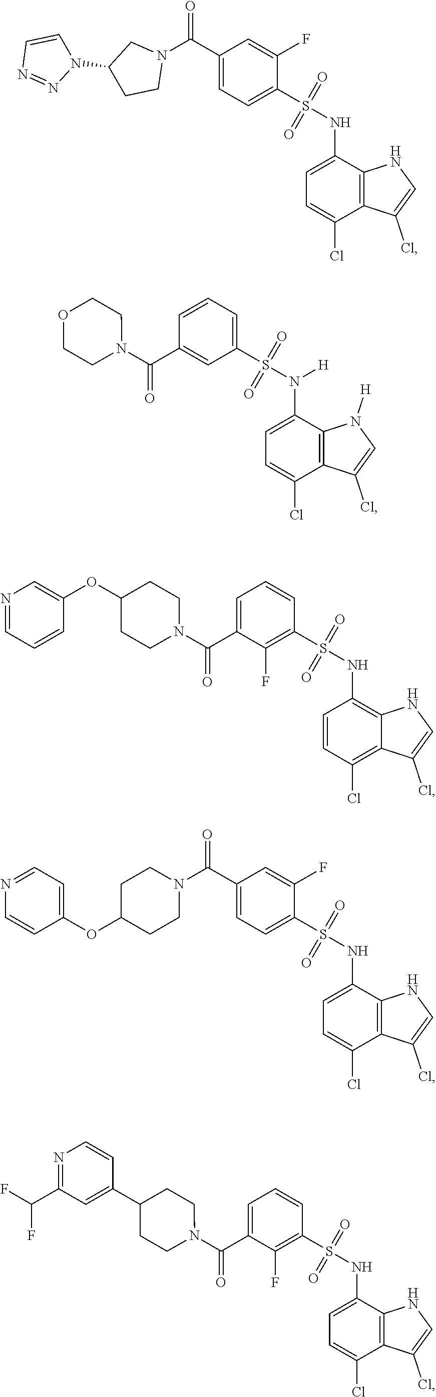 [SMILES: O=C(c1ccc(S(=O)(=O)Nc2ccc(Cl)c3c(Cl)c[nH]c23)c(F)c1)N1CC[C@H](n2ccnn2)C1 ; O=C(c1cccc(S(=O)(=O)Nc2ccc(Cl)c3c(Cl)c[nH]c23)c1)N1CCOCC1 ; O=C(c1cccc(S(=O)(=O)Nc2ccc(Cl)c3c(Cl)c[nH]c23)c1F)N1CCC(Oc2cccnc2)CC1 ; O=C(c1ccc(S(=O)(=O)Nc2ccc(Cl)c3c(Cl)c[nH]c23)c(F)c1)N1CCC(Oc2ccncc2)CC1 ; O=C(c1cccc(S(=O)(=O)Nc2ccc(Cl)c3c(Cl)c[nH]c23)c1F)N1CCC(c2ccnc(C(F)F)c2)CC1] 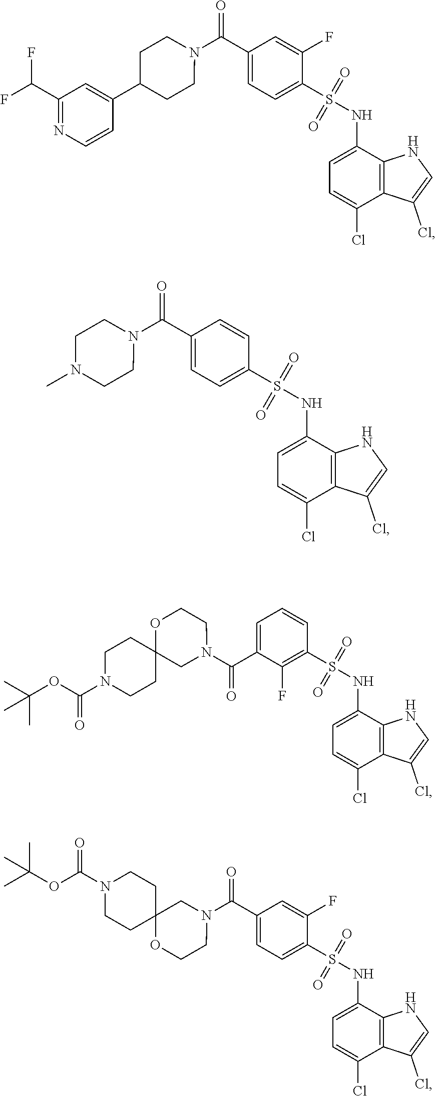 [SMILES: O=C(c1ccc(S(=O)(=O)Nc2ccc(Cl)c3c(Cl)c[nH]c23)c(F)c1)N1CCC(c2ccnc(C(F)F)c2)CC1 ; CN1CCN(C(=O)c2ccc(S(=O)(=O)Nc3ccc(Cl)c4c(Cl)c[nH]c34)cc2)CC1 ; CC(C)(C)OC(=O)N1CCC2(CC1)CN(C(=O)c1cccc(S(=O)(=O)Nc3ccc(Cl)c4c(Cl)c[nH]c34)c1F)CCO2 ; CC(C)(C)OC(=O)N1CCC2(CC1)CN(C(=O)c1ccc(S(=O)(=O)Nc3ccc(Cl)c4c(Cl)c[nH]c34)c(F)c1)CCO2] 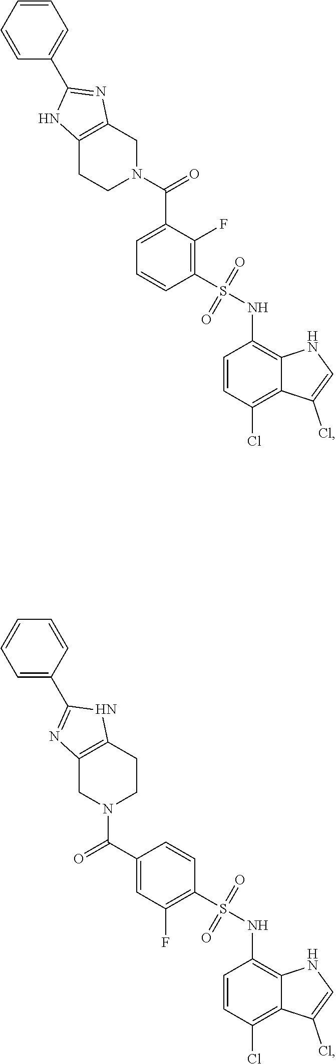 [SMILES: O=C(c1cccc(S(=O)(=O)Nc2ccc(Cl)c3c(Cl)c[nH]c23)c1F)N1CCc2[nH]c(-c3ccccc3)nc2C1 ; O=C(c1ccc(S(=O)(=O)Nc2ccc(Cl)c3c(Cl)c[nH]c23)c(F)c1)N1CCc2[nH]c(-c3ccccc3)nc2C1] 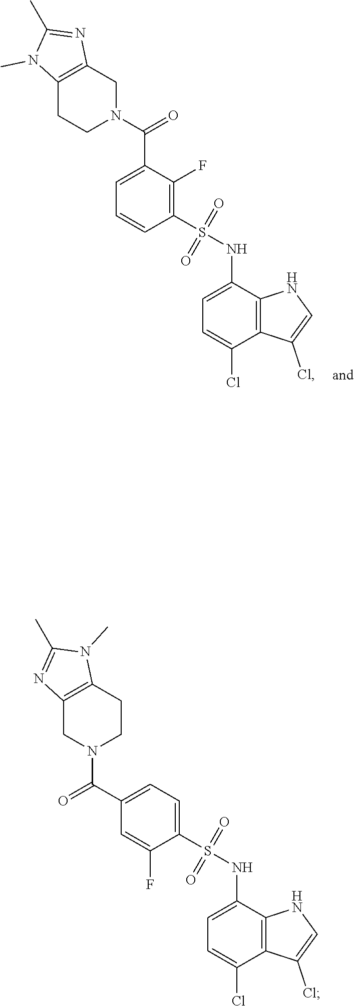 [SMILES: Cc1nc2c(n1C)CCN(C(=O)c1cccc(S(=O)(=O)Nc3ccc(Cl)c4c(Cl)c[nH]c34)c1F)C2 ; Cc1nc2c(n1C)CCN(C(=O)c1ccc(S(=O)(=O)Nc3ccc(Cl)c4c(Cl)c[nH]c34)c(F)c1)C2]
or a pharmaceutically acceptable salt thereof.

12. A compound selected from the group consisting of 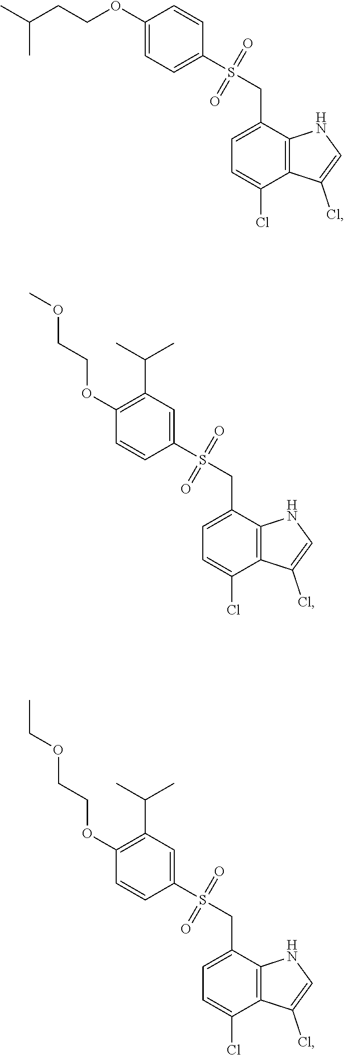 [SMILES: CC(C)CCOc1ccc(S(=O)(=O)Cc2ccc(Cl)c3c(Cl)c[nH]c23)cc1 ; COCCOc1ccc(S(=O)(=O)Cc2ccc(Cl)c3c(Cl)c[nH]c23)cc1C(C)C ; CCOCCOc1ccc(S(=O)(=O)Cc2ccc(Cl)c3c(Cl)c[nH]c23)cc1C(C)C] 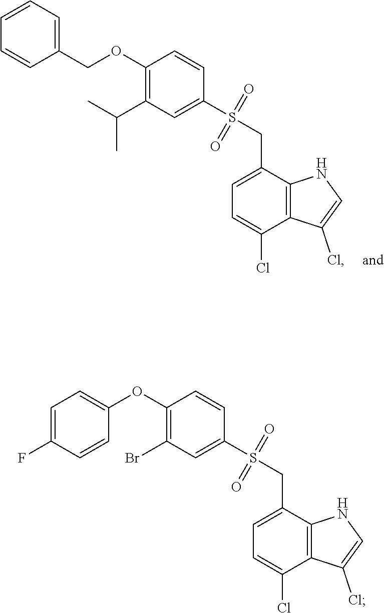 [SMILES: CC(C)c1cc(S(=O)(=O)Cc2ccc(Cl)c3c(Cl)c[nH]c23)ccc1OCc1ccccc1 ; O=S(=O)(Cc1ccc(Cl)c2c(Cl)c[nH]c12)c1ccc(Oc2ccc(F)cc2)c(Br)c1]
or a pharmaceutically acceptable salt thereof.

13. A compound selected from the group consisting of 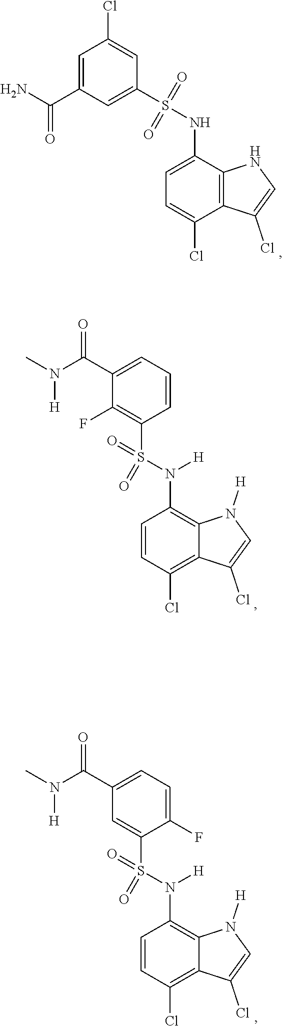 [SMILES: NC(=O)c1cc(Cl)cc(S(=O)(=O)Nc2ccc(Cl)c3c(Cl)c[nH]c23)c1 ; CNC(=O)c1cccc(S(=O)(=O)Nc2ccc(Cl)c3c(Cl)c[nH]c23)c1F ; CNC(=O)c1ccc(F)c(S(=O)(=O)Nc2ccc(Cl)c3c(Cl)c[nH]c23)c1] 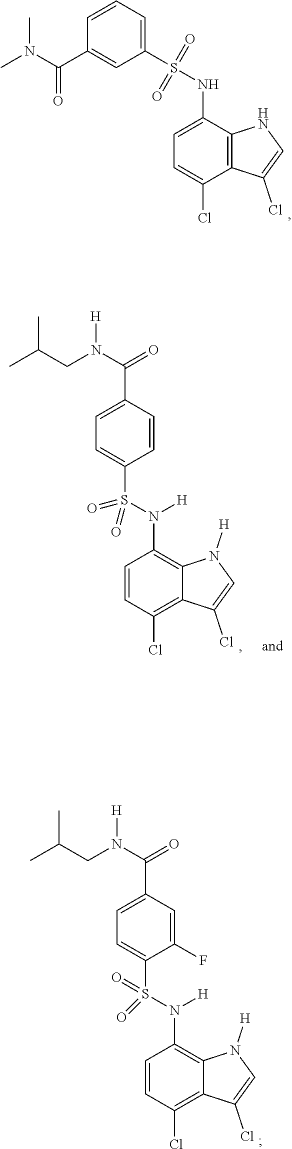 [SMILES: CN(C)C(=O)c1cccc(S(=O)(=O)Nc2ccc(Cl)c3c(Cl)c[nH]c23)c1 ; CC(C)CNC(=O)c1ccc(S(=O)(=O)Nc2ccc(Cl)c3c(Cl)c[nH]c23)cc1 ; CC(C)CNC(=O)c1ccc(S(=O)(=O)Nc2ccc(Cl)c3c(Cl)c[nH]c23)c(F)c1]
or a pharmaceutically acceptable salt thereof.

14. (canceled)

15. A pharmaceutical composition comprising a therapeutically effective amount of a compound of claim 1, or a pharmaceutically acceptable salt thereof.

16-22. (canceled)

23. A method of treating a disease or disorder that results from the function of a target protein in a subject, comprising administering to the subject, a therapeutically effective amount of a compound according to claim 1, or a pharmaceutically acceptable salt thereof, wherein the compound, salt, or composition induces degradation of the target protein thereby treating the disease or disorder.

24-36. (canceled)
